SEQUENCE LISTING (128 sequences)

SEQ ID NO: 1 - protein, 976 aa, Homo sapiens [sequence omitted]

SEQ ID NO: 2 - protein, 976 aa, Macaca fascicularis
MRGARGAWDFLCVLLFLLHVQTGSSQPSVSPGEPSPPSIHPAKSELIVRVGNEIRLLCIDPGFVKWTFEILDETNENKQNEWITEKAEATNTGKYTCTNKHGLSSSIYVFVRDPAKLFLVDRSLYGKEDNDTLVRCPLTDPEVTSYSLKGCQGKPLPKDLRFVPDPKAGITIKSVKRAYHRLCLHCSADQEGKSVLSDKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNASIAFNVYVNTKPEILTYDRLVNGMLQCVAAGFPEPTIDWYFCPGTEQRCSASVLPVDVQTLNASGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAESALYKNLLHSKESSCSDSTNEYMDMKPGVSYVVPTKADKRRSARIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - protein, 979 aa, Mus musculus
MRGARGAWDLLCVLLVLLRGQTATSQPSASPGEPSPPSIHPAQSELIVEAGDTLSLTCIDPDFVRWTFKTYFNEMVENKKNEWIQEKAEATRTGTYTCSNSNGLTSSIYVFVRDPAKLFLVGLPLFGKEDSDALVRCPLTDPQVSNYSLIECDGKSLPTDLTFVPNPKAGITIKNVKRAYHRLCVRCAAQRDGTWLHSDKFTLKVRAAIKAIPVVSVPETSHLLKKGDTFTVVCTIKDVSTSVNSMWLKMNPQPQHIAQVKHNSWHRGDFNYERQETLTISSARVDDSGVFMCYANNTFGSANVTTTLKVVEKGFINISPVKNTTVFVTDGENVDLVVEYEAYPKPEHQQWIYMNRTSANKGKDYVKSDNKSNIRYVNQLRLTRLKGTEGGTYTFLVSNSDASASVTFNVYVNTKPEILTYDRLINGMLQCVAEGFPEPTIDWYFCTGAEQRCTTPVSPVDVQVQNVSVSPFGKLVVQSSIDSSVFRHNGTVECKASNDVGKSSAFFNFAFKGNNKEQIQAHTLFTPLLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKGMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 4 - protein, 121 aa, synthetic construct
  VARIANT 5: note = NOTE = X is A or V or L
  VARIANT 10: note = NOTE = X is D or G
  VARIANT 11: note = NOTE = X is L or V
  VARIANT 16: note = NOTE = X is G or R
  VARIANT 31: note = NOTE = X is S or D
  VARIANT 33: note = NOTE = X is A or G
  VARIANT 35: note = NOTE = X is S or H
  VARIANT 50: note = NOTE = X is A or V
  VARIANT 52: note = NOTE = X is S or G or absent
  VARIANT 53: note = NOTE = X is G or Y
  VARIANT 54: note = NOTE = X is S or G
  VARIANT 57: note = NOTE = X is S or G
  VARIANT 58: note = NOTE = X is T or K
  VARIANT 93: note = NOTE = X is I or V
  VARIANT 98: note = NOTE = X is R or K
  VARIANT 100: note = NOTE = X is L or S or T
  VARIANT 101: note = NOTE = X is R or S or W
  VARIANT 102: note = NOTE = X is V or G or W
  VARIANT 103: note = NOTE = X is L or Y
  VARIANT 104: note = NOTE = X is A or R
  VARIANT 105: note = NOTE = X is M or G
  VARIANT 106: note = NOTE = X is E or D or L
  VARIANT 107: note = NOTE = X is A or absent
  VARIANT 110: note = NOTE = X is I or Y
  VARIANT 116: note = NOTE = X is M or L
EVQLXESGGXXVQPGXSLRLSCAASGFTFSXYXMXWVRQAPGKGLEWVSXIXXXGGXXYYADSVKGRFTISRDNSKNTLYLQMNSLRAEDTAXYYCAXDXXXXXXXXFDXWGQGTXVTVSS

SEQ ID NO: 5 - protein, 105 aa, synthetic construct
  VARIANT 7: note = NOTE = X is D or P
  VARIANT 9: note = NOTE = X is A or S
  VARIANT 13: note = NOTE = X is A or S
  VARIANT 14: note = NOTE = X is L or P
  VARIANT 18: note = NOTE = X is V or A
  VARIANT 19: note = NOTE = X is R or T or S
  VARIANT 23: note = NOTE = X is Q or S
  VARIANT 26: note = NOTE = X is S or K
  VARIANT 27: note = NOTE = X is L or V
  VARIANT 28: note = NOTE = X is R or G
  VARIANT 29: note = NOTE = X is D or E or A
  VARIANT 30: note = NOTE = X is Y or T or K
  VARIANT 32: note = NOTE = X is A or V
  VARIANT 33: note = NOTE = X is N or H or F
  VARIANT 35: note = NOTE = X is F or Y
  VARIANT 37: note = NOTE = X is Q or H
  VARIANT 38: note = NOTE = X is K or Q
  VARIANT 39: note = NOTE = X is P or S
  VARIANT 42: note = NOTE = X is A or S
  VARIANT 44: note = NOTE = X is I or L or V
  VARIANT 47: note = NOTE = X is I or V
  VARIANT 48: note = NOTE = X is F or Y
  VARIANT 49: note = NOTE = X is Y or Q
  VARIANT 51: note = NOTE = X is S or F or N
  VARIANT 52: note = NOTE = X is D or K
  VARIANT 76: note = NOTE = X is R or G
  VARIANT 77: note = NOTE = X is V or T
  VARIANT 78: note = NOTE = X is E or Q
  VARIANT 80: note = NOTE = X is G or M
  VARIANT 94: note = NOTE = X is S or T
  VARIANT 95: note = NOTE = X is D or A
  VARIANT 96: note = NOTE = X is H or V or L
  VARIANT 97: note = NOTE = X is W or Y or absent
  VARIANT 98: note = NOTE = X is V or absent
  VARIANT 101: note = NOTE = X is G or T
  VARIANT 105: note = NOTE = X is L or V
SYELTQXPXVSVXXGQTXXITCXGDXXXXXYXXWXQXXXGQXPXLVXXXDXXRPSGIPERFSGSNSGNTATLTISXXXAXDEADYYCQAWDSSXXXXXFGXGTKX

SEQ ID NO: 6 - protein, 467 aa, synthetic construct
  SIGNAL 1..19
MDWTWRILFLVAAAQPALAEVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 121 aa, synthetic construct
EVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSS

SEQ ID NO: 8 - DNA, 1404 bp, synthetic construct
  source 1..1404: mol_type = other DNA
atggactggacctggcgcatcctgtttctggtggccgctgcccaaccagcgctggccgaagtgcagctggcggagtccgggggagacttagttcagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccatatattactgtgcgagagacctacgggtgtatgctatggaggcttttgatatctggggccaagggacaatggtcaccgtctcctcagcttccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaagtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 9 - protein, 233 aa, synthetic construct
  SIGNAL 1..19
MDWTWRILFLVAFATAVQASYELTQDPAVSVALGQTVRITCQGDSLRDYYANWFQQKPGQAPLLVIFYDSDRPSGIPERFSGSNSGNTATLTISRVEAGDEADYYCQAWDSSSDHWVFGGGTKLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 10 - protein, 105 aa, synthetic construct
SYELTQDPAVSVALGQTVRITCQGDSLRDYYANWFQQKPGQAPLLVIFYDSDRPSGIPERFSGSNSGNTATLTISRVEAGDEADYYCQAWDSSSDHWVFGGGTKL

SEQ ID NO: 11 - DNA, 699 bp, synthetic construct
  source 1..699: mol_type = other DNA
  intron 47..128
atggactggacctggcgcatcctgtttctggtggccttcgccacggccgtgcaggcatcctatgagctgactcaggaccctgctgtgtctgtggccctgggacagacagtcaggatcacatgccaaggggacagcctcagagactattatgcaaactggttccagcagaagccaggccaggcccctttattggtcatcttttatgatagcgaccggccctcagggatccctgagcgattctctggctccaactctgggaatacggccaccctgaccatcagcagggtcgaagccggggatgaggccgactattattgtcaggcgtgggatagtagtagtgaccattgggtgttcggcggagggaccaaactgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 12 - protein, 471 aa, synthetic construct
  SIGNAL 1..19
MDWTWRILFLVAAAQPALAQVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARVGVRYFDWLLYGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 125 aa, synthetic construct
QVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARVGVRYFDWLLYGAFDIWGQGTMVTVSS

SEQ ID NO: 14 - DNA, 1416 bp, synthetic construct
  source 1..1416: mol_type = other DNA
atggactggacctggcgcatcctgtttctggtggccgctgcccaaccagcgctggcccaggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagagtgggggttcgatattttgactggttattatatggcgcttttgatatctggggccaagggacaatggtcactgtctcctcagcttccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaagtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 15 - protein, 231 aa, synthetic construct
  SIGNAL 1..19
MDWTWRILFLVAFATAVQASYELTQPPSVSVSPGQTATITCSGDKLGETYVHWYQHQSGQSPILVVYQDFKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 16 - protein, 103 aa, synthetic construct
SYELTQPPSVSVSPGQTATITCSGDKLGETYVHWYQHQSGQSPILVVYQDFKRPSGIPERFSGSNSGNTATLTISGTQAMDEADYYCQAWDSSTAVFGGGTKL

SEQ ID NO: 17 - DNA, 693 bp, synthetic construct
  source 1..693: mol_type = other DNA
  intron 47..128
atggactggacctggcgcatcctgtttctggtggccttcgccacggccgtgcaggcatcctatgagctgactcagccaccctcagtgtccgtgtcgcctggacagacagccaccatcacctgctctggagataagttgggggaaacttatgttcattggtatcagcatcagtcaggccagtcccctattttagtcgtctatcaagatttcaagcggccctccgggatccctgagcgattctctggctccaactctgggaacacagccactctgaccatcagcgggacccaggctatggatgaggctgactattactgtcaggcgtgggacagcagcactgcggtattcggcggagggaccaagctgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 18 - protein, 465 aa, synthetic construct
  SIGNAL 1..19
MDWTWRILFLVAAAQPALAEVQLVESGGGVVQPGRSLRLSCAASGFTFSSYAMHWVRQAPGKGLEWVSVIYSGGSTYYADSVKGRFTISRDNSKNTLYLQMNSLRAEDTAVYYCARDSSGYRGDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 119 aa, synthetic construct
EVQLVESGGGVVQPGRSLRLSCAASGFTFSSYAMHWVRQAPGKGLEWVSVIYSGGSTYYADSVKGRFTISRDNSKNTLYLQMNSLRAEDTAVYYCARDSSGYRGDFDYWGQGTLVTVSS

SEQ ID NO: 20 - DNA, 1398 bp, synthetic construct
  source 1..1398: mol_type = other DNA
atggactggacctggcgcatcctgtttctggtggccgctgcccaaccagcgctggccgaagtgcagctggtggagtctgggggaggcgtggtccagcctgggaggtccctgagactctcctgtgcagcctctggattcaccttcagtagctatgctatgcactgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagatagtagtggttatcgaggcgactttgactactggggccagggaaccctggtcaccgtctcctcagcttccaccaagggcccatcggtcttcccgctagcgccctgctccaggagcacctccgagagcacagccgccctgggctgcctggtcaaggactacttccccgaaccagtgacggtgtcgtggaactcaggcgccctgaccagcggcgtccacaccttcccggctgtcctacagtcctcaggactctactccctcagcagcgtagtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccacgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagtctctccctgtctctgggtaaatga

SEQ ID NO: 21 - protein, 233 aa, synthetic construct
  SIGNAL 1..19
MDWTWRILFLVAFATAVQASYELTQPPSVSVSPGQTASITCSGDKVGAKYVFWYQQKPGQSPVLVVYQDNKRPSGIPERFSGSNSGNTATLTISGTQAMDEADYYCQAWDSSTALYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 22 - protein, 105 aa, synthetic construct
SYELTQPPSVSVSPGQTASITCSGDKVGAKYVFWYQQKPGQSPVLVVYQDNKRPSGIPERFSGSNSGNTATLTISGTQAMDEADYYCQAWDSSTALYVFGTGTKV

SEQ ID NO: 23 - DNA, 702 bp, synthetic construct
  source 1..702: mol_type = other DNA
  intron 47..128
atggactggacctggcgcatcctgtttctggtggccttcgccacggccgtgcaggcctcctatgagctgactcagccaccctcagtgtccgtgtccccaggacagacagccagcatcacctgctctggagataaggtgggggctaaatatgttttctggtatcagcagaagccaggccagtcccctgtgctggtcgtctatcaagataacaagcggccctcagggatccctgagcgattctctggctccaactctgggaacacagccacactgaccatcagcgggacccaggctatggatgaggctgactattactgtcaggcgtgggacagcagcactgccctttatgtcttcggaactgggaccaaggtcaccgtcctaggtcagcccaaggccaaccccactgtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 24 - protein, 466 aa, synthetic construct
  SIGNAL 1..19
MDWTWRILFLVAAAQPALAEVQLLESGGGLVQPGGSLRLSCAASGFTFSDYGMSWVRQAPGKGLEWVSAIGYGGGGKYYADSVKGRFTISRDNSKNTLYLQMNSLRAEDTAVYYCAKDTWWLAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 120 aa, synthetic construct
EVQLLESGGGLVQPGGSLRLSCAASGFTFSDYGMSWVRQAPGKGLEWVSAIGYGGGGKYYADSVKGRFTISRDNSKNTLYLQMNSLRAEDTAVYYCAKDTWWLAGLFDYWGQGTLVTVSS

SEQ ID NO: 26 - DNA, 1401 bp, synthetic construct
  source 1..1401: mol_type = other DNA
atggactggacctggcgcatcctgtttctggtggccgctgcccaaccagcgctggccgaggtgcagctgttggagtctgggggaggcttggtacagcctggggggtccctgagactctcctgtgcagcctctggattcacctttagcgattacgggatgagctgggtccgccaggctccagggaaggggctggagtgggtatcagccattgggtatggtggaggtggtaaatactacgcagactccgtgaagggccggttcaccatctccagagacaattccaagaacacgctgtatctgcaaatgaacagcctgagagccgaggacacggccgtgtattactgtgctaaggatacttggtggctggcgggtctctttgactactggggccagggaaccctggtcaccgtctcctcagcttccaccaagggcccatcggtcttcccgctagcgccctgctccaggagcacctccgagagcacagccgccctgggctgcctggtcaaggactacttccccgaaccagtgacggtgtcgtggaactcaggcgccctgaccagcggcgtccacaccttcccggctgtcctacagtcctcaggactctactccctcagcagcgtagtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccacgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagtctctccctgtctctgggtaaatga

SEQ ID NO: 27 - protein, 233 aa, synthetic construct
  SIGNAL 1..18
MGWSCIILFLVATATVQAEIVLTQSPGTLSLSPGKRATLSCRASQSVSNSYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 109 aa, synthetic construct
EIVLTQSPGTLSLSPGKRATLSCRASQSVSNSYLAWYQQKPGQPPKLLIYWASTRESGVPDRFSGSGSGTDFTLTISSLQAEDVAVYYCQQYYSTPLTFGGGTKVEIKR

SEQ ID NO: 29 - DNA, 705 bp, synthetic construct
  source 1..705: mol_type = other DNA
  intron 47..128
atggactggacctggcgcatcctgtttctggtggccttcgccacggccgtgcaggccgaaattgtgttgacacagtcgccaggcaccctgtctttgtctccagggaaaagagccaccctctcctgcagggccagtcagagtgttagcaacagctacttagcctggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttag

SEQ ID NO: 30 - protein, 330 aa, synthetic construct [sequence omitted]

SEQ ID NO: 31 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 33 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 34 - protein, 338 aa, synthetic construct
ASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 35 - protein, 109 aa, synthetic construct
TVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 36 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 19 aa, synthetic construct
MDWTWRILFLVAAAQPALA

SEQ ID NO: 38 - protein, 19 aa, synthetic construct
MDWTWRILFLVAFATAVQA

SEQ ID NO: 39 - protein, 19 aa, synthetic construct
MGWSCIILFLVATATGVHA

SEQ ID NO: 40 - protein, 18 aa, synthetic construct
MGWSCIILFLVATATVQA

SEQ ID NO: 41 - DNA, 82 bp, synthetic construct
  source 1..82: mol_type = other DNA
gtaaggggttaacagtagcaggcttgaggtctggacatatatatgggtgacaatgacatccactttgcctttctctccacag

SEQ ID NO: 42 - protein, 5 aa, synthetic construct
SYAMS

SEQ ID NO: 43 - protein, 17 aa, synthetic construct
AISGSGGSTYYADSVKG

SEQ ID NO: 44 - protein, 12 aa, synthetic construct
DLRVYAMEAFDI

SEQ ID NO: 45 - protein, 11 aa, synthetic construct
QGDSLRDYYAN

SEQ ID NO: 46 - protein, 7 aa, synthetic construct
YDSDRPS

SEQ ID NO: 47 - protein, 11 aa, synthetic construct
QAWDSSSDHWV

SEQ ID NO: 48 - protein, 6 aa, synthetic construct
SGYYWG

SEQ ID NO: 49 - protein, 16 aa, synthetic construct
SIYHSGSTYYNPSLKS

SEQ ID NO: 50 - protein, 16 aa, synthetic construct
VGVRYFDWLLYGAFDI

SEQ ID NO: 51 - protein, 11 aa, synthetic construct
SGDKLGETYVH

SEQ ID NO: 52 - protein, 7 aa, synthetic construct
QDFKRPS

SEQ ID NO: 53 - protein, 9 aa, synthetic construct
QAWDSSTAV

SEQ ID NO: 54 - protein, 5 aa, synthetic construct
SYAMH

SEQ ID NO: 55 - protein, 16 aa, synthetic construct
VIYSGGSTYYADSVKG

SEQ ID NO: 56 - protein, 11 aa, synthetic construct
DSSGYRGDFDY

SEQ ID NO: 57 - protein, 11 aa, synthetic construct
SGDKVGAKYVF

SEQ ID NO: 58 - protein, 7 aa, synthetic construct
QDNKRPS

SEQ ID NO: 59 - protein, 11 aa, synthetic construct
QAWDSSTALYV

SEQ ID NO: 60 - protein, 5 aa, synthetic construct
DYGMS

SEQ ID NO: 61 - protein, 17 aa, synthetic construct
AIGYGGGGKYYADSVKG

SEQ ID NO: 62 - protein, 11 aa, synthetic construct
DTWWLAGLFDY

SEQ ID NO: 63 - protein, 12 aa, synthetic construct
RASQSVSNSYLA

SEQ ID NO: 64 - protein, 7 aa, synthetic construct
WASTRES

SEQ ID NO: 65 - protein, 9 aa, synthetic construct
QQYYSTPLT

SEQ ID NO: 66 - protein, 340 aa, synthetic construct
  VARIANT 47: note = NOTE = X is N or G
  VARIANT 102: note = NOTE = X is P or R
  VARIANT 179: note = NOTE = X is C or S
  VARIANT 205: note = NOTE = X is N or T
  VARIANT 206: note = NOTE = X is I or L
  VARIANT 207: note = NOTE = X is T or S
  VARIANT 339: note = NOTE = X is C or absent
  VARIANT 340: note = NOTE = X is Y or absent
ASPTSPKVFPLSLDSTPQDGNVVVACLVQGFFPQEPLSVTWSESGQXVTARNFPPSQDASGDLYTTSSQLTLPATQCPDGKSVTCHVKHYTNSSQDVTVPCXVPPPPPCCHPRLSLHRPALEDLLLGSEANLTCTLTGLRDASGATFTWTPSSGKSAVQGPPERDLCGCYSVSSVLPGXAQPWNHGETFTCTAAHPELKTPLTAXXXKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTXX

SEQ ID NO: 67 - protein, 470 aa, synthetic construct
  SIGNAL 1..19
MDWTWRILFLVAAAQPALAEVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - DNA, 1414 bp, synthetic construct
  source 1..1414: mol_type = other DNA
atggactggacctggcgcatcctgtttctggtggccgctgcccaaccagcgctggccgaagtgcagctggcggagtccgggggagacttagttcagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccatatattactgtgcgagagacctacgggtgtatgctatggaggcttttgatatctggggccaagggacaatggtcaccgtctcctcagcttccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaa

SEQ ID NO: 69 - protein, 233 aa, synthetic construct; identical to SEQ ID NO: 9
  SIGNAL 1..19

SEQ ID NO: 70 - DNA, 699 bp, synthetic construct
  source 1..699: mol_type = other DNA
atggactggacctggcgcatcctgtttctggtggccttcgccacggccgtgcaggcctcctatgagctgactcaggaccctgctgtgtctgtggccctgggacagacagtcaggatcacatgccaaggggacagcctcagagactattatgcaaactggttccagcagaagccaggccaggcccctttattggtcatcttttatgatagcgaccggccctcagggatccctgagcgattctctggctccaactctgggaatacggccaccctgaccatcagcagggtcgaagccggggatgaggccgactattattgtcaggcgtgggatagtagtagtgaccattgggtgttcggcggagggaccaaactgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 71 - protein, 480 aa, synthetic construct
  SIGNAL 1..19
MDWTWRILFLVAAAQPALAEVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 72 - DNA, 1446 bp, synthetic construct
  source 1..1446: mol_type = other DNA
atggactggacctggcgcatcctgtttctggtggccgctgcccaaccagcgctggccgaagtgcagctggcggagtccgggggagacttagttcagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccatatattactgtgcgagagacctacgggtgtatgctatggaggcttttgatatctggggccaagggacaatggtcactgtctcctctgcttccccaacctctcctaaggtgttccctctgagcctggacagcacccctcaggatggaaatgtggtggtggcctgtctggtgcagggattcttcccacaagagcccctgtccgtgacttggagcgagtctggacagaacgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggctgtgcccagccttggaatcacggcgagacattcacctgtaccgctgctcaccccgagctgaaaacccctctgaccgccaacatcaccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacacatgtgaatgtgtctgtggtcatggccgaggtggacggcacctgttactgatag

SEQ ID NO: 73 - protein, 233 aa, synthetic construct
  SIGNAL 1..19
MGWSCIILFLVATATGVHASYELTQDPAVSVALGQTVRITCQGDSLRDYYANWFQQKPGQAPLLVIFYDSDRPSGIPERFSGSNSGNTATLTISRVEAGDEADYYCQAWDSSSDHWVFGGGTKLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 74 - DNA, 781 bp, synthetic construct
  source 1..781: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcatcctatgagctgactcaggaccctgctgtgtctgtggccctgggacagacagtcaggatcacatgccaaggggacagcctcagagactattatgcaaactggttccagcagaagccaggccaggcccctttattggtcatcttttatgatagcgaccggccctcagggatccctgagcgattctctggctccaactctgggaatacggccaccctgaccatcagcagggtcgaagccggggatgaggccgactattattgtcaggcgtgggatagtagtagtgaccattgggtgttcggcggagggaccaaactgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 75 - protein, 478 aa, synthetic construct
  SIGNAL 1..19
MDWTWRILFLVAAAQPALAEVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 76 - DNA, 1440 bp, synthetic construct
  source 1..1440: mol_type = other DNA
atggactggacctggcgcatcctgtttctggtggccgctgcccaaccagcgctggccgaagtgcagctggcggagtccgggggagacttagttcagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccatatattactgtgcgagagacctacgggtgtatgctatggaggcttttgatatctggggccaagggacaatggtcactgtctcctctgcttccccaacctctcctaaggtgttccctctgagcctggacagcacccctcaggatggaaatgtggtggtggcctgtctggtgcagggattcttcccacaagagcccctgtccgtgacttggagcgagtctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggcagtgcccagccttggaatcacggcgagacattcacctgtaccgctgctcaccccgagctgaaaacccctctgaccgccaccctcagcaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggctggcaagcccacacatgtgaatgtgtctgtggtcatggccgaggtggacggcacctgatag

SEQ ID NO: 77 - protein, 233 aa, synthetic construct; identical to SEQ ID NO: 73
  SIGNAL 1..19

SEQ ID NO: 78 - DNA, 784 bp, synthetic construct
  source 1..784: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcatcctatgagctgactcaggaccctgctgtgtctgtggccctgggacagacagtcaggatcacatgccaaggggacagcctcagagactattatgcaaactggttccagcagaagccaggccaggcccctttattggtcatcttttatgatagcgaccggccctcagggatccctgagcgattctctggctccaactctgggaatacggccaccctgaccatcagcagggtcgaagccggggatgaggccgactattattgtcaggcgtgggatagtagtagtgaccattgggtgttcggcggagggaccaaactgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 79 - protein, 474 aa, synthetic construct
  SIGNAL 1..19
MDWTWRILFLVAAAQPALAQVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARVGVRYFDWLLYGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 1425 bp, synthetic construct
  source 1..1425: mol_type = other DNA
atggactggacctggcgcatcctgtttctggtggccgctgcccaaccagcgctggcccaggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagagtgggggttcgatattttgactggttattatatggcgcttttgatatctggggccaagggacaatggtcactgtctcctcagcttccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 81 - protein, 231 aa, synthetic construct; identical to SEQ ID NO: 15
  SIGNAL 1..19

SEQ ID NO: 82 - DNA, 693 bp, synthetic construct
  source 1..693: mol_type = other DNA
atggactggacctggcgcatcctgtttctggtggccttcgccacggccgtgcaggcctcctatgagctgactcagccaccctcagtgtccgtgtcgcctggacagacagccaccatcacctgctctggagataagttgggggaaacttatgttcattggtatcagcatcagtcaggccagtcccctattttagtcgtctatcaagatttcaagcggccctccgggatccctgagcgattctctggctccaactctgggaacacagccactctgaccatcagcgggacccaggctatggatgaggctgactattactgtcaggcgtgggacagcagcactgcggtattcggcggagggaccaagctgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 83 - protein, 484 aa, synthetic construct
  SIGNAL 1..19
MDWTWRILFLVAAAQPALAQVQLQESGPGLVKPSETLSLTCTVSGYSISSGYYWGWIRQPPGKGLEWIGSIYHSGSTYYNPSLKSRVTISVDTSKNQFSLKLSSVTAADTAVYYCARVGVRYFDWLLYGAFDIWGQGTMVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 84 - DNA, 1458 bp, synthetic construct
  source 1..1458: mol_type = other DNA
atggactggacctggcgcatcctgtttctggtggccgctgcccaaccagcgctggcccaggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagagtgggggttcgatattttgactggttattatatggcgcttttgatatctggggccaagggacaatggtcactgtctcctcagcttccccaacctctcctaaggtgttccctctgagcctggacagcacccctcaggatggaaatgtggtggtggcctgtctggtgcagggattcttcccacaagagcccctgtccgtgacttggagcgagtctggacagaacgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggctgtgcccagccttggaatcacggcgagacattcacctgtaccgctgctcaccccgagctgaaaacccctctgaccgccaacatcaccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacacatgtgaatgtgtctgtggtcatggccgaggtggacggcacctgttactgatag

SEQ ID NO: 85 - protein, 231 aa, synthetic construct
  SIGNAL 1..19
MGWSCIILFLVATATGVHASYELTQPPSVSVSPGQTATITCSGDKLGETYVHWYQHQSGQSPILVVYQDFKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 86 - DNA, 775 bp, synthetic construct
  source 1..775: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcatcctatgagctgactcagccaccctcagtgtccgtgtcgcctggacagacagccaccatcacctgctctggagataagttgggggaaacttatgttcattggtatcagcatcagtcaggccagtcccctattttagtcgtctatcaagatttcaagcggccctccgggatccctgagcgattctctggctccaactctgggaacacagccactctgaccatcagcgggacccaggctatggatgaggctgactattactgtcaggcgtgggacagcagcactgcggtattcggcggagggaccaagctgaccgtcctaggtcagcccaaggctgccccctcggtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 87 - protein, 468 aa, synthetic construct
  SIGNAL 1..19
MDWTWRILFLVAAAQPALAEVQLVESGGGVVQPGRSLRLSCAASGFTFSSYAMHWVRQAPGKGLEWVSVIYSGGSTYYADSVKGRFTISRDNSKNTLYLQMNSLRAEDTAVYYCARDSSGYRGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 1407 bp, synthetic construct
  source 1..1407: mol_type = other DNA
atggactggacctggcgcatcctgtttctggtggccgctgcccaaccagcgctggccgaagtgcagctggtggagtctgggggaggcgtggtccagcctgggaggtccctgagactctcctgtgcagcctctggattcaccttcagtagctatgctatgcactgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagatagtagtggttatcgaggcgactttgactactggggccagggaaccctggtcaccgtctcctcagcttccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - protein, 233 aa, synthetic construct; identical to SEQ ID NO: 21
  SIGNAL 1..19

SEQ ID NO: 90 - DNA, 702 bp, synthetic construct; identical to SEQ ID NO: 23
  source 1..702: mol_type = other DNA
  intron 47..128

SEQ ID NO: 91 - protein, 478 aa, synthetic construct
  SIGNAL 1..19
MDWTWRILFLVAAAQPALAEVQLVESGGGVVQPGRSLRLSCAASGFTFSSYAMHWVRQAPGKGLEWVSVIYSGGSTYYADSVKGRFTISRDNSKNTLYLQMNSLRAEDTAVYYCARDSSGYRGDFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 92 - DNA, 1440 bp, synthetic construct
  source 1..1440: mol_type = other DNA
atggactggacctggcgcatcctgtttctggtggccgctgcccaaccagcgctggccgaagtgcagctggtggagtctgggggaggcgtggtccagcctgggaggtccctgagactctcctgtgcagcctctggattcaccttcagtagctatgctatgcactgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagatagtagtggttatcgaggcgactttgactactggggccagggaaccctggtcactgtctcttctgcttccccaacctctcctaaggtgttccctctgagcctggacagcacccctcaggatggaaatgtggtggtggcctgtctggtgcagggattcttcccacaagagcccctgtccgtgacttggagcgagtctggacagaacgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggctgtgcccagccttggaatcacggcgagacattcacctgtaccgctgctcaccccgagctgaaaacccctctgaccgccaacatcaccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacacatgtgaatgtgtctgtggtcatggccgaggtggacggcacctgttactgatag

SEQ ID NO: 93 - protein, 233 aa, synthetic construct
  SIGNAL 1..19
MGWSCIILFLVATATGVHASYELTQPPSVSVSPGQTASITCSGDKVGAKYVFWYQQKPGQSPVLVVYQDNKRPSGIPERFSGSNSGNTATLTISGTQAMDEADYYCQAWDSSTALYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 94 - DNA, 781 bp, synthetic construct
  source 1..781: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcatcctatgagctgactcagccaccctcagtgtccgtgtccccaggacagacagccagcatcacctgctctggagataaggtgggggctaaatatgttttctggtatcagcagaagccaggccagtcccctgtgctggtcgtctatcaagataacaagcggccctcagggatccctgagcgattctctggctccaactctgggaacacagccacactgaccatcagcgggacccaggctatggatgaggctgactattactgtcaggcgtgggacagcagcactgccctttatgtcttcggaactgggaccaaggtcaccgtcctaggtcagcccaaggccaaccccactgtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 95 - protein, 476 aa, synthetic construct
  SIGNAL 1..19
MDWTWRILFLVAAAQPALAEVQLVESGGGVVQPGRSLRLSCAASGFTFSSYAMHWVRQAPGKGLEWVSVIYSGGSTYYADSVKGRFTISRDNSKNTLYLQMNSLRAEDTAVYYCARDSSGYRGDFDYWGQGTLVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 96 - DNA, 1434 bp, synthetic construct
  source 1..1434: mol_type = other DNA
atggactggacctggcgcatcctgtttctggtggccgctgcccaaccagcgctggccgaagtgcagctggtggagtctgggggaggcgtggtccagcctgggaggtccctgagactctcctgtgcagcctctggattcaccttcagtagctatgctatgcactgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagagatagtagtggttatcgaggcgactttgactactggggccagggaaccctggtcactgtctcttctgcttccccaacctctcctaaggtgttccctctgagcctggacagcacccctcaggatggaaatgtggtggtggcctgtctggtgcagggattcttcccacaagagcccctgtccgtgacttggagcgagtctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggcagtgcccagccttggaatcacggcgagacattcacctgtaccgctgctcaccccgagctgaaaacccctctgaccgccaccctcagcaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggctggcaagcccacacatgtgaatgtgtctgtggtcatggccgaggtggacggcacctgatag

SEQ ID NO: 97 - protein, 233 aa, synthetic construct; identical to SEQ ID NO: 93
  SIGNAL 1..19

SEQ ID NO: 98 - DNA, 784 bp, synthetic construct
  source 1..784: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcatcctatgagctgactcagccaccctcagtgtccgtgtccccaggacagacagccagcatcacctgctctggagataaggtgggggctaaatatgttttctggtatcagcagaagccaggccagtcccctgtgctggtcgtctatcaagataacaagcggccctcagggatccctgagcgattctctggctccaactctgggaacacagccacactgaccatcagcgggacccaggctatggatgaggctgactattactgtcaggcgtgggacagcagcactgccctttatgtcttcggaactgggaccaaggtcaccgtcctaggtcagcccaaggccaaccccactgtcactctgttcccaccctcctctgaggagctccaagccaacaaggccacactggtgtgtctgatcagtgacttctaccctggagctgtgacagtggcctggaaggcagatggcagccccgtcaaggcgggagtggagacaaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 99 - protein, 469 aa, synthetic construct
  SIGNAL 1..19
MDWTWRILFLVAAAQPALAEVQLLESGGGLVQPGGSLRLSCAASGFTFSDYGMSWVRQAPGKGLEWVSAIGYGGGGKYYADSVKGRFTISRDNSKNTLYLQMNSLRAEDTAVYYCAKDTWWLAGLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1410 bp, synthetic construct
  source 1..1410: mol_type = other DNA
atggactggacctggcgcatcctgtttctggtggccgctgcccaaccagcgctggccgaggtgcagctgttggagtctgggggaggcttggtacagcctggggggtccctgagactctcctgtgcagcctctggattcacctttagcgattacgggatgagctgggtccgccaggctccagggaaggggctggagtgggtatcagccattgggtatggtggaggtggtaaatactacgcagactccgtgaagggccggttcaccatctccagagacaattccaagaacacgctgtatctgcaaatgaacagcctgagagccgaggacacggccgtgtattactgtgctaaggatacttggtggctggcgggtctctttgactactggggccagggaaccctggtcaccgtctcctcagcttccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctccggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - protein, 234 aa, synthetic construct
  SIGNAL 1..19
MDWTWRILFLVAFATAVQAEIVLTQSPGTLSLSPGKRATLSCRASQSVSNSYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 705 bp, synthetic construct; identical to SEQ ID NO: 29
  source 1..705: mol_type = other DNA
  intron 47..128

SEQ ID NO: 103 - protein, 479 aa, synthetic construct
  SIGNAL 1..19
MDWTWRILFLVAAAQPALAEVQLLESGGGLVQPGGSLRLSCAASGFTFSDYGMSWVRQAPGKGLEWVSAIGYGGGGKYYADSVKGRFTISRDNSKNTLYLQMNSLRAEDTAVYYCAKDTWWLAGLFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 104 - DNA, 1443 bp, synthetic construct
  source 1..1443: mol_type = other DNA
atggactggacctggcgcatcctgtttctggtggccgctgcccaaccagcgctggccgaggtgcagctgttggagtctgggggaggcttggtacagcctggggggtccctgagactctcctgtgcagcctctggattcacctttagcgattacgggatgagctgggtccgccaggctccagggaaggggctggagtgggtatcagccattgggtatggtggaggtggtaaatactacgcagactccgtgaagggccggttcaccatctccagagacaattccaagaacacgctgtatctgcaaatgaacagcctgagagccgaggacacggccgtgtattactgtgctaaggatacttggtggctggcgggtctctttgactactggggccagggaaccctggtcactgtctcttctgcttccccaacctctcctaaggtgttccctctgagcctggacagcacccctcaggatggaaatgtggtggtggcctgtctggtgcagggattcttcccacaagagcccctgtccgtgacttggagcgagtctggacagaacgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggctgtgcccagccttggaatcacggcgagacattcacctgtaccgctgctcaccccgagctgaaaacccctctgaccgccaacatcaccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacacatgtgaatgtgtctgtggtcatggccgaggtggacggcacctgttactgatag

SEQ ID NO: 105 - protein, 234 aa, synthetic construct
  SIGNAL 1..19
MGWSCIILFLVATATGVHAEIVLTQSPGTLSLSPGKRATLSCRASQSVSNSYLAWYQQKPGQPPKLLIYWASTRESGVPDRFSGSGSGTDFTLTISSLQAEDVAVYYCQQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - DNA, 787 bp, synthetic construct
  source 1..787: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcagaaattgtgttgacacagtcgccaggcaccctgtctttgtctccagggaaaagagccaccctctcctgcagggccagtcagagtgttagcaacagctacttagcctggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgttag

SEQ ID NO: 107 - protein, 477 aa, synthetic construct
  SIGNAL 1..19
MDWTWRILFLVAAAQPALAEVQLLESGGGLVQPGGSLRLSCAASGFTFSDYGMSWVRQAPGKGLEWVSAIGYGGGGKYYADSVKGRFTISRDNSKNTLYLQMNSLRAEDTAVYYCAKDTWWLAGLFDYWGQGTLVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 108 - DNA, 1437 bp, synthetic construct
  source 1..1437: mol_type = other DNA
atggactggacctggcgcatcctgtttctggtggccgctgcccaaccagcgctggccgaggtgcagctgttggagtctgggggaggcttggtacagcctggggggtccctgagactctcctgtgcagcctctggattcacctttagcgattacgggatgagctgggtccgccaggctccagggaaggggctggagtgggtatcagccattgggtatggtggaggtggtaaatactacgcagactccgtgaagggccggttcaccatctccagagacaattccaagaacacgctgtatctgcaaatgaacagcctgagagccgaggacacggccgtgtattactgtgctaaggatacttggtggctggcgggtctctttgactactggggccagggaaccctggtcactgtctcttctgcttccccaacctctcctaaggtgttccctctgagcctggacagcacccctcaggatggaaatgtggtggtggcctgtctggtgcagggattcttcccacaagagcccctgtccgtgacttggagcgagtctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaactccagccaggacgtgaccgtgccttgtagagttcctcctcctccaccttgctgtcaccctcggctgtctctgcatagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtcctctgtgctgcctggcagtgcccagccttggaatcacggcgagacattcacctgtaccgctgctcaccccgagctgaaaacccctctgaccgccaccctcagcaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggctggcaagcccacacatgtgaatgtgtctgtggtcatggccgaggtggacggcacctgatag

SEQ ID NO: 109 - protein, 234 aa, synthetic construct; identical to SEQ ID NO: 105
  SIGNAL 1..19

SEQ ID NO: 110 - DNA, 784 bp, synthetic construct
  source 1..784: mol_type = other DNA
  intron 47..128
atgggatggagctgtatcatcctcttcttggtagcaacagctacaggtaaggggttaacagtagcaggcttgaggtctggacatatatatgggtgacaatgacatccactttgcctttctctccacaggcgtgcacgcagaaattgtgttgacacagtcgccaggcaccctgtctttgtctccagggaaaagagccaccctctcctgcagggccagtcagagtgttagcaacagctacttagcctggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 111 - protein, 214 aa, synthetic construct
SYELTQDPAVSVALGQTVRITCQGDSLRDYYANWFQQKPGQAPLLVIFYDSDRPSGIPERFSGSNSGNTATLTISRVEAGDEADYYCQAWDSSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - DNA, 642 bp, synthetic construct
  source 1..642: mol_type = other DNA
agttatgaactaactcaagatccagcagtgtccgtggccctgggccagaccgtgcggatcacctgtcagggcgattctctgagagattattacgccaactggttccagcagaagcctggccaagctcctctgctggtgatcttctacgactccgacagaccttctggcatccccgagcggtttagcggatccaactccggcaacaccgctaccctgaccatctccagagtggaagctggcgacgaggccgactactactgccaggcctgggactctagctctgaccactgggtcttcggcggaggcaccaagctgacagtgctcggccagcccaaagccgccccaagcgtcacactgtttcctccctcctctgaagaactgcaggcaaataaggcaactctggtctgtctgatctccgacttctacccaggagcagtgacagtggcatggaaggcagatagctcccccgtgaaggcaggagtggagacaacaacccctagcaagcagtccaacaataagtacgccgcctctagctatctgtctctgacccccgagcagtggaagagccaccggtcctattcttgccaggtgacccacgagggctcaaccgtcgaaaaaaccgtcgcacctactgaatgttcc

SEQ ID NO: 113 - protein, 529 aa, synthetic construct
EVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPELKTPLGDTTHTCPRCPEPKSCDTPPPCPRCPEPKSCDTPPPCPRCPEPKSCDTPPPCPRCPAPELLGGPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 114 - DNA, 1589 bp, synthetic construct
  source 1..1589: mol_type = other DNA
gaagtgcaactagcagaaagtggtggtgatctcgtgcagcctggcggatctctgcggctgtcttgtgccgcttctggcttcaccttctcctcctacgccatgtcctgggtgcggcaagctcccggaaaaggcctggagtgggtgtccgctatctctggctccggcggctctacctactatgccgactccgtgaagggcagatttaccatctcccgggacaactccaagaacacactgtacctgcagatgaacagcctgagagccgaggacaccgccatctactactgcgccagagatctgagagtgtacgctatggaagccttcgacatctggggccagggcaccatggtgaccgtgtcctctgcttctcccacttccccaaaggtgttccctctgtcactggactcaacccctcaggatggcaatgtcgtcgtcgcatgtctggtgcagggcttctttcctcaggagccactgagcgtgacatggtctgagagcggccagggcgtgaccgccagaaatttccccccttcccaggacgcctctggcgatctgtacaccacaagctcccagctgaccctgccagcaacacagtgcccagacggcaagtccgtgacctgtcacgtgaagcactatacaaactctagccaggatgtgaccgtgccctgccgggtgcctgaactaaagactccactaggtgatactactcacacctgtcctagatgccctgaacccaaaagctgcgacacacctccaccttgtcctcggtgtcctgagcctaagtcctgcgatacccctcctccttgcccccggtgccccgagcccaagtcttgcgacacccctccaccctgccctagatgccctgcccctgagctgctgggcggccctcctccacccccttgctgtcacccaaggctgtccctgcacaggcccgccctggaggacctgctgctgggctctgaggccaatctgacatgtaccctgacaggcctgcgggatgccagcggagccacctttacatggaccccctcctctggcaagtccgccgtgcagggaccaccagagcgggacctgtgcggctgttacagcgtgagcagcgtgctgcctggatccgcccagccctggaaccacggcgagacattcacctgtacagcagcacacccagagctgaagacccctctgacagccaccctgagcaagtctggcaataccttccggccagaggtgcacctgctgcctccaccaagcgaggagctggccctgaatgagctggtgaccctgacatgcctggccaggggcttctctcctaaggacgtgctggtgcgctggctgcagggcagccaggagctgcctcgggagaagtatctgacctgggccagcagacaggagccatcccagggcaccacaacctttgccgtgacctccatcctgagggtggcagcagaggactggaagaagggcgataccttctcttgtatggtgggacacgaggccctgcccctggcctttacccagaagacaatcgatcgcctggccggcaagcctactcatgtgaatgtcagtgtcgtgatggcagaggtggacgggacttg

SEQ ID NO: 115 - protein, 528 aa, synthetic construct
EVQLAESGGDLVQPGGSLRLSCAASGFTFSSYAMSWVRQAPGKGLEWVSAISGSGGSTYYADSVKGRFTISRDNSKNTLYLQMNSLRAEDTAIYYCARDLRVYAMEAFDIWGQGTMVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPELKTPLGDTTHTCPRCPEPKSCDTPPPCPRCPEPKSCDTPPPCPRCPEPKSCDTPPPCPRCPAPELLGGPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 116 - DNA, 1584 bp, synthetic construct
  source 1..1584: mol_type = other DNA
gaagtgcaactagcagaaagtggtggtgatctcgtgcagcctggcggatctctgcggctgtcttgtgccgcttctggcttcaccttctcctcctacgccatgtcctgggtgcggcaagctcccggaaaaggcctggagtgggtgtccgctatctctggctccggcggctctacctactatgccgactccgtgaagggcagatttaccatctcccgggacaactccaagaacacactgtacctgcagatgaacagcctgagagccgaggacaccgccatctactactgcgccagagatctgagagtgtacgctatggaagccttcgacatctggggccagggcaccatggtgaccgtgtcctctgcttctcccacttccccaaaggtgttccctctgtcactggactcaacccctcaggatggcaatgtcgtcgtcgcatgtctggtgcagggcttctttcctcaggagccactgagcgtgacatggtctgagagcggccagggcgtgaccgccagaaatttccccccttcccaggacgcctctggcgatctgtacaccacaagctcccagctgaccctgccagcaacacagtgcccagacggcaagtccgtgacctgtcacgtgaagcactatacaaactctagccaggatgtgaccgtgccctgccgggtgcctgaactaaagactccactaggtgatactactcacacctgtcctagatgccctgaacccaaaagctgcgacacacctccaccttgtcctcggtgtcctgagcctaagtcctgcgatacccctcctccttgcccccggtgccccgagcccaagtcttgcgacacccctccaccctgccctagatgccctgcccctgagctgctgggcggccctccacccccttgctgtcacccaaggctgtccctgcacaggcccgccctggaggacctgctgctgggctctgaggccaatctgacatgtaccctgacaggcctgcgggatgccagcggagccacctttacatggaccccctcctctggcaagtccgccgtgcagggaccaccagagcgggacctgtgcggctgttacagcgtgagcagcgtgctgcctggatccgcccagccctggaaccacggcgagacattcacctgtacagcagcacacccagagctgaagacccctctgacagccaccctgagcaagtctggcaataccttccggccagaggtgcacctgctgcctccaccaagcgaggagctggccctgaatgagctggtgaccctgacatgcctggccaggggcttctctcctaaggacgtgctggtgcgctggctgcagggcagccaggagctgcctcgggagaagtatctgacctgggccagcagacaggagccatcccagggcaccacaacctttgccgtgacctccatcctgagggtggcagcagaggactggaagaagggcgataccttctcttgtatggtgggacacgaggccctgcccctggcctttacccagaagacaatcgatcgcctggccggcaagcctactcatgtgaatgtcagtgtcgtgatggcagaggtggacgggact

SEQ ID NO: 117 - protein, 218 aa, synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - DNA, 654 bp, synthetic construct
  source 1..654: mol_type = other DNA
gatatcgtgatgactcaaagtccagatagtctcgctgtgtccctgggcgagagagccacaatcaactgcagagcttctgagtccgtggacatctatggcaactccttcatgcactggtaccagcagaaaccaggccagcctcctaagctgctgatctacctggccagcaatctggaatctggagtccccgaccggttttctggctctggctccggcaccgacttcaccctgaccatctccagcctgcaggccgaggacgtggccgtgtactactgtcaacagaacaacgaggatccttacaccttcggcggcggaaccaaggtggaaatcaagaggaccgtcgccgcccctagtgtctttatcttccccccttccgatgaacagctgaaaagtgggaccgcctccgtcgtgtgcctgctgaacaatttctacccccgggaggccaaggtgcagtggaaggtggacaacgccctgcagtctggcaatagccaggagtccgtgaccgagcaggactctaaggatagcacatattccctgagcagcaccctgaccctgtctaaggccgattacgagaagcacaaggtgtatgcctgcgaagtcacccatcaggggctgtccagtccagtcacaaagtcattcaataggggggagtgt

SEQ ID NO: 119 - protein, 525 aa, synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPELKTPLGDTTHTCPRCPEPKSCDTPPPCPRCPEPKSCDTPPPCPRCPEPKSCDTPPPCPRCPAPELLGGPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 120 - DNA, 1575 bp, synthetic construct
  source 1..1575: mol_type = other DNA
caagtgcaactagtgcaaagtggtgcagaagtcaagaagcctggcgcttctgtgaaagtgtcctgcaaggcctctggctacaccttcacctcctacaacatgcactgggtgcggcaggctcccggacaaggcctggagtggatgggcgtgatctactccggaaatggcgacacatcttataaccagaagttcaagggcagagtgaccatcaccgccgacaagtccacctctacagcctacatggaactgtccagcctgcggtccgaggacaccgctgtgtactactgtgccagagagcgggataccagatttggcaactggggccagggcaccctggtgaccgtgtcttctgcttctcccacttccccaaaggtgttccctctgtcactggactcaacccctcaggatggcaatgtcgtcgtcgcatgtctggtgcagggcttctttcctcaggagccactgagcgtgacatggtctgagagcggccagggcgtgaccgccagaaatttccccccttcccaggacgcctctggcgatctgtacaccacaagctcccagctgaccctgccagcaacacagtgcccagacggcaagtccgtgacctgtcacgtgaagcactatacaaactctagccaggatgtgaccgtgccctgccgggtgcctgaactaaagactccactaggtgatactactcacacctgtcctagatgccctgaacccaaaagctgcgacacacctccaccttgtcctcggtgtcctgagcctaagtcctgcgatacccctcctccttgcccccggtgccccgagcccaagtcttgcgacacccctccaccctgccctagatgccctgcccctgagctgctgggcggccctcctccacccccttgctgtcacccaaggctgtccctgcacaggcccgccctggaggacctgctgctgggctctgaggccaatctgacatgtaccctgacaggcctgcgggatgccagcggagccacctttacatggaccccctcctctggcaagtccgccgtgcagggaccaccagagcgggacctgtgcggctgttacagcgtgagcagcgtgctgcctggatccgcccagccctggaaccacggcgagacattcacctgtacagcagcacacccagagctgaagacccctctgacagccaccctgagcaagtctggcaataccttccggccagaggtgcacctgctgcctccaccaagcgaggagctggccctgaatgagctggtgaccctgacatgcctggccaggggcttctctcctaaggacgtgctggtgcgctggctgcagggcagccaggagctgcctcgggagaagtatctgacctgggccagcagacaggagccatcccagggcaccacaacctttgccgtgacctccatcctgagggtggcagcagaggactggaagaagggcgataccttctcttgtatggtgggacacgaggccctgcccctggcctttacccagaagacaatcgatcgcctggccggcaagcctactcatgtgaatgtcagtgtcgtgatggcagaggtggacgggact

SEQ ID NO: 121 - protein, 524 aa, synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASPTSPKVFPLSLDSTPQDGNVVVACLVQGFFPQEPLSVTWSESGQGVTARNFPPSQDASGDLYTTSSQLTLPATQCPDGKSVTCHVKHYTNSSQDVTVPCRVPELKTPLGDTTHTCPRCPEPKSCDTPPPCPRCPEPKSCDTPPPCPRCPEPKSCDTPPPCPRCPAPELLGGPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 122 - DNA, 1573 bp, synthetic construct
  source 1..1573: mol_type = other DNA
caagtgcaactagtgcaaagtggtgcagaagtcaagaagcctggcgcttctgtgaaagtgtcctgcaaggcctctggctacaccttcacctcctacaacatgcactgggtgcggcaggctcccggacaaggcctggagtggatgggcgtgatctactccggaaatggcgacacatcttataaccagaagttcaagggcagagtgaccatcaccgccgacaagtccacctctacagcctacatggaactgtccagcctgcggtccgaggacaccgctgtgtactactgtgccagagagcgggataccagatttggcaactggggccagggcaccctggtgaccgtgtcttctgcttctcccacttccccaaaggtgttccctctgtcactggactcaacccctcaggatggcaatgtcgtcgtcgcatgtctggtgcagggcttctttcctcaggagccactgagcgtgacatggtctgagagcggccagggcgtgaccgccagaaatttccccccttcccaggacgcctctggcgatctgtacaccacaagctcccagctgaccctgccagcaacacagtgcccagacggcaagtccgtgacctgtcacgtgaagcactatacaaactctagccaggatgtgaccgtgccctgccgggtgcctgaactaaagactccactaggtgatactactcacacctgtcctagatgccctgaacccaaaagctgcgacacacctccaccttgtcctcggtgtcctgagcctaagtcctgcgatacccctcctccttgcccccggtgccccgagcccaagtcttgcgacacccctccaccctgccctagatgccctgcccctgagctgctgggcggccctccacccccttgctgtcacccaaggctgtccctgcacaggcccgccctggaggacctgctgctgggctctgaggccaatctgacatgtaccctgacaggcctgcgggatgccagcggagccacctttacatggaccccctcctctggcaagtccgccgtgcagggaccaccagagcgggacctgtgcggctgttacagcgtgagcagcgtgctgcctggatccgcccagccctggaaccacggcgagacattcacctgtacagcagcacacccagagctgaagacccctctgacagccaccctgagcaagtctggcaataccttccggccagaggtgcacctgctgcctccaccaagcgaggagctggccctgaatgagctggtgaccctgacatgcctggccaggggcttctctcctaaggacgtgctggtgcgctggctgcagggcagccaggagctgcctcgggagaagtatctgacctgggccagcagacaggagccatcccagggcaccacaacctttgccgtgacctccatcctgagggtggcagcagaggactggaagaagggcgataccttctcttgtatggtgggacacgaggccctgcccctggcctttacccagaagacaatcgatcgcctggccggcaagcctactcatgtgaatgtcagtgtcgtgatggcagaggtggacgggactt

SEQ ID NO: 123 - protein, 5 aa, synthetic construct
PCRVP

SEQ ID NO: 124 - protein, 12 aa, synthetic construct
ELKTPLGDTTHT

SEQ ID NO: 125 - protein, 50 aa, synthetic construct
CPRCPEPKSCDTPPPCPRCPEPKSCDTPPPCPRCPEPKSCDTPPPCPRCP

SEQ ID NO: 126 - protein, 8 aa, synthetic construct
APELLGGP

SEQ ID NO: 127 - protein, 9 aa, synthetic construct
PPPPCCHPR

SEQ ID NO: 128 - protein, 8 aa, synthetic construct
PPPCCHPR

(19) United States
(12) Patent Application Publication
(10) Pub. No.: US 2025/0171536 A1
(43) Pub. Date: May 29, 2025

(54) Methods of Bone Marrow Conditioning

(71) Applicant: CSL Innovation Pty Ltd, Melbourne, Victoria (AU)

(72) Inventors: Con Panousis, Melbourne (AU); Veronika Rayzman, Melbourne (AU)

(73) Assignee: CSL Innovation Pty Ltd, Melbourne, Victoria (AU)

(21) Appl. No.: 18/842,670

(22) PCT Filed: Mar. 1, 2023
(86) PCT No.: PCT/AU2023/050138

§ 371 Date: Aug. 29, 2024

(30) Foreign Application Priority Data
Mar. 1, 2022 (AU) 2022900484

(51) Int. Cl. C07K 16/28; A61K 35/28

(52) U.S. Cl. CPC C07K 16/2803; A61K 35/28; C07K 2317/33; C07K 2317/53; C07K 2317/565; C07K 2317/73; C07K 2317/76; C07K 2317/77; C07K 2317/92

(57) ABSTRACT

The present disclosure relates to methods of conditioning a subject and/or depleting a population of cells in the bone marrow of a subject in need of a hematopoietic cell transplantation (HCT) comprising administering an antibody that binds to a target on a endogenous hematopoietic stem cell (HSC) in the subject. In a preferred embodiment, the target is CD 117 (c-kit).

### RELATED APPLICATION DATA

The present application claims priority from Australian Patent Application No. 2022900484 entitled ‘Methods of Bone Marrow Conditioning’ filed 1 Mar. 2022. The entire contents of which is hereby incorporated by reference.

### SEQUENCE LISTING

The present application is filed with a Sequence Listing in electronic form. The entire contents of the Sequence Listing are hereby incorporated by reference.

### FIELD

The present disclosure relates to methods of conditioning a subject and/or depleting a population of cells in the bone marrow of a subject in need of a hematopoietic cell transplantation (HCT) comprising administering an antibody that binds to a target on an endogenous hematopoietic stem cell (HSC) in the subject.

### BACKGROUND

Hematopoietic cell transplantation (HCT) is the administration of hematopoietic stem and progenitor cells to patients with a variety of acquired and inherited malignant and non-malignant disorders to establish marrow and immune function. These disorders include hematologic malignancies (e.g., leukaemia, lymphoma, and myeloma), non-malignant acquired bone marrow disorders (e.g., aplastic anaemia), and genetic diseases associated with abnormal haematopoiesis and function (e.g., thalassemia, sickle cell anaemia, and severe combined immunodeficiency). HCT may also be used to support patients undergoing high-dose chemotherapy for the treatment of certain solid tumors for whom hematologic toxicity would otherwise limit drug administration (e.g., germ cell tumours, soft tissue sarcomas, and neuroblastoma).

Prior to a HCT, conditioning therapy is used to reduce disease burden, creation of space for engraftment and immunosuppression. Currently, conditioning regimens are myeloablative or non-myeloablative. Myeloablative regimens include high-dose chemotherapy (e.g., cyclophosphamide, busulfan, and etoposide) and/or radiation therapy (e.g., total body irradiation) in combination with an immunosuppressive component. Whilst these regimens are designed to suppress the immune system, they can also adversely affect multiple organ systems, frequently leading to life-threatening complications. Non-myeloablative regimens (or reduced-intensity regimens) use doses of chemotherapy and radiation too low to eradicate all bone-marrow cells of the patient and as such, patients have reduced risk of infection and transplant-related mortality. However, relapse has remained a major problem following reduced intensity conditioning regimens.

More recently, non-myeloablative regimens have been combined with other immunotherapy conditioning regimens, including chimeric antigen receptor (CAR)-modified T cells against hematopoietic stem cell markers. However, a major limitation of this regimen is the complete elimination of all CAR-modified infused T cells prior to the donor cell transplantation.

Accordingly, there is a need for the development of conditioning regimens that avoid undesirable toxicity and minimise the incidence of serious adverse reactions, whilst also reducing a patient's risk of relapse.

### SUMMARY

In producing the present invention, the inventors identified antibodies useful in conditioning regimens for patients preparing for a hematopoietic cell transplantation (HCT). The inventors found that antibodies that bind a target (e.g., CD117) on endogenous hematopoietic stem cells (HSCs) successfully deplete and/or ablate the HSCs. Advantageously, the inventors identified that depletion of HSCs was enhanced with antibodies that mediate antibody dependent cellular trogocytosis (ADCT), as well as with antibodies comprising an IgA₂ heavy chain.

Accordingly, the findings by the inventors provide the basis for methods of conditioning a subject in need of a HCT. In particular, the findings provide the basis for methods of conditioning a subject in need of a HCT comprising administering to the subject an antibody that binds or specifically binds to a target on an endogenous HSC in the subject.

The findings by the inventors also provide the basis for methods of reducing, depleting and/or ablating a population of cells in the bone marrow of a subject in need of a HCT. In particular, the findings provide the basis for methods of reducing, depleting and/or ablating a population of cells in the bone marrow of a subject in need of a HCT, comprising administering to the subject an antibody that binds or specifically binds to a target on an endogenous HSC in the subject.

In one example, the antibody mediates antibody-dependent cell-mediated cytotoxicity (ADCC), and/or antibody-dependent cell mediated phagocytosis (ADCP), and/or ADCT, and/or complement dependent cytotoxicity (CDC) and combinations thereof. For example, the antibody mediates ADCC. In another example, the antibody mediates ADCP. In a further example, the antibody mediates ADCT. In one example, the antibody mediates CDC. In one example, the antibody mediates ADCC and ADCP. In another example, the antibody mediates ADCC and ADCT. In a further example, the antibody mediates ADCC and CDC. In one example, the antibody mediates ADCP and ADCT. In another example, the antibody mediates ADCP and CDC. In a further example, the antibody mediates ADCT and CDC. In one example, the antibody mediates ADCC, ADCP and ADCT. In another example, the antibody mediates ADCC, ADCP and CDC. In a further example, the antibody mediates ADCC, ADCT and CDC. In one example, the antibody mediates ADCP, ADCT and CDC. In one example, the antibody mediates ADCC, ADCP, ADCT and CDC.

The present disclosure provides a method of conditioning a subject in need of a HCT, the method comprising administering to the subject an antibody that binds or specifically binds to a target on an endogenous HSC in the subject, wherein the antibody mediates ADCT.

The present disclosure also provides a method of reducing, depleting and/or ablating a population of cells in the bone marrow of a subject in need of a HCT, the method comprising administering to the subject an antibody that binds or specifically binds to a target on an endogenous HSC in the subject, wherein the antibody mediates ADCT.

The present disclosure provides a method of conditioning a subject in need of a HCT, the method comprising administering to the subject an antibody that comprises a modified hinge region.

The present disclosure also provides a method of reducing, depleting and/or ablating a population of cells in the bone marrow of a subject in need of a HCT, comprising administering to the subject an antibody that comprises a modified hinge region, e.g., an extended hinge region. For example, the extended hinge region comprises a portion of an IgG₃ hinge region inserted into an IgA₂ hinge region after the first proline.

In one example, the antigen binding site of an antibody of the disclosure comprising an extended hinge region is better able to bind to its epitope relative to the antigen binding site of an antibody without the extended hinge region.

The present disclosure provides a method of conditioning a subject in need of a HCT, the method comprising administering to the subject an antibody that comprises an extended hinge region, wherein the antigen binding site of the antibody is better able to bind to its epitope relative to the antibody without the extended hinge region.

The present disclosure also provides a method of reducing, depleting and/or ablating a population of cells in the bone marrow of a subject in need of a HCT, comprising administering to the subject an antibody that comprises an extended hinge region, wherein the antigen binding site of the antibody has better access to its epitope relative to the antibody without the extended hinge region.

In one example, the antibody comprises an IgA antibody heavy chain. For example, the IgA antibody heavy chain is an IgA₂ heavy chain.

In one example, the antibody comprises an antibody light chain. For example, the antibody comprises a lambda or a kappa light chain. In one example, the antibody comprises a lambda light chain. In another example, the antibody comprises a kappa light chain.

In one example, the antibody comprises an IgA₂ heavy chain and a lambda or a kappa light chain.

In one example, the IgA₂ heavy chain is a modified IgA₂ heavy chain. For example, the IgA₂ heavy chain comprises one or more modifications selected from the group consisting of:
(i) a N to G mutation at position 166 according to the myeloma IgA1 protein (Bur) scheme;
(ii) a P to R mutation at position 221 according to the myeloma IgA1 protein (Bur) scheme;
(iii) a C to S mutation at position 311 according to the myeloma IgA1 protein (Bur) scheme;
(iv) a NIT to TLS mutation at positions 337 to 339 according to the myeloma IgA1 protein (Bur) scheme;
(v) a deletion of a C residue at position 472 according to the myeloma IgA1 protein (Bur) scheme; and
(vi) a deletion of a Y residue at position 473 according to the myeloma IgA1 protein (Bur) scheme.

In one example, the IgA₂ heavy chain comprises a N to G mutation at position 166 according to the myeloma IgA1 protein (Bur) scheme. For example, the IgA₂ comprises a N to G mutation at a residue corresponding to amino acid 47 of SEQ ID NO: 32.

In one example, the IgA₂ heavy chain comprises a P to R mutation at position 221 according to the myeloma IgA₁ protein (Bur) scheme. For example, the IgA₂ comprises a P to R mutation at a residue corresponding to amino acid 102 of SEQ ID NO: 32. In one example, the IgA₂ comprises a sequence set forth in SEQ ID NO: 33.

In one example, the IgA₂ heavy chain comprises a C to S mutation at position 311 according to the myeloma IgA₁ protein (Bur) scheme. For example, the IgA₂ comprises a C to S mutation at a residue corresponding to amino acid 179 of SEQ ID NO: 32.

In one example, the IgA₂ heavy chain comprises a NIT to TLS mutation at positions 337 to 339 according to the myeloma IgA₁ protein (Bur) scheme. For example, the IgA₂ comprises a NIT to TLS mutation at residues corresponding to amino acids 205 to 207 of SEQ ID NO: 32.

In one example, the IgA₂ heavy chain comprises a deletion of a C residue at position 472 according to the myeloma IgA₁ protein (Bur) scheme. For example, the IgA₂ comprises deletion of a C residue corresponding to amino acid 339 of SEQ ID NO: 32.

In one example, the IgA₂ heavy chain comprises a deletion of a deletion of a Y residue at position 473 according to the myeloma IgA₁ protein (Bur) scheme. For example, the IgA₂ comprises deletion of a Y residue corresponding to amino acid 340 of SEQ ID NO: 32.

In one example, the IgA₂ heavy chain comprises (i) a N to G mutation at position 166 according to the myeloma IgA1 protein (Bur) scheme; and (ii) a P to R mutation at position 221 according to the myeloma IgA1 protein (Bur) scheme; and (iii) a C to S mutation at position 311 according to the myeloma IgA1 protein (Bur) scheme; and (iv) a NIT to TLS mutation at positions 337 to 339 according to the myeloma IgA1 protein (Bur) scheme; and (v) a deletion of a C residue at position 472 according to the myeloma IgA1 protein (Bur) scheme; and (vi) a deletion of a Y residue at position 473 according to the myeloma IgA1 protein (Bur) scheme.

In one example, the IgA₂ heavy chain comprises (i) a N to G mutation at a residue corresponding to amino acid 47 of SEQ ID NO: 32; and (ii) a P to R mutation at a residue corresponding to amino acid 102 of SEQ ID NO: 32; and (iii) a C to S mutation at a residue corresponding to amino acid 179 of SEQ ID NO: 32; and (iv) a NIT to TLS mutation at residues corresponding to amino acids 205 to 207 of SEQ ID NO: 32; and (v) deletion of a C residue corresponding to amino acid 339 of SEQ ID NO: 32; and (vi) deletion of a Y residue corresponding to amino acid 340 of SEQ ID NO: 32.

In one example, the IgA₂ heavy chain comprises a sequence set forth in SEQ ID NO: 33 or SEQ ID NO: 34 or SEQ ID NO: 66. For example, the IgA₂ heavy chain comprises a sequence set forth in SEQ ID NO: 33. In another example, the IgA₂ heavy chain comprises a sequence set forth in SEQ ID NO: 34. In a further example, the IgA₂ heavy chain comprises a sequence set forth in SEQ ID NO: 66.

In one example, the IgA₂ heavy chain comprises a sequence set forth in SEQ ID NO: 66, wherein the amino acid sequence comprises Asparagine (N) or Glycine (G) at position 47 and/or Proline (P) or Arginine (R) at position 102 and/or Cysteine (C) or Serine(S) at position 179 and/or Asparagine (N) or Threonine (T) at position 205 and/or Isoleucine (I) or Leucine (L) at position 206 and/or Threonine (T) or Serine(S) at position 207 and/or Cysteine (C) or absent at position 339 and/or Tyrosine (Y) or absent at position 340.

In one example, the IgA₂ heavy chain comprises a sequence set forth in SEQ ID NO: 66, wherein the amino acid sequence comprises Asparagine (N) at position 47 and Arginine (R) at position 102 and Cysteine (C) at position 179 and Asparagine (N) at position 205 and Isoleucine (I) at position 206 and Threonine (T) at position 207 and Cysteine (C) at position 339 and Tyrosine (Y) at position 340. For example, the IgA₂ heavy chain comprises a sequence set forth in SEQ ID NO: 33.

In one example, the IgA₂ heavy chain comprises a sequence set forth in SEQ ID NO: 66, wherein the amino acid sequence comprises Glycine (G) at position 47 and Arginine (R) at position 102 and Serine(S) at position 179 and Threonine (T) at position 205 and Leucine (L) at position 206 and Serine(S) at position 207 and absent at position 339 and absent at position 340. For example, the IgA₂ heavy chain comprises a sequence set forth in SEQ ID NO: 34.

In one example, the antibody comprising an extended hinge region comprises a C-terminal CH1 sequence from an IgA, an upper hinge sequence from an IgG₃, a middle hinge sequence from an IgG₃, a lower hinge sequence from an IgG₃, and/or a sequence past the lower hinge sequence from an IgA and combinations thereof. In one example, the antibody comprising an extended hinge region comprises a C-terminal CH1 sequence from an IgA. In one example, the antibody comprising an extended hinge region comprises an upper hinge sequence from an IgG₃. In one example, the antibody comprising an extended hinge region comprises a middle hinge sequence from an IgG₃. In one example, the antibody comprising an extended hinge region comprises a lower hinge sequence from an IgG₃. In one example, the antibody comprising an extended hinge region comprises a sequence past the lower hinge sequence from an IgA.

In one example, the antibody comprising an extended hinge region comprises a C-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 127 and combinations thereof. In one example, the antibody comprising an extended hinge region comprises a C-terminal CH1 comprising a sequence set forth in SEQ ID NO: 123. In one example, the antibody comprising an extended hinge region comprises an upper hinge comprising a sequence set forth in SEQ ID NO: 124. In one example, the antibody comprising an extended hinge region comprises a middle hinge comprising a sequence set forth in SEQ ID NO: 125. In one example, the antibody comprising an extended hinge region comprises an upper hinge comprising a lower hinge comprising a sequence set forth in SEQ ID NO: 126. In one example, the antibody comprising an extended hinge region comprises a sequence past the lower hinge comprising a sequence set forth in SEQ ID NO: 127.

In one example, the antibody comprising an extended hinge region comprises a-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 128 and combinations thereof. In one example, the antibody comprising an extended hinge region comprises a C-terminal CH1 comprising a sequence set forth in SEQ ID NO: 123. In one example, the antibody comprising an extended hinge region comprises an upper hinge comprising a sequence set forth in SEQ ID NO: 124. In one example, the antibody comprising an extended hinge region comprises a middle hinge comprising a sequence set forth in SEQ ID NO: 125. In one example, the antibody comprising an extended hinge region comprises an upper hinge comprising a lower hinge comprising a sequence set forth in SEQ ID NO: 126. In one example, the antibody comprising an extended hinge region comprises a sequence past the lower hinge comprising a sequence set forth in SEQ ID NO: 128.

In one example of any method described herein, the antibody binds or specifically binds to a target on an endogenous HSC in the subject. In one example, the target on the endogenous HSC is selected from the group consisting of CD2, CD5, CD7, CD11a, CDw12, CD13, CD15, CD18, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d (VLA-4), CD49e, CD49f (VLA-6), CD50, CD51, CD53, CD55, CD58, CD64a, CD68, CD71, CD72, CD73, CD81, CD82, CD84, CD85A, CD85K, CD90, CD97, CD99, CD104, CD105, CD109, CD110, CD111, CD112, CD114, CD115, CD117 (c-kit), CD123, CD124, CD126, CD127, CD130, CD131, CD133, CD134, CD135, CD137, CD138, CD151, CD157, CD162, CD164, CD166, CD168, CD172a, CD173, CD174, CD175, CD175s, CD176, CD183, CD184 (CXCR4), CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, CD350, CD361 and combinations thereof.

In one example, the target on the endogenous HSC is CD2. In one example, the target on the endogenous HSC is CD5. In one example, the target on the endogenous HSC is CD7. In one example, the target on the endogenous HSC is CD11a. In one example, the target on the endogenous HSC is CDw12. In one example, the target on the endogenous HSC is CD13. In one example, the target on the endogenous HSC is CD15. In one example, the target on the endogenous HSC is CD18. In one example, the target on the endogenous HSC is CD19. In one example, the target on the endogenous HSC is CD21. In one example, the target on the endogenous HSC is CD22. In one example, the target on the endogenous HSC is CD29. In one example, the target on the endogenous HSC is CD30. In one example, the target on the endogenous HSC is CD33. In one example, the target on the endogenous HSC is CD34. In one example, the target on the endogenous HSC is CD36. In one example, the target on the endogenous HSC is CD38. In one example, the target on the endogenous HSC is CD40. In one example, the target on the endogenous HSC is CD41. In one example, the target on the endogenous HSC is CD42a. In one example, the target on the endogenous HSC is CD42b. In one example, the target on the endogenous HSC is CD42c. In one example, the target on the endogenous HSC is CD42d. In one example, the target on the endogenous HSC is CD43. In one example, the target on the endogenous HSC is CD45. In one example, the target on the endogenous HSC is CD45RA. In one example, the target on the endogenous HSC is CD45RB. In one example, the target on the endogenous HSC is CD45RC. In one example, the target on the endogenous HSC is CD45RO. In one example, the target on the endogenous HSC is CD48. In one example, the target on the endogenous HSC is CD49b. In one example, the target on the endogenous HSC is CD49d (VLA-4). In one example, the target on the endogenous HSC is CD49e. In one example, the target on the endogenous HSC is CD49f (VLA-6). In one example, the target on the endogenous HSC is CD50. In one example, the target on the endogenous HSC is CD51. In one example, the target on the endogenous HSC is CD53. In one example, the target on the endogenous HSC is CD55. In one example, the target on the endogenous HSC is CD58. In one example, the target on the endogenous HSC is CD64a. In one example, the target on the endogenous HSC is CD68. In one example, the target on the endogenous HSC is CD71. In one example, the target on the endogenous HSC is CD72. In one example, the target on the endogenous HSC is CD73. In one example, the target on the endogenous HSC is CD81. In one example, the target on the endogenous HSC is CD82. In one example, the target on the endogenous HSC is CD84. In one example, the target on the endogenous HSC is CD85A. In one example, the target on the endogenous HSC is CD85K. In one example, the target on the endogenous HSC is CD90. In one example, the target on the endogenous HSC is CD97. In one example, the target on the endogenous HSC is CD99. In one example, the target on the endogenous HSC is CD104. In one example, the target on the endogenous HSC is CD105. In one example, the target on the endogenous HSC is CD109. In one example, the target on the endogenous HSC is CD110. In one example, the target on the endogenous HSC is CD111. In one example, the target on the endogenous HSC is CD112. In one example, the target on the endogenous HSC is CD114. In one example, the target on the endogenous HSC is CD115. In one example, the target on the endogenous HSC is CD117 (c-kit). In one example, the target on the endogenous HSC is CD123. In one example, the target on the endogenous HSC is CD124. In one example, the target on the endogenous HSC is CD126. In one example, the target on the endogenous HSC is CD127. In one example, the target on the endogenous HSC is CD130. In one example, the target on the endogenous HSC is CD131. In one example, the target on the endogenous HSC is CD133. In one example, the target on the endogenous HSC is CD134. In one example, the target on the endogenous HSC is CD135. In one example, the target on the endogenous HSC is CD137. In one example, the target on the endogenous HSC is CD138. In one example, the target on the endogenous HSC is CD151. In one example, the target on the endogenous HSC is CD157. In one example, the target on the endogenous HSC is CD162. In one example, the target on the endogenous HSC is CD164. In one example, the target on the endogenous HSC is CD166. In one example, the target on the endogenous HSC is CD168. In one example, the target on the endogenous HSC is CD172a. In one example, the target on the endogenous HSC is CD173. In one example, the target on the endogenous HSC is CD174. In one example, the target on the endogenous HSC is CD175. In one example, the target on the endogenous HSC is CD175s. In one example, the target on the endogenous HSC is CD176. In one example, the target on the endogenous HSC is CD183. In one example, the target on the endogenous HSC is CD184 (CXCR4). In one example, the target on the endogenous HSC is CD191. In one example, the target on the endogenous HSC is CD200. In one example, the target on the endogenous HSC is CD201. In one example, the target on the endogenous HSC is CD205. In one example, the target on the endogenous HSC is CD217. In one example, the target on the endogenous HSC is CD220. In one example, the target on the endogenous HSC is CD221. In one example, the target on the endogenous HSC is CD222. In one example, the target on the endogenous HSC is CD223. In one example, the target on the endogenous HSC is CD224. In one example, the target on the endogenous HSC is CD225. In one example, the target on the endogenous HSC is CD226. In one example, the target on the endogenous HSC is CD227. In one example, the target on the endogenous HSC is CD228. In one example, the target on the endogenous HSC is CD229. In one example, the target on the endogenous HSC is CD230. In one example, the target on the endogenous HSC is CD235a. In one example, the target on the endogenous HSC is CD235b. In one example, the target on the endogenous HSC is CD236. In one example, the target on the endogenous HSC is CD236R. In one example, the target on the endogenous HSC is CD238. In one example, the target on the endogenous HSC is CD240. In one example, the target on the endogenous HSC is CD242. In one example, the target on the endogenous HSC is CD243. In one example, the target on the endogenous HSC is CD277. In one example, the target on the endogenous HSC is CD292. In one example, the target on the endogenous HSC is CDw293. In one example, the target on the endogenous HSC is CD295. In one example, the target on the endogenous HSC is CD298. In one example, the target on the endogenous HSC is CD309. In one example, the target on the endogenous HSC is CD318. In one example, the target on the endogenous HSC is CD324. In one example, the target on the endogenous HSC is CD325. In one example, the target on the endogenous HSC is CD338. In one example, the target on the endogenous HSC is CD344. In one example, the target on the endogenous HSC is CD349. In one example, the target on the endogenous HSC is CD350. In one example, the target on the endogenous HSC is CD361.

In one example, the antibody is administered in an amount sufficient to:
(i) block binding of CD117 by stem cell factor (SCF); and/or
(ii) inhibit activation of CD117 by SCF; and/or
(iii) inhibit CD117+ cell proliferation mediated by SCF; and/or
(iv) deplete a population of CD117+ cells in the subject.

In one example, the antibody is administered in an amount sufficient to block binding of CD117 by SCF. For example, the antibody is administered in an amount sufficient to inhibit binding of CD117 by SCF. It will be apparent to the skilled person that the antibody need not completely block or inhibit binding of CD117 by SCF, rather it need only reduce it by a statistically significant amount, for example, by at least about 10% or 20% or 30% or 40% or 50% or 60% or 70% or 80% or 90% or 95%. In one example, the antibody blocks or inhibits binding of CD117 by SCF by at least about 10%. In another example, the antibody blocks or inhibits binding of CD117 by SCF by at least about 20%. In a further example, the antibody blocks or inhibits binding of CD117 by SCF by at least about 30%. In one example, the antibody blocks or inhibits binding of CD117 by SCF by at least about 40%. In another example, the antibody blocks or inhibits binding of CD117 by SCF by at least about 50%. In a further example, the antibody blocks or inhibits binding of CD117 by SCF by at least about 60%. In one example, the antibody blocks or inhibits binding of CD117 by SCF by at least about 70%. In another example, the antibody blocks or inhibits binding of CD117 by SCF by at least about 80%. In a further example, the antibody blocks or inhibits binding of CD117 by SCF by at least about 90%. In one example, the antibody blocks or inhibits binding of CD117 by SCF by at least about 95%. In one example, the antibody completely blocks or inhibits binding of CD117 by SCF.

Methods for determining blocking or inhibition of binding are known in the art and/or described herein. In one example, the antibody blocks or inhibits binding of CD117 by SCF as determined in a TF-1 proliferation assay.

In one example, the antibody is administered in an amount sufficient to inhibit activation of CD117 by SCF. It will be apparent to the skilled person that the antibody need not completely inhibit activation of CD117 by SCF, rather it need only reduce it by a statistically significant amount, for example, by at least about 10% or 20% or 30% or 40% or 50% or 60% or 70% or 80% or 90% or 95%. In one example, the antibody inhibits activation of CD117 by SCF by at least about 10%. In another example, the antibody inhibits activation of CD117 by SCF by at least about 20%. In a further example, the antibody inhibits activation of CD117 by SCF by at least about 30%. In one example, the antibody inhibits activation of CD117 by SCF by at least about 40%. In another example, the antibody inhibits activation of CD117 by SCF by at least about 50%. In a further example, the antibody inhibits activation of CD117 by SCF by at least about 60%. In one example, the antibody inhibits activation of CD117 by SCF by at least about 70%. In another example, the antibody inhibits activation of CD117 by SCF by at least about 80%. In a further example, the antibody inhibits activation of CD117 by SCF by at least about 90%. In one example, the antibody inhibits activation of CD117 by SCF by at least about 95%. In one example, the antibody completely inhibits activation of CD117 by SCF.

Methods for determining activation inhibition are known in the art and/or described herein. In one example, the antibody inhibits activation of CD117 by SCF as determined in a TF-1 proliferation assay.

In one example, the antibody is administered in an amount sufficient to inhibit CD117+ cell proliferation mediated by SCF. It will be apparent to the skilled person that the antibody need not completely inhibit CD117+ cell proliferation by SCF, rather it need only reduce it by a statistically significant amount, for example, by at least about 10% or 20% or 30% or 40% or 50% or 60% or 70% or 80% or 90% or 95%. In one example, the antibody inhibits CD117+ cell proliferation by SCF by at least about 10%. In another example, the antibody inhibits CD117+ cell proliferation by SCF by at least about 20%. In a further example, the antibody inhibits CD117+ cell proliferation by SCF by at least about 30%. In one example, the antibody inhibits CD117+ cell proliferation by SCF by at least about 40%. In another example, the antibody inhibits CD117+ cell proliferation by SCF by at least about 50%. In a further example, the antibody inhibits CD117+ cell proliferation by SCF by at least about 60%. In one example, the antibody inhibits CD117+ cell proliferation by SCF by at least about 70%. In another example, the antibody inhibits CD117+ cell proliferation by SCF by at least about 80%. In a further example, the antibody inhibits CD117+ cell proliferation by SCF by at least about 90%. In one example, the antibody inhibits CD117+ cell proliferation by SCF by at least about 95%. In one example, the antibody completely inhibits CD117+ cell proliferation by SCF.

Methods for determining inhibition CD117+ cell proliferation by SCF are known in the art and/or described herein. In one example, the antibody inhibits CD117+ cell proliferation by SCF as determined in a TF-1 proliferation assay.

In one example, the antibody is administered in an amount sufficient to deplete a population of CD117+ cells in the subject. It will be apparent to the skilled person that the antibody need not completely deplete a population of CD117+ cells, rather it need only reduce it by a statistically significant amount, for example, by at least about 10% or 20% or 30% or 40% or 50% or 60% or 70% or 80% or 90% or 95%. In one example, the antibody depletes a population of CD117+ cells by at least about 10%. In another example, the antibody depletes a population of CD117+ cells by at least about 20%. In a further example, the antibody depletes a population of CD117+ cells by at least about 30%. In one example, the antibody depletes a population of CD117+ cells by at least about 40%. In another example, the antibody depletes a population of CD117+ cells by at least about 50%. In a further example, the antibody depletes a population of CD117+ cells by at least about 60%. In one example, the antibody depletes a population of CD117+ cells by at least about 70%. In another example, the antibody depletes a population of CD117+ cells at least about 80%. In a further example, the antibody depletes a population of CD117+ cells by at least about 90%. In one example, the antibody depletes a population of CD117+ cells by at least about 95%. In one example, the antibody completely depletes a population of CD117+ cells.

Methods for determining CD117+ cell depletion are known in the art and/or described herein. For example, CD117+ cell depletion is determined using flow cytometry of CD89Tg positive bone marrow cells (e.g., neutrophils and/or macrophages) cultured in the presence of antibody.

In one example, the antibody is an anti-CD117 antibody.

In one example, the antibody binds or specifically binds to the soluble N-terminal of human CD117 and/or the C-terminal of human CD117 and/or full length human CD117.

In one example, the antibody binds or specifically binds to the soluble N-terminal of human CD117. For example, the antibody binds or specifically binds to the soluble N-terminal of human CD117 with an affinity dissociation constant (K_{D}) of 1.5 UM or less. For example, the antibody binds or specifically binds to the soluble N-terminal of human CD117 with a K_{D} of 1 μM of less, such as for example, 900 nM or less, or about 800 nM or less, or 700 nM or less, or 600 nM or less, or 500 nM or less, or 400 nM or less, or 300 nM or less, or 200 nM or less, or 100 nM or less. In one example, the antibody binds or specifically binds to the soluble N-terminal of human CD117 with a K_{D} of 100 nM or less, such as for example, 90 nM or less, or 80 nM or less, or 70 nM or less, or 60 nM or less, or 50 nM or less, or 40 nM or less, or 30 nM or less, or 20 nM or less. For example, the antibody binds or specifically binds to the soluble N-terminal of human CD117 with a K_{D} of about 49 nM. In one example, the antibody binds or specifically binds to the soluble N-terminal of human CD117 with a K_{D} of 20 nM or less. In one example, the antibody binds or specifically binds to the soluble N-terminal of human CD117 with a K_{D} of 20 nM or less, such as 18 nM or less, for example, 15 nM or less, or 12 nM or less, or 10 nM or less. For example, the antibody binds or specifically binds to the soluble N-terminal of human CD117 with a K_{D} of about 14 nM. In another example, the antibody binds or specifically binds to the soluble N-terminal of human CD117 with a K_{D} of about 9 nM. In one example, the antibody binds or specifically binds to the soluble N-terminal of human CD117 with a K_{D} of 10 nM or less, or 8 nM or less, or 5 nM or less, or 3 nM or less, or 2 nM or less, or 1 nM or less, or 0.5 nM or less. For example, the antibody binds or specifically binds to the soluble N-terminal of human CD117 with a K_{D} of between 10 nM and 1 nM. For example, a K_{D} of between about 10 nM and 8 nM, such as a K_{D} of about 8.6 nM or about 9 nM. In one example, the antibody binds or specifically binds to the soluble N-terminal of human CD117 with a K_{D} of between 2 nM and 3 nM, for example a K_{D} of about 2.5 nM. In one example, the antibody binds or specifically binds to the soluble N-terminal of human CD117 with a K_{D} of about 0.5 nM.

In one example, the antibody binds or specifically binds to the C-terminal of human CD117. For example, the antibody binds or specifically binds to the C-terminal of human CD117 with a K_{D} of 200 nM or less. For example, the antibody binds or specifically binds to the soluble N-terminal of human CD117 with a K_{D} of about 191 nM. In one example, the antibody binds or specifically binds to the C-terminal of human CD117 with a K_{D} of 200 nM or less, such as for example, 175 nM or less, or 150 nM or less, or 125 nM or less, or 100 nM or less, or 90 nM or less, or 80 nM or less, or 70 nM or less, or 60 nM or less. In one example, the antibody binds or specifically binds to the C-terminal of human CD117 with a K_{D} of 60 nM or less. For example, the antibody binds or specifically binds to the C-terminal of human CD117 with a K_{D} of 60 nM or less, such as for example, 50 nM or less, or 40 nM or less, or 30 nM or less, or 20 nM or less, or 10 nM or less. For example, the antibody binds or specifically binds to the C-terminal of human CD117 with a K_{D} of 40 nM or less, such as for example, a K_{D} of about 35 nM. For example, the antibody binds or specifically binds to the C-terminal of human CD117 with a K_{D} of 20 nM or less, such as for example, a K_{D} of about 18 nM. For example, the antibody binds or specifically binds to the C-terminal of human CD117 with a K_{D} of 10 nM or less, such as for example, a K_{D} of about 9 nM. For example, the antibody binds or specifically binds to the C-terminal of human CD117 with a K_{D} of 5 nM or less, such as for example, a K_{D} of about 2.5 nM.

In one example, the antibody binds or specifically binds to full length human CD117. For example, the antibody binds or specifically binds to the C-terminal of human CD117 with a K_{D} of 60 nM or less. For example, the antibody binds or specifically binds to the C-terminal of human CD117 with a K_{D} of 60 nM or less, such as for example, a K_{D} of 55 nM or less, or 50 nM or less, or 45 nM or less, or 40 nM or less, or 35 nM or less, or 30 nM or less. For example, the antibody binds or specifically binds to the C-terminal of human CD117 with a K_{D} of about 55 nM. For example, the antibody binds or specifically binds to the C-terminal of human CD117 with a K_{D} of about 38 nM. For example, the antibody binds or specifically binds to the C-terminal of human CD117 with a K_{D} of about 36 nM. For example, the antibody binds or specifically binds to the C-terminal of human CD117 with a K_{D} of about 32 nM. In one example, the antibody binds or specifically binds to the C-terminal of human CD117 with a K_{D} of 30 nM or less, such as for example, a K_{D} of 25 nM or less, or 20 nM or less, or 15 nM or less, or 10 nM or less. For example, the antibody binds or specifically binds to the C-terminal of human CD117 with a K_{D} of about 18 nM. For example, the antibody binds or specifically binds to the C-terminal of human CD117 with a K_{D} of about 14 nM. For example, the antibody binds or specifically binds to the C-terminal of human CD117 with a K_{D} of about 9 nM. In one example, the antibody binds or specifically binds to the C-terminal of human CD117 with a K_{D} of about 5 nM or less, or 4 nM or less, or 3 nM or less, or 2 nM or less, or 1 nM or less. For example, the antibody binds or specifically binds to the C-terminal of human CD117 with a K_{D} of about 4 nM. For example, the antibody binds or specifically binds to the C-terminal of human CD117 with a K_{D} of about 2.5 nM.

In one example, the anti-CD117 antibody binds:
(i) the soluble N-terminal of human CD117 with an affinity dissociation constant (K_{D}) of 1.5 UM or less; and/or
(ii) the C-terminal of human CD117 with a K_{D} of 200 nM or less; and/or
(iii) full length human CD117 with a K_{D} of 60 nM or less.

In one example, the anti-CD117 antibody binds:
(i) the soluble N-terminal of human CD117 with an affinity dissociation constant (K_{D}) of 20 nM or less; and/or
(ii) the C-terminal of human CD117 with a K_{D} of 15 nM or less; and/or
(iii) full length human CD117 with a K_{D} of 60 nM or less.

In one example, the anti-CD117 antibody inhibits SCF mediated proliferation of TF-1 cells. For example, the anti-CD117 antibody inhibits SCF mediated proliferation of TF-1 cells with an IC₅₀ of at least 1 nM. For example, the IC₅₀ is at least 1 nM, or 0.9 nM, or 0.8 nM, or 0.7 nM, or 0.6 nM or 0.5 nM or 0.4 nM, or 0.3 nM, or 0.2 nM, or 0.1 nM. For example, the IC₅₀ is 0.19 nM. For example, the IC₅₀ is 0.65 nM. In one example, the anti-CD117 antibody inhibits SCF mediated proliferation of TF-1 cells with an IC₅₀ of at least 100 μM. For example, the IC₅₀ is at least 90 μM, or 80 μM, or 70 μM, or 60 μM, or 50μ, or 40μ, or 30μ, or 20μ, or 10μ. For example, the IC₅₀ is 90 μM. For example, the IC₅₀ is 45 μM. For example, the IC₅₀ is 38 μM. For example, the IC₅₀ is 18 μM.

Methods for determining the IC₅₀ are described herein and include culturing TF-1 cells (e.g., about 2×10⁵ TF-1 cells) in the presence of the antibody (e.g., anti-CD117 antibody) prior to adding the human SCF and culturing the cells (e.g., for at least about 48 hours or at least about 72 hours or at least about 96 hours, e.g., for about 72 hours) and then determining cell proliferation. Cell proliferation can be determined by using a commercially available kit, such as e.g., a Vialight Plus kit. By determining proliferation in a variety of concentrations of the antibody an IC₅₀ can be determined.

In one example, the anti-CD117 antibody mediates ADCT with a normalised maximum membrane uptake (Bₘₐₓ) of at least 50% as determined by mean fluorescent intensity (MFI) of DILC18 on neutrophils. For example, the antibody mediates ADCT with a normalised Bₘₐₓ of 51%. In one example, the antibody mediates ADCT with a normalised Bₘₐₓ of at least 60%. For example, the antibody mediates ADCT with a normalised Bₘₐₓ of 64%. For example, the antibody mediates ADCT with a normalised Bₘₐₓ of 68%. In one example, the antibody mediates ADCT with a normalised Bₘₐₓ of at least 70%. For example, the antibody mediates ADCT with a normalised Bₘₐₓ of 71%. In one example, the antibody mediates ADCT with a normalised Bₘₐₓ of at least 80%. In one example, the antibody mediates ADCT with a normalised Bₘₐₓ of at least 90%. For example, the antibody mediates ADCT with a normalised Bₘₐₓ of 95%.

Methods for determining the IC₅₀ are described herein and include, for example, flow cytometry. For example, the method includes culturing a cell line transfected with the target (e.g., human CD117) and labelled with a membrane-dye DILC18 (1, 1″-dioctadexyl-3,3, 3″, 3″-tetramethylindocarbocyanine perchlorate) in the presence of peripheral blood neutrophils. Live neutrophils can be identified by forward and side scatter, followed by live CD66b positive cells. Membrane uptake by neutrophils can be measured by an increase of DILC18 mean fluorescence intensity (MFI). DILC18 MFI is normalised to the maximum MFI per donor neutrophil. In one example, the normalised maximum membrane uptake (Bₘₐₓ) in percent facilitated by the antibody inducing trogocytosis by peripheral blood neutrophils is determined.

In one example, the anti-CD117 antibody binds to a cell (e.g., a TF-1 cell or a HEK293FS cell) expressing CD117 (e.g., human CD117 or cynomolgus monkey CD117) with an EC₅₀ of 25 nM or less. In one example, the EC₅₀ is 20 nM or less, or 15 nM or less, or 10 nM or less, or 5 nM or less. In one example, the EC₅₀ is 1 nM or less, or 0.5 nM or less, or 0.4 nM or less, or 0.3 nM or less, or 0.2 nM or less.

In one example, the anti-CD117 antibody competitively inhibits binding of any one or more of the following antibodies to CD117:
(i) an antibody comprising a heavy chain variable region (V_{H}) comprising a sequence set forth in SEQ ID NO: 4 or SEQ ID NO: 13; and a light chain variable region (V_{L}) comprising a sequence set forth in SEQ ID NO: 5 or SEQ ID NO: 28;
(ii) an antibody comprising a V_{H} comprising a sequence set forth in SEQ ID NO: 7; and a V_{L} comprising a sequence set forth in SEQ ID NO: 10;
(iii) an antibody comprising a V_{H} comprising a sequence set forth in SEQ ID NO: 13; and a V_{L} comprising a sequence set forth in SEQ ID NO: 16;
(iv) an antibody comprising a V_{H} comprising a sequence set forth in SEQ ID NO: 19; and a V_{L} comprising a sequence set forth in SEQ ID NO: 22; and
(v) an antibody comprising a V_{H} comprising a sequence set forth in SEQ ID NO: 25; and a V_{L} comprising a sequence set forth in SEQ ID NO: 28.

In one example, the anti-CD117 antibody competitively inhibits binding of an antibody comprising a V_{H} comprising a sequence set forth in SEQ ID NO: 4 or SEQ ID NO: 13; and a V_{L} comprising a sequence set forth in SEQ ID NO: 5 or SEQ ID NO: 28, to CD117.

In one example, the anti-CD117 antibody competitively inhibits binding of an antibody comprising a V_{H} comprising a sequence set forth in SEQ ID NO: 4 and SEQ ID NO: 5, to CD117.

In one example, the anti-CD117 antibody competitively inhibits binding of an antibody comprising a V_{H} comprising a sequence set forth in SEQ ID NO: 4 and SEQ ID NO: 28, to CD117.

In one example, the anti-CD117 antibody competitively inhibits binding of an antibody comprising a V_{H} comprising a sequence set forth in SEQ ID NO: 13; and a V_{L} comprising a sequence set forth in SEQ ID NO: 5 to CD117.

In one example, the anti-CD117 antibody competitively inhibits binding of an antibody comprising a V_{H} comprising a sequence set forth in SEQ ID NO: 7; and a V_{L} comprising a sequence set forth in SEQ ID NO: 10 to CD117.

In one example, the anti-CD117 antibody competitively inhibits binding of an antibody comprising a V_{H} comprising a sequence set forth in SEQ ID NO: 13; and a V_{L} comprising a sequence set forth in SEQ ID NO: 16 to CD117.

In one example, the anti-CD117 antibody competitively inhibits binding of an antibody comprising a V_{H} comprising a sequence set forth in SEQ ID NO: 19; and a V_{L} comprising a sequence set forth in SEQ ID NO: 22 to CD117.

In one example, the anti-CD117 antibody competitively inhibits binding of an antibody comprising a V_{H} comprising a sequence set forth in SEQ ID NO: 25; and a V_{L} comprising a sequence set forth in SEQ ID NO: 28 to CD117.

In one example, the anti-CD117 antibody comprises:
(i) a V_{H} comprising a sequence set forth in SEQ ID NO: 4 or SEQ ID NO: 13; and a V_{L} comprising a sequence set forth in SEQ ID NO: 5 or SEQ ID NO: 28; or
   a. a V_{H} comprising three complementarity determining regions (CDRs) of a V_{H} comprising a sequence set forth in SEQ ID NO: 4 or SEQ ID NO: 13; and a V_{L} comprising three CDRs of a V_{L} comprising a sequence set forth in SEQ ID NO: 5 or SEQ ID NO: 28.

In one example, the anti-CD117 antibody comprises a V_{H} comprising a sequence set forth in SEQ ID NO: 4 or SEQ ID NO: 13; and a V_{L} comprising a sequence set forth in SEQ ID NO: 5 or SEQ ID NO: 28.

In one example, the anti-CD117 antibody comprises a V_{H} comprising a sequence set forth in SEQ ID NO: 4; and a V_{L} comprising a sequence set forth in SEQ ID NO: 5.

In one example, the anti-CD117 antibody comprises a V_{H} comprising a sequence set forth in SEQ ID NO: 4; and a V_{L} comprising a sequence set forth in SEQ ID NO: 28.

In one example, the anti-CD117 antibody comprises a V_{H} comprising a sequence set forth in SEQ ID NO: 13; and a V_{L} comprising a sequence set forth in SEQ ID NO: 5.

In one example, the anti-CD117 antibody comprises a V_{H} comprising three complementarity determining regions (CDRs) of a V_{H} comprising a sequence set forth in SEQ ID NO: 4 or SEQ ID NO: 13; and a V_{L} comprising three CDRs of a V_{L} comprising a sequence set forth in SEQ ID NO: 5 or SEQ ID NO: 28.

In one example, the anti-CD117 antibody comprises a:
(a) V_{H} comprising:
   (i) a CDR1 comprising a sequence set forth in amino acids 31-35 of SEQ ID NO: 4 or SEQ ID NO: 13;
   (ii) a CDR2 comprising a sequence set forth in amino acids 50-66 of SEQ ID NO: 4 or SEQ ID NO: 13; and
   (c) a CDR3 comprising a sequence set forth in amino acids 99-110 of SEQ ID NO: 4 or SEQ ID NO: 13; and
(ii) a V_{L} comprising:
   (a) a CDR1 comprising a sequence set forth in amino acids 23-33 of SEQ ID NO: 5 or SEQ ID NO: 28;
   (b) a CDR2 comprising a sequence set forth in amino acids 49-55 of SEQ ID NO: 5 or SEQ ID NO: 28; and
   (c) a CDR3 comprising a sequence set forth in amino acids 88-98 of SEQ ID NO: 5 or SEQ ID NO: 28.

In one example, the anti-CD117 antibody comprises a V_{H} comprising three CDRs of a V_{H} comprising a sequence set forth in SEQ ID NO: 4; and a V_{L} comprising three CDRs of a V_{L} comprising a sequence set forth in SEQ ID NO: 5.

In one example, the anti-CD117 antibody comprises a:
(a) V_{H} comprising:
   (i) a CDR1 comprising a sequence set forth in amino acids 31-35 of SEQ ID NO: 4, wherein the amino acid sequence comprises Serine(S) or Aspartic acid (D) at position 31 and/or Alanine (A) or Glycine (G) at position 33 and/or Serine(S) or Histidine (H) at position 35;
   (ii) a CDR2 comprising a sequence set forth in amino acids 50-66 of SEQ ID NO: 4, wherein the amino acid sequence comprises Alanine (A) or Valine (V) at position 50 and/or Serine(S) or absent or Glycine G) at position 52 and/or Glycine (G) or Tyrosine (Y) at position 53 and/or Serine(S) or Glycine (G) at position 54 and/or Serine(S) or Glycine (G) at position 57 and/or Threonine (T) or Lysine (K) at position 58; and
   (c) a CDR3 comprising a sequence set forth in amino acids 99-110 of SEQ ID NO: 4, wherein the amino acid sequence comprises Leucine (L) or Serine(S) or Threonine (T) at position 100 and/or Arginine (R) or Serine(S) or Tryptophan (W) at position 101 and/or Valine (V) or Glycine (G) or Tryptophan (W) at position 102 and/or Tyrosine (Y) or Leucine (L) at position 103 and/or Alanine (A) or Arginine (R) at position 104 and/or Methionine (M) or Glycine (G) at position 105 and/or Glutamic acid (E) or Aspartic acid (D) or Leucine (L) at position 106 and/or Alanine (A) or absent at position 107 and/or Isoleucine (I) or Tyrosine (Y) at position 110; and
(ii) a V_{L} comprising:
   (a) a CDR1 comprising a sequence set forth in amino acids 23-33 of SEQ ID NO: 5, wherein the amino acid sequence comprises Glutamine (Q) or Serine(S) at position 23 and/or Serine(S) or Lysine (K) at position 26 and/or Leucine (L) or Valine (V) at position 27 and/or Arginine (R) or Glycine (G) at position 28 and/or Aspartic Acid (D) or Glutamic Acid (E) or Alanine (A) at position 29 and/or Tyrosine (Y) or Threonine (T) or Lysine (K) at position 30 and/or Alanine (A) or Valine (V) at position 32 and/or Asparagine (N) or Histidine (H) or Phenylalanine (F) at position 33;
   (b) a CDR2 comprising a sequence set forth in amino acids 49-55 of SEQ ID NO: 5, wherein the amino acid sequence comprises Tyrosine (Y) or Glutamine (Q) at position 49 and/or Serine(S) or Phenylalanine (F) or Asparagine (N) at position 51 and/or Aspartic Acid (D) or Lysine (K) at position 52; and
   (c) a CDR3 comprising a sequence set forth in amino acids 88-98 of SEQ ID NO: 5, wherein the amino acid sequence comprises Serine(S) or Threonine (T) at position 94 and/or Aspartic acid (D) or Alanine (A) at position 95 and/or Histidine (H) or Valine (V) or Leucine (L) at position 96 and/or Tryptophan (W) or Tyrosine (Y) or absent at position 97 and/or Valine (V) or absent at position 98.

In one example, the anti-CD117 antibody comprises a:
(a) V_{H} comprising:
   (i) a CDR1 comprising a sequence set forth in amino acids 31-35 of SEQ ID NO: 4, wherein the amino acid sequence comprises Serine(S) at position 31 and Alanine (A) at position 33 and Serine(S) at position 35;
   (ii) a CDR2 comprising a sequence set forth in amino acids 50-66 of SEQ ID NO: 4, wherein the amino acid sequence comprises Alanine (A) at position 50 and Serine(S) at position 52 and Glycine (G) at position 53 and Serine(S) at position 54 and Serine(S) at position 57 and Threonine (T) at position 58; and
   (c) a CDR3 comprising a sequence set forth in amino acids 99-110 of SEQ ID NO: 4, wherein the amino acid sequence comprises Leucine (L) at position 100 and Arginine (R) at position 101 and Valine (V) at position 102 and Tyrosine (Y) at position 103 and Alanine (A) at position 104 and Methionine (M) at position 105 and Glutamic acid (E) at position 106 and Alanine (A) at position 107 and Isoleucine (I) at position 110; and
(ii) a V_{L} comprising:
   (a) a CDR1 comprising a sequence set forth in amino acids 23-33 of SEQ ID NO: 5, wherein the amino acid sequence comprises Glutamine (Q) at position 23 and Serine(S) at position 26 and Leucine (L) at position 27 and Arginine (R) at position 28 and Aspartic Acid (D) at position 29 and Tyrosine (Y) at position 30 and Alanine (A) at position 32 and Asparagine (N) at position 33;
   (b) a CDR2 comprising a sequence set forth in amino acids 49-55 of SEQ ID NO: 5, wherein the amino acid sequence comprises Tyrosine (Y) at position 49 and Serine(S) at position 51 and Aspartic Acid (D) at position 52; and
   (c) a CDR3 comprising a sequence set forth in amino acids 88-98 of SEQ ID NO: 5, wherein the amino acid sequence comprises Serine(S) at position 94 and Aspartic acid (D) at position 95 and Histidine (H) at position 96 and Tryptophan (W) at position 97 and Valine (V) at position 98.

In one example, the anti-CD117 antibody comprises a:
(a) V_{H} comprising:
   (i) a CDR1 comprising a sequence set forth in amino acids 31-35 of SEQ ID NO: 4, wherein the amino acid sequence comprises Serine(S) at position 31 and Alanine (A) at position 33 and Histidine (H) at position 35;
   (ii) a CDR2 comprising a sequence set forth in amino acids 50-66 of SEQ ID NO: 4, wherein the amino acid sequence comprises Valine (V) at position 50 and absent at position 52 and Tyrosine (Y) at position 53 and Serine(S) at position 54 and Serine(S) at position 57 and Threonine (T) at position 58; and
   (c) a CDR3 comprising a sequence set forth in amino acids 99-110 of SEQ ID NO: 4, wherein the amino acid sequence comprises Serine(S) at position 100 and Serine(S) at position 101 and Glycine (G) at position 102 and Tyrosine (Y) at position 103 and Arginine (R) at position 104 and Glycine (G) at position 105 and Aspartic acid (D) at position 106 and absent at position 107 and Tyrosine (Y) at position 110; and
(ii) a V_{L} comprising:
   (a) a CDR1 comprising a sequence set forth in amino acids 23-33 of SEQ ID NO: 5, wherein the amino acid sequence comprises Serine(S) at position 23 and Lysine (K) at position 26 and Valine (V) at position 27 and Glycine (G) at position 28 and Alanine (A) at position 29 and Lysine (K) at position 30 and Valine (V) at position 32 and Phenylalanine (F) at position 33;
   (b) a CDR2 comprising a sequence set forth in amino acids 49-55 of SEQ ID NO: 5, wherein the amino acid sequence comprises Glutamine (Q) at position 49 and Asparagine (N) at position 51 and Lysine (K) at position 52; and
   (c) a CDR3 comprising a sequence set forth in amino acids 88-98 of SEQ ID NO: 5, wherein the amino acid sequence comprises Threonine (T) at position 94 and Alanine (A) at position 95 and Leucine (L) at position 96 and Tyrosine (Y) at position 97 and Valine (V) at position 98.

In one example, the anti-CD117 antibody comprises a V_{H} comprising three CDRs of a V_{H} comprising a sequence set forth in SEQ ID NO: 4; and a V_{L} comprising three CDRs of a V_{L} comprising a sequence set forth in SEQ ID NO: 28.

In one example, the anti-CD117 antibody comprises a:
(a) V_{H} comprising:
   (i) a CDR1 comprising a sequence set forth in amino acids 31-35 of SEQ ID NO: 4, wherein the amino acid sequence comprises Aspartic acid (D) at position 31 and Glycine (G) at position 33 and Serine(S) at position 35;
   (ii) a CDR2 comprising a sequence set forth in amino acids 50-66 of SEQ ID NO: 4, wherein the amino acid sequence comprises Alanine (A) at position 50 and Glycine (G) at position 52 and Tyrosine (Y) at position 53 and Glycine (G) at position 54 and Glycine (G) at position 57 and Lysine (K) at position 58; and
   (c) a CDR3 comprising a sequence set forth in amino acids 99-110 of SEQ ID NO: 4, wherein the amino acid sequence comprises Threonine (T) at position 100 and Tryptophan (W) at position 101 and Tryptophan (W) at position 102 and Leucine (L) at position 103 and Alanine (A) at position 104 and Glycine (G) at position 105 and Leucine (L) at position 106 and absent at position 107 and Tyrosine (Y) at position 110; and
(ii) a V_{L} comprising:
   (a) a CDR1 comprising a sequence set forth in amino acids 23-33 of SEQ ID NO: 28;
   (b) a CDR2 comprising a sequence set forth in amino acids 49-55 of SEQ ID NO: 28; and
   (c) a CDR3 comprising a sequence set forth in amino acids 88-98 of SEQ ID NO: 28.

In one example, the anti-CD117 antibody comprises a V_{H} comprising three CDRs of a V_{H} comprising a sequence set forth in SEQ ID NO: 13; and a V_{L} comprising three CDRs of a V_{L} comprising a sequence set forth in SEQ ID NO: 5.

In one example, the anti-CD117 antibody comprises a:
(a) V_{H} comprising:
   (i) a CDR1 comprising a sequence set forth in amino acids 31-35 of SEQ ID NO: 13;
   (ii) a CDR2 comprising a sequence set forth in amino acids 50-66 of SEQ ID NO: 13; and
   (c) a CDR3 comprising a sequence set forth in amino acids 99-110 of SEQ ID NO: 13; and
   (a) a CDR1 comprising a sequence set forth in amino acids 23-33 of SEQ ID NO: 5, wherein the amino acid sequence comprises Serine(S) at position 23 and Lysine (K) at position 26 and Leucine (L) at position 27 and Glycine (G) at position 28 and Glutamic Acid (E) at position 29 and Threonine (T) at position 30 and Valine (V) at position 32 and Histidine (H) at position 33;
   (b) a CDR2 comprising a sequence set forth in amino acids 49-55 of SEQ ID NO: 5, wherein the amino acid sequence comprises Glutamine (Q) at position 49 and Phenylalanine (F) at position 51 and Lysine (K) at position 52; and
   (c) a CDR3 comprising a sequence set forth in amino acids 88-98 of SEQ ID NO: 5, wherein the amino acid sequence comprises Threonine (T) at position 94 and Alanine (A) at position 95 and Valine (V) at position 96 and absent at position 97 and absent at position 98.

In one example, the amino acid sequence of SEQ ID NO: 4 comprises Alanine (A) or Valine (V) or Leucine (L) at position 5 and/or Aspartic acid (D) or Glycine (G) at position 10 and/or Leucine (L) or Valine (V) at position 11 and/or Glycine (G) or Arginine (R) at position 16 and/or Serine(S) or Aspartic acid (D) at position 31 and/or Alanine (A) or Glycine (G) at position 33 and/or Serine(S) or Histidine (H) at position 35 and/or Alanine (A) or Valine (V) at position 50 and/or Serine(S) or absent or Glycine G) at position 52 and/or Glycine (G) or Tyrosine (Y) at position 53 and/or Serine(S) or Glycine (G) at position 54 and/or Serine(S) or Glycine (G) at position 57 and/or Threonine (T) or Lysine (K) at position 58 and/or Isoleucine (I) or Valine (V) at position 93 and/or Arginine (R) or Lysine (K) at position 98 and/or Leucine (L) or Serine(S) or Threonine (T) at position 100 and/or Arginine (R) or Serine(S) or Tryptophan (W) at position 101 and/or Valine (V) or Glycine (G) or Tryptophan (W) at position 102 and/or Tyrosine (Y) or Leucine (L) at position 103 and/or Alanine (A) or Arginine (R) at position 104 and/or Methionine (M) or Glycine (G) at position 105 and/or Glutamic acid (E) or Aspartic acid (D) or Leucine (L) at position 106 and/or Alanine (A) or absent at position 107 and/or Isoleucine (I) or Tyrosine (Y) at position 110 and/or Methionine (M) or Leucine (L) at position 116.

In one example, the amino acid sequence of SEQ ID NO: 4 comprises Alanine (A) at position 5 and Aspartic acid (D) at position 10 and Leucine (L) at position 11 and Glycine (G) at position 16 and Serine(S) at position 31 and Alanine (A) at position 33 and Serine(S) at position 35 and Alanine (A) at position 50 and Serine(S) at position 52 and Glycine (G) at position 53 and Serine(S) at position 54 and Serine(S) at position 57 and Threonine (T) at position 58 and Isoleucine (I) at position 93 and Arginine (R) at position 98 and Leucine (L) at position 100 and Arginine (R) at position 101 and Valine (V) at position 102 and Tyrosine (Y) at position 103 and Alanine (A) at position 104 and Methionine (M) at position 105 and Glutamic acid (E) at position 106 and Alanine (A) at position 107 and Isoleucine (I) at position 110 and Methionine (M) at position 116.

In one example, the amino acid sequence of SEQ ID NO: 4 comprises Valine (V) at position 5 and Glycine (G) at position 10 and Valine (V) at position 11 and Arginine (R) at position 16 and Serine(S) at position 31 and Alanine (A) at position 33 and Histidine (H) at position 35 and Valine (V) at position 50 and absent at position 52 and Tyrosine (Y) at position 53 and Serine(S) at position 54 and Serine(S) at position 57 and Threonine (T) at position 58 and Valine (V) at position 93 and Arginine (R) at position 98 and Serine(S) at position 100 and Serine(S) at position 101 and Glycine (G) at position 102 and Tyrosine (Y) at position 103 and Arginine (R) at position 104 and Glycine (G) at position 105 and Aspartic acid (D) at position 106 and absent at position 107 and Tyrosine (Y) at position 110 and Leucine (L) at position 116.

In one example, the amino acid sequence of SEQ ID NO: 4 comprises Leucine (L) at position 5 and Glycine (G) at position 10 and Leucine (L) at position 11 and Glycine (G) at position 16 and Aspartic acid (D) at position 31 and Glycine (G) at position 33 and Serine(S) at position 35 and Alanine (A) at position 50 and Glycine (G) at position 52 and Tyrosine (Y) at position 53 and Glycine (G) at position 54 and Glycine (G) at position 57 and Lysine (K) at position 58 and Valine (V) at position 93 and Lysine (K) at position 98 and Threonine (T) at position 100 and Tryptophan (W) at position 101 and Tryptophan (W) at position 102 and Leucine (L) at position 103 and Alanine (A) at position 104 and Glycine (G) at position 105 and Leucine (L) at position 106 and absent at position 107 and Tyrosine (Y) at position 110 and Leucine (L) at position 116.

In one example, the amino acid sequence of SEQ ID NO: 5 comprises Aspartic Acid (D) or Proline (P) at position 7 and/or Alanine (A) or Serine(S) at position 9 and/or Alanine (A) or Serine(S) at position 13 and/or Leucine (L) or Proline (P) at position 14 and/or Valine (V) or Alanine (A) at position 18 and/or Arginine (R) or Threonine (T) or Serine(S) at position 19 and/or Glutamine (Q) or Serine(S) at position 23 and/or Serine(S) or Lysine (K) at position 26 and/or Leucine (L) or Valine (V) at position 27 and/or Arginine (R) or Glycine (G) at position 28 and/or Aspartic Acid (D) or Glutamic Acid (E) or Alanine (A) at position 29 and/or Tyrosine (Y) or Threonine (T) or Lysine (K) at position 30 and/or Alanine (A) or Valine (V) at position 32 and/or Asparagine (N) or Histidine (H) or Phenylalanine (F) at position 33 and/or Phenylalanine (F) or Tyrosine (Y) at position 35 and/or Glutamine (Q) or Histidine (H) at position 37 and/or Glutamine (Q) or Lysine (K) at position 38 and/or Proline (P) or Serine(S) at position 39 and/or Alanine (A) or Serine(S) at position 42 and/or Leucine (L) or Isoleucine (I) or Valine (V) at position 44 and/or Isoleucine (I) or Valine (V) at position 47 and/or Phenylalanine (F) or Tyrosine (Y) at position 48 and/or Tyrosine (Y) or Glutamine (Q) at position 49 and/or Serine(S) or Phenylalanine (F) or Asparagine (N) at position 51 and/or Aspartic Acid (D) or Lysine (K) at position 52 and/or Arginine (R) or Glycine (G) at position 76 and/or Valine (V) or Threonine (T) at position 77 and/or Glutamic acid (E) or Glutamine (Q) at position 78 and/or Glycine (G) or Methionine (M) at position 80 and/or Serine(S) or Threonine (T) at position 94 and/or Aspartic acid (D) or Alanine (A) at position 95 and/or Histidine (H) or Valine (V) or Leucine (L) at position 96 and/or Tryptophan (W) or Tyrosine (Y) or absent at position 97 and/or Valine (V) or absent at position 98 and/or Glycine (G) or Threonine (T) at position 101 and/or Leucine (L) or Valine (V) at position 105.

In one example, the amino acid sequence of SEQ ID NO: 5 comprises Aspartic Acid (D) at position 7 and Alanine (A) at position 9 and Alanine (A) at position 13 and Leucine (L) at position 14 and Valine (V) at position 18 and Arginine (R) at position 19 and Glutamine (Q) and Serine(S) position 26 and Leucine (L) at position 27 and Arginine (R) at position 28 and Aspartic Acid (D) at position 29 and Tyrosine (Y) at position 30 and Alanine (A) at position 32 and Asparagine (N) at position 33 and Phenylalanine (F) at position 35 and Glutamine (Q) at position 37 and Lysine (K) at position 38 and Proline (P) at position 39 and Alanine (A) at position 42 and Leucine (L) at position 44 and Isoleucine (I) at position 47 and Phenylalanine (F) and Tyrosine (Y) at position 49 and Serine(S) at position 51 and Aspartic Acid (D) at position 52 and Arginine (R) at position 76 and Valine (V) at position 77 and Glutamic acid (E) at position 78 and Glycine (G) at position 80 and Serine(S) at position 94 and Aspartic acid (D) at position 95 and Histidine (H) at position 96 and Tryptophan (W) at position 97 and Valine (V) at position 98 and Glycine (G) at position 101 and Leucine (L) at position 105.

In one example, the amino acid sequence of SEQ ID NO: 5 comprises Proline (P) at position 7 and Serine(S) at position 9 and Serine(S) at position 13 and Proline (P) at position 14 and Alanine (A) at position 18 and Threonine (T) at position 19 and Serine(S) at position 23 and Lysine (K) at position 26 and Leucine (L) at position 27 and Glycine (G) at position 28 and Glutamic Acid (E) at position 29 and Threonine (T) at position 30 and Valine (V) at position 32 and Histidine (H) at position 33 and Tyrosine (Y) at position 35 and Histidine (H) at position 37 and Glutamine (Q) at position 38 and Serine(S) at position 39 and Serine(S) at position 42 and Isoleucine (I) at position 44 and Valine (V) at position 47 and Tyrosine (Y) at position 48 and Glutamine (Q) at position 49 and Phenylalanine (F) at position 51 and Lysine (K) at position 52 and Glycine (G) at position 76 and Threonine (T) at position 77 and Glutamine (Q) at position 78 and Methionine (M) at position 80 and Threonine (T) at position 94 and Alanine (A) at position 95 and Valine (V) at position 96 and absent at position 97 and absent at position 98 and Glycine (G) at position 101 and Leucine (L) at position 105.

In one example, the amino acid sequence of SEQ ID NO: 5 comprises Proline (P) at position 7 and Serine(S) at position 9 and Serine(S) at position 13 and Proline (P) at position 14 and Alanine (A) at position 18 and Serine(S) at position 19 and Serine(S) at position 23 and Lysine (K) at position 26 and Valine (V) at position 27 and Glycine (G) at position 28 and Alanine (A) at position 29 and Lysine (K) at position 30 and Valine (V) at position 32 and Phenylalanine (F) at position 33 and Tyrosine (Y) at position 35 and Glutamine (Q) at position 37 and Lysine (K) at position 38 and Proline (P) at position 39 and Serine(S) at position 42 and Valine (V) at position 44 and Valine (V) at position 47 and Tyrosine (Y) at position 48 and Glutamine (Q) at position 49 and Asparagine (N) at position 51 and Lysine (K) at position 52 and Glycine (G) at position 76 and Threonine (T) at position 77 and Glutamine (Q) at position 78 and Methionine (M) at position 80 and Threonine (T) at position 94 and Alanine (A) at position 95 and Leucine (L) at position 96 and Tyrosine (Y) at position 97 and Valine (V) at position 98 and Threonine (T) at position 101 and Valine (V) at position 105.

In one example, the anti-CD117 antibody comprises:
(i) a V_{H} comprising three CDRs of a V_{H} comprising a sequence set forth in SEQ ID NO: 7; and a V_{L} comprising three CDRs of a V_{L} comprising a sequence set forth in SEQ ID NO: 10;
(ii) a V_{H} comprising three CDRs of a V_{H} comprising a sequence set forth in SEQ ID NO: 13; and a V_{L} comprising three CDRs of a V_{L} comprising a sequence set forth in SEQ ID NO: 16;
(iii) a V_{H} comprising three CDRs of a V_{H} comprising a sequence set forth in SEQ ID NO: 19; and a V_{L} comprising three CDRs of a V_{L} comprising a sequence set forth in SEQ ID NO: 22; or
(iv) a V_{H} comprising three CDRs of a V_{H} comprising a sequence set forth in SEQ ID NO: 25; and a V_{L} comprising three CDRs of a V_{L} comprising a sequence set forth in SEQ ID NO: 28; or
(v) a V_{H} comprising a CDR1 comprising a sequence set forth in SEQ ID NO: 42, a CDR2 comprising a sequence set forth in SEQ ID NO: 43 and a CDR3 comprising a sequence set forth in SEQ ID NO: 44; and a V_{L} comprising a CDR1 comprising a sequence set forth in SEQ ID NO: 45, a CDR2 comprising a sequence set forth in SEQ ID NO: 46 and a CDR3 comprising a sequence set forth in SEQ ID NO: 47; or
(vi) a V_{H} comprising a CDR1 comprising a sequence set forth in SEQ ID NO: 48, a CDR2 comprising a sequence set forth in SEQ ID NO: 49 and a CDR3 comprising a sequence set forth in SEQ ID NO: 50; and a V_{L} comprising a CDR1 comprising a sequence set forth in SEQ ID NO: 51, a CDR2 comprising a sequence set forth in SEQ ID NO: 52 and a CDR3 comprising a sequence set forth in SEQ ID NO: 53; or
(vii) a V_{H} comprising a CDR1 comprising a sequence set forth in SEQ ID NO: 54, a CDR2 comprising a sequence set forth in SEQ ID NO: 55 and a CDR3 comprising a sequence set forth in SEQ ID NO: 56; and a V_{L} comprising a CDR1 comprising a sequence set forth in SEQ ID NO: 57, a CDR2 comprising a sequence set forth in SEQ ID NO: 58 and a CDR3 comprising a sequence set forth in SEQ ID NO: 59; or
(viii) a V_{H} comprising a CDR1 comprising a sequence set forth in SEQ ID NO: 60, a CDR2 comprising a sequence set forth in SEQ ID NO: 61 and a CDR3 comprising a sequence set forth in SEQ ID NO: 62; and a V_{L} comprising a CDR1 comprising a sequence set forth in SEQ ID NO: 63, a CDR2 comprising a sequence set forth in SEQ ID NO: 64 and a CDR3 comprising a sequence set forth in SEQ ID NO: 65.

In one example, the anti-CD117 antibody comprises a V_{H} comprising three CDRs of a V_{H} comprising a sequence set forth in SEQ ID NO: 7; and a V_{L} comprising three CDRs of a V_{L} comprising a sequence set forth in SEQ ID NO: 10.

In one example, the anti-CD117 antibody comprises a V_{H} comprising a CDR1 comprising amino acids 31-35 of SEQ ID NO: 7, a CDR2 comprising amino acids 50-66 of SEQ ID NO: 7, and a CDR3 comprising amino acids 99-110 of SEQ ID NO: 7; and a V_{L} comprising a CDR1 comprising amino acids 23-33 of SEQ ID NO: 10, a CDR2 comprising amino acids 49-55 of SEQ ID NO: 10, and a CDR3 comprising amino acids 88-98 of SEQ ID NO: 10.

In one example, the anti-CD117 antibody comprises a V_{H} comprising three CDRs of a V_{H} comprising a sequence set forth in SEQ ID NO: 13; and a V_{L} comprising three CDRs of a V_{L} comprising a sequence set forth in SEQ ID NO: 16.

In one example, the anti-CD117 antibody comprises a V_{H} comprising a CDR1 comprising amino acids 31-36 of SEQ ID NO: 13, a CDR2 comprising amino acids 51-66 of SEQ ID NO: 13, and a CDR3 comprising amino acids 99-114 of SEQ ID NO: 13; and a V_{L} comprising a CDR1 comprising amino acids 23-33 of SEQ ID NO: 16, a CDR2 comprising amino acids 49-55 of SEQ ID NO: 16, and a CDR3 comprising amino acids 88-96 of SEQ ID NO: 16.

In one example, the anti-CD117 antibody comprises a V_{H} comprising three CDRs of a V_{H} comprising a sequence set forth in SEQ ID NO: 19; and a V_{L} comprising three CDRs of a V_{L} comprising a sequence set forth in SEQ ID NO: 22.

In one example, the anti-CD117 antibody comprises a V_{H} comprising a CDR1 comprising amino acids 31-35 of SEQ ID NO: 19, a CDR2 comprising amino acids 50-65 of SEQ ID NO: 19, and a CDR3 comprising amino acids 98-108 of SEQ ID NO: 19; and a V_{L} comprising a CDR1 comprising amino acids 23-33 of SEQ ID NO: 22, a CDR2 comprising amino acids 49-55 of SEQ ID NO: 22, and a CDR3 comprising amino acids 88-98 of SEQ ID NO: 22.

In one example, the anti-CD117 antibody comprises a V_{H} comprising three CDRs of a V_{H} comprising a sequence set forth in SEQ ID NO: 25; and a V_{L} comprising three CDRs of a V_{L} comprising a sequence set forth in SEQ ID NO: 28.

In one example, the anti-CD117 antibody comprises a V_{H} comprising a CDR1 comprising amino acids 31-35 of SEQ ID NO: 25, a CDR2 comprising amino acids 50-66 of SEQ ID NO: 25, and a CDR3 comprising amino acids 99-109 of SEQ ID NO: 25; and a V_{L} comprising a CDR1 comprising amino acids 24-35 of SEQ ID NO: 28, a CDR2 comprising amino acids 51-57 of SEQ ID NO: 28, and a CDR3 comprising amino acids 90-98 of SEQ ID NO: 28.

In one example, the anti-CD117 antibody comprises a V_{H} comprising a CDR1 comprising a sequence set forth in SEQ ID NO: 42, a CDR2 comprising a sequence set forth in SEQ ID NO: 43 and a CDR3 comprising a sequence set forth in SEQ ID NO: 44; and a V_{L} comprising a CDR1 comprising a sequence set forth in SEQ ID NO: 45, a CDR2 comprising a sequence set forth in SEQ ID NO: 46 and a CDR3 comprising a sequence set forth in SEQ ID NO: 47.

In one example, the anti-CD117 antibody comprises a V_{H} comprising a CDR1 comprising a sequence set forth in SEQ ID NO: 48, a CDR2 comprising a sequence set forth in SEQ ID NO: 49 and a CDR3 comprising a sequence set forth in SEQ ID NO: 50; and a V_{L} comprising a CDR1 comprising a sequence set forth in SEQ ID NO: 51, a CDR2 comprising a sequence set forth in SEQ ID NO: 52 and a CDR3 comprising a sequence set forth in SEQ ID NO: 53.

In one example, the anti-CD117 antibody comprises a V_{H} comprising a CDR1 comprising a sequence set forth in SEQ ID NO: 54, a CDR2 comprising a sequence set forth in SEQ ID NO: 55 and a CDR3 comprising a sequence set forth in SEQ ID NO: 56; and a V_{L} comprising a CDR1 comprising a sequence set forth in SEQ ID NO: 57, a CDR2 comprising a sequence set forth in SEQ ID NO: 58 and a CDR3 comprising a sequence set forth in SEQ ID NO: 59.

In one example, the anti-CD117 antibody comprises a V_{H} comprising a CDR1 comprising a sequence set forth in SEQ ID NO: 60, a CDR2 comprising a sequence set forth in SEQ ID NO: 61 and a CDR3 comprising a sequence set forth in SEQ ID NO: 62; and a V_{L} comprising a CDR1 comprising a sequence set forth in SEQ ID NO: 63, a CDR2 comprising a sequence set forth in SEQ ID NO: 64 and a CDR3 comprising a sequence set forth in SEQ ID NO: 65.

In one example, the anti-CD117 antibody comprises:
(i) a V_{H} comprising a sequence set forth in SEQ ID NO: 7; and a V_{L} comprising a sequence set forth in SEQ ID NO: 10;
(ii) a V_{H} comprising a sequence set forth in SEQ ID NO: 13; and a V_{L} comprising a sequence set forth in SEQ ID NO: 16;
(iii) a V_{H} comprising a sequence set forth in SEQ ID NO: 19; and a V_{L} comprising a sequence set forth in SEQ ID NO: 22; or
(iv) a V_{H} comprising a sequence set forth in SEQ ID NO: 25; and a V_{L} comprising a sequence set forth in SEQ ID NO: 28.

In one example, the anti-CD117 antibody comprises a V_{H} comprising a sequence set forth in SEQ ID NO: 7; and a V_{L} comprising a sequence set forth in SEQ ID NO: 10.

In one example, the anti-CD117 antibody comprises a V_{H} comprising a sequence set forth in SEQ ID NO: 13; and a V_{L} comprising a sequence set forth in SEQ ID NO: 16.

In one example, the anti-CD117 antibody comprises a V_{H} comprising a sequence set forth in SEQ ID NO: 19; and a V_{L} comprising a sequence set forth in SEQ ID NO: 22.

In one example, the anti-CD117 antibody comprises a V_{H} comprising a sequence set forth in SEQ ID NO: 25; and a V_{L} comprising a sequence set forth in SEQ ID NO: 28.

In one example, the anti-CD117 antibody comprises:
(i) an IgA₂ heavy chain comprising a V_{H} comprising a sequence set forth in SEQ ID NO: 7; and a lambda light chain comprising a V_{L} comprising a sequence set forth in SEQ ID NO: 10;
(ii) an IgA₂ heavy chain comprising a V_{H} comprising a sequence set forth in SEQ ID NO: 13; and a lambda light chain comprising a V_{L} comprising a sequence set forth in SEQ ID NO: 16;
(iii) an IgA₂ heavy chain comprising a V_{H} comprising a sequence set forth in SEQ ID NO: 19; and a lambda light chain comprising a V_{L} comprising a sequence set forth in SEQ ID NO: 22; or
(iv) an IgA₂ heavy chain comprising a V_{H} comprising a sequence set forth in SEQ ID NO: 25; and a kappa light chain comprising a V_{L} comprising a sequence set forth in SEQ ID NO: 28.

In one example, the anti-CD117 antibody comprises an IgA₂ heavy chain comprising a V_{H} comprising a sequence set forth in SEQ ID NO: 7; and a lambda light chain comprising a V_{L} comprising a sequence set forth in SEQ ID NO: 10.

In one example, the anti-CD117 antibody comprises an IgA₂ heavy chain comprising a V_{H} comprising a sequence set forth in SEQ ID NO: 13; and a lambda light chain comprising a V_{L} comprising a sequence set forth in SEQ ID NO: 16.

In one example, the anti-CD117 antibody comprises an IgA₂ heavy chain comprising a V_{H} comprising a sequence set forth in SEQ ID NO: 19; and a lambda light chain comprising a V_{L} comprising a sequence set forth in SEQ ID NO: 22.

In one example, the anti-CD117 antibody comprises an IgA₂ heavy chain comprising a V_{H} comprising a sequence set forth in SEQ ID NO: 25; and a kappa light chain comprising a V_{L} comprising a sequence set forth in SEQ ID NO: 28.

In one example, the anti-CD117 antibody comprises:
(i) a heavy chain comprising a sequence set forth in SEQ ID NO: 6; and a light chain comprising a sequence set forth in SEQ ID NO: 9;
(ii) a heavy chain comprising a sequence set forth in SEQ ID NO: 12; and a light chain comprising a sequence set forth in SEQ ID NO: 15;
(iii) a heavy chain comprising a sequence set forth in SEQ ID NO: 18; and a light chain comprising a sequence set forth in SEQ ID NO: 21;
(iv) a heavy chain comprising a sequence set forth in SEQ ID NO: 24; and a light chain comprising a sequence set forth in SEQ ID NO: 27;
(v) a heavy chain comprising a sequence set forth in SEQ ID NO: 67; and a light chain comprising a sequence set forth in SEQ ID NO: 69;
(vi) a heavy chain comprising a sequence set forth in SEQ ID NO: 71; and a light chain comprising a sequence set forth in SEQ ID NO: 73;
(vii) a heavy chain comprising a sequence set forth in SEQ ID NO: 75; and a light chain comprising a sequence set forth in SEQ ID NO: 77;
(viii) a heavy chain comprising a sequence set forth in SEQ ID NO: 79; and a light chain comprising a sequence set forth in SEQ ID NO: 81;
(ix) a heavy chain comprising a sequence set forth in SEQ ID NO: 83; and a light chain comprising a sequence set forth in SEQ ID NO: 85;
(x) a heavy chain comprising a sequence set forth in SEQ ID NO: 87; and a light chain comprising a sequence set forth in SEQ ID NO: 89;
(xi) a heavy chain comprising a sequence set forth in SEQ ID NO: 91; and a light chain comprising a sequence set forth in SEQ ID NO: 93;
(xii) a heavy chain comprising a sequence set forth in SEQ ID NO: 95; and a light chain comprising a sequence set forth in SEQ ID NO: 97;
(xiii) a heavy chain comprising a sequence set forth in SEQ ID NO: 99; and a light chain comprising a sequence set forth in SEQ ID NO: 101;
(xiv) a heavy chain comprising a sequence set forth in SEQ ID NO: 103; and a light chain comprising a sequence set forth in SEQ ID NO: 105; or
(xv) a heavy chain comprising a sequence set forth in SEQ ID NO: 107; and a light chain comprising a sequence set forth in SEQ ID NO: 109; or
(xvi) a heavy chain comprising a sequence set forth in SEQ ID NO: 113; and a light chain comprising a sequence set forth in SEQ ID NO: 111; or
(xvii) a heavy chain comprising a sequence set forth in SEQ ID NO: 115; and a light chain comprising a sequence set forth in SEQ ID NO: 111.

In one example, the anti-CD117 antibody comprises:
(i) a heavy chain comprising amino acids 20 to 467 of SEQ ID NO: 6; and a light chain comprising amino acids 20 to 233 of SEQ ID NO: 9;
(ii) a heavy chain comprising amino acids 20 to 471 of SEQ ID NO: 12; and a light chain comprising amino acids 20 to 231 of SEQ ID NO: 15;
(iii) a heavy chain comprising amino acids 20 to 465 of SEQ ID NO: 18; and a light chain comprising amino acids 20 to 233 of SEQ ID NO: 21; or
(iv) a heavy chain comprising amino acids 20 to 466 of SEQ ID NO: 24; and a light chain comprising amino acids 20 to 233 of SEQ ID NO: 27; or
(v) a heavy chain comprising amino acids 20 to 470 of SEQ ID NO: 67; and a light chain comprising amino acids 20 to 233 of SEQ ID NO: 69;
(vi) a heavy chain comprising amino acids 20 to 480 of SEQ ID NO: 71; and a light chain comprising amino acids 20 to 233 of SEQ ID NO: 73;
(vii) a heavy chain comprising amino acids 20 to 478 of SEQ ID NO: 75; and a light chain comprising amino acids 20 to 233 of SEQ ID NO: 77;
(viii) a heavy chain comprising amino acids 20 to 474 SEQ ID NO: 79; and a light chain comprising amino acids 20 to 231 of SEQ ID NO: 81;
(ix) a heavy chain comprising amino acids 20 to 484 of SEQ ID NO: 83; and a light chain comprising amino acids 20 to 231 of SEQ ID NO: 85;
(x) a heavy chain comprising amino acids 20 to 468 of SEQ ID NO: 87; and a light chain comprising amino acids 20 to 233 of SEQ ID NO: 89;
(xi) a heavy chain comprising amino acids 20 to 478 of SEQ ID NO: 91; and a light chain comprising amino acids 20 to 233 of SEQ ID NO: 93;
(xii) a heavy chain comprising amino acids 20 to 476 of SEQ ID NO: 95; and a light chain comprising amino acids 20 to 233 of SEQ ID NO: 97;
(xiii) a heavy chain comprising amino acids 20 to 469 of SEQ ID NO: 99; and a light chain comprising amino acids 20 to 234 of SEQ ID NO: 101;
(xiv) a heavy chain comprising amino acids 20 to 479 of SEQ ID NO: 103; and a light chain comprising amino acids 20 to 234 of SEQ ID NO: 105; or
(xv) a heavy chain comprising amino acids 20 to 477 of SEQ ID NO: 107; and a light chain comprising amino acids 20 to 234 of SEQ ID NO: 109.

In one example, the anti-CD117 antibody comprises a heavy chain comprising a sequence set forth in SEQ ID NO: 6; and a light chain comprising a sequence set forth in SEQ ID NO: 9. For example, the anti-CD117 antibody comprises a heavy chain comprising amino acids 20 to 467 of SEQ ID NO: 6; and a light chain comprising amino acids 20 to 233 of SEQ ID NO: 9.

In one example, the anti-CD117 antibody comprises a heavy chain comprising a sequence set forth in SEQ ID NO: 12; and a light chain comprising a sequence set forth in SEQ ID NO: 15. For example, the anti-CD117 antibody comprises a heavy chain comprising amino acids 20 to 471 of SEQ ID NO: 12; and a light chain comprising amino acids 20 to 231 of SEQ ID NO: 15.

In one example, the anti-CD117 antibody comprises a heavy chain comprising a sequence set forth in SEQ ID NO: 18; and a light chain comprising a sequence set forth in SEQ ID NO: 21. For example, the anti-CD117 antibody comprises a heavy chain comprising amino acids 20 to 465 of SEQ ID NO: 18; and a light chain comprising amino acids 20 to 233 of SEQ ID NO: 21.

In one example, the anti-CD117 antibody comprises a heavy chain comprising a sequence set forth in SEQ ID NO: 24; and a light chain comprising a sequence set forth in SEQ ID NO: 27. For example, the anti-CD117 antibody comprises a heavy chain comprising amino acids 20 to 466 of SEQ ID NO: 24; and a light chain comprising amino acids 20 to 233 of SEQ ID NO: 27.

In one example, the anti-CD117 antibody comprises a heavy chain comprising a sequence set forth in SEQ ID NO: 67; and a light chain comprising a sequence set forth in SEQ ID NO: 69. For example, the anti-CD117 antibody comprises a heavy chain comprising amino acids 20 to 470 of SEQ ID NO: 67; and a light chain comprising amino acids 20 to 233 of SEQ ID NO: 69.

In one example, the anti-CD117 antibody comprises a heavy chain comprising a sequence set forth in SEQ ID NO: 71; and a light chain comprising a sequence set forth in SEQ ID NO: 73. For example, the anti-CD117 antibody comprises a heavy chain comprising amino acids 20 to 480 of SEQ ID NO: 71; and a light chain comprising amino acids 20 to 233 of SEQ ID NO: 73.

In one example, the anti-CD117 antibody comprises a heavy chain comprising a sequence set forth in SEQ ID NO: 75; and a light chain comprising a sequence set forth in SEQ ID NO: 77. For example, the anti-CD117 antibody comprises a heavy chain comprising amino acids 20 to 478 of SEQ ID NO: 75; and a light chain comprising amino acids 20 to 233 of SEQ ID NO: 77.

In one example, the anti-CD117 antibody comprises a heavy chain comprising a sequence set forth in SEQ ID NO: 79; and a light chain comprising a sequence set forth in SEQ ID NO: 81. For example, the anti-CD117 antibody comprises a heavy chain comprising amino acids 20 to 474 SEQ ID NO: 79; and a light chain comprising amino acids 20 to 231 of SEQ ID NO: 81.

In one example, the anti-CD117 antibody comprises a heavy chain comprising a sequence set forth in SEQ ID NO: 83; and a light chain comprising a sequence set forth in SEQ ID NO: 85. For example, the anti-CD117 antibody comprises a heavy chain comprising amino acids 20 to 484 of SEQ ID NO: 83; and a light chain comprising amino acids 20 to 231 of SEQ ID NO: 85.

In one example, the anti-CD117 antibody comprises a heavy chain comprising a sequence set forth in SEQ ID NO: 87; and a light chain comprising a sequence set forth in SEQ ID NO: 89. For example, the anti-CD117 antibody comprises a heavy chain comprising amino acids 20 to 468 of SEQ ID NO: 87; and a light chain comprising amino acids 20 to 233 of SEQ ID NO: 89.

In one example, the anti-CD117 antibody comprises a heavy chain comprising a sequence set forth in SEQ ID NO: 91; and a light chain comprising a sequence set forth in SEQ ID NO: 93. For example, the anti-CD117 antibody comprises a heavy chain comprising amino acids 20 to 478 of SEQ ID NO: 91; and a light chain comprising amino acids 20 to 233 of SEQ ID NO: 93.

In one example, the anti-CD117 antibody comprises a heavy chain comprising a sequence set forth in SEQ ID NO: 95; and a light chain comprising a sequence set forth in SEQ ID NO: 97. For example, the anti-CD117 antibody comprises a heavy chain comprising amino acids 20 to 476 of SEQ ID NO: 95; and a light chain comprising amino acids 20 to 233 of SEQ ID NO: 97.

In one example, the anti-CD117 antibody comprises a heavy chain comprising a sequence set forth in SEQ ID NO: 99; and a light chain comprising a sequence set forth in SEQ ID NO: 101 For example, the anti-CD117 antibody comprises a heavy chain comprising amino acids 20 to 469 of SEQ ID NO: 99; and a light chain comprising amino acids 20 to 234 of SEQ ID NO: 101.

In one example, the anti-CD117 antibody comprises a heavy chain comprising a sequence set forth in SEQ ID NO: 103; and a light chain comprising a sequence set forth in SEQ ID NO: 105. For example, the anti-CD117 antibody comprises a heavy chain comprising amino acids 20 to 479 of SEQ ID NO: 103; and a light chain comprising amino acids 20 to 234 of SEQ ID NO: 105.

In one example, the anti-CD117 antibody comprises a heavy chain comprising a sequence set forth in SEQ ID NO: 107; and a light chain comprising a sequence set forth in SEQ ID NO: 109. For example, the anti-CD117 antibody comprises a heavy chain comprising amino acids 20 to 477 of SEQ ID NO: 107; and a light chain comprising amino acids 20 to 234 of SEQ ID NO: 109.

In one example, the subject is suffering from a disease or condition that requires treatment with a HCT. For example, the subject is suffering from a cancer, a hemoglobinopathy disorder, a myelodysplastic disorder, a primary immune deficiency (PID), an autoimmune disorder, an immunodeficiency disorder, or a metabolic disorder.

In one example, the subject is suffering from a cancer. For example, the cancer is a hematologic (or blood) cancer. In one example, the hematologic/blood cell cancer is leukemia, lymphoma or other hematologic cancer. For example, the leukemia is acute lymphoblastic leukemia, acute myeloid leukemia, chronic lymphocytic leukemia, chronic myelogenous leukemia, or hairy cell leukemia. In one example, the lymphoma is an AIDS-related lymphoma, a cutaneous T-cell lymphoma, Hodgkin's lymphoma, non-Hodgkin's lymphoma mycosis fungoides, Sezary syndrome, Waldenstrom's macroglobulinemia, or primary central nervous system lymphoma. In one example, the other hematologic cancer is a chronic myeloproliferative disorder, a multiple myeloma/plasma cell neoplasm, a myelodysplastic syndrome, a myelodysplastic/myeloproliferative disorder, a neuroblastoma, or a Ewing sarcoma.

In one example, the subject is suffering from a myelodysplastic disorder.

In one example, the subject is suffering from a hemoglobinopathy disorder. For example, the hemoglobinopathy disorder is sickle cell anemia, thalassemia, Fanconi anemia, or aplastic anemia.

In one example, the subject is suffering from a primary immune deficiency (PID). For example, the PID is Activated PI3K Delta Syndrome (APDS), X-linked Agammaglobulinemia (XLA), Ataxia Telangiectasia, Chronic Granulomatous Disease (CGD) and Other Phagocytic Cell Disorders, Common Variable Immune Deficiency (CVID), Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, Toll-like Receptor (TLR) Deficiencies (including MyD88 Deficiency, IRAK4 Deficiency, UNC93B Deficiency and TLR3 Mutations), Human Natural Killer Cell Deficiencies, Defects in Interferon-γ (IFN-γ) and Interleukin-12 (IL-12) Signaling, Leukocyte Adhesion Deficiency (LAD), NEMO Deficiency Syndrome, Selective IgA Deficiency, Selective IgM Deficiency, Severe Combined Immune Deficiency (SCID) and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Antibody Deficiency with Normal or Elevated Immunoglobulins, Immunodeficiency with Thymoma (Good's Syndrome), Transcobalamin II Deficiency, Kappa Chain Deficiency, Heavy Chain Deficiencies, Post-Meiotic Segregation (PMS2) Disorder, Unspecified Hypogammaglobulinemia, Chronic Mucocutaneous Candidiasis (CMC), Cartilage Hair Hypoplasia (CHH), X-linked Lymphoproliferative (XLP) Syndromes 1 and 2, X-linked Immune Dysregulation with Polyendocrinopathy (IPEX) Syndrome, Veno-occlusive Disease (VODI), Hoyeraal-Hreidarsson Syndrome (Dyskeratosis Congenita), Immunodeficiency with Centromeric Instability and Facial Anomalies (ICF), Schimke Syndrome, Comel-Netherton Syndrome, Diamond Blackfan Anemia, Schwachmann Diamond Syndrome, Deficiency of Adenosine Deaminase 2, Krabbe Disease (GLD), Alpha-mannosidosis, Nijmegen Breakage Syndrome, or graft-versus-host disease (GvHD). In one example, the PID is Wiskott-Aldrich syndrome, X-linked agammaglobulinemia (XLA), Diamond Blackfan Anemia, Schwachmann Diamond Syndrome, Deficiency of Adenosine Deaminase 2, Krabbe Disease (GLD), Alpha-mannosidosis, Nijmegen Breakage Syndrome, or graft-versus-host disease (GvHD).

In one example, the subject is suffering from an autoimmune disorder. For example, the autoimmune disorder is multiple sclerosis or diabetes. For example, the diabetes is Type I diabetes.

In one example, the subject is suffering from an immunodeficiency disorder. For example, the immunodeficiency disorder is human immunodeficiency virus (HIV) or acquired immunodeficiency syndrome (AIDS).

In one example, the subject is suffering from a metabolic disorder. For example, the metabolic disorder is a glycogen storage disease, mucopolysaccharidoses, Gaucher's Disease, Hurlers Disease, sphingolipidoses, or metachromatic leukodystrophy.

In one example, the subject has received, is receiving, or will receive an additional conditioning regimen. For example, the subject has received an additional conditioning regimen. In one example, the subject is receiving an additional conditioning regimen. In a further example, the subject will receive an additional conditioning regimen.

In one example, the additional conditioning regimen is a non-myeloablative or reduced-intensity conditioning regimen. For example, the additional conditioning regimen is a non-myeloablative conditioning regimen. In one example, the additional conditioning regimen is a reduced-intensity conditioning regimen.

Non-myeloablative and reduced-intensity conditioning regimens suitable for use in the present disclosure will be apparent to the skilled person and/or are described herein.

In one example of any method described herein, the subject further receives a HCT following administration of the antibody.

The present disclosure provides a method of HCT in a subject in need thereof, wherein the subject has previously received treatment with an antibody that binds or specifically binds to a target on an endogenous HSC in the subject.

The present disclosure also provides a method of HCT in a subject in need thereof, wherein the subject has previously received treatment with an antibody that binds or specifically binds to a target on an endogenous HSC in the subject, wherein the antibody mediates ADCT.

The present disclosure further provides a method of HCT in a subject in need thereof, the method comprising:
(i) conditioning a patient with an antibody that binds or specifically binds to a target on an endogenous HSC in the subject; and
(ii) administering a hematopoietic cell graft to the subject.

The present disclosure also provides a method of reducing side effects of a myeloablative conditioning regimen in a subject, the method comprising administering to the subject an antibody that binds or specifically binds to a target on an endogenous HSC in the subject.

The present disclosure further provides a method of reducing side effects of a myeloablative conditioning regimen in a subject, the method comprising administering to the subject an antibody that binds or specifically binds to a target on an endogenous HSC in the subject, wherein the antibody mediates ADCT.

The present disclosure also provides a method of reducing side effects of a myeloablative conditioning regimen in a subject, the method comprising administering to the subject an antibody comprising an extended hinge region.

The present disclosure further provides a method of reducing side effects of a myeloablative conditioning regimen in a subject, the method comprising administering to the subject an antibody comprising an extended hinge region.

The present disclosure provides an anti-CD117 antibody, wherein the antibody comprises:
(i) a V_{H} comprising a sequence set forth in SEQ ID NO: 7; and a V_{L} comprising a sequence set forth in SEQ ID NO: 10;
(ii) a V_{H} comprising a sequence set forth in SEQ ID NO: 13; and a V_{L} comprising a sequence set forth in SEQ ID NO: 16;
(iii) a V_{H} comprising a sequence set forth in SEQ ID NO: 19; and a V_{L} comprising a sequence set forth in SEQ ID NO: 22; or
(iv) a V_{H} comprising a sequence set forth in SEQ ID NO: 25; and a V_{L} comprising a sequence set forth in SEQ ID NO: 28.

The present disclosure provides an anti-CD117 antibody, wherein the antibody comprises a V_{H} comprising a sequence set forth in SEQ ID NO: 7; and a V_{L} comprising a sequence set forth in SEQ ID NO: 10.

The present disclosure provides an anti-CD117 antibody, wherein the antibody comprises a V_{H} comprising a sequence set forth in SEQ ID NO: 13; and a V_{L} comprising a sequence set forth in SEQ ID NO: 16.

The present disclosure provides an anti-CD117 antibody, wherein the antibody comprises a V_{H} comprising a sequence set forth in SEQ ID NO: 19; and a V_{L} comprising a sequence set forth in SEQ ID NO: 22.

The present disclosure provides an anti-CD117 antibody, wherein the antibody comprises a V_{H} comprising a sequence set forth in SEQ ID NO: 25; and a V_{L} comprising a sequence set forth in SEQ ID NO: 28.

The present disclosure further provides an anti-CD117 antibody, wherein the antibody comprises:
(i) an IgA₂ heavy chain comprising a V_{H} comprising a sequence set forth in SEQ ID NO: 7; and a lambda light chain comprising a V_{L} comprising a sequence set forth in SEQ ID NO: 10;
(ii) an IgA₂ heavy chain comprising a V_{H} comprising a sequence set forth in SEQ ID NO: 13; and a lambda light chain comprising a V_{L} comprising a sequence set forth in SEQ ID NO: 16;
(iii) an IgA₂ heavy chain comprising a V_{H} comprising a sequence set forth in SEQ ID NO: 19; and a lambda light chain comprising a V_{L} comprising a sequence set forth in SEQ ID NO: 22; or
(iv) an IgA₂ heavy chain comprising a V_{H} comprising a sequence set forth in SEQ ID NO: 25; and a kappa light chain comprising a V_{L} comprising a sequence set forth in SEQ ID NO: 28.

The present disclosure provides an anti-CD117 antibody, wherein the antibody comprises an IgA₂ heavy chain comprising a V_{H} comprising a sequence set forth in SEQ ID NO: 7; and a lambda light chain comprising a V_{L} comprising a sequence set forth in SEQ ID NO: 10.

The present disclosure provides an anti-CD117 antibody, wherein the antibody comprises an IgA₂ heavy chain comprising a V_{H} comprising a sequence set forth in SEQ ID NO: 13; and a lambda light chain comprising a V_{L} comprising a sequence set forth in SEQ ID NO: 16.

The present disclosure provides an anti-CD117 antibody, wherein the antibody comprises an IgA₂ heavy chain comprising a V_{H} comprising a sequence set forth in SEQ ID NO: 19; and a lambda light chain comprising a V_{L} comprising a sequence set forth in SEQ ID NO: 22.

The present disclosure provides an anti-CD117 antibody, wherein the antibody comprises an IgA₂ heavy chain comprising a V_{H} comprising a sequence set forth in SEQ ID NO: 25; and a kappa light chain comprising a V_{L} comprising a sequence set forth in SEQ ID NO: 28.

The present disclosure also provides an anti-CD117 antibody, wherein the antibody comprises:
(i) a heavy chain comprising a sequence set forth in SEQ ID NO: 6; and a light chain comprising a sequence set forth in SEQ ID NO: 9;
(ii) a heavy chain comprising a sequence set forth in SEQ ID NO: 12; and a light chain comprising a sequence set forth in SEQ ID NO: 15;
(iii) a heavy chain comprising a sequence set forth in SEQ ID NO: 18; and a light chain comprising a sequence set forth in SEQ ID NO: 21;
(iv) a heavy chain comprising a sequence set forth in SEQ ID NO: 24; and a light chain comprising a sequence set forth in SEQ ID NO: 27;
(v) a heavy chain comprising a sequence set forth in SEQ ID NO: 67; and a light chain comprising a sequence set forth in SEQ ID NO: 69;
(vi) a heavy chain comprising a sequence set forth in SEQ ID NO: 71; and a light chain comprising a sequence set forth in SEQ ID NO: 73;
(vii) a heavy chain comprising a sequence set forth in SEQ ID NO: 75; and a light chain comprising a sequence set forth in SEQ ID NO: 77;
(viii) a heavy chain comprising a sequence set forth in SEQ ID NO: 79; and a light chain comprising a sequence set forth in SEQ ID NO: 81;
(ix) a heavy chain comprising a sequence set forth in SEQ ID NO: 83; and a light chain comprising a sequence set forth in SEQ ID NO: 85;
(x) a heavy chain comprising a sequence set forth in SEQ ID NO: 87; and a light chain comprising a sequence set forth in SEQ ID NO: 89;
(xi) a heavy chain comprising a sequence set forth in SEQ ID NO: 91; and a light chain comprising a sequence set forth in SEQ ID NO: 93;
(xii) a heavy chain comprising a sequence set forth in SEQ ID NO: 95; and a light chain comprising a sequence set forth in SEQ ID NO: 97;
(xiii) a heavy chain comprising a sequence set forth in SEQ ID NO: 99; and a light chain comprising a sequence set forth in SEQ ID NO: 101;
(xiv) a heavy chain comprising a sequence set forth in SEQ ID NO: 103; and a light chain comprising a sequence set forth in SEQ ID NO: 105; or
(xv) a heavy chain comprising a sequence set forth in SEQ ID NO: 107; and a light chain comprising a sequence set forth in SEQ ID NO: 109.

In one example, the heavy chain and/or light chain sequence does not comprise a signal sequence.

The present disclosure also provides an anti-CD117 antibody, wherein the antibody comprises:
(i) a heavy chain comprising amino acids 20 to 467 of SEQ ID NO: 6; and a light chain comprising amino acids 20 to 233 of SEQ ID NO: 9;
(ii) a heavy chain comprising amino acids 20 to 471 of SEQ ID NO: 12; and a light chain comprising amino acids 20 to 231 of SEQ ID NO: 15;
(iii) a heavy chain comprising amino acids 20 to 465 of SEQ ID NO: 18; and a light chain comprising amino acids 20 to 233 of SEQ ID NO: 21; or
(iv) a heavy chain comprising amino acids 20 to 466 of SEQ ID NO: 24; and a light chain comprising amino acids 20 to 233 of SEQ ID NO: 27; or
(v) a heavy chain comprising amino acids 20 to 470 of SEQ ID NO: 67; and a light chain comprising amino acids 20 to 233 of SEQ ID NO: 69;
(vi) a heavy chain comprising amino acids 20 to 480 of SEQ ID NO: 71; and a light chain comprising amino acids 20 to 233 of SEQ ID NO: 73;
(vii) a heavy chain comprising amino acids 20 to 478 of SEQ ID NO: 75; and a light chain comprising amino acids 20 to 233 of SEQ ID NO: 77;
(viii) a heavy chain comprising amino acids 20 to 474 SEQ ID NO: 79; and a light chain comprising amino acids 20 to 231 of SEQ ID NO: 81;
(ix) a heavy chain comprising amino acids 20 to 484 of SEQ ID NO: 83; and a light chain comprising amino acids 20 to 231 of SEQ ID NO: 85;
(x) a heavy chain comprising amino acids 20 to 468 of SEQ ID NO: 87; and a light chain comprising amino acids 20 to 233 of SEQ ID NO: 89;
(xi) a heavy chain comprising amino acids 20 to 478 of SEQ ID NO: 91; and a light chain comprising amino acids 20 to 233 of SEQ ID NO: 93;
(xii) a heavy chain comprising amino acids 20 to 476 of SEQ ID NO: 95; and a light chain comprising amino acids 20 to 233 of SEQ ID NO: 97;
(xiii) a heavy chain comprising amino acids 20 to 469 of SEQ ID NO: 99; and a light chain comprising amino acids 20 to 234 of SEQ ID NO: 101;
(xiv) a heavy chain comprising amino acids 20 to 479 of SEQ ID NO: 103; and a light chain comprising amino acids 20 to 234 of SEQ ID NO: 105; or
(xv) a heavy chain comprising amino acids 20 to 477 of SEQ ID NO: 107; and a light chain comprising amino acids 20 to 234 of SEQ ID NO: 109.

The present disclosure also provides an anti-CD117 antibody, wherein the antibody comprises a heavy chain comprising a sequence set forth in SEQ ID NO: 6; and a light chain comprising a sequence set forth in SEQ ID NO: 9. For example, the anti-CD117 antibody comprises a heavy chain comprising amino acids 20 to 467 of SEQ ID NO: 6; and a light chain comprising amino acids 20 to 233 of SEQ ID NO: 9.

The present disclosure also provides an anti-CD117 antibody, wherein the antibody comprises a heavy chain comprising a sequence set forth in SEQ ID NO: 12; and a light chain comprising a sequence set forth in SEQ ID NO: 15. For example, the anti-CD117 antibody comprises a heavy chain comprising amino acids 20 to 471 of SEQ ID NO: 12; and a light chain comprising amino acids 20 to 231 of SEQ ID NO: 15.

The present disclosure also provides an anti-CD117 antibody, wherein the antibody comprises a heavy chain comprising a sequence set forth in SEQ ID NO: 18; and a light chain comprising a sequence set forth in SEQ ID NO: 21. For example, the anti-CD117 antibody comprises a heavy chain comprising amino acids 20 to 465 of SEQ ID NO: 18; and a light chain comprising amino acids 20 to 233 of SEQ ID NO: 21.

The present disclosure also provides an anti-CD117 antibody, wherein the antibody comprises a heavy chain comprising a sequence set forth in SEQ ID NO: 24; and a light chain comprising a sequence set forth in SEQ ID NO: 27. For example, the anti-CD117 antibody comprises a heavy chain comprising amino acids 20 to 466 of SEQ ID NO: 24; and a light chain comprising amino acids 20 to 233 of SEQ ID NO: 27.

The present disclosure also provides an anti-CD117 antibody, wherein the antibody comprises a heavy chain comprising a sequence set forth in SEQ ID NO: 67; and a light chain comprising a sequence set forth in SEQ ID NO: 69. For example, the anti-CD117 antibody comprises a heavy chain comprising amino acids 20 to 470 of SEQ ID NO: 67; and a light chain comprising amino acids 20 to 233 of SEQ ID NO: 69.

The present disclosure also provides an anti-CD117 antibody, wherein the antibody comprises a heavy chain comprising a sequence set forth in SEQ ID NO: 71; and a light chain comprising a sequence set forth in SEQ ID NO: 73. For example, the anti-CD117 antibody comprises a heavy chain comprising amino acids 20 to 480 of SEQ ID NO: 71; and a light chain comprising amino acids 20 to 233 of SEQ ID NO: 73.

The present disclosure also provides an anti-CD117 antibody, wherein the antibody comprises a heavy chain comprising a sequence set forth in SEQ ID NO: 75; and a light chain comprising a sequence set forth in SEQ ID NO: 77. For example, the anti-CD117 antibody comprises a heavy chain comprising amino acids 20 to 478 of SEQ ID NO: 75; and a light chain comprising amino acids 20 to 233 of SEQ ID NO: 77.

The present disclosure also provides an anti-CD117 antibody, wherein the antibody comprises a heavy chain comprising a sequence set forth in SEQ ID NO: 79; and a light chain comprising a sequence set forth in SEQ ID NO: 81. For example, the anti-CD117 antibody comprises a heavy chain comprising amino acids 20 to 474 SEQ ID NO: 79; and a light chain comprising amino acids 20 to 231 of SEQ ID NO: 81.

The present disclosure also provides an anti-CD117 antibody, wherein the antibody comprises a heavy chain comprising a sequence set forth in SEQ ID NO: 83; and a light chain comprising a sequence set forth in SEQ ID NO: 85. For example, the anti-CD117 antibody comprises a heavy chain comprising amino acids 20 to 484 of SEQ ID NO: 83; and a light chain comprising amino acids 20 to 231 of SEQ ID NO: 85.

The present disclosure also provides an anti-CD117 antibody, wherein the antibody comprises a heavy chain comprising a sequence set forth in SEQ ID NO: 87; and a light chain comprising a sequence set forth in SEQ ID NO: 89. For example, the anti-CD117 antibody comprises a heavy chain comprising amino acids 20 to 468 of SEQ ID NO: 87; and a light chain comprising amino acids 20 to 233 of SEQ ID NO: 89.

The present disclosure also provides an anti-CD117 antibody, wherein the antibody comprises a heavy chain comprising a sequence set forth in SEQ ID NO: 91; and a light chain comprising a sequence set forth in SEQ ID NO: 93. For example, the anti-CD117 antibody comprises a heavy chain comprising amino acids 20 to 478 of SEQ ID NO: 91; and a light chain comprising amino acids 20 to 233 of SEQ ID NO: 93.

The present disclosure also provides an anti-CD117 antibody, wherein the antibody comprises a heavy chain comprising a sequence set forth in SEQ ID NO: 95; and a light chain comprising a sequence set forth in SEQ ID NO: 97. For example, the anti-CD117 antibody comprises a heavy chain comprising amino acids 20 to 476 of SEQ ID NO: 95; and a light chain comprising amino acids 20 to 233 of SEQ ID NO: 97.

The present disclosure also provides an anti-CD117 antibody, wherein the antibody comprises a heavy chain comprising a sequence set forth in SEQ ID NO: 99; and a light chain comprising a sequence set forth in SEQ ID NO: 101 For example, the anti-CD117 antibody comprises a heavy chain comprising amino acids 20 to 469 of SEQ ID NO: 99; and a light chain comprising amino acids 20 to 234 of SEQ ID NO: 101.

The present disclosure also provides an anti-CD117 antibody, wherein the antibody comprises a heavy chain comprising a sequence set forth in SEQ ID NO: 103; and a light chain comprising a sequence set forth in SEQ ID NO: 105. For example, the anti-CD117 antibody comprises a heavy chain comprising amino acids 20 to 479 of SEQ ID NO: 103; and a light chain comprising amino acids 20 to 234 of SEQ ID NO: 105.

The present disclosure also provides an anti-CD117 antibody, wherein the antibody comprises a heavy chain comprising a sequence set forth in SEQ ID NO: 107; and a light chain comprising a sequence set forth in SEQ ID NO: 109. For example, the anti-CD117 antibody comprises a heavy chain comprising amino acids 20 to 477 of SEQ ID NO: 107; and a light chain comprising amino acids 20 to 234 of SEQ ID NO: 109.

The present disclosure also provides an anti-CD117 antibody, wherein the antibody comprises:
(i) a heavy chain comprising a sequence expressed from or encoded by a nucleic acid comprising SEQ ID NO: 8; and a light chain comprising a sequence expressed from or encoded by a nucleic acid comprising SEQ ID NO: 11;
(ii) a heavy chain comprising a sequence expressed from or encoded by a nucleic acid comprising SEQ ID NO: 14; and a light chain comprising a sequence expressed from or encoded by a nucleic acid comprising SEQ ID NO: 17;
(iii) a heavy chain comprising a sequence expressed from or encoded by a nucleic acid comprising SEQ ID NO: 20; and a light chain comprising a sequence expressed from or encoded by a nucleic acid comprising SEQ ID NO: 23;
(iv) a heavy chain comprising a sequence expressed from or encoded by a nucleic acid comprising SEQ ID NO: 26; and a light chain comprising a sequence expressed from or encoded by a nucleic acid comprising SEQ ID NO: 29;
(v) a heavy chain comprising a sequence expressed from or encoded by a nucleic acid comprising SEQ ID NO: 68; and a light chain comprising a sequence expressed from or encoded by a nucleic acid comprising SEQ ID NO: 70;
(vi) a heavy chain comprising a sequence expressed from or encoded by a nucleic acid comprising SEQ ID NO: 72; and a light chain comprising a sequence expressed from or encoded by a nucleic acid comprising SEQ ID NO: 74;
(vii) a heavy chain comprising a sequence expressed from or encoded by a nucleic acid comprising SEQ ID NO: 76; and a light chain comprising a sequence expressed from or encoded by a nucleic acid comprising SEQ ID NO: 78;
(viii) a heavy chain comprising a sequence expressed from or encoded by a nucleic acid comprising SEQ ID NO: 80; and a light chain comprising a sequence expressed from or encoded by a nucleic acid comprising SEQ ID NO: 82;
(ix) a heavy chain comprising a sequence expressed from or encoded by a nucleic acid comprising SEQ ID NO: 84; and a light chain comprising a sequence expressed from or encoded by a nucleic acid comprising SEQ ID NO: 86;
(x) a heavy chain comprising a sequence expressed from or encoded by a nucleic acid comprising SEQ ID NO: 88; and a light chain comprising a sequence expressed from or encoded by a nucleic acid comprising SEQ ID NO: 90;
(xi) a heavy chain comprising a sequence expressed from or encoded by a nucleic acid comprising SEQ ID NO: 92; and a light chain comprising a sequence expressed from or encoded by a nucleic acid comprising SEQ ID NO: 94;
(xii) a heavy chain comprising a sequence expressed from or encoded by a nucleic acid comprising SEQ ID NO: 96; and a light chain comprising a sequence expressed from or encoded by a nucleic acid comprising SEQ ID NO: 98;
(xiii) a heavy chain comprising a sequence expressed from or encoded by a nucleic acid comprising SEQ ID NO: 100; and a light chain comprising a sequence expressed from or encoded by a nucleic acid comprising SEQ ID NO: 102;
(xiv) a heavy chain comprising a sequence expressed from or encoded by a nucleic acid comprising SEQ ID NO: 104; and a light chain comprising a sequence expressed from or encoded by a nucleic acid comprising SEQ ID NO: 106; or
(xv) a heavy chain comprising a sequence expressed from or encoded by a nucleic acid comprising SEQ ID NO: 108; and a light chain comprising a sequence expressed from or encoded by a nucleic acid comprising SEQ ID NO: 110; or
(xvi) a heavy chain comprising a sequence expressed from or encoded by a nucleic acid comprising SEQ ID NO: 114; and a light chain comprising a sequence expressed from or encoded by a nucleic acid comprising SEQ ID NO: 112; or
(xvii) a heavy chain comprising a sequence expressed from or encoded by a nucleic acid comprising SEQ ID NO: 116; and a light chain comprising a sequence expressed from or encoded by a nucleic acid comprising SEQ ID NO: 112.

The present disclosure also provides an anti-CD117 antibody, wherein the antibody comprises a heavy chain comprising a sequence expressed from or encoded by a nucleic acid comprising SEQ ID NO: 8; and a light chain comprising a sequence expressed from or encoded by a nucleic acid comprising SEQ ID NO: 11.

In one example, the disclosure provides an anti-CD117 antibody, wherein the antibody comprises a heavy chain comprising a sequence expressed from or encoded by a nucleic acid comprising SEQ ID NO: 8; and a light chain comprising a sequence expressed from or encoded by a nucleic acid comprising SEQ ID NO: 11, optionally wherein the light chain lacks the intronic sequence corresponding to nucleotides 46 to 128 of SEQ ID NO: 11.

In one example, the disclosure provides an anti-CD117 antibody, wherein the antibody comprises a heavy chain comprising a sequence expressed from or encoded by a nucleic acid comprising SEQ ID NO: 14; and a light chain comprising a sequence expressed from or encoded by a nucleic acid comprising SEQ ID NO: 17.

In one example, the disclosure provides an anti-CD117 antibody, wherein the antibody comprises a heavy chain comprising a sequence expressed from or encoded by a nucleic acid comprising SEQ ID NO: 14; and a light chain comprising a sequence expressed from or encoded by a nucleic acid comprising SEQ ID NO: 17, optionally wherein the light chain lacks the intronic sequence corresponding to nucleotides 46 to 128 of SEQ ID NO: 17.

In one example, the disclosure provides an anti-CD117 antibody, wherein the antibody comprises a heavy chain comprising a sequence expressed from or encoded by a nucleic acid comprising SEQ ID NO: 20; and a light chain comprising a sequence expressed from or encoded by a nucleic acid comprising SEQ ID NO: 23.

In one example, the disclosure provides an anti-CD117 antibody, wherein the antibody comprises a heavy chain comprising a sequence expressed from or encoded by a nucleic acid comprising SEQ ID NO: 20; and a light chain comprising a sequence expressed from or encoded by a nucleic acid comprising SEQ ID NO: 23, optionally wherein the light chain lacks the intronic sequence corresponding to nucleotides 46 to 128 of SEQ ID NO: 23.

In one example, the disclosure provides an anti-CD117 antibody, wherein the antibody comprises a heavy chain comprising a sequence expressed from or encoded by a nucleic acid comprising SEQ ID NO: 26; and a light chain comprising a sequence expressed from or encoded by a nucleic acid comprising SEQ ID NO: 29.

In one example, the disclosure provides an anti-CD117 antibody, wherein the antibody comprises a heavy chain comprising a sequence expressed from or encoded by a nucleic acid comprising SEQ ID NO: 26; and a light chain comprising a sequence expressed from or encoded by a nucleic acid comprising SEQ ID NO: 29, optionally wherein the light chain lacks the intronic sequence corresponding to nucleotides 46 to 128 of SEQ ID NO: 29.

The present disclosure also provides an anti-CD117 antibody, wherein the antibody comprises a heavy chain comprising a sequence expressed from or encoded by a nucleic acid comprising SEQ ID NO: 68; and a light chain comprising a sequence expressed from or encoded by a nucleic acid comprising SEQ ID NO: 70.

The present disclosure also provides an anti-CD117 antibody, wherein the antibody comprises a heavy chain comprising a sequence expressed from or encoded by a nucleic acid comprising SEQ ID NO: 68; and a light chain comprising a sequence expressed from or encoded by a nucleic acid comprising SEQ ID NO: 70, optionally wherein the light chain lacks the intronic sequence corresponding to nucleotides 46 to 128 of SEQ ID NO: 70.

The present disclosure also provides an anti-CD117 antibody, wherein the antibody comprises a heavy chain comprising a sequence expressed from or encoded by a nucleic acid comprising SEQ ID NO: 72; and a light chain comprising a sequence expressed from or encoded by a nucleic acid comprising SEQ ID NO: 74.

The present disclosure also provides an anti-CD117 antibody, wherein the antibody comprises a heavy chain comprising a sequence expressed from or encoded by a nucleic acid comprising SEQ ID NO: 72; and a light chain comprising a sequence expressed from or encoded by a nucleic acid comprising SEQ ID NO: 74, optionally wherein the light chain lacks the intronic sequence corresponding to nucleotides 46 to 128 of SEQ ID NO: 74.

The present disclosure also provides an anti-CD117 antibody, wherein the antibody comprises a heavy chain comprising a sequence expressed from or encoded by a nucleic acid comprising SEQ ID NO: 76; and a light chain comprising a sequence expressed from or encoded by a nucleic acid comprising SEQ ID NO: 78.

The present disclosure also provides an anti-CD117 antibody, wherein the antibody comprises a heavy chain comprising a sequence expressed from or encoded by a nucleic acid comprising SEQ ID NO: 76; and a light chain comprising a sequence expressed from or encoded by a nucleic acid comprising SEQ ID NO: 78, optionally wherein the light chain lacks the intronic sequence corresponding to nucleotides 46 to 128 of SEQ ID NO: 78.

The present disclosure also provides an anti-CD117 antibody, wherein the antibody comprises a heavy chain comprising a sequence expressed from or encoded by a nucleic acid comprising SEQ ID NO: 80; and a light chain comprising a sequence expressed from or encoded by a nucleic acid comprising SEQ ID NO: 82.

The present disclosure also provides an anti-CD117 antibody, wherein the antibody comprises a heavy chain comprising a sequence expressed from or encoded by a nucleic acid comprising SEQ ID NO: 80; and a light chain comprising a sequence expressed from or encoded by a nucleic acid comprising SEQ ID NO: 82, optionally wherein the light chain lacks the intronic sequence corresponding to nucleotides 46 to 128 of SEQ ID NO: 82.

The present disclosure also provides an anti-CD117 antibody, wherein the antibody comprises a heavy chain comprising a sequence expressed from or encoded by a nucleic acid comprising SEQ ID NO: 84; and a light chain comprising a sequence expressed from or encoded by a nucleic acid comprising SEQ ID NO: 86.

The present disclosure also provides an anti-CD117 antibody, wherein the antibody comprises a heavy chain comprising a sequence expressed from or encoded by a nucleic acid comprising SEQ ID NO: 84; and a light chain comprising a sequence expressed from or encoded by a nucleic acid comprising SEQ ID NO: 86, optionally wherein the light chain lacks the intronic sequence corresponding to nucleotides 46 to 128 of SEQ ID NO: 86.

The present disclosure also provides an anti-CD117 antibody, wherein the antibody comprises a heavy chain comprising a sequence expressed from or encoded by a nucleic acid comprising SEQ ID NO: 88; and a light chain comprising a sequence expressed from or encoded by a nucleic acid comprising SEQ ID NO: 90.

The present disclosure also provides an anti-CD117 antibody, wherein the antibody comprises a heavy chain comprising a sequence expressed from or encoded by a nucleic acid comprising SEQ ID NO: 88; and a light chain comprising a sequence expressed from or encoded by a nucleic acid comprising SEQ ID NO: 90, optionally wherein the light chain lacks the intronic sequence corresponding to nucleotides 46 to 128 of SEQ ID NO: 90.

The present disclosure also provides an anti-CD117 antibody, wherein the antibody comprises a heavy chain comprising a sequence expressed from or encoded by a nucleic acid comprising SEQ ID NO: 92; and a light chain comprising a sequence expressed from or encoded by a nucleic acid comprising SEQ ID NO: 94.

The present disclosure also provides an anti-CD117 antibody, wherein the antibody comprises a heavy chain comprising a sequence expressed from or encoded by a nucleic acid comprising SEQ ID NO: 92; and a light chain comprising a sequence expressed from or encoded by a nucleic acid comprising SEQ ID NO: 94, optionally wherein the light chain lacks the intronic sequence corresponding to nucleotides 46 to 128 of SEQ ID NO: 94.

The present disclosure also provides an anti-CD117 antibody, wherein the antibody comprises a heavy chain comprising a sequence expressed from or encoded by a nucleic acid comprising SEQ ID NO: 96; and a light chain comprising a sequence expressed from or encoded by a nucleic acid comprising SEQ ID NO: 98.

The present disclosure also provides an anti-CD117 antibody, wherein the antibody comprises a heavy chain comprising a sequence expressed from or encoded by a nucleic acid comprising SEQ ID NO: 96; and a light chain comprising a sequence expressed from or encoded by a nucleic acid comprising SEQ ID NO: 98, optionally wherein the light chain lacks the intronic sequence corresponding to nucleotides 46 to 128 of SEQ ID NO: 98.

The present disclosure also provides an anti-CD117 antibody, wherein the antibody comprises a heavy chain comprising a sequence expressed from or encoded by a nucleic acid comprising SEQ ID NO: 100; and a light chain comprising a sequence expressed from or encoded by a nucleic acid comprising SEQ ID NO: 102.

The present disclosure also provides an anti-CD117 antibody, wherein the antibody comprises a heavy chain comprising a sequence expressed from or encoded by a nucleic acid comprising SEQ ID NO: 100; and a light chain comprising a sequence expressed from or encoded by a nucleic acid comprising SEQ ID NO: 102, optionally wherein the light chain lacks the intronic sequence corresponding to nucleotides 46 to 128 of SEQ ID NO: 102.

The present disclosure also provides an anti-CD117 antibody, wherein the antibody comprises a heavy chain comprising a sequence expressed from or encoded by a nucleic acid comprising SEQ ID NO: 104; and a light chain comprising a sequence expressed from or encoded by a nucleic acid comprising SEQ ID NO: 106.

The present disclosure also provides an anti-CD117 antibody, wherein the antibody comprises a heavy chain comprising a sequence expressed from or encoded by a nucleic acid comprising SEQ ID NO: 104; and a light chain comprising a sequence expressed from or encoded by a nucleic acid comprising SEQ ID NO: 106, optionally wherein the light chain lacks the intronic sequence corresponding to nucleotides 46 to 128 of SEQ ID NO: 106.

The present disclosure also provides an anti-CD117 antibody, wherein the antibody comprises a heavy chain comprising a sequence expressed from or encoded by a nucleic acid comprising SEQ ID NO: 108; and a light chain comprising a sequence expressed from or encoded by a nucleic acid comprising SEQ ID NO: 110.

The present disclosure also provides an anti-CD117 antibody, wherein the antibody comprises a heavy chain comprising a sequence expressed from or encoded by a nucleic acid comprising SEQ ID NO: 108; and a light chain comprising a sequence expressed from or encoded by a nucleic acid comprising SEQ ID NO: 110, optionally wherein the light chain lacks the intronic sequence corresponding to nucleotides 46 to 128 of SEQ ID NO: 110.

The present disclosure also provides an anti-CD117 antibody comprising an extended hinge region comprises a heavy chain comprising a sequence set forth in SEQ ID NO: 113; and a light chain comprising a sequence set forth in SEQ ID NO: 111.

The present disclosure also provides an anti-CD117 antibody comprising an extended hinge region comprises a heavy chain comprising a sequence set forth in SEQ ID NO: 115; and a light chain comprising a sequence set forth in SEQ ID NO: 111.

The present disclosure also provides an anti-CD117 antibody comprising an extended hinge region, wherein the antibody comprises a heavy chain comprising a sequence expressed from or encoded by a nucleic acid comprising SEQ ID NO: 114; and a light chain comprising a sequence expressed from or encoded by a nucleic acid comprising SEQ ID NO: 112.

The present disclosure also provides an anti-CD117 antibody extended hinge region, wherein the antibody comprises a heavy chain comprising a sequence expressed from or encoded by a nucleic acid comprising SEQ ID NO: 116; and a light chain comprising a sequence expressed from or encoded by a nucleic acid comprising SEQ ID NO: 112.

The present disclosure also provides an anti-CD117 antibody comprising an extended hinge region comprising:
(i) a C-terminal CH1 sequence from an IgA, an upper hinge sequence from an IgG₃, a middle hinge sequence from an IgG₃, a lower hinge sequence from an IgG₃, and a sequence past the lower hinge sequence from an IgA; or
(ii) a C-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and a sequence past the lower hinge comprising a sequence set forth in SEQ ID NO: 127; or
(iii) a C-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and a sequence past the lower hinge comprising a sequence set forth in SEQ ID NO: 128.

In one example, the anti-CD117 antibody comprising an extended hinge region comprises a C-terminal CH1 sequence from an IgA, an upper hinge sequence from an IgG₃, a middle hinge sequence from an IgG₃, a lower hinge sequence from an IgG₃, and a sequence past the lower hinge sequence from an IgA. In one example, the anti-CD117 antibody comprising an extended hinge region comprises a C-terminal CH1 sequence from an IgA. In one example, the anti-CD117 antibody comprising an extended hinge region comprises an upper hinge sequence from an IgG₃. In one example, the anti-CD117 antibody comprising an extended hinge region comprises a middle hinge sequence from an IgG₃. In one example, the anti-CD117 antibody comprising an extended hinge region comprises a lower hinge sequence from an IgG₃. In one example, the anti-CD117 antibody comprising an extended hinge region comprises a sequence past the lower hinge sequence from an IgA.

In one example, the anti-CD117 antibody comprising an extended hinge region comprises a C-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 127 and combinations thereof. In one example, the anti-CD117 antibody comprising an extended hinge region comprises a C-terminal CH1 comprising a sequence set forth in SEQ ID NO: 123. In one example, the anti-CD117 antibody comprising an extended hinge region comprises an upper hinge comprising a sequence set forth in SEQ ID NO: 124. In one example, the anti-CD117 antibody comprising an extended hinge region comprises a middle hinge comprising a sequence set forth in SEQ ID NO: 125. In one example, the anti-CD117 antibody comprising an extended hinge region comprises an upper hinge comprising a lower hinge comprising a sequence set forth in SEQ ID NO: 126. In one example, the anti-CD117 antibody comprising an extended hinge region comprises a sequence past the lower hinge comprising a sequence set forth in SEQ ID NO: 127.

In one example, the anti-CD117 antibody comprising an extended hinge region comprises a-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 128 and combinations thereof. In one example, the anti-CD117 antibody comprising an extended hinge region comprises a C-terminal CH1 comprising a sequence set forth in SEQ ID NO: 123. In one example, the anti-CD117 antibody comprising an extended hinge region comprises an upper hinge comprising a sequence set forth in SEQ ID NO: 124. In one example, the anti-CD117 antibody comprising an extended hinge region comprises a middle hinge comprising a sequence set forth in SEQ ID NO: 125. In one example, the anti-CD117 antibody comprising an extended hinge region comprises an upper hinge comprising a lower hinge comprising a sequence set forth in SEQ ID NO: 126. In one example, the anti-CD117 antibody comprising an extended hinge region comprises a sequence past the lower hinge comprising a sequence set forth in SEQ ID NO: 128.

In one example, the level of effector function induced by the anti-CD117 antibody comprising an extended hinge region is enhanced relative to an antibody without the extended hinge region. In one example, the anti-CD117 antibody comprising an extended hinge region enhances the pharmacokinetic properties of the antibody relative to an antibody without the extended hinge region. In one example, the anti-CD117 antibody comprising an extended hinge region enhances the half-life of the antibody relative the anti-CD117 antibody comprising an extended hinge region. In one example, the anti-CD117 antibody comprising an extended hinge region enhances the stability of the anti-CD117 antibody comprising an extended hinge region relative to an antibody without the extended hinge region. In one example, the anti-CD117 antibody comprising an extended hinge region increases the affinity of the hinge region for the neonatal Fc region (FcRn) relative to an antibody without the extended hinge region. In one example, the anti-CD117 antibody comprising an extended hinge region has better access to its epitope relative to an antibody without the extended hinge region.

The present disclosure further provides a method of modifying a hinge region of an antibody of the present disclosure, the method comprising replacing the hinge region of the antibody with an extended hinge region.

In one example, the hinge region is modified by inserting an additional hinge region into a hinge region of the antibody.

In another example, the hinge region is modified by inserting at least of a portion of the additional hinge region into the hinge region of the antibody after the first proline. For example, the hinge region is modified by inserting at least of a portion of an IgG₃ hinge region into an IgA₂ hinge region of the antibody after the first proline.

In one example, the modified hinge region is an extended hinge region.

In one example, the extended hinge region is capable of inducing an enhanced level of effector function.

In one example, the level of effector function induced by the extended hinge region is enhanced relative to an antibody without the extended hinge region. In one example, the extended hinge region enhances the pharmacokinetic properties of the antibody relative to an antibody without the extended hinge region. In one example, the extended hinge region enhances the half-life of the antibody relative an antibody without the extended hinge region. In one example, the extended hinge region enhances the stability of the antibody relative to an antibody without the extended hinge region. In one example, the extended hinge region increases the affinity of the hinge region for the neonatal Fc region (FcRn) relative to an antibody without the extended hinge region. In one example, the extended hinge region provides better access to its epitope relative to an antibody without the extended hinge region.

In one example, the extended hinge region comprises a C-terminal CH1 sequence from an IgA, an upper hinge sequence from an IgG₃, a middle hinge sequence from an IgG₃, a lower hinge sequence from an IgG₃, and/or a sequence past the lower hinge sequence from an IgA and combinations thereof. In one example, the extended hinge region comprises a C-terminal CH1 sequence from an IgA. In one example, the extended hinge region comprises an upper hinge sequence from an IgG₃. In one example, the extended hinge region comprises a middle hinge sequence from an IgG₃. In one example, the extended hinge region comprises a lower hinge sequence from an IgG₃. In one example, the extended hinge region comprises a sequence past the lower hinge sequence from an IgA.

In one example, the extended hinge region comprises a C-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 127 and combinations thereof. In one example, the extended hinge region comprises a C-terminal CH1 comprising a sequence set forth in SEQ ID NO: 123. In one example, the extended hinge region comprises an upper hinge comprising a sequence set forth in SEQ ID NO: 124. In one example, the extended hinge region comprises a middle hinge comprising a sequence set forth in SEQ ID NO: 125. In one example, the extended hinge region comprises an upper hinge comprising a lower hinge comprising a sequence set forth in SEQ ID NO: 126. In one example, the extended hinge region comprises a sequence past the lower hinge comprising a sequence set forth in SEQ ID NO: 127.

In one example, the extended hinge region comprises a-terminal CH1 comprising a sequence set forth in SEQ ID NO: 123, an upper hinge comprising a sequence set forth in SEQ ID NO: 124, a middle hinge comprising a sequence set forth in SEQ ID NO: 125, a lower hinge comprising a sequence set forth in SEQ ID NO: 126, and/or a sequence past the lower hinge comprising a sequence set forth in SEQ ID NO: 128 and combinations thereof. In one example, the extended hinge region comprises a C-terminal CH1 comprising a sequence set forth in SEQ ID NO: 123. In one example, the extended hinge region comprises an upper hinge comprising a sequence set forth in SEQ ID NO: 124. In one example, the extended hinge region comprises a middle hinge comprising a sequence set forth in SEQ ID NO: 125. In one example, the extended hinge region comprises an upper hinge comprising a lower hinge comprising a sequence set forth in SEQ ID NO: 126. In one example, the extended hinge region comprises a sequence past the lower hinge comprising a sequence set forth in SEQ ID NO: 128.

The present disclosure further provides a polynucleotide encoding the anti-CD117 as disclosed herein. In one example, the polynucleotide is operably linked to a heterologous promoter.

The present disclosure additionally provides an expression construct comprising the nucleic acid of the disclosure operably linked to a promoter. Such an expression construct can be in a vector, e.g., a plasmid.

In one example, an expression construct of the disclosure comprises a nucleic acid encoding one of the polypeptides (e.g., comprising a V_{H}) operably linked to a promoter and a nucleic acid encoding another of the polypeptides (e.g., comprising a V_{L}) operably linked to another promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide.

For example, the first polypeptide comprises a V_{H} and the second polypeptide comprises a V_{L}, or the first polypeptide comprises a V_{L} and the second polypeptide comprises a V_{H}.

The present disclosure also contemplates separate expression constructs one of which encodes a first polypeptide (e.g., comprising a V_{H}) and another of which encodes a second polypeptide (e.g., comprising a V_{L}). For example, the present disclosure also provides a composition comprising:
(i) a first expression construct comprising a nucleic acid encoding a polypeptide (e.g., comprising a V_{H} operably linked to a promoter); and
(ii) a second expression construct comprising a nucleic acid encoding a polypeptide (e.g., comprising a V_{L} operably linked to a promoter),
   wherein the first and second polypeptides associate to form an antibody of the present disclosure.

The present disclosure additionally provides an isolated cell expressing the antibody of the present disclosure or a recombinant cell genetically-modified to express an antibody of the disclosure. For example, the disclosure provides use of an isolated cell for preparing the antibody of the disclosure. In one example, the cell is an isolated hybridoma. In another example, the cell comprises the nucleic acid of the disclosure or the expression construct of the disclosure or:
(i) a first expression construct comprising a nucleic acid encoding a polypeptide (e.g., comprising a V_{H}) operably linked to a promoter; and
(ii) a second expression construct comprising a nucleic acid encoding a polypeptide (e.g., comprising a V_{L}) operably linked to a promoter,
   wherein the first and second polypeptides associate to form an antibody of the present disclosure.

The present disclosure additionally provides a pharmaceutical composition comprising the antibody or the nucleic acid or the expression construct or the cell of the present disclosure and a suitable carrier. In one example, the pharmaceutical composition comprises the antibody of the present disclosure.

In one example, the carrier is pharmaceutically acceptable.

The present disclosure additionally provides the antibody or the nucleic acid or the expression construct or the cell or the composition of the present disclosure for use as a medicament.

The present disclosure also provides the antibody or the nucleic acid or the expression construct or the cell or the composition of the present disclosure for use in conditioning a subject in need of a HCT.

The present disclosure also provides use of an antibody that binds or specifically binds to a target on an endogenous HSC in the manufacture of a medicament for conditioning a subject in need of a HCT.

The present disclosure also provides use of an antibody that binds or specifically binds to a target on an endogenous HSC in the manufacture of a medicament for conditioning a subject in need of a HCT, wherein the antibody mediates ADCT.

The present disclosure further provides the antibody or the nucleic acid or the expression construct or the cell or the composition of the present disclosure for use in depleting a population of cells in the bone marrow of a subject in need of a HCT.

The present disclosure also provides use of an antibody that binds or specifically binds to a target on an endogenous HSC in the manufacture of a medicament for depleting a population of cells in the bone marrow of a subject in need of a HCT.

The present disclosure also provides use of an antibody that binds or specifically binds to a target on an endogenous HSC in the manufacture of a medicament for depleting a population of cells in the bone marrow of a subject in need of a HCT, wherein the antibody mediates antibody dependent cellular trogocytosis (ADCT).

The present disclosure further provides the antibody or the nucleic acid or the expression construct or the cell or the composition of the present disclosure for use in a method of HCT.

The present disclosure further provides the antibody or the nucleic acid or the expression construct or the cell or the composition of the present disclosure for use in reducing side effects of a myeloablative conditioning regimen.

The present disclosure also provides use of an antibody that binds or specifically binds to a target on an endogenous HSC in the manufacture of a medicament for reducing side effects of a myeloablative conditioning regimen in a subject.

The present disclosure also provides use of an antibody that binds or specifically binds to a target on an endogenous HSC in the manufacture of a medicament for reducing side effects of a myeloablative conditioning regimen in a subject, wherein the antibody mediates ADCT.

The present disclosure also provides use of an antibody comprising an extended hinge region in the manufacture of a medicament for conditioning a subject in need of a HCT.

The present disclosure also provides use of an antibody comprising an extended hinge region in the manufacture of a medicament for conditioning a subject in need of a HCT, wherein the antibody mediates ADCT.

The present disclosure also provides use of an antibody comprising an extended hinge region in the manufacture of a medicament for depleting a population of cells in the bone marrow of a subject in need of a HCT.

The present disclosure also provides use of an antibody comprising an extended hinge region in the manufacture of a medicament for depleting a population of cells in the bone marrow of a subject in need of a HCT, wherein the antibody mediates antibody dependent cellular trogocytosis (ADCT).

The present disclosure also provides use of an antibody comprising an extended hinge region in the manufacture of a medicament for reducing side effects of a myeloablative conditioning regimen in a subject.

The present disclosure also provides use of an antibody comprising an extended hinge region in the manufacture of a medicament for reducing side effects of a myeloablative conditioning regimen in a subject, wherein the antibody mediates ADCT.

The present disclosure additionally provides an antibody comprising an extended hinge region, the antigen binding site of an antibody of the disclosure comprising an extended hinge region is better able to bind to its epitope relative to the antigen binding site of an antibody without the extended hinge region.

The present disclosure additionally provides an anti-CD117 antibody comprising an extended hinge region. In one example, the antigen binding site of an antibody of the disclosure comprising an extended hinge region is better able to bind to its epitope relative to the antigen binding site of an antibody without the extended hinge region.

The present disclosure additionally provides a method for improving the ability of an antigen binding site of an antibody to bind to its epitope, the method comprising replacing the hinge region of the antibody with an extended hinge region.

Exemplary extended hinge regions and antibodies are described herein and shall be taken to apply equally to the foregoing examples of the disclosure.

The present disclosure also provides a kit comprising the antibody or the nucleic acid or the expression construct or the cell or the composition of the present disclosure packaged with instructions for use in any method described herein (e.g., a method of HCT or depleting a population of cells in the bone marrow of a subject).

In one example, the subject is a mammal, for example a primate, such as a human.

### BRIEF DESCRIPTION OF THE DRAWINGS

FIG. 1 is a series of graphical representations showing binding of anti-CD117 antibodies to cells expressing human, cynomolgus monkey and mouse CD117. (A) Binding curves of varying concentrations of anti-CD117-IgG1 antibodies to cell-surface CD117 on TF-1 cells. (B) EC₅₀ (nM) data from a single representative experiment. Specific binding curves of anti-CD117-IgG1 antibodies to HEK293FS cells stably expressing (C) human CD117, (D) cynomolgus monkey CD117 and (E) mouse CD117, generated by subtracting off the non-specific binding observed on parental HEK293FS cells. EC₅₀ (nM) data from representative experiments of anti-CD117-IgG1 antibodies to HEK293FS cells stably expressing (F) human CD117, (G) cynomolgus monkey CD117 and (H) mouse CD117, where * indicates EC₅₀ values could not be determined.

FIG. 2 is a graphical representation showing inhibition of human stem cell factor mediated proliferation on TF-1 cells by anti-CD117-IgG1 antibodies.

FIG. 3 is a series of graphical representations showing internalisation of selected anti-CD117-IgG1 antibodies using (A) an anti-AF488 quenching antibody assay and (B) an IncuCyte® live-cell analysis.

FIG. 4 is a graphical representation showing the inability of anti-CD117-IgG1 antibodies to activate CD117 receptor and induce proliferation of TF-1 cells in the absence of stem cell factor.

FIG. 5 is a series of graphical representations showing screening of anti-human CD117 antibodies against soluble N-terminal (HuCD117₁₋₃₀₇) and C-terminal (HuCD117₃₀₈₋₅₂₄) fragments of the human CD117 receptor. (A) Positive binding responses are indicated in shaded bars. Overall distribution of binding affinities of anti-human CD117 antibodies (dark circles) tested against (B) soluble N-terminal (HuCD117₁₋₃₀₇) and (C) C-terminal (HuCD117₃₀₈₋₅₂₄) fragments of the human CD117 receptor. Diagonal lines indicate isoaffinity regions.

FIG. 6 is a series of graphical representations showing SPR screening of anti-human CD117 antibodies against soluble (A) human CD117 and (B) cynoCD117 receptors. Antibody ligands were prepared on an IgG1 (black bars), IgG4 (white bars) or IgA2 (grey bars) format.

FIG. 7 is a series of graphical representations showing anti CD117-IgA2 antibodies induce antibody-dependent cytotoxic trogocytosis (ADCT). (A) Normalised DILC18 MFI±SD (B) EC₅₀ and (C) Bₘₐₓ from n=5 donors pooled from three experiments.

### KEY TO SEQUENCE LISTING

SEQ ID NO: 1 Human CD117
SEQ ID NO: 2 Cynomolgus monkey CD117
SEQ ID NO: 3 Murine CD117
SEQ ID NO: 4 Consensus amino acid sequence of heavy chain variable region for 3C2, 3B3 and 4A2
SEQ ID NO: 5 Consensus amino acid sequence of light chain variable region for 3C2, 3B2 and 3B3
SEQ ID NO: 6 Antibody 3C2 heavy chain amino acid sequence (hu3C2-hLG4p)
SEQ ID NO: 7 Antibody 3C2 heavy chain VH amino acid sequence
SEQ ID NO: 8 Antibody 3C2 heavy chain nucleotide sequence (hu3C2-hLG4p)
SEQ ID NO: 9 Antibody 3C2 light chain amino acid sequence (hu3C2-hLG4p)
SEQ ID NO: 10 Antibody 3C2 light chain VL amino acid sequence
SEQ ID NO: 11 Antibody 3C2 light chain nucleotide sequence (hu3C2-hLG4p)
SEQ ID NO: 12 Antibody 3B2 heavy chain amino acid sequence (hu3B2-hLG4p)
SEQ ID NO: 13 Antibody 3B2 heavy chain VH amino acid sequence
SEQ ID NO: 14 Antibody 3B2 heavy chain nucleotide sequence (hu3B2-hLG4p)
SEQ ID NO: 15 Antibody 3B2 light chain amino acid sequence (hu3B2-hLG4p)
SEQ ID NO: 16 Antibody 3B2 light chain VL amino acid sequence
SEQ ID NO: 17 Antibody 3B2 light chain nucleotide sequence (hu3B2-hLG4p)
SEQ ID NO: 18 Antibody 3B3 heavy chain amino acid sequence (hu3B3-hLG4)
SEQ ID NO: 19 Antibody 3B3 heavy chain VH amino acid sequence
SEQ ID NO: 20 Antibody 3B3 heavy chain nucleotide sequence (hu3B3-hLG4)
SEQ ID NO: 21 Antibody 3B3 light chain amino acid sequence (hu3B3-hLG4)
SEQ ID NO: 22 Antibody 3B3 light chain VL amino acid sequence
SEQ ID NO: 23 Antibody 3B3 light chain nucleotide sequence (hu3B3-hLG4)
SEQ ID NO: 24 Antibody 4A2 heavy chain amino acid sequence (hu4A2-hKG4p)
SEQ ID NO: 25 Antibody 4A2 heavy chain VH amino acid sequence
SEQ ID NO: 26 Antibody 4A2 heavy chain nucleotide sequence (hu4A2-hKG4p)
SEQ ID NO: 27 Antibody 4A2 light chain amino acid sequence (hu4A2-hKG4p)
SEQ ID NO: 28 Antibody 4A2 light chain VL amino acid sequence
SEQ ID NO: 29 Antibody 4A2 light chain nucleotide sequence (hu4A2-hKG4p)
SEQ ID NO: 30 IgG₁ heavy chain constant region
SEQ ID NO: 31 IgG₄ heavy chain constant region
SEQ ID NO: 32 IgA₂ wild-type constant region sequence (IgA₂m (1) allotype)
SEQ ID NO: 33 IgA₂ heavy chain constant region (modified)
SEQ ID NO: 34 IgA₂ heavy chain constant region (optimised)
SEQ ID NO: 35 Lambda light chain constant region
SEQ ID NO: 36 Kappa light chain constant region
SEQ ID NO: 37 Heavy chain signal sequence
SEQ ID NO: 38 Light chain signal sequence
SEQ ID NO: 39 Light chain signal sequence
SEQ ID NO: 40 Light chain signal sequence
SEQ ID NO: 41 Light chain intron sequence
SEQ ID NO: 42 Antibody 3C2 V_{H} CDR1 amino acid sequence
SEQ ID NO: 43 Antibody 3C2 V_{H} CDR2 amino acid sequence
SEQ ID NO: 44 Antibody 3C2 V_{H} CDR3 amino acid sequence
SEQ ID NO: 45 Antibody 3C2 V_{L} CDR1 amino acid sequence
SEQ ID NO: 46 Antibody 3C2 V_{L} CDR2 amino acid sequence
SEQ ID NO: 47 Antibody 3C2 V_{L} CDR3 amino acid sequence
SEQ ID NO: 48 Antibody 3B2 V_{H} CDR1 amino acid sequence
SEQ ID NO: 49 Antibody 3B2 V_{H} CDR2 amino acid sequence
SEQ ID NO: 50 Antibody 3B2 V_{H} CDR3 amino acid sequence
SEQ ID NO: 51 Antibody 3B2 V_{L} CDR1 amino acid sequence
SEQ ID NO: 52 Antibody 3B2 V_{L} CDR2 amino acid sequence
SEQ ID NO: 53 Antibody 3B2 V_{L} CDR3 amino acid sequence
SEQ ID NO: 54 Antibody 3B3 V_{H} CDR1 amino acid sequence
SEQ ID NO: 55 Antibody 3B3 V_{H} CDR2 amino acid sequence
SEQ ID NO: 56 Antibody 3B3 V_{H} CDR3 amino acid sequence
Antibody 3B3 V_{L} CDR1 amino acid sequence SEQ ID NO: 57
SEQ ID NO: 58 Antibody 3B3 V_{L} CDR2 amino acid sequence
SEQ ID NO: 59 Antibody 3B3 V_{L} CDR3 amino acid sequence
SEQ ID NO: 60 Antibody 4A2 V_{H} CDR1 amino acid sequence
SEQ ID NO: 61 Antibody 4A2 V_{H} CDR2 amino acid sequence
SEQ ID NO: 62 Antibody 4A2 V_{H} CDR3 amino acid sequence
SEQ ID NO: 63 Antibody 4A2 V_{L} CDR1 amino acid sequence
SEQ ID NO: 64 Antibody 4A2 V_{L} CDR2 amino acid sequence
SEQ ID NO: 65 Antibody 4A2 V_{L} CDR3 amino acid sequence
SEQ ID NO: 66 IgA₂ heavy chain constant region consensus sequence
SEQ ID NO: 67 Antibody 3C2 heavy chain amino acid sequence (hu3C2-hLG1)
SEQ ID NO: 68 Antibody 3C2 heavy chain nucleotide sequence (hu3C2-hLG1)
SEQ ID NO: 69 Antibody 3C2 light chain amino acid sequence (hu3C2-hLG1)
SEQ ID NO: 70 Antibody 3C2 light chain nucleotide sequence (hu3C2-hLG1)
SEQ ID NO: 71 Antibody 3C2 heavy chain amino acid sequence (hu3C2-hLhA2 (P221R))
SEQ ID NO: 72 Antibody 3C2 heavy chain nucleotide sequence (hu3C2-hLhA2 (P221R))
SEQ ID NO: 73 Antibody 3C2 light chain amino acid sequence (hu3C2-hLhA2 (P221R))
SEQ ID NO: 74 Antibody 3C2 light chain nucleotide sequence (hu3C2-hLhA2 (P221R))
SEQ ID NO: 75 Antibody 3C2 heavy chain amino acid sequence (hu3C2-hLhA2opt)
SEQ ID NO: 76 Antibody 3C2 heavy chain nucleotide sequence (hu3C2-hLhA2opt)
SEQ ID NO: 77 Antibody 3C2 light chain amino acid sequence (hu3C2-hLhA2opt)
SEQ ID NO: 78 Antibody 3C2 light chain nucleotide sequence (hu3C2-hLhA2opt)
SEQ ID NO: 79 Antibody 3B2 heavy chain amino acid sequence (hu3B2-hLG1)
SEQ ID NO: 80 Antibody 3B2 heavy chain nucleotide sequence (hu3B2-hLG1)
SEQ ID NO: 81 Antibody 3B2 light chain amino acid sequence (hu3B2-hLG1)
SEQ ID NO: 82 Antibody 3B2 light chain nucleotide sequence (hu3B2-hLG1)
SEQ ID NO: 83 Antibody 3B2 heavy chain amino acid sequence (hu3B2-hLhA2 (P221R))
SEQ ID NO: 84 Antibody 3B2 heavy chain nucleotide sequence (hu3B2-hLhA2 (P221R))
SEQ ID NO: 85 Antibody 3B2 light chain amino acid sequence (hu3B2-hLhA2 (P221R))
SEQ ID NO: 86 Antibody 3B2 light chain nucleotide sequence (hu3B2-hLhA2 (P221R))
SEQ ID NO: 87 Antibody 3B3 heavy chain amino acid sequence (hu3B3-hLG1)
SEQ ID NO: 88 Antibody 3B3 heavy chain nucleotide sequence (hu3B3-hLG1)
SEQ ID NO: 89 Antibody 3B3 light chain amino acid sequence (hu3B3-hLG1)
SEQ ID NO: 90 Antibody 3B3 light chain nucleotide sequence (hu3B3-hLG1)
SEQ ID NO: 91 Antibody 3B3 heavy chain amino acid sequence (hu3B3-hLhA2 (P221R))
SEQ ID NO: 92 Antibody 3B3 heavy chain nucleotide sequence (hu3B3-hLhA2 (P221R))
SEQ ID NO: 93 Antibody 3B3 light chain amino acid sequence (hu3B3-hLhA2 (P221R))
SEQ ID NO: 94 Antibody 3B3 light chain nucleotide sequence (hu3B3-hLhA2 (P221R))
SEQ ID NO: 95 Antibody 3B3 heavy chain amino acid sequence (hu3B3-hLhA2opt)
SEQ ID NO: 96 Antibody 3B3 heavy chain nucleotide sequence (hu3B3-hLhA2opt)
SEQ ID NO: 97 Antibody 3B3 light chain amino acid sequence (hu3B3-hLhA2opt)
SEQ ID NO: 98 Antibody 3B3 light chain nucleotide sequence (hu3B3-hLhA2opt)
SEQ ID NO: 99 Antibody 4A2 heavy chain amino acid sequence (hu4A2-hKG1)
SEQ ID NO: 100 Antibody 4A2 heavy chain nucleotide sequence (hu4A2-hKG1)
SEQ ID NO: 101 Antibody 4A2 light chain amino acid sequence (hu4A2-hKG1)
SEQ ID NO: 102 Antibody 4A2 light chain nucleotide sequence (hu4A2-hKG1)
SEQ ID NO: 103 Antibody 4A2 heavy chain amino acid sequence (hu4A2-hKhA2 (P221R))
SEQ ID NO: 104 Antibody 4A2 heavy chain nucleotide sequence (hu4A2-hKhA2 (P221R))
SEQ ID NO: 105 Antibody 4A2 light chain amino acid sequence (hu4A2-hKhA2 (P221R))
SEQ ID NO: 106 Antibody 4A2 light chain nucleotide sequence (hu4A2-hKhA2 (P221R))
SEQ ID NO: 107 Antibody 4A2 heavy chain amino acid sequence (hu4A2-hKhA2opt)
SEQ ID NO: 108 Antibody 4A2 heavy chain nucleotide sequence (hu4A2-hKhA2opt)
SEQ ID NO: 109 Antibody 4A2 light chain amino acid sequence (hu4A2-hKhA2opt)
SEQ ID NO: 110 Antibody 4A2 light chain nucleotide sequence (hu4A2-hKhA2opt)
SEQ ID NO: 111 Antibody 3C2 with extended hinge V1 and V2 light chain amino acid sequence (hu3C2-huLA2opt-G3hingeV1) and (hu3C2-huLA2opt-G3hingeV2)
SEQ ID NO: 112 Antibody 3C2 with extended hinge V1 and V2 light chain nucleotide sequence (hu3C2-huLA2opt-G3hingeV1) and (hu3C2-huLA2opt-G3hingeV2)
SEQ ID NO: 113 Antibody 3C2 with extended hinge V1 heavy chain amino acid sequence (hu3C2-huLA2opt-G3hingeV1)
SEQ ID NO: 114 Antibody 3C2 with extended hinge V1 heavy chain nucleotide sequence (hu3C2-huLA2opt-G3hingeV1)
SEQ ID NO: 115 Antibody 3C2 with extended hinge V2 heavy chain amino acid sequence (hu3C2-huLA2opt-G3hingeV2)
SEQ ID NO: 116 Antibody 3C2 with extended hinge V1 heavy chain nucleotide sequence (hu3C2-huLA2opt-G3hingeV2)
SEQ ID NO: 117 Antibody SR1 with extended hinge V1 and V2 light chain amino acid sequence (hzSR1-huKA2opt-G3hingeV1) and (hzSR1-huKA2opt-G3hingeV2)
SEQ ID NO: 118 Antibody SR1 with extended hinge V1 and V2 light chain nucleotide sequence (hzSR1-huKA2opt-G3hingeV1) and (hzSR1-huKA2opt-G3hingeV2)
SEQ ID NO: 119 Antibody SR1 with extended hinge V1 heavy chain amino acid sequence (hzSR1-huKA2opt-G3hingeV1)
SEQ ID NO: 120 Antibody SR1 with extended hinge V1 heavy chain nucleotide sequence (hzSR1-huKA2opt-G3hingeV1)
SEQ ID NO: 121 Antibody SR1 with extended hinge V2 heavy chain amino acid sequence (hzSR1-huKA2opt-G3hingeV2)
SEQ ID NO: 122 Antibody SR1 with extended hinge V2 heavy chain nucleotide sequence (hzSR1-huKA2opt-G3hingeV2)
SEQ ID NO: 123 IgA₂/IgG₃ hinge V1 and V2 C-terminal CH1 (IgA based) amino acid sequence
SEQ ID NO: 124 IgA₂/IgG3 hinge V1 and V2 Upper Hinge (IgG₃) amino acid sequence
SEQ ID NO: 125 IgA₂/IgG₃ hinge V1 and V2 Middle Hinge (IgG3) amino acid sequence
SEQ ID NO: 126 IgA₂/IgG₃ hinge V1 and V2 Lower Hinge (IgG3) amino acid sequence
SEQ ID NO: 127 IgA₂/IgG₃ hinge V1 IgA sequence past extended hinge amino acid sequence
SEQ ID NO: 128 IgA₂/IgG₃ hinge V2 IgA sequence past extended hinge amino acid sequence

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989, Al-Lazikani et al., J Mol Biol 273, 927-948, 1997 and/or Lohse et al., Cancer Research 76 (2); 403-17, 2015.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

The term “antibody dependent cellular trogocytosis” or “antibody dependent cell-mediated trogocytosis” (ADCT) refers to the rapid intercellular transfer of membrane fragments and their associated molecules from a donor cell to an acceptor cell (e.g., effector cell), during intercellular contact or through the release of vesicles. Exemplary acceptor cells include T and B cells, monocytes/macrophages, dendritic cells, neutrophils and natural killer (NK) cells. In the context of the present disclosure, trogocytosis-mediated transfer of a cell surface molecule such as, e.g., CD117, from a donor cell to an acceptor cell results in the transfer of an antibody-antigen complex from the donor cell to an acceptor cell, i.e., an antibody-antigen complex where an antibody is bound to the cell surface molecule (e.g., an antibody-CD117 complex). A specialized form of trogocytosis may occur when the acceptor cells are Fc-gamma-receptor (FcγR) expressing effector cells; these acceptor cells take up and internalize donor cell-associated immune complexes composed of specific antibodies bound to target antigens on donor cells, typically after binding of FcγRs to the Fc regions of the antibodies.

The terms “reduce” or “reducing” as used herein in reference to a population of cells in the bone marrow refers to administering an antibody described herein to stop or hinder the differentiation and/or maturation of the cells in the subject.

The terms “deplete” or “depleting” as used herein in reference to a population of cells in the bone marrow refers to administering an antibody described herein to stop or hinder the survival of bone marrow cells in the subject.

As used herein the term “ablate” and “ablation” refers to the partial or complete removal of a population of cells (e.g., HSCs) from the target tissues (e.g., bone marrow). It will be apparent to the skilled person that ablation comprises a complete removal or depletion of such cells from the target tissue. Alternatively, the ablation is a partial removal or depletion of such cells (e.g., HSCs) from the target tissue (i.e., bone marrow). For example, the methods disclosed herein result in at least about 5%, 10%, 12.5%, 15%, 20%, 25%, 30%, 35%, 40%, 45%, 50%, 55%, 60%, 65%, 70%, 75%, 80%, 85%, 90%, 92.5%, 95%, 97.5%, 98% or 99% depletion of the cells (e.g., HSCs) of the target tissue (e.g., bone marrow).

For the purposes of nomenclature only and not limitation an exemplary sequence of a human cluster of differentiation (CD117) (also known as c-kit or stem cell factor receptor (SCFR)) is set out in NCBI Reference Sequence NP_000213.1 and RefSeq Accession NM_000222.3 and SEQ ID NO: 1. The sequence of CD117 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). For the purposes of nomenclature only and not limitation an exemplary sequence of a cynomolgus monkey CD117 is set out in RefSeq Accession XP_005555330 and SEQ ID NO: 2. For the purposes of nomenclature only and not limitation an exemplary sequence of a murine CD117 is set out in NCBI Reference Sequence NP_001116205.1 and SEQ ID NO: 3. Reference to human CD117 may be abbreviated to hCD117. Reference herein to CD117 includes all isoforms of thereof.

As used herein, the term “subject” shall be taken to mean any animal including humans, for example a mammal. Exemplary subjects include but are not limited to humans and non-human primates. In one example, the subject is a human.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some examples, an antigen binding site is or comprises a V_{H} or a V_{L} or a Fv. In some examples, the antigen binding site comprises one or more CDRs of an antibody. The antigen binding site need not be in the context of an entire antibody, e.g., it can be in isolation (e.g., a domain antibody) or in another form, e.g., as described herein, such as a scFv.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies. In one example, the antibody is an IgA antibody. For example, the IgA antibody is a class IgA₂ antibody.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

The term “hinge region” as used herein, refers to a proline-rich portion of an antibody heavy chain constant region that links the Fc and Fab regions and confers mobility on the two Fab arms of the antibody as defined herein.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196:901-917, 1987; Chothia et al. Nature 342, 877-883, 1989; and/or Al-Lazikani et al., J Mol Biol 273:927-948, 1997; the IMGT numbering system of Lefranc et al., Devel. And Compar. Immunol., 27:55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means.

As used herein, the term “specifically binds” or “binds specifically” shall be taken to mean that an antibody of the disclosure reacts or associates more frequently, more rapidly, with greater duration and/or with greater affinity with a particular antigen or cell expressing same than it does with alternative antigens or cells. For example, a protein binds to CD117 with materially greater affinity (e.g., 20 fold or 40 fold or 60 fold or 80 fold to 100 fold or 150 fold or 200 fold greater affinity) avidity, more readily, and/or with greater duration than it binds to other antigens or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

An antibody may be considered to “preferentially bind” to a polypeptide if it binds that polypeptide with a dissociation constant (K_{D}) that is less than the antibody's K_{D} for another polypeptide. In one example, an antibody is considered to preferentially bind to a polypeptide if it binds the polypeptide with an affinity (i.e., K_{D}) that is at least about fold or 40 fold or 60 fold or 80 fold or 100 fold or 120 fold or 140 fold or 160 fold more than the antibody's K_{D} for another polypeptide.

For the purposes of clarification and as will be apparent to the skilled artisan based on the exemplified subject matter herein, reference to “affinity” in this specification is a reference to K_{D} of an antibody.

For the purposes of clarification and as will be apparent to the skilled artisan based on the description herein, reference to “a K_{D} of X nM or less” will be understood to mean that the numerical value of the K_{D} is equal to X nM or is lower in numerical value. As a skilled person would understand a lower numerical value of a K_{D} corresponds to a higher (i.e., stronger) affinity, i.e., an affinity of 2 nM is stronger than an affinity of 3 nM.

An “IC₅₀ of at least” will be understood to mean that the IC₅₀ is equal to the recited value or greater (i.e., the numerical value recited as the IC₅₀ is lower), i.e., an IC₅₀ of 2 nM is greater than an IC₅₀ of 3 nM. Stated another way, this term could be “an IC₅₀ of X or less”, wherein X is a value recited herein.

The term “competitively inhibits” shall be understood to mean that an antibody (i.e., anti-CD117 antibody) of the disclosure reduces or prevents binding of a recited antibody or protein to e.g., CD117. This may be due to the antibody binding to the same or an overlapping epitope. It will be apparent from the foregoing that the antibody need not completely inhibit binding of the antibody, rather it need only reduce binding by a statistically significant amount, for example, by at least about 10% or 20% or 30% or 40% or 50% or 60% or 70% or 80% or 90% or 95%. Preferably, the antibody reduces binding of the antibody to e.g., CD117 by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to CD117 either in the presence or absence of the compound. If less antibody binds in the presence of the antibody than in the absence of the antibody, the antibody is considered to competitively inhibit binding of the antibody. In one example, the competitive inhibition is not due to steric hindrance.

As used herein, the term “block” shall be taken to mean that an antibody that binds CD117 is capable of blocking, reducing or preventing CD117-mediated signaling in a cell by SCF. It will be apparent from the foregoing that the antibody need not completely block CD117-mediated signalling by SCF, rather it need only reduce it by a statistically significant amount, for example, by at least about 10% or 20% or 30% or 40% or 50% or 60% or 70% or 80% or 90% or 95%. Methods for determining blocking are known in the art and/or described herein.

The term “inhibit” in reference to activation of CD117 and/or CD117 cell proliferation shall be understood to mean that an antibody (i.e., anti-CD117 antibody) of the disclosure reduces or prevents CD117 activation and/or CD117 cell proliferation. It will be apparent from the foregoing that the antibody need not completely inhibit activation or cell proliferation, rather it need only reduce it by a statistically significant amount, for example, by at least about 10% or 20% or 30% or 40% or 50% or 60% or 70% or 80% or 90% or 95%. Methods for determining CD117 activation and/or CD117 cell proliferation are known in the art and/or described herein.

### Methods of Bone Marrow Conditioning

The methods described herein provide methods of conditioning a subject and/or methods of depleting a population of cells in the bone marrow of a subject in need of a hematopoietic cell transplantation (HCT), comprising administering to the subject an antibody that binds or specifically binds to a target on an endogenous hematopoietic stem cell (HSC) in the subject.

The methods herein also provide methods of HCT and/or methods of reducing side effects of a myeloablative conditioning regimen in a subject, comprising administering to the subject an antibody that binds or specifically binds to a target on an endogenous HSC in the subject.

### Hematopoietic Cell Transplantation (HCT)

As used herein, the term “hematopoietic cell transplantation” or “HCT” refers to the infusion, engraftment or transplantation of hematopoietic stem cells (HSC) and progenitor cells into a subject. It will be apparent to the skilled person that this term is used interchangeably with “bone marrow transplantation”, “stem cell transplantation” and “hematopoietic stem cell transplantation”.

In one example of any method described herein, the subject is in need of a HCT. For example, the subject has not yet received a HCT. For example, the antibody is administered prior to the subject receiving a HCT.

In one example, the subject is receiving a HCT. For example, the antibody is administered to a subject receiving a HCT. In one example, the antibody and the HCT are administered to the subject sequentially (i.e., one after the other). In another example, the antibody and the HCT are administered to the subject simultaneously (i.e., at the same time).

In one example of any method described herein, administering the antibody enhances or improves the engraftment efficiency of the HCT. As used herein, the phrases “engraftment efficiency” and “efficiency of engraftment” refers to the efficiency with which an administered stem/progenitor cell population (i.e., HSC graft) engrafts in the conditioned target tissue (i.e., bone marrow) of the subject. In certain embodiments, the efficiency of engraftment is increased by at least about 5%, 7.5%, 10%, 12.5%, 15%, 20%, 25%, 30%, 35%, 40%, 50%, 60%, 70%, 75%, 80%, 90%, 95%, 100% or more. It will be apparent to the skilled person that the determination of engraftment efficiency is assessed relative to the engraftment efficiency of a method in which the HSC engraftment is performed without the methods disclosed herein.

In one example, engraftment efficiency is monitored by determining chimerism in the subject. As used herein, the term “chimerism” refers to the percentage of HSCs in the subject that are of donor origin. It will be apparent to the skilled person that “donor” chimerism means that 100% of bone marrow and blood cells in the subject are of donor origin, whilst “mixed” or “partial” chimerism means that the subject's cells are also present. Methods of determining chimerism will be apparent to the skilled person and/or are described herein. For example, chimerism is determined using real-time quantitative PCT, fluorescent in situ hybridization (FISH) and chromosome markers.

In one example of any method described herein, administration of the antibody induces chimerism in the subject.

In one example, administration of the antibody comprising an extended hinge region facilitates or enables at least partial depletion, substantial depletion or elimination of cells expressing the target (e.g., cells expressing CD117).

In one example, administration of the antibody comprising an extended hinge region

In one example, administration of the antibody comprising a heavy chain comprising a sequence set forth in SEQ ID NO: 113; and a light chain comprising a sequence set forth in SEQ ID NO: 111.

In one example, the anti-CD117 antibody comprising a heavy chain comprising a sequence set forth in SEQ ID NO: 115; and a light chain comprising a sequence set forth in SEQ ID NO: 111.

In one example, administration of the antibody induces HSC transplant tolerance. For example, induction of chimerism in the subject induces HSC transplant tolerance.

In one example, the subject is receiving or will receive an autologous HCT. The term “autologous” in reference to a HCT refers to HCT with cells collected or obtained from the subject to be or receiving treatment (i.e., the same individual).

In one example, the subject is receiving or will receive an allogeneic HCT. The term “allogeneic” in reference to a HCT refers to a HCT with cells that are collected or obtained from another person to the subject to be or receiving treatment (i.e., a different individual).

Subjects in need of a HCT will be apparent to the skilled person and/or described herein. For example, a subject in need of a HCT is a subject having a disease or condition that may benefit from a HCT.

In one example, the subject is suffering from a disease or condition that requires treatment with a HCT. For example, the subject is suffering from a cancer, a hemoglobinopathy disorder, a myelodysplastic disorder, a primary immune deficiency (PID), an autoimmune disorder, an immunodeficiency disorder, or a metabolic disorder.

In one example, the subject is suffering from a cancer. The term “cancer” refers to or describes the physiological condition in mammals that is typically characterized by unregulated cell growth/proliferation. For example, the cancer is a hematologic (or blood) cancer. Hematologic/blood cell cancers include, but are not limited to, leukemia (e.g., acute lymphoblastic leukemia in adults and children; acute myeloid leukemia, e.g., in adults and children; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including Hodgkin's lymphoma in adults and children; Hodgkin's lymphoma during pregnancy; non-Hodgkin's lymphoma including non-Hodgkin's lymphoma in adults and children; non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; myelodysplastic/myeloproliferative disorders, neuroblastoma, and Ewing sarcoma).

In one example, the subject is suffering from a myelodysplastic disorder.

In one example, the subject is suffering from a hemoglobinopathy disorder. For example, the hemoglobinopathy disorder is sickle cell anemia, thalassemia, Fanconi anemia, or aplastic anemia.

In one example, the subject is suffering from a primary immune deficiency (PID). For example, the PID is Activated PI3K Delta Syndrome (APDS), X-linked Agammaglobulinemia (XLA), Ataxia Telangiectasia, Chronic Granulomatous Disease (CGD) and Other Phagocytic Cell Disorders, Common Variable Immune Deficiency (CVID), Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, Toll-like Receptor (TLR) Deficiencies (including MyD88 Deficiency, IRAK4 Deficiency, UNC93B Deficiency and TLR3 Mutations), Human Natural Killer Cell Deficiencies, Defects in Interferon-γ (IFN-γ) and Interleukin-12 (IL-12) Signaling, Leukocyte Adhesion Deficiency (LAD), NEMO Deficiency Syndrome, Selective IgA Deficiency, Selective IgM Deficiency, Severe Combined Immune Deficiency (SCID) and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Antibody Deficiency with Normal or Elevated Immunoglobulins, Immunodeficiency with Thymoma (Good's Syndrome), Transcobalamin II Deficiency, Kappa Chain Deficiency, Heavy Chain Deficiencies, Post-Meiotic Segregation (PMS2) Disorder, Unspecified Hypogammaglobulinemia, Chronic Mucocutaneous Candidiasis (CMC), Cartilage Hair Hypoplasia (CHH), X-linked Lymphoproliferative (XLP) Syndromes 1 and 2, X-linked Immune Dysregulation with Polyendocrinopathy (IPEX) Syndrome, Veno-occlusive Disease (VODI), Hoyeraal-Hreidarsson Syndrome (Dyskeratosis Congenita), Immunodeficiency with Centromeric Instability and Facial Anomalies (ICF), Schimke Syndrome, Comel-Netherton Syndrome, Diamond Blackfan Anemia, Schwachmann Diamond Syndrome, Deficiency of Adenosine Deaminase 2, Krabbe Disease (GLD), Alpha-mannosidosis, Nijmegen Breakage Syndrome, or graft-versus-host disease (GvHD). In one example, the PID is Wiskott-Aldrich syndrome (WAS), X-linked agammaglobulinemia (XLA), Diamond Blackfan Anemia, Schwachmann Diamond Syndrome, Deficiency of Adenosine Deaminase 2, Krabbe Disease (GLD), Alpha-mannosidosis, Nijmegen Breakage Syndrome, or graft-versus-host disease (GvHD).

In one example, the subject is suffering from an autoimmune disorder. For example, the autoimmune disorder is multiple sclerosis or diabetes. For example, the diabetes is Type I diabetes.

In one example, the subject is suffering from an immunodeficiency disorder. For example, the immunodeficiency disorder is human immunodeficiency virus (HIV) or acquired immunodeficiency syndrome (AIDS).

In one example, the subject is suffering from a metabolic disorder. For example, the metabolic disorder is a glycogen storage disease, mucopolysaccharidoses, Gaucher's Disease, Hurlers Disease, sphingolipidoses, or metachromatic leukodystrophy.

### Targeting Endogenous Hematopoietic Stem Cells

As described herein, the present disclosure relates to targeting, reducing, ablating and/or depleting a population of hematopoietic stem cells (HSC) residing in the target tissue (e.g., bone marrow or peripheral blood) of a subject. For example, the methods described herein relate to targeting, reducing, ablating and/or depleting an endogenous HSC population in the bone marrow of the subject to condition the subject for engraftment of a transplanted HSC population.

As used herein, the term “hematopoietic stem cell” (HSC) refers to an immature cell that can differentiate into all types of hematopoietic cells (i.e., blood cells), including white blood cells, red blood cells (including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and platelets. Hematopoietic stem cells are found in the peripheral blood and the bone marrow.

The term “endogenous” as used herein in relation to a HSC, refers to a HSC originating within the subject (i.e., within the subject's blood and/or bone marrow).

In one example of any method described herein, the method comprises administering to the subject an antibody that binds or specifically binds to a target on an endogenous HSC in the subject. It will be apparent to the skilled person that reference to a “target” on an endogenous HSC is reference to any protein, receptor, antigen, carbohydrate, lipid or other moiety that is located or expressed on the surface of the HSC and that can be used to discriminate a cell population. For example, the target is selectively expressed on the surface of the target cell population (i.e., HSC). In one example, the target is only expressed on the targeted cell population (i.e., the endogenous HSC population), thereby limiting or avoiding “off-target” effects.

In one example, selection of a target is made based on comparing the detected expression of such a target (e.g., a cell surface marker) on a HSC relative to the expression of such target on a control population of cells. For example, the expression of a target on a HSC or progenitor cell is compared to the mean expression of the same target on other cells (i.e., non-HSC or progenitor cells).

In one example, the target on the endogenous HSC is selected from the group consisting of CD2, CD5, CD7, CD11a, CDw12, CD13, CD15, CD18, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d (VLA-4), CD49e, CD49f (VLA-6), CD50, CD51, CD53, CD55, CD58, CD64a, CD68, CD71, CD72, CD73, CD81, CD82, CD84, CD85A, CD85K, CD90, CD97, CD99, CD104, CD105, CD109, CD110, CD111, CD112, CD114, CD115, CD117, CD123, CD124, CD126, CD127, CD130, CD131, CD133, CD135, CD137, CD138, CD151, CD157, CD162, CD164, CD166, CD168, CD172a, CD173, CD174, CD175, CD175s, CD176, CD183, CD184 (CXCR4), CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, CD350 and CD361.

In one example, the target on the endogenous HSC is selected from the group consisting of CD11a, CD18, CD34, CD37, CD45, CD47, CD52, CD58, CD62L, CD69, CD74, CD97, CD103, CD117, CD132, CD156a, CD179a, CD179b, CD184, CD232, CD244, CD252, CD302, CD305, CD317, and CD361.

In one example, the target on the endogenous HSC is CD117.

Antibodies that Bind to a Target on a HSC

The present disclosure provides an antibody that binds or specifically binds to a target on an endogenous HSC in the subject. In one example of any method described herein, the antibody mediates antibody dependent cellular trogocytosis (ADCT).

Methods for generating antibodies are known in the art and/or described in Harlow and Lane (editors) Antibodies: A Laboratory Manual, Cold Spring Harbor Laboratory, (1988). Generally, in such methods CD117 or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197:85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

An antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567; and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

### Mutations

The present disclosure also contemplates mutant forms of an antibody of the disclosure. For example, such a mutant antibody comprises one or more conservative amino acid substitutions compared to a sequence set forth herein. In some examples, the antibody comprises 30 or fewer or 20 or fewer or 10 or fewer, e.g., 9 or 8 or 7 or 6 or 5 or 4 or 3 or 2 conservative amino acid substitutions. A “conservative amino acid substitution” is one in which the amino acid residue is replaced with an amino acid residue having a similar side chain and/or hydropathicity and/or hydrophilicity.

In one example of any antibody described herein, the antibody comprises an amino acid modification numbered according to the myeloma IgA1 protein (Bur) scheme. For example, the myeloma IgA1 protein (Bur) scheme as described in Lohse et al., Cancer Research 76(2); 403-17, 2015.

In one example, a mutant antibody has only, or not more than, one or two or three or four or five or six conservative amino acid changes when compared to a naturally occurring antibody. Details of conservative amino acid changes are provided below. As the skilled person would be aware, e.g., from the disclosure herein, such minor changes can reasonably be predicted not to alter the activity of the antibody.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

The present disclosure also contemplates non-conservative amino acid changes (e.g., substitutions) in an antibody of the present disclosure, e.g., in a CDR, such as CDR3. In one example, the antibody comprises fewer than 6 or 5 or 4 or 3 or 2 or 1 non-conservative amino acid substitutions, e.g., in a CDR3, such as in a CDR3.

In one example, the mutation(s) occur within a FR of an antigen binding domain of an antibody of the disclosure. In another example, the mutation(s) occur within a CDR of an antibody of the disclosure.

Exemplary methods for producing mutant forms of an antibody include:
mutagenesis of DNA (Thie et al., Methods Mol. Biol. 525:309-322, 2009) or RNA (Kopsidas et al., Immunol. Lett. 107:163-168, 2006; Kopsidas et al. BMC Biotechnology, 7:18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

The present disclosure also contemplates one or more insertions or deletions compared to a sequence set forth herein. In some examples, the antibody comprises 10 or fewer, e.g., 9 or 8 or 7 or 6 or 5 or 4 or 3 or 2 insertions and/or deletions.

### Constant Regions

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant domain or portion thereof may be derived from any antibody class, including IgM, IgG, IgD, IgA and IgE, and any antibody isotype. In one example, the human antibody class IgA is used. In another example, the human isotype IgA₂ is used.

In one example, the Fc region of the constant region has or displays an effector function that facilitates or enables at least partial depletion, substantial depletion or elimination of cells expressing the target (e.g., cells expressing CD117). Such an effector function may be enhanced binding affinity to Fc receptors, and/or antibody-dependent cell-mediated cytotoxicity (ADCC), and/or antibody-dependent cell mediated phagocytosis (ADCP), and/or antibody dependent cellular trogocytosis (ADCT), and/or complement dependent cytotoxicity (CDC) and combinations thereof. Methods for assessing the level of effector function of an Fc region containing protein are known in the art and/or described herein.

In one example, the antibody of the disclosure is capable of inducing an enhanced level of effector function.

In one example, the level of effector function induced by the constant region is enhanced relative to a wild-type Fc region of an IgG₁ antibody or a wild-type Fc region of an IgG₄ antibody.

In another example, the constant region is modified to increase the level of effector function it is capable of inducing compared to the constant region without the modification. Such modifications can be at the amino acid level and/or the secondary structural level and/or the tertiary structural level and/or to the glycosylation of the Fc region.

The skilled addressee will appreciate that greater effector function may be manifested in any of a number of ways, for example as a greater level of effect, a more sustained effect or a faster rate of effect.

Exemplary constant region modifications include amino acid substitutions, such as, a N166G mutation, a P221R mutation, a C311S mutation, a NIT337-339TLS mutation, delC472 and delY473, numbered according to the myeloma IgA1 protein (Bur) scheme.

It will be apparent to the skilled person that the antibody described herein according to any example may be further modified to enhance the pharmacokinetic properties of the antibody. For example, the antibody may be further modified to enhance the half-life and/or the stability of the antibody.

In one example, the antibody comprises one or more amino acid modifications that increase the half-life of the antibody. For example, the antibody comprises a Fc region comprising one or more amino acid substitutions that increase the affinity of the Fc region for the neonatal Fc region (FcRn). These amino acid substitutions are useful for extending the half-life of a protein, by reducing clearance from the blood.

In one example, the antibody comprises one or more amino acid modifications that increase the stability of the antibody. For example, the antibody comprises amino acid modifications at an asparagine and/or a glutamine residue prone to deamidation and/or a glutamine residue prone to pyroglutamate formation.

In one example, the antibody is conjugated to a compound that increases the half-life of the antibody. For example, the antibody is conjugated to a half-life extender, such as polyethylene glycol (PEG), glycerol, glucose and/or albumin.

### Hinge Region

The present disclosure encompasses proteins and/or antibodies described herein comprising a modified hinge region.

Sequences of hinges useful in the proteins of the present disclosure may be obtained from a number of different sources. In some examples, the hinge region of the protein is derived from a human antibody. The hinge region thereof may be derived from any antibody class, including IgM, IgG, IgD, IgA and IgE, and any antibody isotype. In one example, the human antibody class IgA is used. In another example, the human isotype IgA₂ is used. In further example, the human isotype IgG is used. For example, the human isotype IgG is an IgG₁, IgG₂, IgG₃ or IgG₄. In one example, the human isotype IgG is an IgG₁. In one example, the human isotype IgG is an IgG₂. In one example, the human isotype IgG is an IgG₃. In one example, the human isotype IgG is an IgG₄.

In one example, the hinge region is modified to increase the level of effector function it is capable of inducing compared to a hinge region without the modification. Such modifications can be substitutions and/or insertions at the amino acid level and/or the secondary structural level and/or the tertiary structural level and/or to the glycosylation of the hinge region.

In one example, an antibody comprising the modified hinge region has or displays an effector function that facilitates or enables at least partial depletion, substantial depletion or elimination of cells expressing the target (e.g., cells expressing CD117). Such an effector function may be enhanced binding affinity to Fc receptors, and/or antibody-dependent cell-mediated cytotoxicity (ADCC), and/or antibody-dependent cell mediated phagocytosis (ADCP), and/or antibody dependent cellular trogocytosis (ADCT), and/or complement dependent cytotoxicity (CDC) and combinations thereof. Methods for assessing the level of effector function of an antibody are known in the art and/or described herein.

In one example, the antibody of the disclosure comprising a modified hinge region is capable of inducing an enhanced level of effector function.

In one example, the level of effector function induced by the antibody comprising a modified hinge region is enhanced relative to a wild-type IgA₂ antibody or a wild-type IgG₃ antibody.

The skilled addressee will appreciate that greater effector function may be manifested in any of a number of ways, for example as a greater level of effect, a more sustained effect or a faster rate of effect.

It will be apparent to the skilled person that the hinge regions described herein according to any example may be modified or further modified to enhance the pharmacokinetic properties of the antibody. For example, the hinge region may be modified to enhance the half-life and/or the stability of the antibody.

In one example, the hinge region comprises one or more amino acid modifications and/or insertions that increase the half-life of the antibody. For example, the antibody comprises a hinge region comprising one or more amino acid substitutions and/or insertions that increase the affinity of the hinge region for the neonatal Fc region (FcRn). These amino acid substitutions and/or insertions are useful for extending the half-life of a protein and/or antibody, by reducing clearance from the blood.

In one example, the hinge region comprises one or more amino acid modifications and/or insertions that increase the stability of the antibody.

In one example, the hinge region is modified by inserting one or more amino acids to produce an extended hinge region. For example, the hinge region is modified by inserting another hinge region to produce an extended hinge region.

In one example, the antibody comprising an extended hinge region has better access to its epitope relative to a wild-type antibody. In one example, the antibody comprising an extended hinge region has better access to its epitope relative to an antibody without the extended hinge region.

In one example, an IgA₂ hinge region is modified by inserting at least of a portion of an IgG₃ hinge region after the first proline in the IgA₂ hinge region. For example, the modified hinge region comprises a chimeric IgA₂/IgG₃ hinge region.

In one example, an IgA₂ hinge region is modified by inserting at least a portion of an IgA₁ hinge region after the first proline in the IgA₂ hinge region. For example, the modified hinge region comprises a chimeric IgA₂/IgA₁ hinge region.

Molecular cloning techniques to achieve these ends are known in the art and described, for example in Senior et al., Cleavage of a Recombinant Human Immunoglobulin A2 (IgA2)-IgA1 Hybrid Antibody by Certain Bacterial IgA1 Proteases (2000) or Woof & Kerr. The function of immunoglobulin A in immunity (2006).

### Protein Production

In one example, an antibody described herein according to any example is produced by culturing a hybridoma under conditions sufficient to produce the protein, e.g., as described herein and/or as is known in the art.

### Recombinant Expression

In another example, an antibody described herein according to any example is recombinant.

In the case of a recombinant antibody, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO1999/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Assaying Activity

### Binding Assays

Methods for assessing binding of a candidate antibody to a target protein (e.g., CD117) are known in the art, e.g., as described in Scopes (In: Protein purification: principles and practice, Third Edition, Springer Verlag, 1994). Such a method generally involves labelling the protein and contacting it with immobilized antibody. Following washing to remove non-specific bound protein, the amount of label and, as a consequence, bound protein is detected. Of course, the protein can be immobilized and the antibody labelled. Panning-type assays can also be used. Alternatively, or additionally, surface plasmon resonance assays can be used. The level of binding can also be conveniently determined using a biosensor.

### Affinity Assays

Optionally, Optionally, the dissociation constant (Kd) or association constant (Ka) or binding constant (K_{D}, i.e., Ka/Kd) of an antibody for e.g., CD117 or an epitope thereof is determined. The “Kd” or “Kd value” for a compound that binds to e.g., CD117 is in one example measured by a radiolabeled or fluorescently-labeled CD117 binding assay. This assay equilibrates the antibody with a minimal concentration of labelled CD117 in the presence of a titration series of unlabelled CD117. Following washing to remove unbound CD117, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized CD117 or a region thereof.

### Determining Competitive Binding

Assays for determining antibody that competitively inhibits binding of antibody described herein will be apparent to the skilled artisan. For example, an exemplary antibody is conjugated to a detectable label, e.g., a fluorescent label or a radioactive label. The labelled antibody and the test protein are then mixed and contacted with e.g., CD117 or a region thereof (e.g., a polypeptide comprising SEQ ID NO: 1) or a cell expressing same. The level of labelled antibody is then determined and compared to the level determined when the labelled antibody is contacted with the CD117, region or cells in the absence of the protein. If the level of labelled CSL117 is reduced in the presence of the test protein compared to the absence of the protein, the protein is considered to competitively inhibit binding of an antibody to CD117.

Optionally, the test protein is conjugated to a different label to the antibody. This alternate labelling permits detection of the level of binding of the test protein to CD117 or the region thereof or the cell.

In another example, the protein is permitted to bind to CD117 or a region thereof (e.g., a polypeptide comprising SEQ ID NO: 1) or a cell expressing same prior to contacting the CD117, region or cell with the antibody. A reduction in the amount of bound antibody in the presence of the protein compared to in the absence of the protein indicates that the protein competitively inhibits binding of the antibody to CD117. A reciprocal assay can also be performed using labelled protein and first allowing the antibody to bind to CD117. In this case, a reduced amount of labelled protein bound to CD117 in the presence of the antibody compared to in the absence of the antibody indicates that the protein competitively inhibits binding of the antibody to CD117.

### Internalisation Assay

Assays for assessing the internalisation of the antibodies of the disclosure will be apparent to the skilled person and/or described herein. Exemplary assays include an anti-AF488 quenching antibody assay and an IncuCyte® live-cell high-throughput internalisation assay.

In one example, internalization is assessed using an anti-AF488 quenching antibody assay. Briefly, cells are incubated with a test antibody pre-complexed with AF488-labelled anti-human IgG Fab fragments, in the presence or absence of human stem cell factor. Following incubation, mean fluorescence intensity (MFI) values are obtained and the percentage of internalised antibody calculated by dividing the internalised signal (MFI from samples treated with anti-AF488 quenching antibody) with the total cell bound signal (MFI from unquenched samples) at each time point.

In another example, internalization is assessed using an IncuCyte® live-cell high-throughput internalisation assay. Briefly, TF-1 cells are seeded in a 96 well plate pre-coated with 0.001% (V/V) poly-ornithine and allowed to adhere. Test antibodies are pre-complexed with FabFluor-pH Red and added to cells in the absence and presence of human stem cell factor. Internalised antibodies are immediately captured in the Incucyte® S3 (Sartorius) and analysed for red fluorescence object area (measure of FabFluor labelled internalized antibody), with any background fluorescence signal subtracted.

In one example, antibodies of the disclosure have slow internalising kinetics in the anti-AF488 quenching antibody assay and/or the IncuCyte® live cell analysis.

### Inhibition Assay

In one example, the antibody that binds or specifically binds to a target on an endogenous HSC blocks binding of CD117 by stem cell factor (SCF) and/or inhibits activation of CD117 by SCF. For example, the antibody of the disclosure is assessed for its ability to inhibit TF-1 proliferation mediated by human stem cell factor.

Briefly, TF-1 cells are propagated and plated at a density of 2×10⁴ cells/well. The antibodies of the disclosure are added to each corresponding well in the presence of Human Stem Cell Factor. Following incubation, inhibition of TF-1 cell proliferation was measured using a commercially available kit e.g., a Vialight Plus kit.

### Determining Effector Function

As discussed herein, antibodies of the disclosure may have effector function (or enhanced effector function). Methods for assessing effector function, such as ADCC or ADCT activity are known in the art.

In one example, the level of ADCC activity is assessed using a ⁵¹Cr release assay, an europium release assay or a ³⁵S release assay. In each of these assays, cells expressing e.g., CD117 are cultured with one or more of the recited compounds (i.e., anti-CD117 antibodies) for a time and under conditions sufficient for the compound to be taken up by the cell. In the case of a ³⁵S release assay, cells expressing CD117 can be cultured with ³⁵S-labeled methionine and/or cysteine for a time sufficient for the labeled amino acids to be incorporated into newly synthesized proteins. Cells are then cultured in the presence or absence of the anti-CD117 antibody and in the presence of immune effector cells, e.g., peripheral blood mononuclear cells (PBMC) and/or NK cells. The amount of ⁵¹Cr, europium and/or ³⁵S in cell culture medium is then detected, and little or no change in the presence of the anti-CD117 antibody compared to in the absence of the anti-CD117 antibody indicates that the protein has reduced effector function and an increased amount compared to in the absence of the anti-CD117 antibody (or increased compared to in the presence of the anti-CD117 antibody comprising an IgG1 Fc region) indicating effector function or enhanced effector function. Exemplary publications disclosing assays for assessing the level of ADCC induced by a protein include Hellstrom, et al. Proc. Natl Acad. Sci. USA 83:7059-7063, 1986 and Bruggemann, et al., J. Exp. Med. 166:1351-1361, 1987.

Other assays for assessing the level of ADCC induced by a protein include ACTI™ nonradioactive cytotoxicity assay for flow cytometry (CellTechnology, Inc. CA, USA) or CytoTox 96® non-radioactive cytotoxicity assay (Promega, WI, USA).

In one example, the level of ADCT activity is assessed by flow cytometry. Briefly, a cell line transfected with human CD117 is labelled with the membrane-dye DILC18 (1, 1″-dioctadexyl-3,3, 3″, 3″-tetramethylindocarbocyanine perchlorate), mixed with freshly isolated peripheral blood neutrophils as effector cells and incubated with an antibody of the disclosure or control antibodies. Subsequently, cells are stained with Alexa fluor 647-conjugated anti CD66b and the LIVE/DEAD™ Fixable Green Stain, washed and fixed with formaldehyde. Cells are acquired on a flow cytometer and live neutrophils identified by forward and side scatter, followed by live CD66b positive cells. Membrane uptake by neutrophils is measured by an increase of DILC18 mean fluorescence intensity (MFI). DILC18 MFI is normalised to the maximum MFI per donor neutrophil. In one example, the half maximal effective concentration (EC₅₀±SD) in [nM] and the normalised maximum membrane uptake (Bₘₐₓ) in percent facilitated by the antibody inducing trogocytosis by peripheral blood neutrophils is determined.

Any of the foregoing assays can be performed with any target as described herein.

### Compositions

In one example, an antibody as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, intrapolyp and intracranial injection or infusion techniques.

Methods for preparing an antibody into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of an antibody dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antibody of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antibody of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antibody of the present disclosure.

WO2002/080967 describes compositions and methods for administering aerosolized compositions comprising antibodies for the treatment of respiratory conditions, which are also suitable for administration of compounds in accordance with the methods of the present disclosure.

### Combination Therapies

In one example, an antibody of the present disclosure is administered in combination with an additional conditioning regimen. For example, the subject is receiving, or will receive an additional conditioning regimen.

In one example, the additional conditioning regimen is a non-myeloablative or reduced-intensity conditioning regimen.

As used herein, the terms “non-myeloablative conditioning” and “reduced-intensity conditioning” refer to a conditioning regimen comprising of low dose chemotherapy and/or radiotherapy. Non-myeloablative and reduced-intensity conditioning regimens differ in their ability to produce cytopenia.

In one example, the non-myeloablative or reduced-intensity conditioning regimen comprises low-dose chemotherapy. For example, the low-dose chemotherapy is fludarabine, cyclophosphamide, busulfan, anti-thymocyte globulin equine, thiopeta, melphalan, alemtuzumab, cladribine, idarubicin, cytarabine, cisplatin, azacitidine, rituximab, etoposide and combinations thereof. In one example, the low-dose chemotherapy is busulfan. In another example, the low dose chemotherapy is busulfan, fludarabine and rituximab.

In one example, the non-myeloablative or reduced-intensity conditioning regimen comprises low-dose radiotherapy. For example, low-dose total body irradiation or total lymphoid irradiation. In one example, the low-dose total body irradiation is at a dose of ≤2 Grays (Gy) or 1 Gy.

In one example, the additional conditioning regimen is total body irradiation at a dose of ≤2 Gy, with or without a purine analog.

In one example, the additional conditioning regimen is fludarabine, and cyclophosphamide, with or without anti-thymocyte globulin.

In one example, the additional conditioning regimen is fludarabine, cytarabine and idarubicine.

In one example, the additional conditioning regimen is cladribine and cytarabine.

In one example, the additional conditioning regimen is total lymphoid irradiation and anti-thymocyte globulin.

### Dosages and Timing of Administration

Suitable dosages of an antibody of the present disclosure will vary depending on the specific antibody, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically or prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present disclosure comprises administering an effective amount of an antibody described herein.

The term “effective amount” is the quantity which, when administered to a subject in need of treatment, improves the condition of the subject's target tissue (e.g., bone marrow) for transplant. For example, the effective amount is an amount sufficient to ablate or deplete or reduce the endogenous HSC population in the subject. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present disclosure to a specific quantity, e.g., weight or amount of protein(s), rather the present disclosure encompasses any amount of the antibody sufficient to achieve the stated result in a subject.

In one example, an effective amount of the antibody disclosed herein achieves a maximal stem cell depletion. For example, about 90%, 91%, 92%, 93%, 94%, 95%, 96%, 97%, 97.5%, 98%, 99%, 99.5% or more depletion of endogenous HSC from the target tissues, e.g., bone marrow, of the subject.

For in vivo administration of the antibody described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the antibody is administered at an initial (or loading) dose of between about 1 mg/kg to about 30 mg/kg, such as from about 1 mg/kg to about 10 mg/kg, or about 1 mg/kg or about 2 mg/kg or 5 mg/kg. The antibody can then be administered at a lower maintenance dose of between about 0.01 mg/kg to about 2 mg/kg, such as from about 0.05 mg/kg to about 1 mg/kg, for example, from about 0.1 mg/kg to about 1 mg/kg, such as about 0.1 mg/kg or 0.5 mg/kg or 1 mg/kg. The maintenance doses may be administered every 7-30 days, such as, every 10-15 days, for example, every 10 or 11 or 12 or 13 or 14 or 15 days.

In some examples, the antibody is administered at a dose of between about 0.01 mg/kg to about 50 mg/kg, such as between about 0.05 mg/kg to about 30 mg/kg, for example, between about 0.1 mg/kg to about 20 mg/kg, for example, between about 0.1 mg/kg to about 10 mg/kg, such as between about 0.1 mg/kg to about 2 mg/kg. For example, the antibody is administered at a dose of between about 0.01 mg/kg to about 5 mg/kg, such as from about 0.1 mg/kg to about 2 mg/kg, such as about 0.2 mg/kg or 0.3 mg/kg or 0.5 mg/kg or 1 mg/kg or 1.5 mg/kg (e.g., without a higher loading dose or a lower maintenance dose). In some examples, numerous doses are administered, e.g., every 7-30 days, such as, every 10-22 days, for example, every 10-15 days, for example, every 10 or 11 or 12 or 13 or 14 or 15 or 16 or 17 or 18 or 19 or 20 or 21 or 22 days. For example, the antibody is administered every 7 days or every 14 days or every 21 days.

In some examples, at the time of commencing therapy, the subject is administered the antibody on no more than 7 consecutive days or 6 consecutive days or 5 consecutive days or 4 consecutive days.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of an antibody according to the methods of the present disclosure can be continuous or intermittent, depending, for example, on the recipient's physiological condition, and other factors known to skilled practitioners. The administration of an antibody may be essentially continuous over a preselected period of time or may be in a series of spaced doses, e.g., either during or after development of a condition.

### Kits

Another example of the disclosure provides kits containing antibodies useful for a method of conditioning a subject in need of a HCT as described herein.

In one example, the kit comprises (a) a container comprising an antibody as described herein, optionally in a pharmaceutically acceptable carrier or diluent; and (b) a package insert with instructions for conditioning a subject in need of a HCT.

In one example, the kit comprises (a) a container comprising an antibody as described herein, optionally in a pharmaceutically acceptable carrier or diluent; and (b) a package insert with instructions for reducing, depleting and/or ablating a population of cells in the bone marrow of a subject in need of a HCT.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for conditioning a subject and may have a sterile access port (for example, the container may be an intravenous solution bag or a vial having a stopper pierceable by a hypodermic injection needle). At least one active agent in the composition is the compound that binds to a target on an endogenous HSC. The label or package insert indicates that the composition is administered to a subject eligible for treatment, e.g., a subject in need of or receiving a HCT, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Anti-CD117-IgG1 Monoclonal Antibody Screening

To identify antibodies against CD117, an in-house antibody phage display library was panned against recombinant human CD117 (recHuCD117). Three rounds of phage display selection were undertaken against biotinylated recHuCD117 ECD in solution, where the antigen concentration was kept constant for Selections A, C and D; or in the cases of Selections B, E and F antigen concentration was decreased with each round of panning. The phage/HuCD117 complexes were collected by incubating them with streptavidin magnetic beads. Following each round, beads were washed, and bound phage was amplified and purified for use as input for the next panning round. At the end of the panning process, individual phage clones from the Round 3 outputs were screened for binding to recHuCD117 ECD and recombinant murine CD117 ECD (c-Kit Protein, Mouse, Recombinant (His Tag) Sino Biologicals) by Fab-phage ELISA. Sequence analysis of clones reactive to human and mouse CD117 revealed a total of 49 positive unique clones.

Forty-seven of the HuCD117-positive clones were able to be reformatted into intact human antibodies (IgG₁ format).

### Example 2: Anti-CD117-IgG1 Antibodies Bind to Human, Mouse and Cyno CD117

To assess binding of anti-CD117-IgG1 antibodies to cell-surface expressed CD117, TF-1 or HEK293FS stably expressing Human, Mouse or cynomolgus monkey CD117 were resuspended in Dulbecco's PBS (Sigma Aldrich, D8537) containing 1% FCS and incubated with a titration of anti-CD117-IgG1 antibodies (5.7 pM-12.5 nM for TF-1 cells; 6.1 pM-25 nM for HEK293FS). After a one hour incubation on ice, cells were washed once prior to fixation.

Primary anti-CD117-IgG1 antibodies were detected by addition of secondary anti-human IgG Fab fragments conjugated to Alexa-Fluor 488 (Jackson labs, 109-547-008), with a one hour incubation on ice, before their cell bound fluorescence analysed on a LSRFortessa™ cell analyser (BD Biosciences). Mean fluorescence intensities (MFI) were obtained using Flowjo v10 and analysed using GraphPad Prism. Total binding curves for TF-1 cells and specific binding curves for HEK293FS expressing CD117 (generated by subtracting non-specific binding to parental cells) were plotted. EC50 values for each anti-CD117-IgG1 antibody tested was determined using a non-linear regression model.

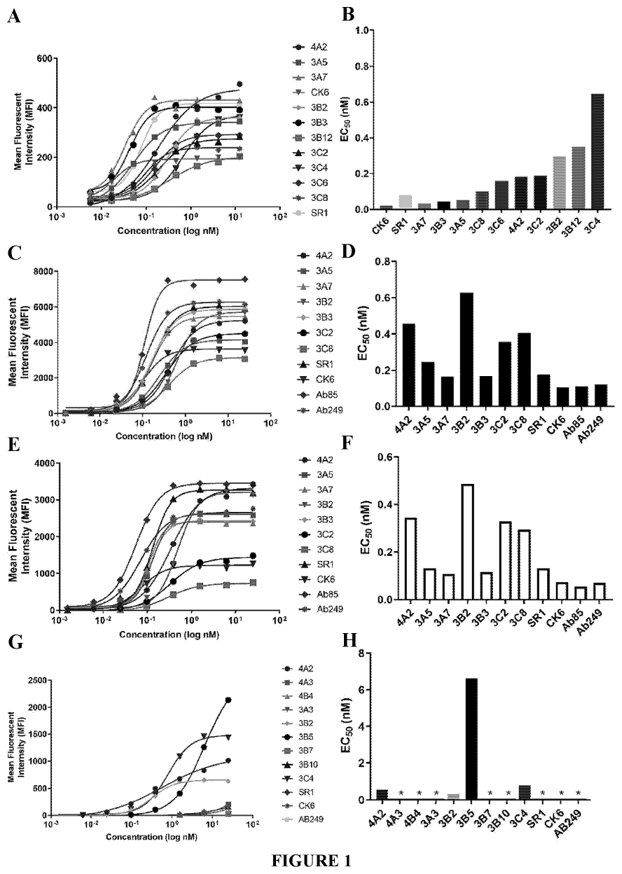

Of the 47 anti-CD117-IgG1 antibodies generated, 28 bound cell-surface CD117 on TF-1 cells with an EC₅₀<25 nM (FIG. 1A-B). All antibodies bound human and cynomolgus monkey CD117 when expressed on the surface of HEK293FS cells, with 4 were cross-reactive with murine CD117 when expressed on the surface of HEK293FS cells (FIG. 1C-H).

### Example 3: Anti-CD117-IgG1 Antibodies Inhibit TF-1 Cell Proliferation Mediated by Human Stem Cell Factor

To assess antibodies with potential inhibitory activity, inhibition of TF-1 cell proliferation mediated by human stem cell factor (SCF) was assessed.

TF-1 cells were propagated in RPMI complete media (RPMI 1640 (Sigma #R0883) supplemented with 10% FBS, 50U/ml Penicillin and 50 mg/ml Streptomycin (Pen-Strep, Gibco #15070-063), 2 mM Glutamax (Gibco #35050)), with the addition of hGM-CSF (5 ng/ml, R&D Systems, #215-GM), at 37° C. at 5% CO₂. The cells were pelleted by centrifugation (1200 rpm, 5 minutes) and resuspended in 5 ml of RPMI complete media, counted using a haemocytometer and prepared to a final cell density of 2×105 cells/ml. Cells (100 ul) were then plated in 96 well plates to achieve a final cell density of 2×104 cells/well. The antibody clones were prepared in RPMI complete media at 4× concentration (4 ug/ml, 26.68 nM) and serially diluted 3-fold for 12 points. The titrated antibodies were then added to each corresponding well (50 ul) to give a final starting concentration of 1 ug/ml (6.67 nM). Human Stem Cell Factor (hSCF, Peprotech, #300-07) was prepared in RPMI complete media at 4× the EC50 concentration (328 pM) and added to all wells (50 ul) to achieve a final concentration of 82 pM/well. The plates were then placed in the incubator for 72 hours, 37° C. at 5% CO₂. An end point measurement was made after the 72 hour incubation using a Vialight Plus kit (Lonza, #LT07-121) following the manufacturer's instructions.

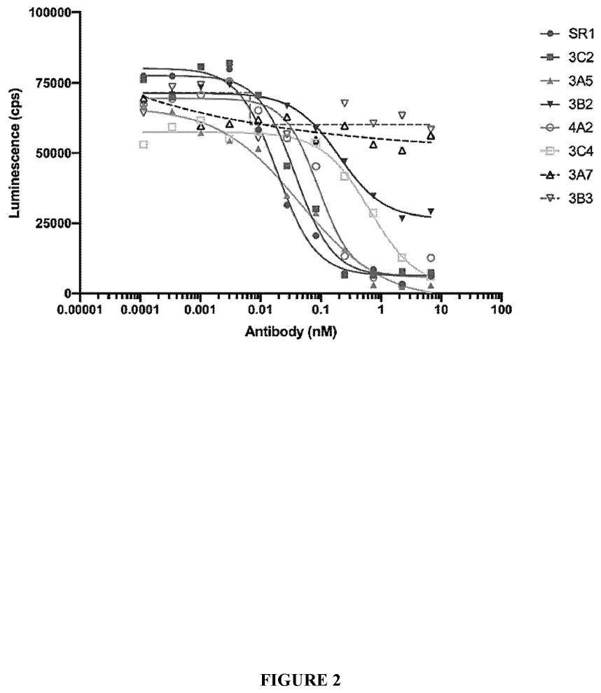

Of the 47 anti-CD117-IgG1 antibodies generated, 13 inhibited hSCF-induced TF1 cell proliferation (FIG. 2).

### Example 4: Internalisation of Anti-CD117-IgG1 Antibodies

### Anti-AF488 Quenching Antibody Assay

To assess the internalisation of anti-CD117-IgG1 antibodies, an assay based on quenching cell surface bound fluorescence using an anti-AF488 antibody was established using TF-1 cells. Briefly, cells were incubated with 5 nM of anti-CD117-IgG1 antibodies pre-complexed with AF488-labelled anti-human IgG Fab fragments (Jackson labs, 109-547-008), at a molar ratio of 1:3, in the presence or absence of 56 nM human stem cell factor (Genscript, Z02692-1), and incubated at 37° C. for 0, 15, 30, 60 and 120 minutes. Two samples were processed at each time-point: one sample was resuspended in PBS containing 1% FCS and the other in PBS containing 1% FCS and 20 μg/mL anti-AF488 quenching IgG (Thermo-Fisher, A-11094). Cells were incubated for 30 minutes on ice, prior to analysis on the BD LSRFortessa cytometer. Mean fluorescence intensity (MFI) values were obtained using Flowjo v10 and the percentage of internalised antibody was calculated by dividing the internalised signal (MFI from samples treated with anti-AF488 quenching antibody) with the total cell bound signal (MFI from unquenched samples) at each time point.

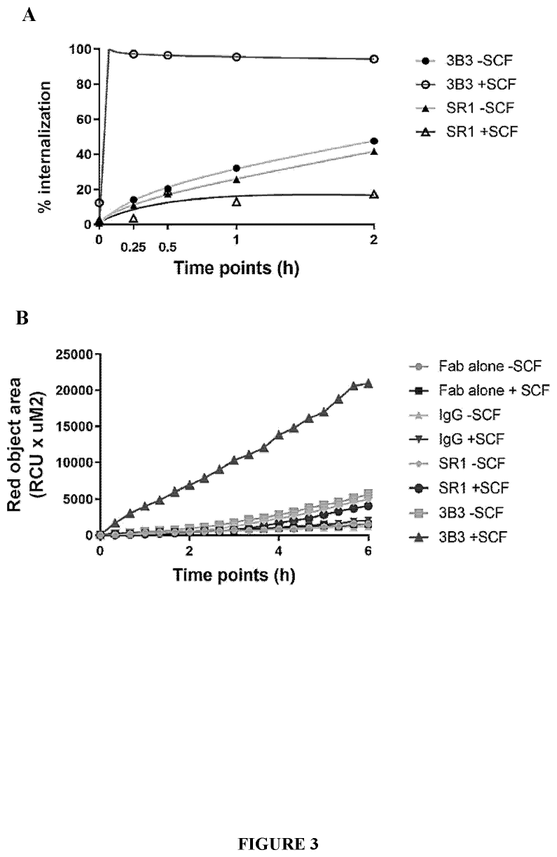

As shown in FIG. 3A the selected anti-CD117-IgG₁ antibodies had slow internalising kinetics.

### IncuCyte® Live-Cell High-Throughput Internalisation Assay

To compare the internalisation of anti-CD117-IgG₁ antibodies, an integrated assay based on IncuCyte® live-cell analysis and a pH-sensitive dye coupled antibody fragment (IncuCyte® human FabFluor-pH Red antibody labeling reagent, Sartorius, 4722) was performed using TF-1 cells and according to the manufacturer's directions. Briefly, TF-1 cells were seeded in wells of a 96 well plate pre-coated with 0.001% (V/V) poly-ornithine (Sigma-Aldrich, A004M), and allowed to adhere for 1 hour at 37° C. Anti-CD117 antibodies pre-complexed with FabFluor-pH Red, at a molar ratio of 1:3, were added to cells (serially diluted from 25 nM to 3.125 nM). Experiments were performed in the absence and presence of 56 nM human stem cell factor. Images of internalised antibodies were immediately captured using a 20× objective every 10 minutes for 6 hours in the Incucyte® S3 (Sartorius) and analysed using integrated software for red fluorescence object area (measure of FabFluor labelled internalized antibody). Top Hat subtraction was used to minimize any background fluorescence signal.

Similar to the anti-AF488 quenching antibody assay, the selected anti-CD117-IgG1 antibodies had slow internalising kinetics in the IncuCyte® live cell analysis (FIG. 3B).

### Example 5: Absence of Activation of TF-1 Cells Mediated by Anti CD117-IgG Antibodies in the Absence of Human SCF

Anti-CD117-IgG₁ antibodies were assessed for their ability to activate TF-1 proliferation in the absence of human stem cell factor.

TF-1 cells were propagated as described above. The cells were pelleted by centrifugation (1200 rpm, 5 minutes) and resuspended in 5 ml of RPMI complete media, counted using a haemocytometer and prepared to a final cell density of 2×10⁵ cells/ml. Cells (100 ul) were then plated in 96 well plates to achieve a final cell density of 2×10⁴ cells/well. The antibodies were prepared in RPMI complete media at 4× concentration (400 μg/ml, 2668 nM) and serially diluted 3-fold for 12 points and added to each corresponding well (50 ul) to give a final starting concentration of 100 ug/ml (667 nM). To prevent initial cell death a low level of hGM-CSF (R&D Systems, #215-GM) was prepared in RPMI complete media at 4× concentration (0.04 ng/ml) and added to all wells (100 ul) to give a final concentration of 0.01 ng/ml. The plates were then placed in the incubator for 48 hours, 37° C. at 5% CO₂. After incubation an end point measurement was taken using a Vialight Plus kit (Lonza, #LT07-121) following the manufacturer's instructions.

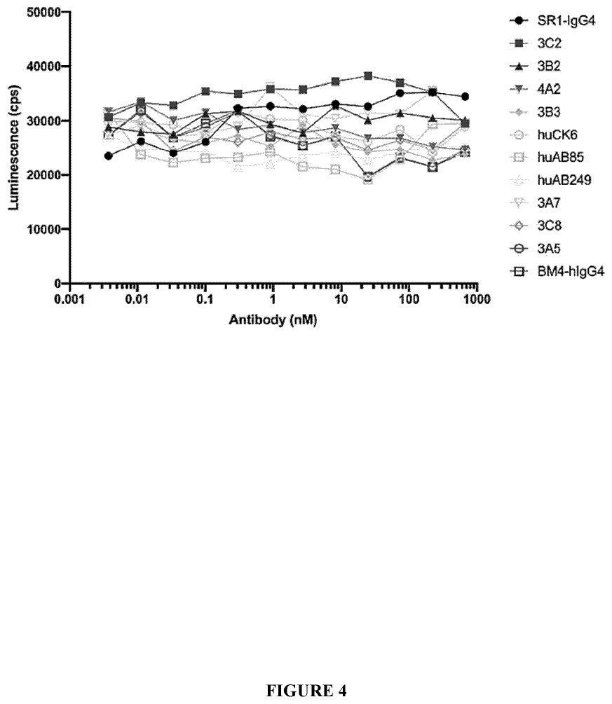

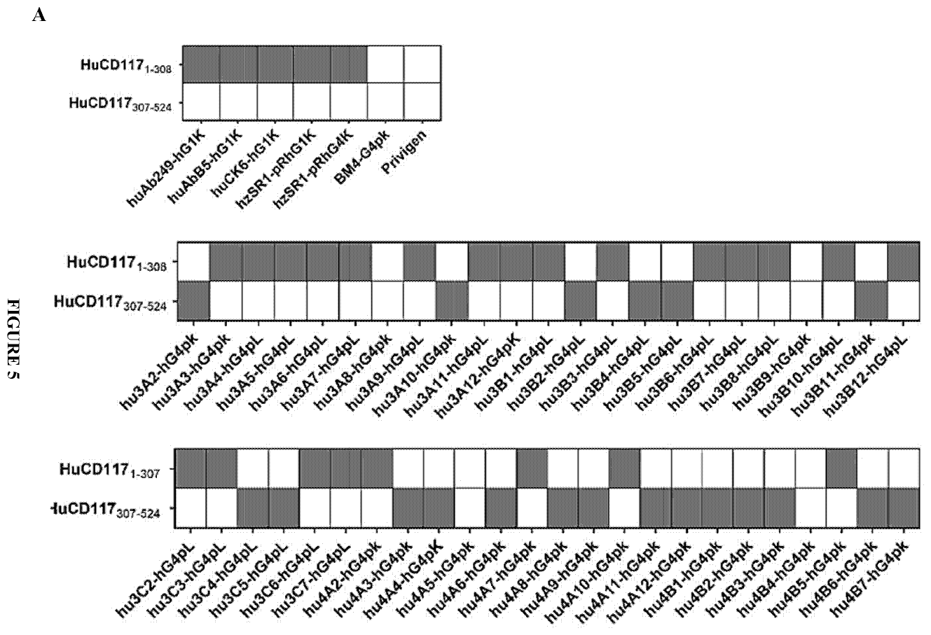

As shown in FIG. 4, none of the 47 anti-CD117-IgG1 antibodies were able to activate CD117 receptor and induce proliferation of TF-1 cells in the absence of stem cell factor.

### Example 6: Binding Affinity of Anti-CD117 Antibodies Against the Soluble N-Terminal (HuCD117₁₋₃₀₇) and C-Terminal (HuCD117₃₀₈₋₅₂₄)

Biacore 4000 was used for all binding analyses. Experiments were performed at pH 7.3, 37° C. and under flow rate of 30 μL/min. Human anti-CD117 antibodies were surface captured using protein G directly immobilised onto the carboxymethyl dextran surface of CM5 sensorchips to approximately 1,500 RU using standard NHS/EDC chemistry at pH 4.5. The immobilised protein G surface was pre-conditioned with ten injections of human polyclonal IgG (Privigen 60s, 10 μg/mL) each followed by a regeneration cycle with 10 mM glycine pH 1.6.

A total of 52 anti-CD117 antibodies (2 ug/mL) were tested (N=1) as captured ligands against soluble 8×his-tagged N-terminal (HuCD117₁₋₃₀₇) and C-terminal (HuCD117₃₀₈₋₅₂₄) fragments of human CD117 receptor. Among these, 47 candidates were reformatted into an IgG4 backbone. Four antibodies (huAb249, huAb85, huCK6 and huhzSR1) were reformatted into an IgG1 backbone and included as benchmark controls. HuhzSR1 was also reformatted into an IgG4 backbone. BM4 and Privigen were used as negative controls.

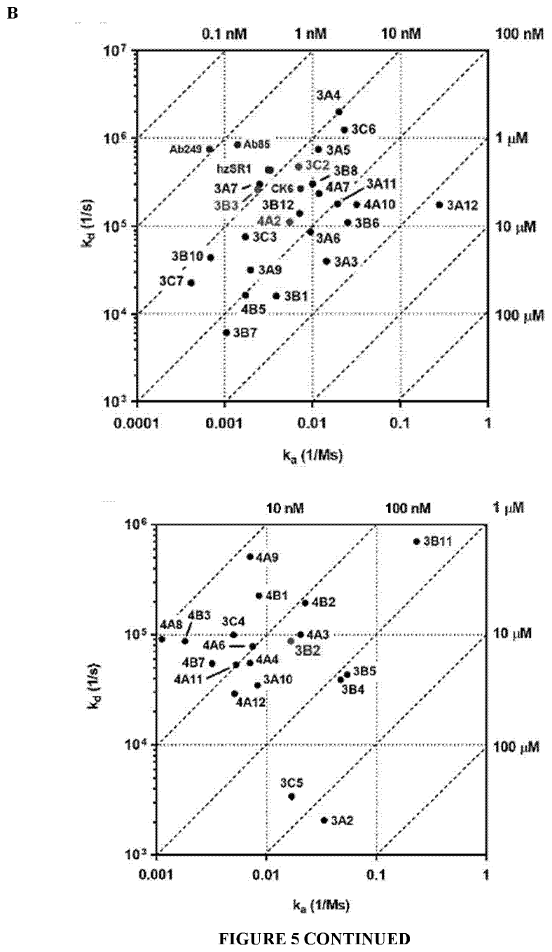

HuCD117 fragments were prepared in three-fold dilutions of 25, 75, 225 nM. Anti-CD117 antibodies huAb249, huAb85, huCK6 and huhzSR1 were used as positive controls. Association and dissociation were monitored for 120 seconds and 180 seconds. No significant difference was observed from huhzSR1 antibodies prepared in either IgG₁ or IgG₄ backbones. Anti-CD117 antibodies bound either the N-terminal or C-terminal domains, not both. Positive binding responses to a soluble domain are illustrated in green while negative responses shown in white (FIG. 5A). Approximate binding affinities were obtained from sensorgrams double-referenced using reference surface and blank buffer injection data gathered within each experiment. Rate constants and binding affinities were calculated using a 1:1 kinetic model with local Rmax and null refraction index (RI=0).

48 anti-CD117 antibodies bound either domain, not both (FIG. 5A). The N-terminal domain bound 23 anti-CD117 antibodies at affinities ranging from 8 nM (hu3A7) to 1.5 μM (hu3A12) while the C-terminal domain bound the remaining 20 anti-CD117 antibodies at affinities ranging from 12 nM (hu4A8) to 16 μM (hu3A2). Positive control anti-CD117 antibodies huAb249, huAb85, huCK6 and huhzSR1 only bound the N-terminal domain. Four antibodies (hu3A8, hu3B9, hu4A5 and hu4B4) did not bind either CD117 domain suggesting that their epitopes sit at the junction of N and C-terminal regions. No response was detected from either BM4 (negative control) or Privigen (polyclonal human IgG). The binding affinity of the antibody panel to each domain is illustrated in FIG. 5A and the overall distribution of binding affinities is shown in FIGS. 5B and C.

Four antibodies were shortlisted for further studies based on their cell-based activity and cross reactivity. Among these, hu3B3 (9 nM), hu3C2 (14 nM) and hu4A2 (49 nM) bound the N-terminal domain while hu3B2 (191 nM) bound the C-terminal domain of CD117.

### Example 7: Comparison Anti-CD117 Antibodies Prepared on an IgG1, IgG4 or IgA2 Backbones

Biacore 4000 was used for all binding analyses. Experiments were performed at pH 7.3, 37° C. and under flow rate of 30 μL/min. Human anti-CD117 antibodies were surface captured using either polyclonal goat anti-human IgG or IgA antibodies directly immobilised onto the carboxymethyl dextran surface of CM5 sensorchips to approximately 13,000 RU using standard NHS/EDC chemistry at pH 5. The immobilised antibody surface was pre-conditioned with ten injections of human polyclonal IgG (Privigen 60s, 10 μg/mL) each followed by a regeneration cycle with 100 mM H3PO4.

A total of 18 anti-CD117 antibodies (2 μg/mL) were tested as captured ligands against four soluble mammalian CD117 receptor variants: the N-terminal domain of human CD117 (HuCD1₁₇₁₋₃₀₈), full length human CD117 (HuCD1₁₇₁₋₅₂₄), cyno CD117 (cynoCD117₁₋₅₂₀), and murine CD117 (MuCD117₁₋₅₂₇). The 18 antibodies corresponded to 4 parental mAbs (hu3B2, hu3B3, hu3C2 and hu4Aa) and four control antibodies (huCK6, huAb249, huAb85 and hzSR1) prepared on either an IgG1 or IgG4 and IgA2 backbones.

Antibodies were captured for 60 seconds to approximately 2,000 RU at the active spots (1 and 5) of each flow cell while inner spots (2 and 4) were left blank and used for reference subtraction. Association and dissociation phases were monitored for 120 and 180 seconds, respectively.

CD117 proteins were prepared in two-fold dilutions ranging from 0.8 to 200 nM. Anti-CD117 antibodies huAb249, huAb85, huCK6 and huhzSR1 were used as positive controls. BM4 was prepared in an IgG and IgA2 format and used as negative control. Binding affinities were calculated from sensorgrams double-referenced using reference surface and blank buffer injection data gathered within each experiment. Rate constants and binding affinities were calculated using a 1:1 kinetic model with local Rmax and null refraction index (RI=0).

Briefly, human anti-CD117 antibodies bound the N-terminal domain of CD117 at ˜2-fold higher affinity compared to the full-length protein. Anti-CD117 antibodies bound the human and cynomolgus monkey isoforms of CD117 at comparable affinities, which is consistent considering the high degree of similarity between these two receptors.

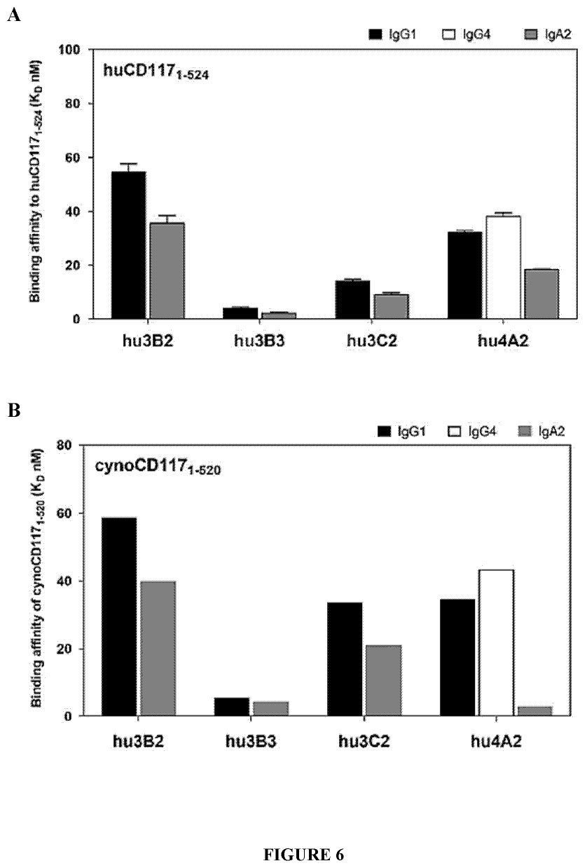

The overall binding affinity (K_{D}) of these antibodies to the full-length human and cyno receptors can be expressed as Hu3B3 (2-5 nM)<hu3C2 (10-30 nM)<hu4A2 (˜3-40 nM)<hu3B2 (35-59 nM) (FIG. 6). Antibodies produced on an IgA₂ backbone bound their antigen at increased affinities compared to those prepared on an IgG1 or IgG4. Hu4A2 showed the largest gains in affinity (˜2-fold against huCD117 and ˜10-fold against cynoCD117).

It was not possible to determine the binding affinity of these antibodies to the murine isoform of CD117. As shown in Table 1 murine CD117 displayed weak binding affinities (i.e., KD>1 μM) to antibodies hu3B2 and hu4A2 and no binding to the remaining antibodies. Control antibodies huCK6, huAb249, huAb85 and hzSR1 bound human and cyno receptors at low nanomolar affinities (Table 1).

TABLE 1
Binding affinity (K_{D}) of soluble human, cynomolgus monkey and
murine CD117 to a panel of anti-human CD117 antibodies prepared on
a human IgG1/IgG4 or human IgA2 (hLhA2_{P221R}) backbone.

| | | HuCD117₁₋₃₀₈ | HuCD117₁₋₅₂₄ | cynoCD117₁₋₅₂₀ | muCD117₁₋₅₂₇ |
|---|---|---|---|---|---|
| Antibody | Isotype | K_{D} (nM) | K_{D} (nM) | K_{D} (nM) | K_{D} (nM) |
| hu3B2 | IgG1 | NB | 54.6 | 58.7 | WB |
| hu3B2 | IgA2 | NB | 35.9 | 39.9 | WB |
| hu3B3 | IgG1 | 1.07 | 4.2 | 5.7 | NB |
| hu3B3 | IgA2 | 0.49 | 2.5 | 4.4 | NB |
| hu3C2 | IgG1 | 4.14 | 14.4 | 33.7 | NB |
| hu3C2 | IgA2 | 2.44 | 9.3 | 20.9 | NB |
| hu4A2 | IgG1 | 14.45 | 32.4 | 34.6 | WB |
| hu4A2 | IgG4 | 19.86 | 38.3 | 43.4 | WB |
| hu4A2 | IgA2 | 8.61 | 18.6 | 3.0 | WB |
| huAb249 | IgG4 | 0.14 | 0.3 | 0.3 | NB |
| huAb249 | IgA2 | 0.08 | 0.3 | 0.1 | NB |
| huAb85 | IgG4 | 0.31 | 0.7 | 0.8 | NB |
| huAb85 | IgA2 | 0.16 | 0.4 | 0.5 | NB |
| huCK6 | IgG4 | 2.16 | 6.3 | 7.8 | NB |
| huCK6 | IgA2 | 1.20 | 2.6 | 2.9 | NB |
| hzSR1 | IgG1 | 0.66 | 3.9 | 3.9 | NB |
| hzSR1 | IgG4 | 1.18 | 3.4 | 3.6 | NB |
| hzSR1 | IgA2 | 1.08 | 2.0 | 2.8 | NB |

Binding values were calculated from sensorgram data fit to a 1:1 binding model. Values shown in nanomolar as mean of at least two experimental replicates. WB: weak binding; NB: no binding.

### Example 8: In Vitro Antibody-Dependent Cytotoxic Trogocytosis (ADCT) Assay with Exemplary Candidates

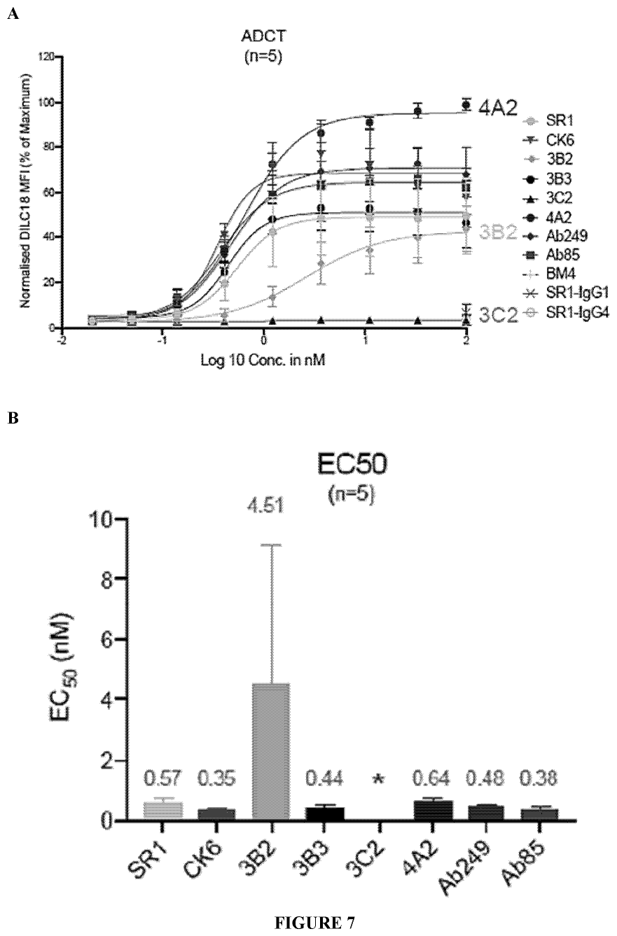

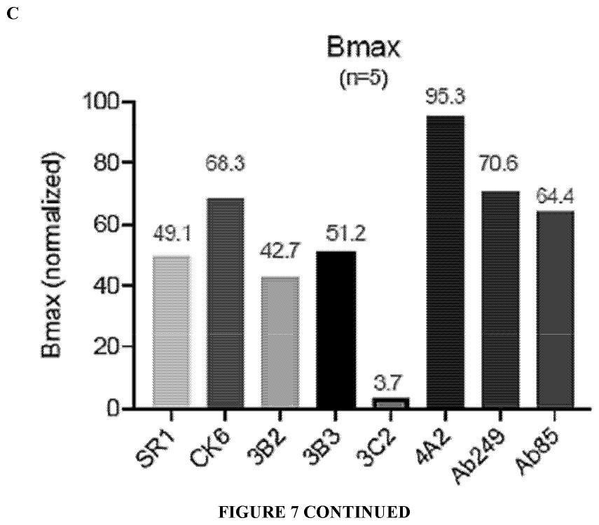

The IgA2-anti CD117 antibody trogocytosis activity of the newly generated clones 3B2, 3B3, 3C2, 4A2, Ab249, Ab85 was determined by flow cytometry and compared to previously published clones SR1 and CK6. In brief, the 293-target cell line transfected with human CD117 was labelled with the membrane-dye DILC18 (1, 1″-dioctadexyl-3,3, 3″, 3″-tetramethylindocarbocyanine perchlorate, Thermo Fisher), mixed 1:10 with freshly isolated peripheral blood neutrophils as effector cells and incubated with human IgA2 anti CD117 antibodies (0.02-100 nM) or control antibodies (100 nM) for 4 hrs at 37° C., 5% CO2 in a total volume of 150 μL. Subsequently, cells were stained with Alexa fluor 647-conjugated anti CD66b and the LIVE/DEAD™ Fixable Green Stain (Invitrogen), washed and fixed with formaldehyde (BD Cytofix). Cells were acquired on the Fortessa and live neutrophils were identified by forward and side scatter, followed by live CD66b positive cells. Membrane uptake by neutrophils was measured on the YG586-15 detector by increase of DILC18 mean fluorescence intensity (MFI). DILC18 MFI was normalised to the maximum MFI per donor neutrophil (FIG. 7A).

The half maximal effective concentration (EC₅₀±SD) in [nM] and the normalised maximum membrane uptake (Bₘₐₓ) in percent facilitated by IgA2 anti CD117 antibodies inducing trogocytosis by peripheral blood neutrophils from n=5 donors are shown in Table 1 and Figure B and C. EC50 and normalised Bmax (%) were calculated using Prism 8 Graphphad Software utilising a 4-parameter logics curve based on experiment shown in Graph 7.1.

TABLE 2
EC₅₀ and Bₘₐₓ facilitated by anti CD117-IgA2 antibodies
inducing trogocytosis by peripheral blood neutrophils

| | | Normalised uptake |
|---|---|---|
| Antibody | EC50 ± SD [nM] | (B max) in % |
| 3B2 | 4.51 ± 4.12 | 42.74 |
| 3B3 | 0.44 ± 0.07 | 51.15 |
| 3C2 | non-detectable | 3.377 |
| 4A2 | 0.64 ± 0.08 | 95.26 |
| Ab249 | 0.48 ± 0.05 | 70.59 |
| Ab85 | 0.38 ± 0.09 | 64.4 |
| SR1 | 0.58 ± 0.13 | 49.19 |
| CK6 | 0.35 ± 0.07 | 68.25 |

### Example 9: Modifying Hinge Region of Anti-CD117-IgG1 Antibodies

Two IgA₂ candidates prepared with an extended IgA₂/IgG₃ hinge region. Antibody 3C2 (CDRs were not modified, applicable CDRs are SEQ ID Nos. 42-47) and SR1 (Commercial antibody as control).

The IgG₃ hinge region was inserted after the first Pro of the IgA₂ hinge (Senior et al., 2000 and Woof & Kerr, 2006).

Two versions of the extended hinge region were produced (see Table 3). Version 1 (V1) had 5× proline motifs in the heavy chain, with the first proline from the lower hinge and 4 additional proline from the IgA₂ sequence, Version 2 (V2) had 4× proline motifs in the heavy chain. For both of these versions the light chain sequences remained the same.

TABLE 3
Annotated IgA2/IgG3 extended
hinge Versions 1 (V1) and 2 (V2)

| | | | | | IgA |
|---|---|---|---|---|---|
| | C- | | | | sequence |
| | terminal | Upper | Middle | Lower | past |
| Construct | CH1 (IgA | Hinge | Hinge | Hinge | extended |
| name | based) | (IgG3) | (IgG3) | (IgG3) | hinge |
| IgA2/IgG3 | PCRVP | ELKTPLG | CPRCP | APELL | PPPPCCHP |
| hinge V1 | (SEQ ID | DTTHT | (EPKSC | GGP | R |
| | NO: 123) | (SEQ ID | DTPPPC | (SEQ | (SEQ ID |
| | | NO: 124) | PRCP)₃ | ID NO: | NO: 127) |
| | | | (SEQ ID | 126) | |
| | | | NO: 125) | | |
| IgA2/IgG3 | PCRVP | ELKTPLG | CPRCP | APELL | PPPCCHPR |
| hinge V2 | (SEQ ID | DTTHT | (EPKSC | GGP | (SEQ ID |
| | NO: 123) | (SEQ ID | DTPPPC | (SEQ | NO: 128) |
| | | NO: 124) | PRCP)₃ | ID NO: | |
| | | | (SEQ ID | 126) | |
| | | | NO: 125) | | |

*R corresponds to stabilising P221R mutation

TABLE 4
Sequences

| SEQ | |
|---|---|
| ID NO | Sequence |
| 1 | MRGARGAWDFLCVLLLLLRVQTGSSQPSVSPGEPSPPSIHPGKSDLIVRVGDEIRLL |
| | CTDPGFVKWTFEILDETNENKQNEWITEKAEATNTGKYTCTNKHGLSNSIYVFVRD |
| | PAKLFLVDRSLYGKEDNDTLVRCPLTDPEVTNYSLKGCQGKPLPKDLRFIPDPKAGI |
| | MIKSVKRAYHRLCLHCSVDQEGKSVLSEKFILKVRPAFKAVPVVSVSKASYLLREG |
| | EEFTVTCTIKDVSSSVYSTWKRENSQTKLQEKYNSWHHGDFNYERQATLTISSARV |
| | NDSGVFMCYANNTFGSANVTTTLEVVDKGFINIFPMINTTVFVNDGENVDLIVEYE |
| | AFPKPEHQQWIYMNRTFTDKWEDYPKSENESNIRYVSELHLTRLKGTEGGTYTFLV |
| | SNSDVNAAIAFNVYVNTKPEILTYDRLVNGMLQCVAAGFPEPTIDWYFCPGTEQRC |
| | SASVLPVDVQTLNSSGPPFGKLVVQSSIDSSAFKHNGTVECKAYNDVGKTSAYFNF |
| | AFKGNNKEQIHPHTLFTPLLIGFVIVAGMMCIIVMILTYKYLQKPMYEVQWKVVEE |
| | INGNNYVYIDPTQLPYDHKWEFPRNRLSFGKTLGAGAFGKVVEATAYGLIKSDAA |
| | MTVAVKMLKPSAHLTEREALMSELKVLSYLGNHMNIVNLLGACTIGGPTLVITEY |
| | CCYGDLLNFLRRKRDSFICSKQEDHAEAALYKNLLHSKESSCSDSTNEYMDMKPG |
| | VSYVVPTKADKRRSVRIGSYIERDVTPAIMEDDELALDLEDLLSFSYQVAKGMAFL |
| | ASKNCIHRDLAARNILLTHGRITKICDFGLARDIKNDSNYVVKGNARLPVKWMAPE |
| | SIFNCVYTFESDVWSYGIFLWELFSLGSSPYPGMPVDSKFYKMIKEGFRMLSPEHAP |
| | AEMYDIMKTCWDADPLKRPTFKQIVQLIEKQISESTNHIYSNLANCSPNRQKPVVD |
| | HSVRINSVGSTASSSQPLLVHDDV |
| 2 | MRGARGAWDFLCVLLFLLHVQTGSSQPSVSPGEPSPPSIHPAKSELIVRVGNEIRLL |
| | CIDPGFVKWTFEILDETNENKQNEWITEKAEATNTGKYTCTNKHGLSSSIYVFVRDP |
| | AKLFLVDRSLYGKEDNDTLVRCPLTDPEVTSYSLKGCQGKPLPKDLRFVPDPKAGI |
| | TIKSVKRAYHRLCLHCSADQEGKSVLSDKFILKVRPAFKAVPVVSVSKASYLLREG |
| | EEFTVTCTIKDVSSSVYSTWKRENSQTKLQEKYNSWHHGDFNYERQATLTISSARV |
| | NDSGVFMCYANNTFGSANVTTTLEVVDKGFINIFPMINTTVFVNDGENVDLIVEYE |
| | AFPKPEHQQWIYMNRTFTDKWEDYPKSENESNIRYVSELHLTRLKGTEGGTYTFLV |
| | SNSDVNASIAFNVYVNTKPEILTYDRLVNGMLQCVAAGFPEPTIDWYFCPGTEQRC |
| | SASVLPVDVQTLNASGPPFGKLVVQSSIDSSAFKHNGTVECKAYNDVGKTSAYFNF |
| | AFKGNNKEQIHPHTLFTPLLIGFVIVAGMMCIIVMILTYKYLQKPMYEVQWKVVEE |
| | INGNNYVYIDPTQLPYDHKWEFPRNRLSFGKTLGAGAFGKVVEATAYGLIKSDAA |
| | MTVAVKMLKPSAHLTEREALMSELKVLSYLGNHMNIVNLLGACTIGGPTLVITEY |
| | CCYGDLLNFLRRKRDSFICSKQEDHAESALYKNLLHSKESSCSDSTNEYMDMKPG |
| | VSYVVPTKADKRRSARIGSYIERDVTPAIMEDDELALDLEDLLSFSYQVAKGMAFL |
| | ASKNCIHRDLAARNILLTHGRITKICDFGLARDIKNDSNYVVKGNARLPVKWMAPE |
| | SIFNCVYTFESDVWSYGIFLWELFSLGSSPYPGMPVDSKFYKMIKEGFRMLSPEHAP |
| | AEMYDIMKTCWDADPLKRPTFKQIVQLIEKQISESTNHIYSNLANCSPNRQKPVVD |
| | HSVRINSVGSTASSSQPLLVHDDV |
| 3 | MRGARGAWDLLCVLLVLLRGQTATSQPSASPGEPSPPSIHPAQSELIVEAGDTLSLT |
| | CIDPDFVRWTFKTYFNEMVENKKNEWIQEKAEATRTGTYTCSNSNGLTSSIYVFVR |
| | DPAKLFLVGLPLFGKEDSDALVRCPLTDPQVSNYSLIECDGKSLPTDLTFVPNPKAG |
| | ITIKNVKRAYHRLCVRCAAQRDGTWLHSDKFTLKVRAAIKAIPVVSVPETSHLLKK |
| | GDTFTVVCTIKDVSTSVNSMWLKMNPQPQHIAQVKHNSWHRGDFNYERQETLTIS |
| | SARVDDSGVFMCYANNTFGSANVTTTLKVVEKGFINISPVKNTTVFVTDGENVDL |
| | VVEYEAYPKPEHQQWIYMNRTSANKGKDYVKSDNKSNIRYVNQLRLTRLKGTEG |
| | GTYTFLVSNSDASASVTFNVYVNTKPEILTYDRLINGMLQCVAEGFPEPTIDWYFCT |
| | GAEQRCTTPVSPVDVQVQNVSVSPFGKLVVQSSIDSSVFRHNGTVECKASNDVGKS |
| | SAFFNFAFKGNNKEQIQAHTLFTPLLIGFVVAAGAMGIIVMVLTYKYLQKPMYEVQ |
| | WKVVEEINGNNYVYIDPTQLPYDHKWEFPRNRLSFGKTLGAGAFGKVVEATAYGL |
| | IKSDAAMTVAVKMLKPSAHLTEREALMSELKVLSYLGNHMNIVNLLGACTVGGPT |
| | LVITEYCCYGDLLNFLRRKRDSFIFSKQEEQAEAALYKNLLHSTEPSCDSSNEYMD |
| | MKPGVSYVVPTKTDKRRSARIDSYIERDVTPAIMEDDELALDLDDLLSFSYQVAKG |
| | MAFLASKNCIHRDLAARNILLTHGRITKICDFGLARDIRNDSNYVVKGNARLPVKW |
| | MAPESIFSCVYTFESDVWSYGIFLWELFSLGSSPYPGMPVDSKFYKMIKEGFRMVSP |
| | EHAPAEMYDVMKTCWDADPLKRPTFKQVVQLIEKQISDSTKHIYSNLANCNPNPE |
| | NPVVVDHSVRVNSVGSSASSTQPLLVHEDA |
| 4 | EVQLXESGGXXVQPGXSLRLSCAASGFTFSXYXMXWVRQAPGKGLEWVSXIXXX |
| | GGXXYYADSVKGRFTISRDNSKNTLYLQMNSLRAEDTAXYYCAXDXXXXXXXXF |
| | DXWGQGTXVTVSS |
| | Wherein |
| | X₅ is A or V or L; X₁₀ is D or G; X₁₁ is L or V; X₁₆ is G or R; X₃₁ is S or D; X₃₃ is A or G; |
| | X₃₅ is S or H; X₅₀ is A or V; X₅₂ is S or G or absent; X₅₃ is G or Y; X₅₄ is S or G; |
| | X₅₇ is S or G; X₅₈ is T or K; X₉₃ is I or V; X₉₈ is R or K; X₁₀₀ is L or S or T; |
| | X₁₀₁ is R or S or W; X₁₀₂ is V or G or W; X₁₀₃ is L or Y; X₁₀₄ is A or R; X₁₀₅ is M or G; |
| | X₁₀₆ is E or D or L; X₁₀₇ is A or absent; X₁₁₀ is I or Y; X₁₁₆ is M or L |
| 5 | SYELTQXPXVSVXXGQTXXITCXGDXXXXXYXXWXQXXXGQXPXLVXXXDXXR |
| | PSGIPERFSGSNSGNTATLTISXXXAXDEADYYCQAWDSSXXXXXFGXGTKX |
| | Wherein |
| | X₇ is D or P; X₉ is A or S; X₁₃ is A or S; X₁₄ is L or P; X₁₈ is V or A; X₁₉ is R or T or S |
| | X₂₃ is Q or S; X₂₆ is S or K; X₂₇ is L or V; X₂₈ is R or G; X₂₉ is D or E or A; |
| | X₃₀ is Y or T or K; X₃₂ is A or V; X₃₃ is N or H or F; X₃₅ is F or Y; X₃₇ is Q or H; |
| | X₃₈ is K or Q; X₃₉ is P or S; X₄₂ is A or S; X₄₄ is I or L or V; X₄₇ is I or V; X₄₈ is F or Y; |
| | X₄₉ is Y or Q; X₅₁ is S or F or N; X₅₂ is D or K; X₇₆ is R or G; X₇₇ is V or T; X₇₈ is E or Q; |
| | X₈₀ is G or M; X₉₄ is S or T; X₉₅ is D or A; X₉₆ is H or V or L; X₉₇ is W or Y or absent; |
| | X₉₈ is V or absent; X₁₀₁ is G or T; X₁₀₅ is L or V |
| 6 | MDWTWRILFLVAAAQPALAEVQLAESGGDLVQPGGSLRLSCAASGFTFSSYAMS |
| | WVRQAPGKGLEWVSAISGSGGSTYYADSVKGRFTISRDNSKNTLYLQMNSLRAED |
| | TAIYYCARDLRVYAMEAFDIWGQGTMVTVSSASTKGPSVFPLAPCSRSTSESTAAL |
| | GCLVKDYFPEPVTVSWNSGALTSGVHTFPAVLQSSGLYSLSSVVTVPSSSLGTKTY |
| | TCNVDHKPSNTKVDKRVESKYGPPCPPCPAPEFLGGPSVFLFPPKPKDTLMISRTPE |
| | VTCVVVDVSQEDPEVQFNWYVDGVEVHNAKTKPREEQFNSTYRVVSVLTVLHQD |
| | WLNGKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEGNVFS |
| | CSVMHEALHNHYTQKSLSLSLGK |
| 7 | EVQLAESGGDLVQPGGSLRLSCAASGFTFSSYAMSWVRQAPGKGLEWVSAISGSG |
| | GSTYYADSVKGRFTISRDNSKNTLYLQMNSLRAEDTAIYYCARDLRVYAMEAFDI |
| | WGQGTMVTVSS |
| 8 | atggactggacctggcgcatcctgtttctggtggccgctgcccaaccagcgctggccgaagtgcagctggcggagtccggggga |
| | gacttagttcagccgggggggtccctgagactctcctgtgcagcctctggattcacctttagcagctatgccatgagctgggtccgcc |
| | aggctccagggaaggggctggagtgggtctcagctattagtggtagtggtggtagcacatactacgcagactccgtgaagggccg |
| | gttcaccatctccagagacaattccaagaacacgctgtatctgcaaatgaacagcctgagagccgaggacacggccatatattactg |
| | tgcgagagacctacgggtgtatgctatggaggcttttgatatctggggccaagggacaatggtcaccgtctcctcagcttccaccaa |
| | gggcccatcggtcttcccgctagcgccctgctccaggagcacctccgagagcacagccgccctgggctgcctggtcaaggacta |
| | cttccccgaaccggtgacggtgtcgtggaactcaggcgccctgaccagcggcgtccacaccttcccggctgtcctacagtcctccg |
| | gactctactccctagcagcgtagtgaccgtgccctccagcagcttgggcacgaagacctacacctgcaacgtagatcacaagcc |
| | cagcaacaccaaggtggacaagagagttgagtccaaatatggtcccccatgcccaccatgcccagcacctgagttcctgggggga |
| | ccatcagtcttcctgttccccccaaaacccaaggacactctcatgatctcccggacccctgaggtcacgtgcgtggtggtggacgtg |
| | agccaggaagaccccgaggtccagttcaactggtacgtggatggcgtggaggtgcataatgccaagacaaagccgcgggagga |
| | gcagttcaacagcacgtaccgtgtggtcagcgtcctcaccgtcctgcaccaggactggctgaacggcaaggagtacaagtgcaaa |
| | gtctccaacaaaggcctcccgtcctccatcgagaaaaccatctccaaagccaaagggcagccccgagagccacaggtgtacacc |
| | ctgcccccatcccaggaggagatgaccaagaaccaggtcagcctgacctgcctggtcaaaggcttctaccccagcgacatcgcc |
| | gtggagtgggagagcaatgggcagccggagaacaactacaagaccacgcctcccgtgctggactccgacggctccttcttcctct |
| | acagcaggctaaccgtggacaagagcaggtggcaggaggggaatgtcttctcatgctccgtgatgcatgaggctctgcacaacca |
| | ctacacacagaagagcctctccctgtctctgggtaaatga |
| 9 | MDWTWRILFLVAFATAVQASYELTQDPAVSVALGQTVRITCQGDSLRDYYANWF |
| | QQKPGQAPLLVIFYDSDRPSGIPERFSGSNSGNTATLTISRVEAGDEADYYCQAWDS |
| | SSDHWVFGGGTKLTVLGQPKAAPSVTLFPPSSEELQANKATLVCLISDFYPGAVTV |
| | AWKADGSPVKAGVETTKPSKQSNNKYAASSYLSLTPEQWKSHRSYSCQVTHEGST |
| | VEKTVAPTECS |
| 10 | SYELTQDPAVSVALGQTVRITCQGDSLRDYYANWFQQKPGQAPLLVIFYDSDRPSG |
| | IPERFSGSNSGNTATLTISRVEAGDEADYYCQAWDSSSDHWVFGGGTKL |
| 11 | atggactggacctggcgcatcctgtttctggtggccttcgccacggccgtgcaggcatcctatgagctgactcaggaccctgctgtg |
| | tctgtggccctgggacagacagtcaggatcacatgccaaggggacagcctcagagactattatgcaaactggttccagcagaagc |
| | caggccaggcccctttattggtcatcttttatgatagcgaccggccctcagggatccctgagcgattctctggctccaactctgggaat |
| | acggccaccctgaccatcagcagggtcgaagccggggatgaggccgactattattgtcaggcgtgggatagtagtagtgaccatt |
| | gggtgttcggcggagggaccaaactgaccgtcctaggtcagcccaaggctgccccctcggtcactctgttcccaccctcctctgag |
| | gagctccaagccaacaaggccacactggtgtgtctgatcagtgacttctaccctggagctgtgacagtggcctggaaggcagatgg |
| | cagccccgtcaaggcgggagtggagacaaccaaaccctccaaacagagcaacaacaagtacgcggccagcagctacctgagc |
| | ctgacgcccgagcagtggaagtcccacagaagctacagctgccaggtcacgcatgaagggagcaccgtggagaagacagtgg |
| | cccctacagaatgttca |
| 12 | MDWTWRILFLVAAAQPALAQVQLQESGPGLVKPSETLSLTCTVSGYSISSGYYWG |
| | WIRQPPGKGLEWIGSIYHSGSTYYNPSLKSRVTISVDTSKNQFSLKLSSVTAADTAV |
| | YYCARVGVRYFDWLLYGAFDIWGQGTMVTVSSASTKGPSVFPLAPCSRSTSESTA |
| | ALGCLVKDYFPEPVTVSWNSGALTSGVHTFPAVLQSSGLYSLSSVVTVPSSSLGTK |
| | TYTCNVDHKPSNTKVDKRVESKYGPPCPPCPAPEFLGGPSVFLFPPKPKDTLMISRT |
| | PEVTCVVVDVSQEDPEVQFNWYVDGVEVHNAKTKPREEQFNSTYRVVSVLTVLH |
| | QDWLNGKEYKCKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTVDKSRWQEGN |
| | VFSCSVMHEALHNHYTQKSLSLSLGK |
| 13 | QVQLQESGPGLVKPSETLSLTCTVSGYSISSGYYWGWIRQPPGKGLEWIGSIYHSGS |
| | TYYNPSLKSRVTISVDTSKNQFSLKLSSVTAADTAVYYCARVGVRYFDWLLYGAF |
| | DIWGQGTMVTVSS |
| 14 | atggactggacctggcgcatcctgtttctggtggccgctgcccaaccagcgctggcccaggtgcagctgcaggagtcgggccca |
| | ggactggtgaagccttcggagaccctgtccctcacctgcactgtctctggttactccatcagcagtggttactactggggctggatcc |
| | ggcagcccccagggaaggggctggagtggattgggagtatctatcatagtgggagcacctactacaacccgtccctcaagagtcg |
| | agtcaccatatcagtagacacgtccaagaaccagttctccctgaagctgagctctgtgaccgccgcagacacggccgtgtattactg |
| | tgcgagagtgggggttcgatattttgactggttattatatggcgcttttgatatctggggccaagggacaatggtcactgtctcctcagc |
| | ttccaccaagggcccatcggtcttcccgctagcgccctgctccaggagcacctccgagagcacagccgccctgggctgcctggtc |
| | aaggactacttccccgaaccggtgacggtgtcgtggaactcaggcgccctgaccagcggcgtccacaccttcccggctgtcctac |
| | agtcctccggactctactccctcagcagcgtagtgaccgtgccctccagcagcttgggcacgaagacctacacctgcaacgtagat |
| | cacaagcccagcaacaccaaggtggacaagagagttgagtccaaatatggtcccccatgcccaccatgcccagcacctgagttcc |
| | tggggggaccatcagtcttcctgttccccccaaaacccaaggacactctcatgatctcccggacccctgaggtcacgtgcgtggtg |
| | gtggacgtgagccaggaagaccccgaggtccagttcaactggtacgtggatggcgtggaggtgcataatgccaagacaaagccg |
| | cgggaggagcagttcaacagcacgtaccgtgtggtcagcgtcctcaccgtcctgcaccaggactggctgaacggcaaggagtac |
| | aagtgcaaagtctccaacaaaggcctcccgtcctccatcgagaaaaccatctccaaagccaaagggcagccccgagagccacag |
| | gtgtacaccctgcccccatcccaggaggagatgaccaagaaccaggtcagcctgacctgcctggtcaaaggcttctaccccagcg |
| | acatcgccgtggagtgggagagcaatgggcagccggagaacaactacaagaccacgcctcccgtgctggactccgacggctcc |
| | ttcttcctctacagcaggctaaccgtggacaagagcaggtggcaggaggggaatgtcttctcatgctccgtgatgcatgaggctctg |
| | cacaaccactacacacagaagagcctctccctgtctctgggtaaatga |
| 15 | MDWTWRILFLVAFATAVQASYELTQPPSVSVSPGQTATITCSGDKLGETYVHWYQ |
| | HQSGQSPILVVYQDFKRPSGIPERFSGSNSGNTATLTISGTQAMDEADYYCQAWDS |
| | STAVFGGGTKLTVLGQPKAAPSVTLFPPSSEELQANKATLVCLISDFYPGAVTVAW |
| | KADGSPVKAGVETTKPSKQSNNKYAASSYLSLTPEQWKSHRSYSCQVTHEGSTVE |
| | KTVAPTECS |
| 16 | SYELTQPPSVSVSPGQTATITCSGDKLGETYVHWYQHQSGQSPILVVYQDFKRPSGI |
| | PERFSGSNSGNTATLTISGTQAMDEADYYCQAWDSSTAVFGGGTKL |
| 17 | atggactggacctggcgcatcctgtttctggtggccttcgccacggccgtgcaggcatcctatgagctgactcagccaccctcagtg |
| | tccgtgtcgcctggacagacagccaccatcacctgctctggagataagttgggggaaacttatgttcattggtatcagcatcagtcag |
| | gccagtcccctattttagtcgtctatcaagatttcaagcggccctccgggatccctgagcgattctctggctccaactctgggaacaca |
| | gccactctgaccatcagcgggacccaggctatggatgaggctgactattactgtcaggcgtgggacagcagcactgcggtattcg |
| | gcggagggaccaagctgaccgtcctaggtcagcccaaggctgccccctcggtcactctgttcccaccctcctctgaggagctcca |
| | agccaacaaggccacactggtgtgtctgatcagtgacttctaccctggagctgtgacagtggcctggaaggcagatggcagcccc |
| | gtcaaggcgggagtggagacaaccaaaccctccaaacagagcaacaacaagtacgcggccagcagctacctgagcctgacgc |
| | ccgagcagtggaagtcccacagaagctacagctgccaggtcacgcatgaagggagcaccgtggagaagacagtggcccctaca |
| | gaatgttca |
| 18 | MDWTWRILFLVAAAQPALAEVQLVESGGGVVQPGRSLRLSCAASGFTFSSYAMH |
| | WVRQAPGKGLEWVSVIYSGGSTYYADSVKGRFTISRDNSKNTLYLQMNSLRAEDT |
| | AVYYCARDSSGYRGDFDYWGQGTLVTVSSASTKGPSVFPLAPCSRSTSESTAALGC |
| | LVKDYFPEPVTVSWNSGALTSGVHTFPAVLQSSGLYSLSSVVTVPSSSLGTKTYTC |
| | NVDHKPSNTKVDKRVESKYGPPCPPCPAPEFLGGPSVFLFPPKPKDTLMISRTPEVT |
| | CVVVDVSQEDPEVQFNWYVDGVEVHNAKTKPREEQFNSTYRVVSVLTVLHQDWL |
| | NGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCS |
| | VMHEALHNHYTQKSLSLSLGK |
| 19 | EVQLVESGGGVVQPGRSLRLSCAASGFTFSSYAMHWVRQAPGKGLEWVSVIYSGG |
| | STYYADSVKGRFTISRDNSKNTLYLQMNSLRAEDTAVYYCARDSSGYRGDFDYW |
| | GQGTLVTVSS |
| 20 | atggactggacctggcgcatcctgtttctggtggccgctgcccaaccagcgctggccgaagtgcagctggtggagtctgggggag |
| | gcgtggtccagcctgggaggtccctgagactctcctgtgcagcctctggattcaccttcagtagctatgctatgcactgggtccgcca |
| | ggctccagggaaggggctggagtgggtctcagttatttatagcggtggtagcacatactacgcagactccgtgaagggcagattca |
| | ccatctccagagacaattccaagaacacgctgtatcttcaaatgaacagcctgagagccgaggacacggctgtgtattactgtgcga |
| | gagatagtagtggttatcgaggcgactttgactactggggccagggaaccctggtcaccgtctcctcagcttccaccaagggcccat |
| | cggtcttcccgctagcgccctgctccaggagcacctccgagagcacagccgccctgggctgcctggtcaaggactacttccccga |
| | accagtgacggtgtcgtggaactcaggcgccctgaccagcggcgtccacaccttcccggctgtcctacagtcctcaggactctact |
| | ccctcagcagcgtagtgaccgtgccctccagcagcttgggcacgaagacctacacctgcaacgtagatcacaagcccagcaaca |
| | ccaaggtggacaagagagttgagtccaaatatggtcccccatgcccaccatgcccagcacctgagttcctggggggaccatcagt |
| | cttcctgttccccccaaaacccaaggacactctcatgatctcccggacccctgaggtcacgtgcgtggtggtggacgtgagccagg |
| | aagaccccgaggtccagttcaactggtacgtggatggcgtggaggtgcataatgccaagacaaagccacgggaggagcagttca |
| | acagcacgtaccgtgtggtcagcgtcctcaccgtcctgcaccaggactggctgaacggcaaggagtacaagtgcaaggtgtccaa |
| | caaaggcctcccgtcctccatcgagaaaaccatctccaaagccaaagggcagccccgagagccacaggtgtacaccctgccccc |
| | atcccaggaggagatgaccaagaaccaggtcagcctgacctgcctggtcaaaggcttctaccccagcgacatcgccgtggagtg |
| | ggagagcaatgggcagccggagaacaactacaagaccacgcctcccgtgctggactccgacggctccttcttcctctacagcagg |
| | ctaaccgtggacaagagcaggtggcaggaggggaatgtcttctcatgctccgtgatgcatgaggctctgcacaaccactacacaca |
| | gaagagtctctccctgtctctgggtaaatga |
| 21 | MDWTWRILFLVAFATAVQASYELTQPPSVSVSPGQTASITCSGDKVGAKYVFWYQ |
| | QKPGQSPVLVVYQDNKRPSGIPERFSGSNSGNTATLTISGTQAMDEADYYCQAWD |
| | SSTALYVFGTGTKVTVLGQPKANPTVTLFPPSSEELQANKATLVCLISDFYPGAVTV |
| | AWKADGSPVKAGVETTKPSKQSNNKYAASSYLSLTPEQWKSHRSYSCQVTHEGST |
| | VEKTVAPTECS |
| 22 | SYELTQPPSVSVSPGQTASITCSGDKVGAKYVFWYQQKPGQSPVLVVYQDNKRPS |
| | GIPERFSGSNSGNTATLTISGTQAMDEADYYCQAWDSSTALYVFGTGTKV |
| 23 | atggactggacctggcgcatcctgtttctggtggccttcgccacggccgtgcaggcctcctatgagctgactcagccaccctcagtg |
| | tccgtgtccccaggacagacagccagcatcacctgctctggagataaggtgggggctaaatatgttttctggtatcagcagaagcca |
| | ggccagtcccctgtgctggtcgtctatcaagataacaagcggccctcagggatccctgagcgattctctggctccaactctgggaac |
| | acagccacactgaccatcagcgggacccaggctatggatgaggctgactattactgtcaggcgtgggacagcagcactgcccttta |
| | tgtcttcggaactgggaccaaggtcaccgtcctaggtcagcccaaggccaaccccactgtcactctgttcccaccctcctctgagga |
| | gctccaagccaacaaggccacactggtgtgtctgatcagtgacttctaccctggagctgtgacagtggcctggaaggcagatggca |
| | gccccgtcaaggcgggagtggagacaaccaaaccctccaaacagagcaacaacaagtacgcggccagcagctacctgagcct |
| | gacgcccgagcagtggaagtcccacagaagctacagctgccaggtcacgcatgaagggagcaccgtggagaagacagtggcc |
| | cctacagaatgttcatag |
| 24 | MDWTWRILFLVAAAQPALAEVQLLESGGGLVQPGGSLRLSCAASGFTFSDYGMS |
| | WVRQAPGKGLEWVSAIGYGGGGKYYADSVKGRFTISRDNSKNTLYLQMNSLRAE |
| | DTAVYYCAKDTWWLAGLFDYWGQGTLVTVSSASTKGPSVFPLAPCSRSTSESTAA |
| | LGCLVKDYFPEPVTVSWNSGALTSGVHTFPAVLQSSGLYSLSSVVTVPSSSLGTKT |
| | YTCNVDHKPSNTKVDKRVESKYGPPCPPCPAPEFLGGPSVFLFPPKPKDTLMISRTP |
| | EVTCVVVDVSQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQEGNVF |
| | SCSVMHEALHNHYTQKSLSLSLGK |
| 25 | EVQLLESGGGLVQPGGSLRLSCAASGFTFSDYGMSWVRQAPGKGLEWVSAIGYGG |
| | GGKYYADSVKGRFTISRDNSKNTLYLQMNSLRAEDTAVYYCAKDTWWLAGLFDY |
| | WGQGTLVTVSS |
| 26 | atggactggacctggcgcatcctgtttctggtggccgctgcccaaccagcgctggccgaggtgcagctgttggagtctgggggag |
| | gcttggtacagcctggggggtccctgagactctcctgtgcagcctctggattcacctttagcgattacgggatgagctgggtccgcc |
| | aggctccagggaaggggctggagtgggtatcagccattgggtatggtggaggtggtaaatactacgcagactccgtgaagggcc |
| | ggttcaccatctccagagacaattccaagaacacgctgtatctgcaaatgaacagcctgagagccgaggacacggccgtgtattac |
| | tgtgctaaggatacttggtggctggcgggtctctttgactactggggccagggaaccctggtcaccgtctcctcagcttccaccaag |
| | ggcccatcggtcttcccgctagcgccctgctccaggagcacctccgagagcacagccgccctgggctgcctggtcaaggactact |
| | tccccgaaccagtgacggtgtcgtggaactcaggcgccctgaccagcggcgtccacaccttcccggctgtcctacagtcctcagg |
| | actctactccctcagcagcgtagtgaccgtgccctccagcagcttgggcacgaagacctacacctgcaacgtagatcacaagccca |
| | gcaacaccaaggtggacaagagagttgagtccaaatatggtcccccatgcccaccatgcccagcacctgagttcctggggggacc |
| | atcagtcttcctgttccccccaaaacccaaggacactctcatgatctcccggacccctgaggtcacgtgcgtggtggtggacgtgag |
| | ccaggaagaccccgaggtccagttcaactggtacgtggatggcgtggaggtgcataatgccaagacaaagccacgggaggagc |
| | agttcaacagcacgtaccgtgtggtcagcgtcctcaccgtcctgcaccaggactggctgaacggcaaggagtacaagtgcaaggt |
| | gtccaacaaaggcctcccgtcctccatcgagaaaaccatctccaaagccaaagggcagccccgagagccacaggtgtacaccct |
| | gcccccatcccaggaggagatgaccaagaaccaggtcagcctgacctgcctggtcaaaggcttctaccccagcgacatcgccgt |
| | ggagtgggagagcaatgggcagccggagaacaactacaagaccacgcctcccgtgctggactccgacggctccttcttcctctac |
| | agcaggctaaccgtggacaagagcaggtggcaggaggggaatgtcttctcatgctccgtgatgcatgaggctctgcacaaccact |
| | acacacagaagagtctctccctgtctctgggtaaatga |
| 27 | MGWSCIILFLVATATVQAEIVLTQSPGTLSLSPGKRATLSCRASQSVSNSYLAWYQ |
| | QKPGQPPKLLIYWASTRESGVPDRFSGSGSGTDFTLTISSLQAEDVAVYYCQQYYST |
| | PLTFGGGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQGLSSPVT |
| | KSFNRGEC |
| 28 | EIVLTQSPGTLSLSPGKRATLSCRASQSVSNSYLAWYQQKPGQPPKLLIYWASTRES |
| | GVPDRFSGSGSGTDFTLTISSLQAEDVAVYYCQQYYSTPLTFGGGTKVEIKR |
| 29 | atggactggacctggcgcatcctgtttctggtggccttcgccacggccgtgcaggccgaaattgtgttgacacagtcgccaggcac |
| | cctgtctttgtctccagggaaaagagccaccctctcctgcagggccagtcagagtgttagcaacagctacttagcctggtaccagca |
| | gaaaccaggacagcctcctaagctgctcatttactgggcatctacccgggaatccggggtccctgaccgattcagtggcagcgggt |
| | ctgggacagatttcactctcaccatcagcagcctgcaggctgaagatgtggcagtttattactgtcagcaatattatagtactccgctca |
| | ctttcggcggagggaccaaggtggagatcaaacgaactgtggctgcaccatctgtcttcatcttcccgccatctgatgagcagttga |
| | aatctggaactgcctctgttgtgtgcctgctgaataacttctatcccagagaggccaaagtacagtggaaggtggataacgccctcca |
| | atcgggtaactcccaggagagtgtcacagagcaggacagcaaggacagcacctacagcctcagcagcaccctgacgctgagca |
| | aagcagactacgagaaacacaaagtctacgcctgcgaagtcacccatcagggcctgagttcgcccgtcacaaagagcttcaacag |
| | gggagagtgttag |
| 30 | ASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| 31 | ASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPRE |
| | PQVYTLPPSQEEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGK |
| 32 | ASPTSPKVFPLSLDSTPQDGNVVVACLVQGFFPQEPLSVTWSESGQNVTARNFPPSQ |
| | DASGDLYTTSSQLTLPATQCPDGKSVTCHVKHYTNSSQDVTVPCPVPPPPPCCHPR |
| | LSLHRPALEDLLLGSEANLTCTLTGLRDASGATFTWTPSSGKSAVQGPPERDLCGC |
| | YSVSSVLPGCAQPWNHGETFTCTAAHPELKTPLTANITKSGNTFRPEVHLLPPPSEE |
| | LALNELVTLTCLARGFSPKDVLVRWLQGSQELPREKYLTWASRQEPSQGTTTYAV |
| | TSILRVAAEDWKKGETFSCMVGHEALPLAFTQKTIDRMAGKPTHINVSVVMAEAD |
| | GTCY |
| 33 | ASPTSPKVFPLSLDSTPQDGNVVVACLVQGFFPQEPLSVTWSESGQNVTARNFPPSQ |
| | DASGDLYTTSSQLTLPATQCPDGKSVTCHVKHYTNSSQDVTVPCRVPPPPPCCHPR |
| | LSLHRPALEDLLLGSEANLTCTLTGLRDASGATFTWTPSSGKSAVQGPPERDLCGC |
| | YSVSSVLPGCAQPWNHGETFTCTAAHPELKTPLTANITKSGNTFRPEVHLLPPPSEE |
| | LALNELVTLTCLARGFSPKDVLVRWLQGSQELPREKYLTWASRQEPSQGTTTFAVT |
| | SILRVAAEDWKKGDTFSCMVGHEALPLAFTQKTIDRLAGKPTHVNVSVVMAEVD |
| | GTCY |
| 34 | ASPTSPKVFPLSLDSTPQDGNVVVACLVQGFFPQEPLSVTWSESGQGVTARNFPPSQ |
| | DASGDLYTTSSQLTLPATQCPDGKSVTCHVKHYTNSSQDVTVPCRVPPPPPCCHPR |
| | LSLHRPALEDLLLGSEANLTCTLTGLRDASGATFTWTPSSGKSAVQGPPERDLCGC |
| | YSVSSVLPGSAQPWNHGETFTCTAAHPELKTPLTATLSKSGNTFRPEVHLLPPPSEE |
| | LALNELVTLTCLARGFSPKDVLVRWLQGSQELPREKYLTWASRQEPSQGTTTFAVT |
| | SILRVAAEDWKKGDTFSCMVGHEALPLAFTQKTIDRLAGKPTHVNVSVVMAEVD |
| | GT |
| 35 | TVLGQPKAAPSVTLFPPSSEELQANKATLVCLISDFYPGAVTVAWKADGSPVKAGV |
| | ETTKPSKQSNNKYAASSYLSLTPEQWKSHRSYSCQVTHEGSTVEKTVAPTECS |
| 36 | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 37 | MDWTWRILFLVAAAQPALA |
| 38 | MDWTWRILFLVAFATAVQA |
| 39 | MGWSCIILFLVATATGVHA |
| 40 | MGWSCIILFLVATATVQA |
| 41 | gtaaggggttaacagtagcaggcttgaggtctggacatatatatgggtgacaatgacatccactttgcctttctctccacag |
| 42 | SYAMS |
| 43 | AISGSGGSTYYADSVKG |
| 44 | DLRVYAMEAFDI |
| 45 | QGDSLRDYYAN |
| 46 | YDSDRPS |
| 47 | QAWDSSSDHWV |
| 48 | SGYYWG |
| 49 | SIYHSGSTYYNPSLKS |
| 50 | VGVRYFDWLLYGAFDI |
| 51 | SGDKLGETYVH |
| 52 | QDFKRPS |
| 53 | QAWDSSTAV |
| 54 | SYAMH |
| 55 | VIYSGGSTYYADSVKG |
| 56 | DSSGYRGDFDY |
| 57 | SGDKVGAKYVF |
| 58 | QDNKRPS |
| 59 | QAWDSSTALYV |
| 60 | DYGMS |
| 61 | AIGYGGGGKYYADSVKG |
| 62 | DTWWLAGLFDY |
| 63 | RASQSVSNSYLA |
| 64 | WASTRES |
| 65 | QQYYSTPLT |
| 66 | ASPTSPKVFPLSLDSTPQDGNVVVACLVQGFFPQEPLSVTWSESGQXVTARNFPPSQ |
| | DASGDLYTTSSQLTLPATQCPDGKSVTCHVKHYTNSSQDVTVPCXVPPPPPCCHPR |
| | LSLHRPALEDLLLGSEANLTCTLTGLRDASGATFTWTPSSGKSAVQGPPERDLCGC |
| | YSVSSVLPGXAQPWNHGETFTCTAAHPELKTPLTAXXXKSGNTFRPEVHLLPPPSE |
| | ELALNELVTLTCLARGFSPKDVLVRWLQGSQELPREKYLTWASRQEPSQGTTTFAV |
| | TSILRVAAEDWKKGDTFSCMVGHEALPLAFTQKTIDRLAGKPTHVNVSVVMAEVD |
| | GTXX |
| | Wherein |
| | X₄₇ is N or G; X₁₀₂ is P or R; X₁₇₉ is C or S; X₂₀₅ is N or T; X₂₀₆ is I or L; X₂₀₇ is T or S |
| | X₃₃₉ is C or absent; X₃₄₀ is Y or absent |
| 67 | MDWTWRILFLVAAAQPALAEVQLAESGGDLVQPGGSLRLSCAASGFTFSSYAMS |
| | WVRQAPGKGLEWVSAISGSGGSTYYADSVKGRFTISRDNSKNTLYLQMNSLRAED |
| | TAIYYCARDLRVYAMEAFDIWGQGTMVTVSSASTKGPSVFPLAPSSKSTSGGTAAL |
| | GCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSLT |
| | CLVKGFYPSDIAVEWESNGQPENNYKTTPPVLDSDGSFFLYSKLTVDKSRWQQGN |
| | VFSCSVMHEALHNHYTQKSLSLSPGK |
| 68 | atggactggacctggcgcatcctgtttctggtggccgctgcccaaccagcgctggccgaagtgcagctggcggagtccggggga |
| | gacttagttcagccgggggggtccctgagactctcctgtgcagcctctggattcacctttagcagctatgccatgagctgggtccgcc |
| | aggctccagggaaggggctggagtgggtctcagctattagtggtagtggtggtagcacatactacgcagactccgtgaagggccg |
| | gttcaccatctccagagacaattccaagaacacgctgtatctgcaaatgaacagcctgagagccgaggacacggccatatattactg |
| | tgcgagagacctacgggtgtatgctatggaggcttttgatatctggggccaagggacaatggtcaccgtctcctcagcttccaccaa |
| | gggcccatcggtcttcccgctagcaccctcctccaagagcacctctgggggcacagggccctgggctgcctggtcaaggactac |
| | ttccccgaaccggtgacggtgtgtggaactcaggcgccctgaccagcggcgtccacaccttcccggctgtcctacagtcctccgg |
| | actctactccctcagcagcgtagtgaccgtgccctccagcagcttgggcacccagacctacatctgcaacgtgaatcacaagccca |
| | gcaacaccaaggtggacaagaaagttgagcccaaatcttgtgacaaaactcacacatgcccaccgtgcccagcacctgaactcct |
| | ggggggaccgtcagtcttcctcttccccccaaaacccaaggacaccctcatgatctcccggacccctgaggtcacatgcgtggtgg |
| | tggacgtgagccacgaagaccctgaggtcaagttcaactggtacgtggacggcgtggaggtgcataatgccaagacaaagccgc |
| | gggaggagcagtacaacagcacgtaccgtgtggtcagcgtcctcaccgtcctgcaccaggactggctgaatggcaaggagtaca |
| | agtgcaaggtctccaacaaagccctcccagcccccatcgagaaaaccatctccaaagccaaagggcagccccgagaaccacag |
| | gtgtacaccctgcccccatcccgggatgagctgaccaagaaccaggtcagcctgacctgcctggtcaaaggcttctatcccagcga |
| | catcgccgtggagtgggagagcaatgggcagccggagaacaactacaagaccacgcctcccgtgctggactccgacggctcctt |
| | cttcctctacagcaagctcaccgtggacaagagcaggtggcagcaggggaacgtcttctcatgctccgtgatgcatgaggctctgc |
| | acaaccactacacgcagaagagcctctccctgtctccgggtaaatgaa |
| 69 | MDWTWRILFLVAFATAVQASYELTQDPAVSVALGQTVRITCQGDSLRDYYANWF |
| | QQKPGQAPLLVIFYDSDRPSGIPERFSGSNSGNTATLTISRVEAGDEADYYCQAWDS |
| | SSDHWVFGGGTKLTVLGQPKAAPSVTLFPPSSEELQANKATLVCLISDFYPGAVTV |
| | AWKADGSPVKAGVETTKPSKQSNNKYAASSYLSLTPEQWKSHRSYSCQVTHEGST |
| | VEKTVAPTECS |
| 70 | atggactggacctggcgcatcctgtttctggtggccttcgccacggccgtgcaggcctcctatgagctgactcaggaccctgctgtg |
| | tctgtggccctgggacagacagtcaggatcacatgccaaggggacagcctcagagactattatgcaaactggttccagcagaagc |
| | caggccaggcccctttattggtcatcttttatgatagcgaccggccctcagggatccctgagcgattctctggctccaactctgggaat |
| | acggccaccctgaccatcagcagggtcgaagccggggatgaggccgactattattgtcaggcgtgggatagtagtagtgaccatt |
| | gggtgttcggcggagggaccaaactgaccgtcctaggtcagcccaaggctgccccctcggtcactctgttcccaccctcctctgag |
| | gagctccaagccaacaaggccacactggtgtgtctgatcagtgacttctaccctggagctgtgacagtggcctggaaggcagatgg |
| | cagccccgtcaaggcgggagtggagacaaccaaaccctccaaacagagcaacaacaagtacgcggccagcagctacctgagc |
| | ctgacgcccgagcagtggaagtcccacagaagctacagctgccaggtcacgcatgaagggagcaccgtggagaagacagtgg |
| | cccctacagaatgttca |
| 71 | MDWTWRILFLVAAAQPALAEVQLAESGGDLVQPGGSLRLSCAASGFTFSSYAMS |
| | WVRQAPGKGLEWVSAISGSGGSTYYADSVKGRFTISRDNSKNTLYLQMNSLRAED |
| | TAIYYCARDLRVYAMEAFDIWGQGTMVTVSSASPTSPKVFPLSLDSTPQDGNVVV |
| | ACLVQGFFPQEPLSVTWSESGQNVTARNFPPSQDASGDLYTTSSQLTLPATQCPDG |
| | KSVTCHVKHYTNSSQDVTVPCRVPPPPPCCHPRLSLHRPALEDLLLGSEANLTCTLT |
| | GLRDASGATFTWTPSSGKSAVQGPPERDLCGCYSVSSVLPGCAQPWNHGETFTCT |
| | AAHPELKTPLTANITKSGNTFRPEVHLLPPPSEELALNELVTLTCLARGFSPKDVLV |
| | RWLQGSQELPREKYLTWASRQEPSQGTTTFAVTSILRVAAEDWKKGDTFSCMVGH |
| | EALPLAFTQKTIDRLAGKPTHVNVSVVMAEVDGTCY |
| 72 | atggactggacctggcgcatcctgtttctggtggccgctgcccaaccagcgctggccgaagtgcagctggcggagtccggggga |
| | gacttagttcagccgggggggtccctgagactctcctgtgcagcctctggattcacctttagcagctatgccatgagctgggtccgcc |
| | aggctccagggaaggggctggagtgggtctcagctattagtggtagtggtggtagcacatactacgcagactccgtgaagggccg |
| | gttcaccatctccagagacaattccaagaacacgctgtatctgcaaatgaacagcctgagagccgaggacacggccatatattactg |
| | tgcgagagacctacgggtgtatgctatggaggcttttgatatctggggccaagggacaatggtcactgtctcctctgcttccccaacc |
| | tctcctaaggtgttccctctgagcctggacagcacccctcaggatggaaatgtggtggtggcctgtctggtgcagggattcttcccac |
| | aagagcccctgtccgtgacttggagcgagtctggacagaacgtgaccgccagaaacttcccaccttctcaggacgcctctggcga |
| | cctgtacaccacctcttctcagctgaccctgcctgccacacagtgccctgatggcaagtctgtgacctgccacgtgaagcactacac |
| | caactccagccaggacgtgaccgtgccttgtagagttcctcctcctccaccttgctgtcaccctcggctgtctctgcatagacccgctc |
| | tggaagatctgctgctgggctctgaggccaacctgacatgtaccctgaccggcctgagagatgcttctggcgccacctttacctgga |
| | caccttccagcggaaagtccgctgttcagggacctcctgagagggacctgtgcggctgttactctgtgtcctctgtgctgcctggctg |
| | tgcccagccttggaatcacggcgagacattcacctgtaccgctgctcaccccgagctgaaaacccctctgaccgccaacatcacca |
| | agtccggcaacaccttccggcctgaagtgcatctgctgcctccacctagcgaggaactggccctgaatgagctggtcaccctgacc |
| | tgtctggccaggggctttagccctaaggacgtgctcgttagatggctgcagggctcccaagagctgcccagagagaagtatctgac |
| | ctgggcctctcggcaagagccatctcagggcaccacaacctttgccgtgaccagcatcctgagagtggccgccgaagattggaag |
| | aagggcgacaccttcagctgcatggtcggacatgaagccctgcctctggctttcacccagaaaaccatcgacagactggccggca |
| | agcccacacatgtgaatgtgtctgtggtcatggccgaggtggacggcacctgttactgatag |
| 73 | MGWSCIILFLVATATGVHASYELTQDPAVSVALGQTVRITCQGDSLRDYYANWFQ |
| | QKPGQAPLLVIFYDSDRPSGIPERFSGSNSGNTATLTISRVEAGDEADYYCQAWDSS |
| | SDHWVFGGGTKLTVLGQPKAAPSVTLFPPSSEELQANKATLVCLISDFYPGAVTVA |
| | WKADGSPVKAGVETTKPSKQSNNKYAASSYLSLTPEQWKSHRSYSCQVTHEGSTV |
| | EKTVAPTECS |
| 74 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcatcctatgagctgactcaggaccctgctgtgtctgtggc |
| | cctgggacagacagtcaggatcacatgccaaggggacagcctcagagactattatgcaaactggttccagcagaagccaggcca |
| | ggcccctttattggtcatcttttatgatagcgaccggccctcagggatccctgagcgattctctggctccaactctgggaatacggcca |
| | ccctgaccatcagcagggtcgaagccggggatgaggccgactattattgtcaggcgtgggatagtagtagtgaccattgggtgttc |
| | ggcggagggaccaaactgaccgtcctaggtcagcccaaggctgccccctcggtcactctgttcccaccctcctctgaggagctcc |
| | aagccaacaaggccacactggtgtgtctgatcagtgacttctaccctggagctgtgacagtggcctggaaggcagatggcagccc |
| | cgtcaaggcgggagtggagacaaccaaaccctccaaacagagcaacaacaagtacgcggccagcagctacctgagcctgacg |
| | cccgagcagtggaagtcccacagaagctacagctgccaggtcacgcatgaagggagcaccgtggagaagacagtggcccctac |
| | agaatgttca |
| 75 | MDWTWRILFLVAAAQPALAEVQLAESGGDLVQPGGSLRLSCAASGFTFSSYAMS |
| | WVRQAPGKGLEWVSAISGSGGSTYYADSVKGRFTISRDNSKNTLYLQMNSLRAED |
| | TAIYYCARDLRVYAMEAFDIWGQGTMVTVSSASPTSPKVFPLSLDSTPQDGNVVV |
| | ACLVQGFFPQEPLSVTWSESGQGVTARNFPPSQDASGDLYTTSSQLTLPATQCPDG |
| | KSVTCHVKHYTNSSQDVTVPCRVPPPPPCCHPRLSLHRPALEDLLLGSEANLTCTLT |
| | GLRDASGATFTWTPSSGKSAVQGPPERDLCGCYSVSSVLPGSAQPWNHGETFTCTA |
| | AHPELKTPLTATLSKSGNTFRPEVHLLPPPSEELALNELVTLTCLARGFSPKDVLVR |
| | WLQGSQELPREKYLTWASRQEPSQGTTTFAVTSILRVAAEDWKKGDTFSCMVGHE |
| | ALPLAFTQKTIDRLAGKPTHVNVSVVMAEVDGT |
| 76 | atggactggacctggcgcatcctgtttctggtggccgctgcccaaccagcgctggccgaagtgcagctggcggagtccggggga |
| | gacttagttcagccgggggggtccctgagactctcctgtgcagcctctggattcacctttagcagctatgccatgagctgggtccgcc |
| | aggctccagggaaggggctggagtgggtctcagctattagtggtagtggtggtagcacatactacgcagactccgtgaagggccg |
| | gttcaccatctccagagacaattccaagaacacgctgtatctgcaaatgaacagcctgagagccgaggacacggccatatattactg |
| | tgcgagagacctacgggtgtatgctatggaggcttttgatatctggggccaagggacaatggtcactgtctcctctgcttccccaacc |
| | tctcctaaggtgttccctctgagcctggacagcacccctcaggatggaaatgtggtggtggcctgtctggtgcagggattcttcccac |
| | aagagcccctgtccgtgacttggagcgagtctggacagggcgtgaccgccagaaacttcccaccttctcaggacgcctctggcga |
| | cctgtacaccacctcttctcagctgaccctgcctgccacacagtgccctgatggcaagtctgtgacctgccacgtgaagcactacac |
| | caactccagccaggacgtgaccgtgccttgtagagttcctcctcctccaccttgctgtcaccctcggctgtctctgcatagacccgctc |
| | tggaagatctgctgctgggctctgaggccaacctgacatgtaccctgaccggcctgagagatgcttctggcgccacctttacctgga |
| | caccttccagcggaaagtccgctgttcagggacctcctgagagggacctgtgcggctgttactctgtgtcctctgtgctgcctggca |
| | gtgcccagccttggaatcacggcgagacattcacctgtaccgctgctcaccccgagctgaaaacccctctgaccgccaccctcagc |
| | aagtccggcaacaccttccggcctgaagtgcatctgctgcctccacctagcgaggaactggccctgaatgagctggtcaccctgac |
| | ctgtctggccaggggctttagccctaaggacgtgctcgttagatggctgcagggctcccaagagctgcccagagagaagtatctga |
| | cctgggcctctcggcaagagccatctcagggcaccacaacctttgccgtgaccagcatcctgagagtggccgccgaagattggaa |
| | gaagggcgacaccttcagctgcatggtcggacatgaagccctgcctctggctttcacccagaaaaccatcgacagactggctggc |
| | aagcccacacatgtgaatgtgtctgtggtcatggccgaggtggacggcacctgatag |
| 77 | MGWSCIILFLVATATGVHASYELTQDPAVSVALGQTVRITCQGDSLRDYYANWFQ |
| | QKPGQAPLLVIFYDSDRPSGIPERFSGSNSGNTATLTISRVEAGDEADYYCQAWDSS |
| | SDHWVFGGGTKLTVLGQPKAAPSVTLFPPSSEELQANKATLVCLISDFYPGAVTVA |
| | WKADGSPVKAGVETTKPSKQSNNKYAASSYLSLTPEQWKSHRSYSCQVTHEGSTV |
| | EKTVAPTECS |
| 78 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcatcctatgagctgactcaggaccctgctgtgtctgtggc |
| | cctgggacagacagtcaggatcacatgccaaggggacagcctcagagactattatgcaaactggttccagcagaagccaggcca |
| | ggcccctttattggtcatcttttatgatagcgaccggccctcagggatccctgagcgattctctggctccaactctgggaatacggcca |
| | ccctgaccatcagcagggtcgaagccggggatgaggccgactattattgtcaggcgtgggatagtagtagtgaccattgggtgttc |
| | ggcggagggaccaaactgaccgtcctaggtcagcccaaggctgccccctcggtcactctgttcccaccctcctctgaggagctcc |
| | aagccaacaaggccacactggtgtgtctgatcagtgacttctaccctggagctgtgacagtggcctggaaggcagatggcagccc |
| | cgtcaaggcgggagtggagacaaccaaaccctccaaacagagcaacaacaagtacgcggccagcagctacctgagcctgacg |
| | cccgagcagtggaagtcccacagaagctacagctgccaggtcacgcatgaagggagcaccgtggagaagacagtggcccctac |
| | agaatgttcatag |
| 79 | MDWTWRILFLVAAAQPALAQVQLQESGPGLVKPSETLSLTCTVSGYSISSGYYWG |
| | WIRQPPGKGLEWIGSIYHSGSTYYNPSLKSRVTISVDTSKNQFSLKLSSVTAADTAV |
| | YYCARVGVRYFDWLLYGAFDIWGQGTMVTVSSASTKGPSVFPLAPSSKSTSGGTA |
| | ALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSRWQQ |
| | GNVFSCSVMHEALHNHYTQKSLSLSPGK |
| 80 | atggactggacctggcgcatcctgtttctggtggccgctgcccaaccagcgctggcccaggtgcagctgcaggagtcgggccca |
| | ggactggtgaagccttcggagaccctgtccctcacctgcactgtctctggttactccatcagcagtggttactactggggctggatcc |
| | ggcagcccccagggaaggggctggagtggattgggagtatctatcatagtgggagcacctactacaacccgtccctcaagagtcg |
| | agtcaccatatcagtagacacgtccaagaaccagttctccctgaagctgagctctgtgaccgccgcagacacggccgtgtattactg |
| | tgcgagagtgggggttcgatattttgactggttattatatggcgcttttgatatctggggccaagggacaatggtcactgtctcctcagc |
| | ttccaccaagggcccatcggtcttcccgctagcaccctcctccaagagcacctctgggggcacagcggccctgggctgcctggtc |
| | aaggactacttccccgaaccggtgacggtgtcgtggaactcaggcgccctgaccagcggcgtccacaccttcccggctgtcctac |
| | agtcctccggactctactccctcagcagcgtagtgaccgtgccctccagcagcttgggcacccagacctacatctgcaacgtgaatc |
| | acaagcccagcaacaccaaggtggacaagaaagttgagcccaaatcttgtgacaaaactcacacatgcccaccgtgcccagcac |
| | ctgaactcctggggggaccgtcagtcttcctcttccccccaaaacccaaggacaccctcatgatctcccggacccctgaggtcacat |
| | gcgtggtggtggacgtgagccacgaagaccctgaggtcaagttcaactggtacgtggacggcgtggaggtgcataatgccaaga |
| | caaagccgcgggaggagcagtacaacagcacgtaccgtgtggtcagcgtcctcaccgtcctgcaccaggactggctgaatggca |
| | aggagtacaagtgcaaggtctccaacaaagccctcccagcccccatcgagaaaaccatctccaaagccaaagggcagccccga |
| | gaaccacaggtgtacaccctgcccccatcccgggatgagctgaccaagaaccaggtcagcctgacctgcctggtcaaaggcttct |
| | atcccagcgacatcgccgtggagtgggagagcaatgggcagccggagaacaactacaagaccacgcctcccgtgctggactcc |
| | gacggctccttcttcctctacagcaagctcaccgtggacaagagcaggtggcagcaggggaacgtcttctcatgctccgtgatgcat |
| | gaggctctgcacaaccactacacgcagaagagcctctccctgtctccgggtaaatga |
| 81 | MDWTWRILFLVAFATAVQASYELTQPPSVSVSPGQTATITCSGDKLGETYVHWYQ |
| | HQSGQSPILVVYQDFKRPSGIPERFSGSNSGNTATLTISGTQAMDEADYYCQAWDS |
| | STAVFGGGTKLTVLGQPKAAPSVTLFPPSSEELQANKATLVCLISDFYPGAVTVAW |
| | KADGSPVKAGVETTKPSKQSNNKYAASSYLSLTPEQWKSHRSYSCQVTHEGSTVE |
| | KTVAPTECS |
| 82 | atggactggacctggcgcatcctgtttctggtggccttcgccacggccgtgcaggcctcctatgagctgactcagccaccctcagtg |
| | tccgtgtcgcctggacagacagccaccatcacctgctctggagataagttgggggaaacttatgttcattggtatcagcatcagtcag |
| | gccagtcccctattttagtcgtctatcaagatttcaagcggccctccgggatccctgagcgattctctggctccaactctgggaacaca |
| | gccactctgaccatcagcgggacccaggctatggatgaggctgactattactgtcaggcgtgggacagcagcactgcggtattcg |
| | gcggagggaccaagctgaccgtcctaggtcagcccaaggctgccccctcggtcactctgttcccaccctcctctgaggagctcca |
| | agccaacaaggccacactggtgtgtctgatcagtgacttctaccctggagctgtgacagtggcctggaaggcagatggcagcccc |
| | gtcaaggcgggagtggagacaaccaaaccctccaaacagagcaacaacaagtacgcggccagcagctacctgagcctgacgc |
| | ccgagcagtggaagtcccacagaagctacagctgccaggtcacgcatgaagggagcaccgtggagaagacagtggcccctaca |
| | gaatgttca |
| 83 | MDWTWRILFLVAAAQPALAQVQLQESGPGLVKPSETLSLTCTVSGYSISSGYYWG |
| | WIRQPPGKGLEWIGSIYHSGSTYYNPSLKSRVTISVDTSKNQFSLKLSSVTAADTAV |
| | YYCARVGVRYFDWLLYGAFDIWGQGTMVTVSSASPTSPKVFPLSLDSTPQDGNVV |
| | VACLVQGFFPQEPLSVTWSESGQNVTARNFPPSQDASGDLYTTSSQLTLPATQCPD |
| | GKSVTCHVKHYTNSSQDVTVPCRVPPPPPCCHPRLSLHRPALEDLLLGSEANLTCTL |
| | TGLRDASGATFTWTPSSGKSAVQGPPERDLCGCYSVSSVLPGCAQPWNHGETFTCT |
| | AAHPELKTPLTANITKSGNTFRPEVHLLPPPSEELALNELVTLTCLARGFSPKDVLV |
| | RWLQGSQELPREKYLTWASRQEPSQGTTTFAVTSILRVAAEDWKKGDTFSCMVGH |
| | EALPLAFTQKTIDRLAGKPTHVNVSVVMAEVDGTCY |
| 84 | atggactggacctggcgcatcctgtttctggtggccgctgcccaaccagcgctggcccaggtgcagctgcaggagtcgggccca |
| | ggactggtgaagccttcggagaccctgtccctcacctgcactgtctctggttactccatcagcagtggttactactggggctggatcc |
| | ggcagcccccagggaaggggctggagtggattgggagtatctatcatagtgggagcacctactacaacccgtccctcaagagtcg |
| | agtcaccatatcagtagacacgtccaagaaccagttctccctgaagctgagctctgtgaccgccgcagacacggccgtgtattactg |
| | tgcgagagtgggggttcgatattttgactggttattatatggcgcttttgatatctggggccaagggacaatggtcactgtctcctcagc |
| | ttccccaacctctcctaaggtgttccctctgagcctggacagcacccctcaggatggaaatgtggtggtggcctgtctggtgcaggg |
| | attcttcccacaagagcccctgtccgtgacttggagcgagtctggacagaacgtgaccgccagaaacttcccaccttctcaggacg |
| | cctctggcgacctgtacaccacctcttctcagctgaccctgcctgccacacagtgccctgatggcaagtctgtgacctgccacgtga |
| | agcactacaccaactccagccaggacgtgaccgtgccttgtagagttcctcctcctccaccttgctgtcaccctcggctgtctctgcat |
| | agacccgctctggaagatctgctgctgggctctgaggccaacctgacatgtaccctgaccggcctgagagatgcttctggcgccac |
| | ctttacctggacaccttccagcggaaagtccgctgttcagggacctcctgagagggacctgtgcggctgttactctgtgtcctctgtg |
| | ctgcctggctgtgcccagccttggaatcacggcgagacattcacctgtaccgctgctcaccccgagctgaaaacccctctgaccgc |
| | caacatcaccaagtccggcaacaccttccggcctgaagtgcatctgctgcctccacctagcgaggaactggccctgaatgagctgg |
| | tcaccctgacctgtctggccaggggctttagccctaaggacgtgctcgttagatggctgcagggctcccaagagctgcccagagag |
| | aagtatctgacctgggcctctcggcaagagccatctcagggcaccacaacctttgccgtgaccagcatcctgagagtggccgccg |
| | aagattggaagaagggcgacaccttcagctgcatggtcggacatgaagccctgcctctggctttcacccagaaaaccatcgacag |
| | actggccggcaagcccacacatgtgaatgtgtctgtggtcatggccgaggtggacggcacctgttactgatag |
| 85 | MGWSCIILFLVATATGVHASYELTQPPSVSVSPGQTATITCSGDKLGETYVHWYQH |
| | QSGQSPILVVYQDFKRPSGIPERFSGSNSGNTATLTISGTQAMDEADYYCQAWDSST |
| | AVFGGGTKLTVLGQPKAAPSVTLFPPSSEELQANKATLVCLISDFYPGAVTVAWKA |
| | DGSPVKAGVETTKPSKQSNNKYAASSYLSLTPEQWKSHRSYSCQVTHEGSTVEKT |
| | VAPTECS |
| 86 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcatcctatgagctgactcagccaccctcagtgtccgtgtc |
| | gcctggacagacagccaccatcacctgctctggagataagttgggggaaacttatgttcattggtatcagcatcagtcaggccagtc |
| | ccctattttagtcgtctatcaagatttcaagcggccctccgggatccctgagcgattctctggctccaactctgggaacacagccactc |
| | tgaccatcagcgggacccaggctatggatgaggctgactattactgtcaggcgtgggacagcagcactgcggtattcggcggagg |
| | gaccaagctgaccgtcctaggtcagcccaaggctgccccctcggtcactctgttcccaccctcctctgaggagctccaagccaaca |
| | aggccacactggtgtgtctgatcagtgacttctaccctggagctgtgacagtggcctggaaggcagatggcagccccgtcaaggc |
| | gggagtggagacaaccaaaccctccaaacagagcaacaacaagtacgcggccagcagctacctgagcctgacgcccgagcag |
| | tggaagtcccacagaagctacagctgccaggtcacgcatgaagggagcaccgtggagaagacagtggcccctacagaatgttca |
| 87 | MDWTWRILFLVAAAQPALAEVQLVESGGGVVQPGRSLRLSCAASGFTFSSYAMH |
| | WVRQAPGKGLEWVSVIYSGGSTYYADSVKGRFTISRDNSKNTLYLQMNSLRAEDT |
| | AVYYCARDSSGYRGDFDYWGQGTLVTVSSASTKGPSVFPLAPSSKSTSGGTAALG |
| | CLVKDYFPEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNV |
| | FSCSVMHEALHNHYTQKSLSLSPGK |
| 88 | atggactggacctggcgcatcctgtttctggtggccgctgcccaaccagcgctggccgaagtgcagctggtggagtctgggggag |
| | gcgtggtccagcctgggaggtccctgagactctcctgtgcagcctctggattcaccttcagtagctatgctatgcactgggtccgcca |
| | ggctccagggaaggggctggagtgggtctcagttatttatagcggtggtagcacatactacgcagactccgtgaagggcagattca |
| | ccatctccagagacaattccaagaacacgctgtatcttcaaatgaacagcctgagagccgaggacacggctgtgtattactgtgcga |
| | gagatagtagtggttatcgaggcgactttgactactggggccagggaaccctggtcaccgtctcctcagcttccaccaagggcccat |
| | cggtcttcccgctagcaccctcctccaagagcacctctgggggcacagcggccctgggctgcctggtcaaggactacttccccga |
| | accggtgacggtgtcgtggaactcaggcgccctgaccagcggcgtccacaccttcccggctgtcctacagtcctccggactctact |
| | ccctcagcagcgtagtgaccgtgccctccagcagcttgggcacccagacctacatctgcaacgtgaatcacaagcccagcaacac |
| | caaggtggacaagaaagttgagcccaaatcttgtgacaaaactcacacatgcccaccgtgcccagcacctgaactcctgggggga |
| | ccgtcagtcttcctcttccccccaaaacccaaggacaccctcatgatctcccggacccctgaggtcacatgcgtggtggtggacgtg |
| | agccacgaagaccctgaggtcaagttcaactggtacgtggacggcgtggaggtgcataatgccaagacaaagccgcgggagga |
| | gcagtacaacagcacgtaccgtgtggtcagcgtcctcaccgtcctgcaccaggactggctgaatggcaaggagtacaagtgcaag |
| | gtctccaacaaagccctcccagcccccatcgagaaaaccatctccaaagccaaagggcagccccgagaaccacaggtgtacacc |
| | ctgcccccatcccgggatgagctgaccaagaaccaggtcagcctgacctgcctggtcaaaggcttctatcccagcgacatcgccgt |
| | ggagtgggagagcaatgggcagccggagaacaactacaagaccacgcctcccgtgctggactccgacggctccttcttcctctac |
| | agcaagctcaccgtggacaagagcaggtggcagcaggggaacgtcttctcatgctccgtgatgcatgaggctctgcacaaccact |
| | acacgcagaagagcctctccctgtctccgggtaaatga |
| 89 | MDWTWRILFLVAFATAVQASYELTQPPSVSVSPGQTASITCSGDKVGAKYVFWYQ |
| | QKPGQSPVLVVYQDNKRPSGIPERFSGSNSGNTATLTISGTQAMDEADYYCQAWD |
| | SSTALYVFGTGTKVTVLGQPKANPTVTLFPPSSEELQANKATLVCLISDFYPGAVTV |
| | AWKADGSPVKAGVETTKPSKQSNNKYAASSYLSLTPEQWKSHRSYSCQVTHEGST |
| | VEKTVAPTECS |
| 90 | atggactggacctggcgcatcctgtttctggtggccttcgccacggccgtgcaggcctcctatgagctgactcagccaccctcagtg |
| | tccgtgtccccaggacagacagccagcatcacctgctctggagataaggtgggggctaaatatgttttctggtatcagcagaagcca |
| | ggccagtcccctgtgctggtcgtctatcaagataacaagcggccctcagggatccctgagcgattctctggctccaactctgggaac |
| | acagccacactgaccatcagcgggacccaggctatggatgaggctgactattactgtcaggcgtgggacagcagcactgcccttta |
| | tgtcttcggaactgggaccaaggtcaccgtcctaggtcagcccaaggccaaccccactgtcactctgttcccaccctcctctgagga |
| | gctccaagccaacaaggccacactggtgtgtctgatcagtgacttctaccctggagctgtgacagtggcctggaaggcagatggca |
| | gccccgtcaaggcgggagtggagacaaccaaaccctccaaacagagcaacaacaagtacgcggccagcagctacctgagcct |
| | gacgcccgagcagtggaagtcccacagaagctacagctgccaggtcacgcatgaagggagcaccgtggagaagacagtggcc |
| | cctacagaatgttcatag |
| 91 | MDWTWRILFLVAAAQPALAEVQLVESGGGVVQPGRSLRLSCAASGFTFSSYAMH |
| | WVRQAPGKGLEWVSVIYSGGSTYYADSVKGRFTISRDNSKNTLYLQMNSLRAEDT |
| | AVYYCARDSSGYRGDFDYWGQGTLVTVSSASPTSPKVFPLSLDSTPQDGNVVVAC |
| | LVQGFFPQEPLSVTWSESGQNVTARNFPPSQDASGDLYTTSSQLTLPATQCPDGKS |
| | VTCHVKHYTNSSQDVTVPCRVPPPPPCCHPRLSLHRPALEDLLLGSEANLTCTLTGL |
| | RDASGATFTWTPSSGKSAVQGPPERDLCGCYSVSSVLPGCAQPWNHGETFTCTAA |
| | HPELKTPLTANITKSGNTFRPEVHLLPPPSEELALNELVTLTCLARGFSPKDVLVRW |
| | LQGSQELPREKYLTWASRQEPSQGTTTFAVTSILRVAAEDWKKGDTFSCMVGHEA |
| | LPLAFTQKTIDRLAGKPTHVNVSVVMAEVDGTCY |
| 92 | atggactggacctggcgcatcctgtttctggtggccgctgcccaaccagcgctggccgaagtgcagctggtggagtctgggggag |
| | gcgtggtccagcctgggaggtccctgagactctcctgtgcagcctctggattcaccttcagtagctatgctatgcactgggtccgcca |
| | ggctccagggaaggggctggagtgggtctcagttatttatagcggtggtagcacatactacgcagactccgtgaagggcagattca |
| | ccatctccagagacaattccaagaacacgctgtatcttcaaatgaacagcctgagagccgaggacacggctgtgtattactgtgcga |
| | gagatagtagtggttatcgaggcgactttgactactggggccagggaaccctggtcactgtctcttctgcttccccaacctctcctaag |
| | gtgttccctctgagcctggacagcacccctcaggatggaaatgtggtggtggcctgtctggtgcagggattcttcccacaagagccc |
| | ctgtccgtgacttggagcgagtctggacagaacgtgaccgccagaaacttcccaccttctcaggacgcctctggcgacctgtacac |
| | cacctcttctcagctgaccctgcctgccacacagtgccctgatggcaagtctgtgacctgccacgtgaagcactacaccaactccag |
| | ccaggacgtgaccgtgccttgtagagttcctcctcctccaccttgctgtcaccctcggctgtctctgcatagacccgctctggaagatc |
| | tgctgctgggctctgaggccaacctgacatgtaccctgaccggcctgagagatgcttctggcgccacctttacctggacaccttcca |
| | gcggaaagtccgctgttcagggacctcctgagagggacctgtgcggctgttactctgtgtcctctgtgctgcctggctgtgcccagc |
| | cttggaatcacggcgagacattcacctgtaccgctgctcaccccgagctgaaaacccctctgaccgccaacatcaccaagtccggc |
| | aacaccttccggcctgaagtgcatctgctgcctccacctagcgaggaactggccctgaatgagctggtcaccctgacctgtctggcc |
| | aggggctttagccctaaggacgtgctcgttagatggctgcagggctcccaagagctgcccagagagaagtatctgacctgggcct |
| | ctcggcaagagccatctcagggcaccacaacctttgccgtgaccagcatcctgagagtggccgccgaagattggaagaagggcg |
| | acaccttcagctgcatggtcggacatgaagccctgcctctggctttcacccagaaaaccatcgacagactggccggcaagcccac |
| | acatgtgaatgtgtctgtggtcatggccgaggtggacggcacctgttactgatag |
| 93 | MGWSCIILFLVATATGVHASYELTQPPSVSVSPGQTASITCSGDKVGAKYVFWYQQ |
| | KPGQSPVLVVYQDNKRPSGIPERFSGSNSGNTATLTISGTQAMDEADYYCQAWDSS |
| | TALYVFGTGTKVTVLGQPKANPTVTLFPPSSEELQANKATLVCLISDFYPGAVTVA |
| | WKADGSPVKAGVETTKPSKQSNNKYAASSYLSLTPEQWKSHRSYSCQVTHEGSTV |
| | EKTVAPTECS |
| 94 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcatcctatgagctgactcagccaccctcagtgtccgtgtc |
| | cccaggacagacagccagcatcacctgctctggagataaggtgggggctaaatatgttttctggtatcagcagaagccaggccagt |
| | cccctgtgctggtcgtctatcaagataacaagcggccctcagggatccctgagcgattctctggctccaactctgggaacacagcca |
| | cactgaccatcagcgggacccaggctatggatgaggctgactattactgtcaggcgtgggacagcagcactgccctttatgtcttcg |
| | gaactgggaccaaggtcaccgtcctaggtcagcccaaggccaaccccactgtcactctgttcccaccctcctctgaggagctccaa |
| | gccaacaaggccacactggtgtgtctgatcagtgacttctaccctggagctgtgacagtggcctggaaggcagatggcagccccgt |
| | caaggcgggagtggagacaaccaaaccctccaaacagagcaacaacaagtacgcggccagcagctacctgagcctgacgccc |
| | gagcagtggaagtcccacagaagctacagctgccaggtcacgcatgaagggagcaccgtggagaagacagtggcccctacaga |
| | atgttca |
| 95 | MDWTWRILFLVAAAQPALAEVQLVESGGGVVQPGRSLRLSCAASGFTFSSYAMH |
| | WVRQAPGKGLEWVSVIYSGGSTYYADSVKGRFTISRDNSKNTLYLQMNSLRAEDT |
| | AVYYCARDSSGYRGDFDYWGQGTLVTVSSASPTSPKVFPLSLDSTPQDGNVVVAC |
| | LVQGFFPQEPLSVTWSESGQGVTARNFPPSQDASGDLYTTSSQLTLPATQCPDGKS |
| | VTCHVKHYTNSSQDVTVPCRVPPPPPCCHPRLSLHRPALEDLLLGSEANLTCTLTGL |
| | RDASGATFTWTPSSGKSAVQGPPERDLCGCYSVSSVLPGSAQPWNHGETFTCTAA |
| | HPELKTPLTATLSKSGNTFRPEVHLLPPPSEELALNELVTLTCLARGFSPKDVLVRW |
| | LQGSQELPREKYLTWASRQEPSQGTTTFAVTSILRVAAEDWKKGDTFSCMVGHEA |
| | LPLAFTQKTIDRLAGKPTHVNVSVVMAEVDGT |
| 96 | atggactggacctggcgcatcctgtttctggtggccgctgcccaaccagcgctggccgaagtgcagctggtggagtctgggggag |
| | gcgtggtccagcctgggaggtccctgagactctcctgtgcagcctctggattcaccttcagtagctatgctatgcactgggtccgcca |
| | ggctccagggaaggggctggagtgggtctcagttatttatagcggtggtagcacatactacgcagactccgtgaagggcagattca |
| | ccatctccagagacaattccaagaacacgctgtatcttcaaatgaacagcctgagagccgaggacacggctgtgtattactgtgcga |
| | gagatagtagtggttatcgaggcgactttgactactggggccagggaaccctggtcactgtctcttctgcttccccaacctctcctaag |
| | gtgttccctctgagcctggacagcacccctcaggatggaaatgtggtggtggcctgtctggtgcagggattcttcccacaagagccc |
| | ctgtccgtgacttggagcgagtctggacagggcgtgaccgccagaaacttcccaccttctcaggacgcctctggcgacctgtacac |
| | cacctcttctcagctgaccctgcctgccacacagtgccctgatggcaagtctgtgacctgccacgtgaagcactacaccaactccag |
| | ccaggacgtgaccgtgccttgtagagttcctcctcctccaccttgctgtcaccctcggctgtctctgcatagacccgctctggaagatc |
| | tgctgctgggctctgaggccaacctgacatgtaccctgaccggcctgagagatgcttctggcgccacctttacctggacaccttcca |
| | gcggaaagtccgctgttcagggacctcctgagagggacctgtgcggctgttactctgtgtcctctgtgctgcctggcagtgcccagc |
| | cttggaatcacggcgagacattcacctgtaccgctgctcaccccgagctgaaaacccctctgaccgccaccctcagcaagtccggc |
| | aacaccttccggcctgaagtgcatctgctgcctccacctagcgaggaactggccctgaatgagctggtcaccctgacctgtctggcc |
| | aggggctttagccctaaggacgtgctcgttagatggctgcagggctcccaagagctgcccagagagaagtatctgacctgggcct |
| | ctcggcaagagccatctcagggcaccacaacctttgccgtgaccagcatcctgagagtggccgccgaagattggaagaagggcg |
| | acaccttcagctgcatggtcggacatgaagccctgcctctggctttcacccagaaaaccatcgacagactggctggcaagcccaca |
| | catgtgaatgtgtctgtggtcatggccgaggtggacggcacctgatag |
| 97 | MGWSCIILFLVATATGVHASYELTQPPSVSVSPGQTASITCSGDKVGAKYVFWYQQ |
| | KPGQSPVLVVYQDNKRPSGIPERFSGSNSGNTATLTISGTQAMDEADYYCQAWDSS |
| | TALYVFGTGTKVTVLGQPKANPTVTLFPPSSEELQANKATLVCLISDFYPGAVTVA |
| | WKADGSPVKAGVETTKPSKQSNNKYAASSYLSLTPEQWKSHRSYSCQVTHEGSTV |
| | EKTVAPTECS |
| 98 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcatcctatgagctgactcagccaccctcagtgtccgtgtc |
| | cccaggacagacagccagcatcacctgctctggagataaggtgggggctaaatatgttttctggtatcagcagaagccaggccagt |
| | cccctgtgctggtcgtctatcaagataacaagcggccctcagggatccctgagcgattctctggctccaactctgggaacacagcca |
| | cactgaccatcagcgggacccaggctatggatgaggctgactattactgtcaggcgtgggacagcagcactgccctttatgtcttcg |
| | gaactgggaccaaggtcaccgtcctaggtcagcccaaggccaaccccactgtcactctgttcccaccctcctctgaggagctccaa |
| | gccaacaaggccacactggtgtgtctgatcagtgacttctaccctggagctgtgacagtggcctggaaggcagatggcagccccgt |
| | caaggcgggagtggagacaaccaaaccctccaaacagagcaacaacaagtacgcggccagcagctacctgagcctgacgccc |
| | gagcagtggaagtcccacagaagctacagctgccaggtcacgcatgaagggagcaccgtggagaagacagtggcccctacaga |
| | atgttcatag |
| 99 | MDWTWRILFLVAAAQPALAEVQLLESGGGLVQPGGSLRLSCAASGFTFSDYGMS |
| | WVRQAPGKGLEWVSAIGYGGGGKYYADSVKGRFTISRDNSKNTLYLQMNSLRAE |
| | DTAVYYCAKDTWWLAGLFDYWGQGTLVTVSSASTKGPSVFPLAPSSKSTSGGTA |
| | ALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSRWQQ |
| | GNVFSCSVMHEALHNHYTQKSLSLSPGK |
| 100 | atggactggacctggcgcatcctgtttctggtggccgctgcccaaccagcgctggccgaggtgcagctgttggagtctgggggag |
| | gcttggtacagcctggggggtccctgagactctcctgtgcagcctctggattcacctttagcgattacgggatgagctgggtccgcc |
| | aggctccagggaaggggctggagtgggtatcagccattgggtatggtggaggtggtaaatactacgcagactccgtgaagggcc |
| | ggttcaccatctccagagacaattccaagaacacgctgtatctgcaaatgaacagcctgagagccgaggacacggccgtgtattac |
| | tgtgctaaggatacttggtggctggcgggtctctttgactactggggccagggaaccctggtcaccgtctcctcagcttccaccaag |
| | ggcccatcggtcttcccgctagcaccctcctccaagagcacctctgggggcacagcggccctgggctgcctggtcaaggactactt |
| | ccccgaaccggtgacggtgtcgtggaactcaggcgccctgaccagcggcgtccacaccttcccggctgtcctacagtcctccgga |
| | ctctactccctcagcagcgtagtgaccgtgccctccagcagcttgggcacccagacctacatctgcaacgtgaatcacaagcccag |
| | caacaccaaggtggacaagaaagttgagcccaaatcttgtgacaaaactcacacatgcccaccgtgcccagcacctgaactcctg |
| | gggggaccgtcagtcttcctcttccccccaaaacccaaggacaccctcatgatctcccggacccctgaggtcacatgcgtggtggt |
| | ggacgtgagccacgaagaccctgaggtcaagttcaactggtacgtggacggcgtggaggtgcataatgccaagacaaagccgc |
| | gggaggagcagtacaacagcacgtaccgtgtggtcagcgtcctcaccgtcctgcaccaggactggctgaatggcaaggagtaca |
| | agtgcaaggtctccaacaaagccctcccagcccccatcgagaaaaccatctccaaagccaaagggcagccccgagaaccacag |
| | gtgtacaccctgcccccatcccgggatgagctgaccaagaaccaggtcagcctgacctgcctggtcaaaggcttctatcccagcga |
| | catcgccgtggagtgggagagcaatgggcagccggagaacaactacaagaccacgcctcccgtgctggactccgacggctcctt |
| | cttcctctacagcaagctcaccgtggacaagagcaggtggcagcaggggaacgtcttctcatgctccgtgatgcatgaggctctgc |
| | acaaccactacacgcagaagagcctctccctgtctccgggtaaatga |
| 101 | MDWTWRILFLVAFATAVQAEIVLTQSPGTLSLSPGKRATLSCRASQSVSNSYLAWY |
| | QQKPGQPPKLLIYWASTRESGVPDRFSGSGSGTDFTLTISSLQAEDVAVYYCQQYY |
| | STPLTFGGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPV |
| | TKSFNRGEC |
| 102 | atggactggacctggcgcatcctgtttctggtggccttcgccacggccgtgcaggccgaaattgtgttgacacagtcgccaggcac |
| | cctgtctttgtctccagggaaaagagccaccctctcctgcagggccagtcagagtgttagcaacagctacttagcctggtaccagca |
| | gaaaccaggacagcctcctaagctgctcatttactgggcatctacccgggaatccggggtccctgaccgattcagtggcagcgggt |
| | ctgggacagatttcactctcaccatcagcagcctgcaggctgaagatgtggcagtttattactgtcagcaatattatagtactccgctca |
| | ctttcggcggagggaccaaggtggagatcaaacgaactgtggctgcaccatctgtcttcatcttcccgccatctgatgagcagttga |
| | aatctggaactgcctctgttgtgtgcctgctgaataacttctatcccagagaggccaaagtacagtggaaggtggataacgccctcca |
| | atcgggtaactcccaggagagtgtcacagagcaggacagcaaggacagcacctacagcctcagcagcaccctgacgctgagca |
| | aagcagactacgagaaacacaaagtctacgcctgcgaagtcacccatcagggcctgagttcgcccgtcacaaagagcttcaacag |
| | gggagagtgttag |
| 103 | MDWTWRILFLVAAAQPALAEVQLLESGGGLVQPGGSLRLSCAASGFTFSDYGMS |
| | WVRQAPGKGLEWVSAIGYGGGGKYYADSVKGRFTISRDNSKNTLYLQMNSLRAE |
| | DTAVYYCAKDTWWLAGLFDYWGQGTLVTVSSASPTSPKVFPLSLDSTPQDGNVV |
| | VACLVQGFFPQEPLSVTWSESGQNVTARNFPPSQDASGDLYTTSSQLTLPATQCPD |
| | GKSVTCHVKHYTNSSQDVTVPCRVPPPPPCCHPRLSLHRPALEDLLLGSEANLTCTL |
| | TGLRDASGATFTWTPSSGKSAVQGPPERDLCGCYSVSSVLPGCAQPWNHGETFTCT |
| | AAHPELKTPLTANITKSGNTFRPEVHLLPPPSEELALNELVTLTCLARGFSPKDVLV |
| | RWLQGSQELPREKYLTWASRQEPSQGTTTFAVTSILRVAAEDWKKGDTFSCMVGH |
| | EALPLAFTQKTIDRLAGKPTHVNVSVVMAEVDGTCY |
| 104 | atggactggacctggcgcatcctgtttctggtggccgctgcccaaccagcgctggccgaggtgcagctgttggagtctgggggag |
| | gcttggtacagcctggggggtccctgagactctcctgtgcagcctctggattcacctttagcgattacgggatgagctgggtccgcc |
| | aggctccagggaaggggctggagtgggtatcagccattgggtatggtggaggtggtaaatactacgcagactccgtgaagggcc |
| | ggttcaccatctccagagacaattccaagaacacgctgtatctgcaaatgaacagcctgagagccgaggacacggccgtgtattac |
| | tgtgctaaggatacttggtggctggcgggtctctttgactactggggccagggaaccctggtcactgtctcttctgcttccccaacctct |
| | cctaaggtgttccctctgagcctggacagcacccctcaggatggaaatgtggtggtggcctgtctggtgcagggattcttcccacaa |
| | gagcccctgtccgtgacttggagcgagtctggacagaacgtgaccgccagaaacttcccaccttctcaggacgcctctggcgacct |
| | gtacaccacctcttctcagctgaccctgcctgccacacagtgccctgatggcaagtctgtgacctgccacgtgaagcactacaccaa |
| | ctccagccaggacgtgaccgtgccttgtagagttcctcctcctccaccttgctgtcaccctcggctgtctctgcatagacccgctctgg |
| | aagatctgctgctgggctctgaggccaacctgacatgtaccctgaccggcctgagagatgcttctggcgccacctttacctggacac |
| | cttccagcggaaagtccgctgttcagggacctcctgagagggacctgtgcggctgttactctgtgtcctctgtgctgcctggctgtgc |
| | ccagccttggaatcacggcgagacattcacctgtaccgctgctcaccccgagctgaaaacccctctgaccgccaacatcaccaagt |
| | ccggcaacaccttccggcctgaagtgcatctgctgcctccacctagcgaggaactggccctgaatgagctggtcaccctgacctgt |
| | ctggccaggggctttagccctaaggacgtgctcgttagatggctgcagggctcccaagagctgcccagagagaagtatctgacct |
| | gggcctctcggcaagagccatctcagggcaccacaacctttgccgtgaccagcatcctgagagtggccgccgaagattggaaga |
| | agggcgacaccttcagctgcatggtcggacatgaagccctgcctctggctttcacccagaaaaccatcgacagactggccggcaa |
| | gcccacacatgtgaatgtgtctgtggtcatggccgaggtggacggcacctgttactgatag |
| 105 | MGWSCIILFLVATATGVHAEIVLTQSPGTLSLSPGKRATLSCRASQSVSNSYLAWY |
| | QQKPGQPPKLLIYWASTRESGVPDRFSGSGSGTDFTLTISSLQAEDVAVYYCQQYY |
| | STPLTFGGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPV |
| | TKSFNRGEC |
| 106 | atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcagaaattgtgttgacacagtcgccaggcaccctgtcttt |
| | gtctccagggaaaagagccaccctctcctgcagggccagtcagagtgttagcaacagctacttagcctggtaccagcagaaacca |
| | ggacagcctcctaagctgctcatttactgggcatctacccgggaatccggggtccctgaccgattcagtggcagcgggtctgggac |
| | agatttcactctcaccatcagcagcctgcaggctgaagatgtggcagtttattactgtcagcaatattatagtactccgctcactttcgg |
| | cggagggaccaaggtggagatcaaacgaactgtggctgcaccatctgtcttcatcttcccgccatctgatgagcagttgaaatctgg |
| | aactgcctctgttgtgtgcctgctgaataacttctatcccagagaggccaaagtacagtggaaggtggataacgccctccaatcgggt |
| | aactcccaggagagtgtcacagagcaggacagcaaggacagcacctacagcctcagcagcaccctgacgctgagcaaagcag |
| | actacgagaaacacaaagtctacgcctgcgaagtcacccatcagggcctgagttcgcccgtcacaaagagcttcaacaggggag |
| | agtgttag |
| 107 | MDWTWRILFLVAAAQPALAEVQLLESGGGLVQPGGSLRLSCAASGFTFSDYGMS |
| | WVRQAPGKGLEWVSAIGYGGGGKYYADSVKGRFTISRDNSKNTLYLQMNSLRAE |
| | DTAVYYCAKDTWWLAGLFDYWGQGTLVTVSSASPTSPKVFPLSLDSTPQDGNVV |
| | VACLVQGFFPQEPLSVTWSESGQGVTARNFPPSQDASGDLYTTSSQLTLPATQCPD |
| | GKSVTCHVKHYTNSSQDVTVPCRVPPPPPCCHPRLSLHRPALEDLLLGSEANLTCTL |
| | TGLRDASGATFTWTPSSGKSAVQGPPERDLCGCYSVSSVLPGSAQPWNHGETFTCT |
| | AAHPELKTPLTATLSKSGNTFRPEVHLLPPPSEELALNELVTLTCLARGFSPKDVLV |
| | RWLQGSQELPREKYLTWASRQEPSQGTTTFAVTSILRVAAEDWKKGDTFSCMVGH |
| | EALPLAFTQKTIDRLAGKPTHVNVSVVMAEVDGT |
| 108 | atggactggacctggcgcatcctgtttctggtggccgctgcccaaccagcgctggccgaggtgcagctgttggagtctgggggag |
| | gcttggtacagcctggggggtccctgagactctcctgtgcagcctctggattcacctttagcgattacgggatgagctgggtccgcc |
| | aggctccagggaaggggctggagtgggtatcagccattgggtatggtggaggtggtaaatactacgcagactccgtgaagggcc |
| | ggttcaccatctccagagacaattccaagaacacgctgtatctgcaaatgaacagcctgagagccgaggacacggccgtgtattac |
| | tgtgctaaggatacttggtggctggcgggtctctttgactactggggccagggaaccctggtcactgtctcttctgcttccccaacctct |
| | cctaaggtgttccctctgagcctggacagcacccctcaggatggaaatgtggtggtggcctgtctggtgcagggattcttcccacaa |
| | gagcccctgtccgtgacttggagcgagtctggacagggcgtgaccgccagaaacttcccaccttctcaggacgcctctggcgacc |
| | tgtacaccacctcttctcagctgaccctgcctgccacacagtgccctgatggcaagtctgtgacctgccacgtgaagcactacacca |
| | actccagccaggacgtgaccgtgccttgtagagttcctcctcctccaccttgctgtcaccctcggctgtctctgcatagacccgctctg |
| | gaagatctgctgctgggctctgaggccaacctgacatgtaccctgaccggcctgagagatgcttctggcgccacctttacctggaca |
| | ccttccagcggaaagtccgctgttcagggacctcctgagagggacctgtgcggctgttactctgtgtcctctgtgctgcctggcagtg |
| | cccagccttggaatcacggcgagacattcacctgtaccgctgctcaccccgagctgaaaacccctctgaccgccaccctcagcaa |
| | gtccggcaacaccttccggcctgaagtgcatctgctgcctccacctagcgaggaactggccctgaatgagctggtcaccctgacct |
| | gtctggccaggggctttagccctaaggacgtgctcgttagatggctgcagggctcccaagagctgcccagagagaagtatctgac |
| | ctgggcctctcggcaagagccatctcagggcaccacaacctttgccgtgaccagcatcctgagagtggccgccgaagattggaag |
| | aagggcgacaccttcagctgcatggtcggacatgaagccctgcctctggctttcacccagaaaaccatcgacagactggctggca |
| | agcccacacatgtgaatgtgtctgtggtcatggccgaggtggacggcacctgatag |
| 109 | MGWSCIILFLVATATGVHAEIVLTQSPGTLSLSPGKRATLSCRASQSVSNSYLAWY |
| | QQKPGQPPKLLIYWASTRESGVPDRFSGSGSGTDFTLTISSLQAEDVAVYYCQQYY |
| | STPLTFGGGTKVEIKRTVAAPSVFIFPPSDEQLKSGTASVVCLLNNFYPREAKVQWK |
| | VDNALQSGNSQESVTEQDSKDSTYSLSSTLTLSKADYEKHKVYACEVTHQGLSSPV |
| | TKSFNRGEC |
| 110 | Atgggatggagctgtatcatcctcttcttggtagcaacagctacaggtaaggggttaacagtagcaggcttgaggtctggacatatat |
| | atgggtgacaatgacatccactttgcctttctctccacaggcgtgcacgcagaaattgtgttgacacagtcgccaggcaccctgtcttt |
| | gtctccagggaaaagagccaccctctcctgcagggccagtcagagtgttagcaacagctacttagcctggtaccagcagaaacca |
| | ggacagcctcctaagctgctcatttactgggcatctacccgggaatccggggtccctgaccgattcagtggcagcgggtctgggac |
| | agatttcactctcaccatcagcagcctgcaggctgaagatgtggcagtttattactgtcagcaatattatagtactccgctcactttcgg |
| | cggagggaccaaggtggagatcaaacgaactgtggctgcaccatctgtcttcatcttcccgccatctgatgagcagttgaaatctgg |
| | aactgcctctgttgtgtgcctgctgaataacttctatcccagagaggccaaagtacagtggaaggtggataacgccctccaatcgggt |
| | aactcccaggagagtgtcacagagcaggacagcaaggacagcacctacagcctcagcagcaccctgacgctgagcaaagcag |
| | actacgagaaacacaaagtctacgcctgcgaagtcacccatcagggcctgagttcgcccgtcacaaagagcttcaacaggggag |
| | agtgt |
| 111 | SYELTQDPAVSVALGQTVRITCQGDSLRDYYANWFQQKPGQAPLLVIFYD |
| | SDRPSGIPERFSGSNSGNTATLTISRVEAGDEADYYCQAWDSSSDHWVFGG |
| | GTKLTVLGQPKAAPSVTLFPPSSEELQANKATLVCLISDFYPGAVTVAWKA |
| | DSSPVKAGVETTTPSKQSNNKYAASSYLSLTPEQWKSHRSYSCQVTHEGST |
| | VEKTVAPTECS |
| 112 | AGTTATGAACTAACTCAAGATCCAGCAGTGTCCGTGGCCCTGGGCCAG |
| | ACCGTGCGGATCACCTGTCAGGGCGATTCTCTGAGAGATTATTACGCCA |
| | ACTGGTTCCAGCAGAAGCCTGGCCAAGCTCCTCTGCTGGTGATCTTCTA |
| | CGACTCCGACAGACCTTCTGGCATCCCCGAGCGGTTTAGCGGATCCAAC |
| | TCCGGCAACACCGCTACCCTGACCATCTCCAGAGTGGAAGCTGGCGAC |
| | GAGGCCGACTACTACTGCCAGGCCTGGGACTCTAGCTCTGACCACTGGG |
| | TCTTCGGCGGAGGCACCAAGCTGACAGTGCTCGGCCAGCCCAAAGCCG |
| | CCCCAAGCGTCACACTGTTTCCTCCCTCCTCTGAAGAACTGCAGGCAAA |
| | TAAGGCAACTCTGGTCTGTCTGATCTCCGACTTCTACCCAGGAGCAGTG |
| | ACAGTGGCATGGAAGGCAGATAGCTCCCCCGTGAAGGCAGGAGTGGAG |
| | ACAACAACCCCTAGCAAGCAGTCCAACAATAAGTACGCCGCCTCTAGC |
| | TATCTGTCTCTGACCCCCGAGCAGTGGAAGAGCCACCGGTCCTATTCTT |
| | GCCAGGTGACCCACGAGGGCTCAACCGTCGAAAAAACCGTCGCACCTA |
| | CTGAATGTTCC |
| 113 | EVQLAESGGDLVQPGGSLRLSCAASGFTFSSYAMSWVRQAPGKGLEWVS |
| | AISGSGGSTYYADSVKGRFTISRDNSKNTLYLQMNSLRAEDTAIYYCARDL |
| | RVYAMEAFDIWGQGTMVTVSSASPTSPKVFPLSLDSTPQDGNVVVACLVQ |
| | GFFPQEPLSVTWSESGQGVTARNFPPSQDASGDLYTTSSQLTLPATQCPDG |
| | KSVTCHVKHYTNSSQDVTVPCRVPELKTPLGDTTHTCPRCPEPKSCDTPPP |
| | CPRCPEPKSCDTPPPCPRCPEPKSCDTPPPCPRCPAPELLGGPPPPPCCHPRLS |
| | LHRPALEDLLLGSEANLTCTLTGLRDASGATFTWTPSSGKSAVQGPPERDL |
| | CGCYSVSSVLPGSAQPWNHGETFTCTAAHPELKTPLTATLSKSGNTFRPEV |
| | HLLPPPSEELALNELVTLTCLARGFSPKDVLVRWLQGSQELPREKYLTWAS |
| | RQEPSQGTTTFAVTSILRVAAEDWKKGDTFSCMVGHEALPLAFTQKTIDRL |
| | AGKPTHVNVSVVMAEVDGT |
| 114 | GAAGTGCAACTAGCAGAAAGTGGTGGTGATCTCGTGCAGCCTGGCGGA |
| | TCTCTGCGGCTGTCTTGTGCCGCTTCTGGCTTCACCTTCTCCTCCTACGC |
| | CATGTCCTGGGTGCGGCAAGCTCCCGGAAAAGGCCTGGAGTGGGTGTC |
| | CGCTATCTCTGGCTCCGGCGGCTCTACCTACTATGCCGACTCCGTGAAG |
| | GGCAGATTTACCATCTCCCGGGACAACTCCAAGAACACACTGTACCTGC |
| | AGATGAACAGCCTGAGAGCCGAGGACACCGCCATCTACTACTGCGCCA |
| | GAGATCTGAGAGTGTACGCTATGGAAGCCTTCGACATCTGGGGCCAGG |
| | GCACCATGGTGACCGTGTCCTCTGCTTCTCCCACTTCCCCAAAGGTGTTC |
| | CCTCTGTCACTGGACTCAACCCCTCAGGATGGCAATGTCGTCGTCGCAT |
| | GTCTGGTGCAGGGCTTCTTTCCTCAGGAGCCACTGAGCGTGACATGGTC |
| | TGAGAGCGGCCAGGGCGTGACCGCCAGAAATTTCCCCCCTTCCCAGGA |
| | CGCCTCTGGCGATCTGTACACCACAAGCTCCCAGCTGACCCTGCCAGCA |
| | ACACAGTGCCCAGACGGCAAGTCCGTGACCTGTCACGTGAAGCACTAT |
| | ACAAACTCTAGCCAGGATGTGACCGTGCCCTGCCGGGTGCCTGAACTA |
| | AAGACTCCACTAGGTGATACTACTCACACCTGTCCTAGATGCCCTGAAC |
| | CCAAAAGCTGCGACACACCTCCACCTTGTCCTCGGTGTCCTGAGCCTAA |
| | GTCCTGCGATACCCCTCCTCCTTGCCCCCGGTGCCCCGAGCCCAAGTCT |
| | TGCGACACCCCTCCACCCTGCCCTAGATGCCCTGCCCCTGAGCTGCTGG |
| | GCGGCCCTCCTCCACCCCCTTGCTGTCACCCAAGGCTGTCCCTGCACAG |
| | GCCCGCCCTGGAGGACCTGCTGCTGGGCTCTGAGGCCAATCTGACATGT |
| | ACCCTGACAGGCCTGCGGGATGCCAGCGGAGCCACCTTTACATGGACC |
| | CCCTCCTCTGGCAAGTCCGCCGTGCAGGGACCACCAGAGCGGGACCTG |
| | TGCGGCTGTTACAGCGTGAGCAGCGTGCTGCCTGGATCCGCCCAGCCCT |
| | GGAACCACGGCGAGACATTCACCTGTACAGCAGCACACCCAGAGCTGA |
| | AGACCCCTCTGACAGCCACCCTGAGCAAGTCTGGCAATACCTTCCGGCC |
| | AGAGGTGCACCTGCTGCCTCCACCAAGCGAGGAGCTGGCCCTGAATGA |
| | GCTGGTGACCCTGACATGCCTGGCCAGGGGCTTCTCTCCTAAGGACGTG |
| | CTGGTGCGCTGGCTGCAGGGCAGCCAGGAGCTGCCTCGGGAGAAGTAT |
| | CTGACCTGGGCCAGCAGACAGGAGCCATCCCAGGGCACCACAACCTTT |
| | GCCGTGACCTCCATCCTGAGGGTGGCAGCAGAGGACTGGAAGAAGGGC |
| | GATACCTTCTCTTGTATGGTGGGACACGAGGCCCTGCCCCTGGCCTTTA |
| | CCCAGAAGACAATCGATCGCCTGGCCGGCAAGCCTACTCATGTGAATG |
| | TCAGTGTCGTGATGGCAGAGGTGGACGGGACTTG |
| 115 | EVQLAESGGDLVQPGGSLRLSCAASGFTFSSYAMSWVRQAPGKGLEWVS |
| | AISGSGGSTYYADSVKGRFTISRDNSKNTLYLQMNSLRAEDTAIYYCARDL |
| | RVYAMEAFDIWGQGTMVTVSSASPTSPKVFPLSLDSTPQDGNVVVACLVQ |
| | GFFPQEPLSVTWSESGQGVTARNFPPSQDASGDLYTTSSQLTLPATQCPDG |
| | KSVTCHVKHYTNSSQDVTVPCRVPELKTPLGDTTHTCPRCPEPKSCDTPPP |
| | CPRCPEPKSCDTPPPCPRCPEPKSCDTPPPCPRCPAPELLGGPPPPCCHPRLSL |
| | HRPALEDLLLGSEANLTCTLTGLRDASGATFTWTPSSGKSAVQGPPERDLC |
| | GCYSVSSVLPGSAQPWNHGETFTCTAAHPELKTPLTATLSKSGNTFRPEVH |
| | LLPPPSEELALNELVTLTCLARGFSPKDVLVRWLQGSQELPREKYLTWASR |
| | QEPSQGTTTFAVTSILRVAAEDWKKGDTFSCMVGHEALPLAFTQKTIDRLA |
| | GKPTHVNVSVVMAEVDGT |
| 116 | GAAGTGCAACTAGCAGAAAGTGGTGGTGATCTCGTGCAGCCTGGCGGA |
| | TCTCTGCGGCTGTCTTGTGCCGCTTCTGGCTTCACCTTCTCCTCCTACGC |
| | CATGTCCTGGGTGCGGCAAGCTCCCGGAAAAGGCCTGGAGTGGGTGTC |
| | CGCTATCTCTGGCTCCGGCGGCTCTACCTACTATGCCGACTCCGTGAAG |
| | GGCAGATTTACCATCTCCCGGGACAACTCCAAGAACACACTGTACCTGC |
| | AGATGAACAGCCTGAGAGCCGAGGACACCGCCATCTACTACTGCGCCA |
| | GAGATCTGAGAGTGTACGCTATGGAAGCCTTCGACATCTGGGGCCAGG |
| | GCACCATGGTGACCGTGTCCTCTGCTTCTCCCACTTCCCCAAAGGTGTTC |
| | CCTCTGTCACTGGACTCAACCCCTCAGGATGGCAATGTCGTCGTCGCAT |
| | GTCTGGTGCAGGGCTTCTTTCCTCAGGAGCCACTGAGCGTGACATGGTC |
| | TGAGAGCGGCCAGGGCGTGACCGCCAGAAATTTCCCCCCTTCCCAGGA |
| | CGCCTCTGGCGATCTGTACACCACAAGCTCCCAGCTGACCCTGCCAGCA |
| | ACACAGTGCCCAGACGGCAAGTCCGTGACCTGTCACGTGAAGCACTAT |
| | ACAAACTCTAGCCAGGATGTGACCGTGCCCTGCCGGGTGCCTGAACTA |
| | AAGACTCCACTAGGTGATACTACTCACACCTGTCCTAGATGCCCTGAAC |
| | CCAAAAGCTGCGACACACCTCCACCTTGTCCTCGGTGTCCTGAGCCTAA |
| | GTCCTGCGATACCCCTCCTCCTTGCCCCCGGTGCCCCGAGCCCAAGTCT |
| | TGCGACACCCCTCCACCCTGCCCTAGATGCCCTGCCCCTGAGCTGCTGG |
| | GCGGCCCTCCACCCCCTTGCTGTCACCCAAGGCTGTCCCTGCACAGGCC |
| | CGCCCTGGAGGACCTGCTGCTGGGCTCTGAGGCCAATCTGACATGTACC |
| | CTGACAGGCCTGCGGGATGCCAGCGGAGCCACCTTTACATGGACCCCCT |
| | CCTCTGGCAAGTCCGCCGTGCAGGGACCACCAGAGCGGGACCTGTGCG |
| | GCTGTTACAGCGTGAGCAGCGTGCTGCCTGGATCCGCCCAGCCCTGGAA |
| | CCACGGCGAGACATTCACCTGTACAGCAGCACACCCAGAGCTGAAGAC |
| | CCCTCTGACAGCCACCCTGAGCAAGTCTGGCAATACCTTCCGGCCAGAG |
| | GTGCACCTGCTGCCTCCACCAAGCGAGGAGCTGGCCCTGAATGAGCTG |
| | GTGACCCTGACATGCCTGGCCAGGGGCTTCTCTCCTAAGGACGTGCTGG |
| | TGCGCTGGCTGCAGGGCAGCCAGGAGCTGCCTCGGGAGAAGTATCTGA |
| | CCTGGGCCAGCAGACAGGAGCCATCCCAGGGCACCACAACCTTTGCCG |
| | TGACCTCCATCCTGAGGGTGGCAGCAGAGGACTGGAAGAAGGGCGATA |
| | CCTTCTCTTGTATGGTGGGACACGAGGCCCTGCCCCTGGCCTTTACCCA |
| | GAAGACAATCGATCGCCTGGCCGGCAAGCCTACTCATGTGAATGTCAG |
| | TGTCGTGATGGCAGAGGTGGACGGGACT |
| 117 | DIVMTQSPDSLAVSLGERATINCRASESVDIYGNSFMHWYQQKPGQPPKLL |
| | IYLASNLESGVPDRFSGSGSGTDFTLTISSLQAEDVAVYYCQQNNEDPYTFG |
| | GGTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC |
| 118 | GATATCGTGATGACTCAAAGTCCAGATAGTCTCGCTGTGTCCCTGGGCG |
| | AGAGAGCCACAATCAACTGCAGAGCTTCTGAGTCCGTGGACATCTATG |
| | GCAACTCCTTCATGCACTGGTACCAGCAGAAACCAGGCCAGCCTCCTAA |
| | GCTGCTGATCTACCTGGCCAGCAATCTGGAATCTGGAGTCCCCGACCGG |
| | TTTTCTGGCTCTGGCTCCGGCACCGACTTCACCCTGACCATCTCCAGCCT |
| | GCAGGCCGAGGACGTGGCCGTGTACTACTGTCAACAGAACAACGAGGA |
| | TCCTTACACCTTCGGCGGCGGAACCAAGGTGGAAATCAAGAGGACCGT |
| | CGCCGCCCCTAGTGTCTTTATCTTCCCCCCTTCCGATGAACAGCTGAAA |
| | AGTGGGACCGCCTCCGTCGTGTGCCTGCTGAACAATTTCTACCCCCGGG |
| | AGGCCAAGGTGCAGTGGAAGGTGGACAACGCCCTGCAGTCTGGCAATA |
| | GCCAGGAGTCCGTGACCGAGCAGGACTCTAAGGATAGCACATATTCCC |
| | TGAGCAGCACCCTGACCCTGTCTAAGGCCGATTACGAGAAGCACAAGG |
| | TGTATGCCTGCGAAGTCACCCATCAGGGGCTGTCCAGTCCAGTCACAAA |
| | GTCATTCAATAGGGGGGAGTGT |
| 119 | QVQLVQSGAEVKKPGASVKVSCKASGYTFTSYNMHWVRQAPGQGLEWM |
| | GVIYSGNGDTSYNQKFKGRVTITADKSTSTAYMELSSLRSEDTAVYYCARE |
| | RDTRFGNWGQGTLVTVSSASPTSPKVFPLSLDSTPQDGNVVVACLVQGFFP |
| | QEPLSVTWSESGQGVTARNFPPSQDASGDLYTTSSQLTLPATQCPDGKSVT |
| | CHVKHYTNSSQDVTVPCRVPELKTPLGDTTHTCPRCPEPKSCDTPPPCPRCP |
| | EPKSCDTPPPCPRCPEPKSCDTPPPCPRCPAPELLGGPPPPPCCHPRLSLHRPA |
| | LEDLLLGSEANLTCTLTGLRDASGATFTWTPSSGKSAVQGPPERDLCGCYS |
| | VSSVLPGSAQPWNHGETFTCTAAHPELKTPLTATLSKSGNTFRPEVHLLPPP |
| | SEELALNELVTLTCLARGFSPKDVLVRWLQGSQELPREKYLTWASRQEPSQ |
| | GTTTFAVTSILRVAAEDWKKGDTFSCMVGHEALPLAFTQKTIDRLAGKPT |
| | HVNVSVVMAEVDGT |
| 120 | CAAGTGCAACTAGTGCAAAGTGGTGCAGAAGTCAAGAAGCCTGGCGCT |
| | TCTGTGAAAGTGTCCTGCAAGGCCTCTGGCTACACCTTCACCTCCTACA |
| | ACATGCACTGGGTGCGGCAGGCTCCCGGACAAGGCCTGGAGTGGATGG |
| | GCGTGATCTACTCCGGAAATGGCGACACATCTTATAACCAGAAGTTCAA |
| | GGGCAGAGTGACCATCACCGCCGACAAGTCCACCTCTACAGCCTACAT |
| | GGAACTGTCCAGCCTGCGGTCCGAGGACACCGCTGTGTACTACTGTGCC |
| | AGAGAGCGGGATACCAGATTTGGCAACTGGGGCCAGGGCACCCTGGTG |
| | ACCGTGTCTTCTGCTTCTCCCACTTCCCCAAAGGTGTTCCCTCTGTCACT |
| | GGACTCAACCCCTCAGGATGGCAATGTCGTCGTCGCATGTCTGGTGCAG |
| | GGCTTCTTTCCTCAGGAGCCACTGAGCGTGACATGGTCTGAGAGCGGCC |
| | AGGGCGTGACCGCCAGAAATTTCCCCCCTTCCCAGGACGCCTCTGGCGA |
| | TCTGTACACCACAAGCTCCCAGCTGACCCTGCCAGCAACACAGTGCCCA |
| | GACGGCAAGTCCGTGACCTGTCACGTGAAGCACTATACAAACTCTAGC |
| | CAGGATGTGACCGTGCCCTGCCGGGTGCCTGAACTAAAGACTCCACTA |
| | GGTGATACTACTCACACCTGTCCTAGATGCCCTGAACCCAAAAGCTGCG |
| | ACACACCTCCACCTTGTCCTCGGTGTCCTGAGCCTAAGTCCTGCGATAC |
| | CCCTCCTCCTTGCCCCCGGTGCCCCGAGCCCAAGTCTTGCGACACCCCT |
| | CCACCCTGCCCTAGATGCCCTGCCCCTGAGCTGCTGGGCGGCCCTCCTC |
| | CACCCCCTTGCTGTCACCCAAGGCTGTCCCTGCACAGGCCCGCCCTGGA |
| | GGACCTGCTGCTGGGCTCTGAGGCCAATCTGACATGTACCCTGACAGGC |
| | CTGCGGGATGCCAGCGGAGCCACCTTTACATGGACCCCCTCCTCTGGCA |
| | AGTCCGCCGTGCAGGGACCACCAGAGCGGGACCTGTGCGGCTGTTACA |
| | GCGTGAGCAGCGTGCTGCCTGGATCCGCCCAGCCCTGGAACCACGGCG |
| | AGACATTCACCTGTACAGCAGCACACCCAGAGCTGAAGACCCCTCTGA |
| | CAGCCACCCTGAGCAAGTCTGGCAATACCTTCCGGCCAGAGGTGCACCT |
| | GCTGCCTCCACCAAGCGAGGAGCTGGCCCTGAATGAGCTGGTGACCCT |
| | GACATGCCTGGCCAGGGGCTTCTCTCCTAAGGACGTGCTGGTGCGCTGG |
| | CTGCAGGGCAGCCAGGAGCTGCCTCGGGAGAAGTATCTGACCTGGGCC |
| | AGCAGACAGGAGCCATCCCAGGGCACCACAACCTTTGCCGTGACCTCC |
| | ATCCTGAGGGTGGCAGCAGAGGACTGGAAGAAGGGCGATACCTTCTCT |
| | TGTATGGTGGGACACGAGGCCCTGCCCCTGGCCTTTACCCAGAAGACA |
| | ATCGATCGCCTGGCCGGCAAGCCTACTCATGTGAATGTCAGTGTCGTGA |
| | TGGCAGAGGTGGACGGGACT |
| 121 | QVQLVQSGAEVKKPGASVKVSCKASGYTFTSYNMHWVRQAPGQGLEWM |
| | GVIYSGNGDTSYNQKFKGRVTITADKSTSTAYMELSSLRSEDTAVYYCARE |
| | RDTRFGNWGQGTLVTVSSASPTSPKVFPLSLDSTPQDGNVVVACLVQGFFP |
| | QEPLSVTWSESGQGVTARNFPPSQDASGDLYTTSSQLTLPATQCPDGKSVT |
| | CHVKHYTNSSQDVTVPCRVPELKTPLGDTTHTCPRCPEPKSCDTPPPCPRCP |
| | EPKSCDTPPPCPRCPEPKSCDTPPPCPRCPAPELLGGPPPPCCHPRLSLHRPA |
| | LEDLLLGSEANLTCTLTGLRDASGATFTWTPSSGKSAVQGPPERDLCGCYS |
| | VSSVLPGSAQPWNHGETFTCTAAHPELKTPLTATLSKSGNTFRPEVHLLPPP |
| | SEELALNELVTLTCLARGFSPKDVLVRWLQGSQELPREKYLTWASRQEPSQ |
| | GTTTFAVTSILRVAAEDWKKGDTFSCMVGHEALPLAFTQKTIDRLAGKPT |
| | HVNVSVVMAEVDGT |
| 122 | CAAGTGCAACTAGTGCAAAGTGGTGCAGAAGTCAAGAAGCCTGGCGCT |
| | TCTGTGAAAGTGTCCTGCAAGGCCTCTGGCTACACCTTCACCTCCTACA |
| | ACATGCACTGGGTGCGGCAGGCTCCCGGACAAGGCCTGGAGTGGATGG |
| | GCGTGATCTACTCCGGAAATGGCGACACATCTTATAACCAGAAGTTCAA |
| | GGGCAGAGTGACCATCACCGCCGACAAGTCCACCTCTACAGCCTACAT |
| | GGAACTGTCCAGCCTGCGGTCCGAGGACACCGCTGTGTACTACTGTGCC |
| | AGAGAGCGGGATACCAGATTTGGCAACTGGGGCCAGGGCACCCTGGTG |
| | ACCGTGTCTTCTGCTTCTCCCACTTCCCCAAAGGTGTTCCCTCTGTCACT |
| | GGACTCAACCCCTCAGGATGGCAATGTCGTCGTCGCATGTCTGGTGCAG |
| | GGCTTCTTTCCTCAGGAGCCACTGAGCGTGACATGGTCTGAGAGCGGCC |
| | AGGGCGTGACCGCCAGAAATTTCCCCCCTTCCCAGGACGCCTCTGGCGA |
| | TCTGTACACCACAAGCTCCCAGCTGACCCTGCCAGCAACACAGTGCCCA |
| | GACGGCAAGTCCGTGACCTGTCACGTGAAGCACTATACAAACTCTAGC |
| | CAGGATGTGACCGTGCCCTGCCGGGTGCCTGAACTAAAGACTCCACTA |
| | GGTGATACTACTCACACCTGTCCTAGATGCCCTGAACCCAAAAGCTGCG |
| | ACACACCTCCACCTTGTCCTCGGTGTCCTGAGCCTAAGTCCTGCGATAC |
| | CCCTCCTCCTTGCCCCCGGTGCCCCGAGCCCAAGTCTTGCGACACCCCT |
| | CCACCCTGCCCTAGATGCCCTGCCCCTGAGCTGCTGGGCGGCCCTCCAC |
| | CCCCTTGCTGTCACCCAAGGCTGTCCCTGCACAGGCCCGCCCTGGAGGA |
| | CCTGCTGCTGGGCTCTGAGGCCAATCTGACATGTACCCTGACAGGCCTG |
| | CGGGATGCCAGCGGAGCCACCTTTACATGGACCCCCTCCTCTGGCAAGT |
| | CCGCCGTGCAGGGACCACCAGAGCGGGACCTGTGCGGCTGTTACAGCG |
| | TGAGCAGCGTGCTGCCTGGATCCGCCCAGCCCTGGAACCACGGCGAGA |
| | CATTCACCTGTACAGCAGCACACCCAGAGCTGAAGACCCCTCTGACAG |
| | CCACCCTGAGCAAGTCTGGCAATACCTTCCGGCCAGAGGTGCACCTGCT |
| | GCCTCCACCAAGCGAGGAGCTGGCCCTGAATGAGCTGGTGACCCTGAC |
| | ATGCCTGGCCAGGGGCTTCTCTCCTAAGGACGTGCTGGTGCGCTGGCTG |
| | CAGGGCAGCCAGGAGCTGCCTCGGGAGAAGTATCTGACCTGGGCCAGC |
| | AGACAGGAGCCATCCCAGGGCACCACAACCTTTGCCGTGACCTCCATCC |
| | TGAGGGTGGCAGCAGAGGACTGGAAGAAGGGCGATACCTTCTCTTGTA |
| | TGGTGGGACACGAGGCCCTGCCCCTGGCCTTTACCCAGAAGACAATCG |
| | ATCGCCTGGCCGGCAAGCCTACTCATGTGAATGTCAGTGTCGTGATGGC |
| | AGAGGTGGACGGGACTT |
| 123 | PCRVP |
| 124 | ELKTPLGDTTHT |
| 125 | CPRCPEPKSCDTPPPCPRCPEPKSCDTPPPCPRCPEPKSCDTPPPCPRCP |
| 126 | APELLGGP |
| 127 | PPPPCCHPR |
| 128 | PPPCCHPR |

1. (canceled)

2. A method of targeting, reducing, depleting and/or ablating a population of cells in the bone marrow of a subject in need of a hematopoietic cell transplantation (HCT), the method comprising administering to the subject an antibody that binds or specifically binds to a target on an endogenous hematopoietic stem cell (HSC) in the subject, wherein the antibody mediates antibody dependent cellular trogocytosis (ADCT).

3. (canceled)

4. (canceled)

5. (canceled)

6. The method of claim 2, wherein the target on the endogenous HSC is selected from the group consisting of CD2, CD5, CD7, CD11a, CDw12, CD13, CD15, CD18, CD19, CD21, CD22, CD29, CD30, CD33, CD34, CD36, CD38, CD40, CD41, CD42a, CD42b, CD42c, CD42d, CD43, CD45, CD45RA, CD45RB, CD45RC, CD45RO, CD48, CD49b, CD49d (VLA-4), CD49e, CD49f (VLA-6), CD50, CD51, CD53, CD55, CD58, CD64a, CD68, CD71, CD72, CD73, CD81, CD82, CD84, CD85A, CD85K, CD90, CD97, CD99, CD104, CD105, CD109, CD110, CD111, CD112, CD114, CD115, CD117 (c-kit), CD123, CD124, CD126, CD127, CD130, CD131, CD133, CD134, CD135, CD137, CD138, CD151, CD157, CD162, CD164, CD166, CD168, CD172a, CD173, CD174, CD175, CD175s, CD176, CD183, CD184 (CXCR4), CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, CD350, CD361 and combinations thereof.

7. The method of claim 2, wherein the target on the endogenous HSC is CD117.

8. The method of claim 7, wherein the antibody is administered in an amount sufficient to:
(i) block binding of CD117 by stem cell factor (SCF); and/or
(ii) inhibit activation of CD117 by SCF; and/or
(iii) inhibit CD117+ cell proliferation mediated by SCF; and/or
(iv) deplete a population of CD117+ cells in the subject.

9. The method of claim 2, wherein the antibody is an anti-CD117 antibody.

10. The method of claim 9, wherein the anti-CD117 antibody binds:
(i) the soluble N-terminal of human CD117 with a K_{D} of 20 nM or less; and/or
(ii) the C-terminal of human CD117 with a K_{D} of 15 nM or less; and/or
(iii) full length human CD117 with a K_{D} of 60 nM or less.

11. The method of claim 9, wherein the anti-CD117 antibody inhibits SCF mediated proliferation of TF-1 cells expressing CD117 with an IC₅₀ of at least 1 nM.

12. The method of claim 9, wherein the anti-CD117 antibody binds to TF-1 cells expressing CD117 with an EC₅₀ of 25 nM or less.

13. The method of claim 9, wherein the anti-CD117 antibody mediates ADCT with a normalised maximum membrane uptake (Bₘₐₓ) of at least 50% as determined by mean fluorescent intensity (MFI) of DILC18 on neutrophils.

14. The method of claim 9, wherein the anti-CD117 antibody competitively inhibits binding of any one or more of the following antibodies to CD117:
(i) an antibody comprising a heavy chain variable region (V_{H}) comprising a sequence set forth in SEQ ID NO: 4 or SEQ ID NO: 13; and a light chain variable region (V_{L}) comprising a sequence set forth in SEQ ID NO: 5 or SEQ ID NO: 28;
(ii) an antibody comprising a V_{H} comprising a sequence set forth in SEQ ID NO: 7; and a V_{L} comprising a sequence set forth in SEQ ID NO: 10;
(iii) an antibody comprising a V_{H} comprising a sequence set forth in SEQ ID NO: 13; and a V_{L} comprising a sequence set forth in SEQ ID NO: 16;
(iv) an antibody comprising a V_{H} comprising a sequence set forth in SEQ ID NO: 19; and a V_{L} comprising a sequence set forth in SEQ ID NO: 22; and
(v) an antibody comprising a V_{H} comprising a sequence set forth in SEQ ID NO: 25; and a V_{L} comprising a sequence set forth in SEQ ID NO: 28.

15. The method of claim 9, wherein the anti-CD117 antibody comprises:
(i) a V_{H} comprising a sequence set forth in SEQ ID NO: 4 or SEQ ID NO: 13; and a V_{L} comprising a sequence set forth in SEQ ID NO: 5 or SEQ ID NO: 28;
(ii) a V_{H} comprising three complementarity determining regions (CDRs) of a V_{H} comprising a sequence set forth in SEQ ID NO: 4 or SEQ ID NO: 13; and a V_{L} comprising three CDRs of a V_{L} comprising a sequence set forth in SEQ ID NO: 5 or SEQ ID NO: 28;
(iii) a V_{H} comprising three CDRs of a V_{H} comprising a sequence set forth in SEQ ID NO: 7; and a V_{L} comprising three CDRs of a V_{L} comprising a sequence set forth in SEQ ID NO: 10;
(iv) a V_{H} comprising three CDRs of a V_{H} comprising a sequence set forth in SEQ ID NO: 13; and a V_{L} comprising three CDRs of a V_{L} comprising a sequence set forth in SEQ ID NO: 16;
(v) a V_{H} comprising three CDRs of a V_{H} comprising a sequence set forth in SEQ ID NO: 19; and a V_{L} comprising three CDRs of a V_{L} comprising a sequence set forth in SEQ ID NO: 22;
(vi) a V_{H} comprising three CDRs of a V_{H} comprising a sequence set forth in SEQ ID NO: 25; and a V_{L} comprising three CDRs of a V_{L} comprising a sequence set forth in SEQ ID NO: 28;
(vii) a V_{H} comprising a CDR1 comprising a sequence set forth in SEQ ID NO: 42, a CDR2 comprising a sequence set forth in SEQ ID NO: 43 and a CDR3 comprising a sequence set forth in SEQ ID NO: 44; and a V_{L} comprising a CDR1 comprising a sequence set forth in SEQ ID NO: 45, a CDR2 comprising a sequence set forth in SEQ ID NO: 46 and a CDR3 comprising a sequence set forth in SEQ ID NO: 47;
(viii) a V_{H} comprising a CDR1 comprising a sequence set forth in SEQ ID NO: 48, a CDR2 comprising a sequence set forth in SEQ ID NO: 49 and a CDR3 comprising a sequence set forth in SEQ ID NO: 50; and a V_{L} comprising a CDR1 comprising a sequence set forth in SEQ ID NO: 51, a CDR2 comprising a sequence set forth in SEQ ID NO: 52 and a CDR3 comprising a sequence set forth in SEQ ID NO: 53;
(ix) a V_{H} comprising a CDR1 comprising a sequence set forth in SEQ ID NO: 54, a CDR2 comprising a sequence set forth in SEQ ID NO: 55 and a CDR3 comprising a sequence set forth in SEQ ID NO: 56; and a V_{L} comprising a CDR1 comprising a sequence set forth in SEQ ID NO: 57, a CDR2 comprising a sequence set forth in SEQ ID NO: 58 and a CDR3 comprising a sequence set forth in SEQ ID NO: 59;
(x) a V_{H} comprising a CDR1 comprising a sequence set forth in SEQ ID NO: 60, a CDR2 comprising a sequence set forth in SEQ ID NO: 61 and a CDR3 comprising a sequence set forth in SEQ ID NO: 62; and a V_{L} comprising a CDR1 comprising a sequence set forth in SEQ ID NO: 63, a CDR2 comprising a sequence set forth in SEQ ID NO: 64 and a CDR3 comprising a sequence set forth in SEQ ID NO: 65;
(xi) a V_{H} comprising a sequence set forth in SEQ ID NO: 7; and a V_{L} comprising a sequence set forth in SEQ ID NO: 10;
(xii) a V_{H} comprising a sequence set forth in SEQ ID NO: 13; and a V_{L} comprising a sequence set forth in SEQ ID NO: 16;
(xiii) a V_{H} comprising a sequence set forth in SEQ ID NO: 19; and a V_{L} comprising a sequence set forth in SEQ ID NO: 22;
(xiv) a V_{H} comprising a sequence set forth in SEQ ID NO: 25; and a V_{L} comprising a sequence set forth in SEQ ID NO: 28;
(xv) a heavy chain comprising a sequence set forth in SEQ ID NO: 6; and a light chain comprising a sequence set forth in SEQ ID NO: 9;
(xvi) a heavy chain comprising a sequence set forth in SEQ ID NO: 12; and a light chain comprising a sequence set forth in SEQ ID NO: 15;
(xvii) a heavy chain comprising a sequence set forth in SEQ ID NO: 18; and a light chain comprising a sequence set forth in SEQ ID NO: 21;
(xviii) a heavy chain comprising a sequence set forth in SEQ ID NO: 24; and a light chain comprising a sequence set forth in SEQ ID NO: 27;
(xix) a heavy chain comprising a sequence set forth in SEQ ID NO: 67; and a light chain comprising a sequence set forth in SEQ ID NO: 69;
(xx) a heavy chain comprising a sequence set forth in SEQ ID NO: 71; and a light chain comprising a sequence set forth in SEQ ID NO: 73;
(xxi) a heavy chain comprising a sequence set forth in SEQ ID NO: 75; and a light chain comprising a sequence set forth in SEQ ID NO: 77;
(xxii) a heavy chain comprising a sequence set forth in SEQ ID NO: 79; and a light chain comprising a sequence set forth in SEQ ID NO: 81;
(xxiii) a heavy chain comprising a sequence set forth in SEQ ID NO: 83; and a light chain comprising a sequence set forth in SEQ ID NO: 85;
(xxiv) a heavy chain comprising a sequence set forth in SEQ ID NO: 87; and a light chain comprising a sequence set forth in SEQ ID NO: 89;
(xxv) a heavy chain comprising a sequence set forth in SEQ ID NO: 91; and a light chain comprising a sequence set forth in SEQ ID NO: 93;
(xxvi) a heavy chain comprising a sequence set forth in SEQ ID NO: 95; and a light chain comprising a sequence set forth in SEQ ID NO: 97;
(xxvii) a heavy chain comprising a sequence set forth in SEQ ID NO: 99; and a light chain comprising a sequence set forth in SEQ ID NO: 101;
(xxviii) a heavy chain comprising a sequence set forth in SEQ ID NO: 103; and a light chain comprising a sequence set forth in SEQ ID NO: 105; or
(xxix) a heavy chain comprising a sequence set forth in SEQ ID NO: 107; and a light chain comprising a sequence set forth in SEQ ID NO: 109.

16. (canceled)

17. (canceled)

18. (canceled)

19. (canceled)

20. The method of claim 2, wherein the subject is suffering from a cancer, a hemoglobinopathy disorder, a myelodysplastic disorder, a primary immune deficiency (PID), an autoimmune disorder, an immunodeficiency disorder, or a metabolic disorder.

21. The method of claim 2, wherein the subject has received, is receiving, or will receive an additional conditioning regimen.

22. The method of claim 21, wherein the additional conditioning regimen is a non-myeloablative or reduced-intensity conditioning regimen.

23. The method of claim 2, wherein the subject further receives a HCT following administration of the antibody.

24. (canceled)

25. (canceled)

26. (canceled)

27. An anti-CD117 antibody, wherein the antibody comprises:
(i) a V_{H} comprising a sequence set forth in SEQ ID NO: 7; and a V_{L} comprising a sequence set forth in SEQ ID NO: 10;
(ii) a V_{H} comprising a sequence set forth in SEQ ID NO: 13; and a V_{L} comprising a sequence set forth in SEQ ID NO: 16;
(iii) a V_{H} comprising a sequence set forth in SEQ ID NO: 19; and a V_{L} comprising a sequence set forth in SEQ ID NO: 22;
(iv) a V_{H} comprising a sequence set forth in SEQ ID NO: 25; and a V_{L} comprising a sequence set forth in SEQ ID NO: 28;
(v) a heavy chain comprising a sequence set forth in SEQ ID NO: 6; and a light chain comprising a sequence set forth in SEQ ID NO: 9;
(vi) a heavy chain comprising a sequence set forth in SEQ ID NO: 12; and a light chain comprising a sequence set forth in SEQ ID NO: 15;
(vii) a heavy chain comprising a sequence set forth in SEQ ID NO: 18; and a light chain comprising a sequence set forth in SEQ ID NO: 21;
(viii) a heavy chain comprising a sequence set forth in SEQ ID NO: 24; and a light chain comprising a sequence set forth in SEQ ID NO: 27;
(ix) a heavy chain comprising a sequence set forth in SEQ ID NO: 67; and a light chain comprising a sequence set forth in SEQ ID NO: 69;
(x) a heavy chain comprising a sequence set forth in SEQ ID NO: 71; and a light chain comprising a sequence set forth in SEQ ID NO: 73;
(xi) a heavy chain comprising a sequence set forth in SEQ ID NO: 75; and a light chain comprising a sequence set forth in SEQ ID NO: 77;
(xii) a heavy chain comprising a sequence set forth in SEQ ID NO: 79; and a light chain comprising a sequence set forth in SEQ ID NO: 81;
(xiii) a heavy chain comprising a sequence set forth in SEQ ID NO: 83; and a light chain comprising a sequence set forth in SEQ ID NO: 85;
(xiv) a heavy chain comprising a sequence set forth in SEQ ID NO: 87; and a light chain comprising a sequence set forth in SEQ ID NO: 89;
(xv) a heavy chain comprising a sequence set forth in SEQ ID NO: 91; and a light chain comprising a sequence set forth in SEQ ID NO: 93;
(xvi) a heavy chain comprising a sequence set forth in SEQ ID NO: 95; and a light chain comprising a sequence set forth in SEQ ID NO: 97;
(xvii) a heavy chain comprising a sequence set forth in SEQ ID NO: 99; and a light chain comprising a sequence set forth in SEQ ID NO: 101;
(xviii) a heavy chain comprising a sequence set forth in SEQ ID NO: 103; and a light chain comprising a sequence set forth in SEQ ID NO: 105;
(xix) a heavy chain comprising a sequence set forth in SEQ ID NO: 107; and a light chain comprising a sequence set forth in SEQ ID NO: 109;
(xx) a heavy chain comprising a sequence expressed from or encoded by a nucleic acid comprising SEQ ID NO: 8; and a light chain comprising a sequence expressed from or encoded by a nucleic acid comprising SEQ ID NO: 11;
(xxi) a heavy chain comprising a sequence expressed from or encoded by a nucleic acid comprising SEQ ID NO: 14; and a light chain comprising a sequence expressed from or encoded by a nucleic acid comprising SEQ ID NO: 17;
(xxii) a heavy chain comprising a sequence expressed from or encoded by a nucleic acid comprising SEQ ID NO: 20; and a light chain comprising a sequence expressed from or encoded by a nucleic acid comprising SEQ ID NO: 23;
(xxiii) a heavy chain comprising a sequence expressed from or encoded by a nucleic acid comprising SEQ ID NO: 26; and a light chain comprising a sequence expressed from or encoded by a nucleic acid comprising SEQ ID NO: 29;
(xxiv) a heavy chain comprising a sequence expressed from or encoded by a nucleic acid comprising SEQ ID NO: 68; and a light chain comprising a sequence expressed from or encoded by a nucleic acid comprising SEQ ID NO: 70;
(xxv) a heavy chain comprising a sequence expressed from or encoded by a nucleic acid comprising SEQ ID NO: 72; and a light chain comprising a sequence expressed from or encoded by a nucleic acid comprising SEQ ID NO: 74;
(xxvi) a heavy chain comprising a sequence expressed from or encoded by a nucleic acid comprising SEQ ID NO: 76; and a light chain comprising a sequence expressed from or encoded by a nucleic acid comprising SEQ ID NO: 78;
(xxvii) a heavy chain comprising a sequence expressed from or encoded by a nucleic acid comprising SEQ ID NO: 80; and a light chain comprising a sequence expressed from or encoded by a nucleic acid comprising SEQ ID NO: 82;
(xxviii) a heavy chain comprising a sequence expressed from or encoded by a nucleic acid comprising SEQ ID NO: 84; and a light chain comprising a sequence expressed from or encoded by a nucleic acid comprising SEQ ID NO: 86;
(xxix) a heavy chain comprising a sequence expressed from or encoded by a nucleic acid comprising SEQ ID NO: 88; and a light chain comprising a sequence expressed from or encoded by a nucleic acid comprising SEQ ID NO: 90;
(xxx) a heavy chain comprising a sequence expressed from or encoded by a nucleic acid comprising SEQ ID NO: 92; and a light chain comprising a sequence expressed from or encoded by a nucleic acid comprising SEQ ID NO: 94;
(xxxi) a heavy chain comprising a sequence expressed from or encoded by a nucleic acid comprising SEQ ID NO: 96; and a light chain comprising a sequence expressed from or encoded by a nucleic acid comprising SEQ ID NO: 98;
(xxxii) a heavy chain comprising a sequence expressed from or encoded by a nucleic acid comprising SEQ ID NO: 100; and a light chain comprising a sequence expressed from or encoded by a nucleic acid comprising SEQ ID NO: 102;
(xxxiii) a heavy chain comprising a sequence expressed from or encoded by a nucleic acid comprising SEQ ID NO: 104; and a light chain comprising a sequence expressed from or encoded by a nucleic acid comprising SEQ ID NO: 106;
(xxxiv) a heavy chain comprising a sequence expressed from or encoded by a nucleic acid comprising SEQ ID NO: 108; and a light chain comprising a sequence expressed from or encoded by a nucleic acid comprising SEQ ID NO: 110;
(xxxv) a V_{H} comprising three complementarity determining regions (CDRs) of a V_{H} comprising a sequence set forth in SEQ ID NO: 4 or SEQ ID NO: 13; and a V_{L} comprising three CDRs of a V_{L} comprising a sequence set forth in SEQ ID NO: 5 or SEQ ID NO: 28;
(xxxvi) a V_{H} comprising three CDRs of a V_{H} comprising a sequence set forth in SEQ ID NO: 7; and a V_{L} comprising three CDRs of a V_{L} comprising a sequence set forth in SEQ ID NO: 10;
(xxxvii) a V_{H} comprising three CDRs of a V_{H} comprising a sequence set forth in SEQ ID NO: 13; and a V_{L} comprising three CDRs of a V_{L} comprising a sequence set forth in SEQ ID NO: 16;
(xxxviii) a V_{H} comprising three CDRs of a V_{H} comprising a sequence set forth in SEQ ID NO: 19; and a V_{L} comprising three CDRs of a V_{L} comprising a sequence set forth in SEQ ID NO: 22;
(xxxix) a V_{H} comprising three CDRs of a V_{H} comprising a sequence set forth in SEQ ID NO: 25; and a V_{L} comprising three CDRs of a V_{L} comprising a sequence set forth in SEQ ID NO: 28;
(xl) a V_{H} comprising a CDR1 comprising a sequence set forth in SEQ ID NO: 42, a CDR2 comprising a sequence set forth in SEQ ID NO: 43 and a CDR3 comprising a sequence set forth in SEQ ID NO: 44; and a V_{L} comprising a CDR1 comprising a sequence set forth in SEQ ID NO: 45, a CDR2 comprising a sequence set forth in SEQ ID NO: 46 and a CDR3 comprising a sequence set forth in SEQ ID NO: 47;
(xli) a V_{H} comprising a CDR1 comprising a sequence set forth in SEQ ID NO: 48, a CDR2 comprising a sequence set forth in SEQ ID NO: 49 and a CDR3 comprising a sequence set forth in SEQ ID NO: 50; and a V_{L} comprising a CDR1 comprising a sequence set forth in SEQ ID NO: 51, a CDR2 comprising a sequence set forth in SEQ ID NO: 52 and a CDR3 comprising a sequence set forth in SEQ ID NO: 53;
(xlii) a V_{H} comprising a CDR1 comprising a sequence set forth in SEQ ID NO: 54, a CDR2 comprising a sequence set forth in SEQ ID NO: 55 and a CDR3 comprising a sequence set forth in SEQ ID NO: 56; and a V_{L} comprising a CDR1 comprising a sequence set forth in SEQ ID NO: 57, a CDR2 comprising a sequence set forth in SEQ ID NO: 58 and a CDR3 comprising a sequence set forth in SEQ ID NO: 59; or
(xliii) a V_{H} comprising a CDR1 comprising a sequence set forth in SEQ ID NO: 60, a CDR2 comprising a sequence set forth in SEQ ID NO: 61 and a CDR3 comprising a sequence set forth in SEQ ID NO: 62; and a V_{L} comprising a CDR1 comprising a sequence set forth in SEQ ID NO: 63, a CDR2 comprising a sequence set forth in SEQ ID NO: 64 and a CDR3 comprising a sequence set forth in SEQ ID NO: 65.

28. (canceled)

29. (canceled)

30. (canceled)

31. A pharmaceutical composition comprising the anti-CD117 antibody of claim 27, and a pharmaceutically acceptable carrier.

32. (canceled)

33. The anti-CD117 antibody of claim 27, wherein the protein is encoded by a polynucleotide, and preferably wherein the polynucleotide is operably linked to a heterologous promoter.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. The anti-CD117 antibody of claim 27, wherein the antibody comprises a modified hinge region.

47. The anti-CD117 antibody of claim 46, wherein the modified hinge region comprises a chimeric IgA₂/IgG₃ hinge region.
